(19) United States
(12) Patent Application Publication
(10) Pub. No.: US 2020/0392161 A1
(43) Pub. Date: Dec. 17, 2020

(54) SHP2 PHOSPHATASE INHIBITORS AND METHODS OF USE THEREOF

(71) Applicants: Relay Therapeutics, Inc., Cambridge, MA (US); D.R. Shaw Research, LLC, New York, NY (US)

(72) Inventors: W. Patrick Walters, Westborough, MA (US); André Lescarbeau, Somerville, MA (US); Elizabeth H. Kelley, Cambridge, MA (US); Kelley C. Shortsleeves, Maynard, MA (US); Alexander M. Taylor, Cambridge, MA (US); Levi Charles Thomas Pierce, Somerville, MA (US); Mark Andrew Murcko, Holliston, MA (US); Fabrizio Giordanetto, New York, NY (US); Jack Benjamin Greisman, New York, NY (US); Paul Maragakis, New York, NY (US); Sathesh Bhat, Jersey City, NJ (US); Kyle Konze, New York, NY (US); Markus Kristofer Dahlgren, Shelton, CT (US); Eric Therrien, Bronx, NY (US)

(21) Appl. No.: 16/971,435

(22) PCT Filed: Feb. 21, 2019
(86) PCT No.: PCT/US2019/018960

§ 371 Date: Aug. 20, 2020

Related U.S. Application Data
(60) Provisional application No. 62/633,221, filed on Feb. 21, 2018.
(60) Provisional application No. 62/633,222, filed on Feb. 21, 2018.
(60) Provisional application No. 62/633,224, filed on Feb. 21, 2018.
(60) Provisional application No. 62/633,226, filed on Feb. 21, 2018.
(60) Provisional application No. 62/633,223, filed on Feb. 21, 2018.
(60) Provisional application No. 62/633,228, filed on Feb. 21, 2018.

(51) Int. Cl. C07D 513/04; C07D 498/04; C07D 487/04; C07D 495/04; C07D 417/04; C07D 471/04; C07D 417/12; C07D 277/56; C07D 417/14; C07D 519/00; A61K 47/68; A61K 31/519; A61K 31/454; A61K 31/433; A61K 31/4545; A61K 31/496; A61K 31/427; A61K 31/426; A61K 31/5377; C07D 417/08; A61K 31/55; A61K 31/424; A61K 45/06

(52) U.S. Cl. CPC C07D 513/04; A61K 45/06; C07D 487/04; C07D 495/04; C07D 417/04; C07D 471/04; C07D 417/12; C07D 277/56; C07D 417/14; C07D 519/00; A61K 47/6803; A61K 31/519; A61K 31/454; A61K 31/433; A61K 31/4545; A61K 31/496; A61K 31/427; A61K 31/426; A61K 31/5377; C07D 417/08; A61K 31/55; A61K 31/424; C07D 498/04

(57) ABSTRACT

The present disclosure relates to novel compounds and pharmaceutical compositions thereof, and methods for inhibiting the activity of SHP2 phosphatase with the compounds and compositions of the disclosure. The present disclosure further relates to, but is not limited to, methods for treating disorders associated with SHP2 deregulation with the compounds and compositions of the disclosure.

### CROSS REFERENCE TO RELATED APPLICATIONS

This application claims the benefit of, and priority to, U.S. provisional application Ser. No. 62/633,221, filed Feb. 21, 2018; 62/633,222, filed Feb. 21, 2018; 62/633,224, filed Feb. 21, 2018; 62/633,226, filed Feb. 21, 2018; 62/633,223, filed Feb. 21, 2018; and 62/633,228, filed Feb. 21, 2018; the contents of each of which are hereby incorporated by reference herein in their entirety.

### BACKGROUND

Src homology region 2 (SH2)-containing protein tyrosine phosphatase 2 (SHP2) is a protein tyrosine phosphatase encoded by the PTPN11 gene. SHP2 contains two Src homology 2 (SH2) NH₂-terminal domains and a C-terminal protein-tyrosine phosphatase domain. It is ubiquitously expressed in various tissues and cell types. SHP2 plays an important role in diverse signaling pathways to regulate cellular biological processes and is involved in the signaling pathways of a variety of growth factors and cytokines. Within a single signaling pathway, SHP2 can play both positive (signal enhancing) and negative (signal diminishing) roles in intracellular signaling processes. SHP2 is believed to function by dephosphorylating its associated signaling molecules, thereby attenuating the local signaling flow. However, the main effect of SHP2 action in most signaling pathways (e.g., growth factor, cytokine, and extracellular matrix receptors) is to enhance signal transduction. For example, SHP2 is a positive regulator of the ERK/MAPK signaling pathway, playing a key role in regulating cellular proliferation and survival. (For a review of SHP2 phosphatase, see, e.g, K. S. Grossman et al., Adv. Cancer Res. 2010, 106, 53-89; and references cited therein.)

In the basal state, SHP2 is normally auto-inhibited due to intramolecular interactions between its N-terminal SH2 (N—SH2) domain and its catalytic (PTP) domain, which blocks access to the catalytic site. Activating proteins that interact with the SH2 domains induce a conformational change that reverses this inhibition and allows substrate access to the catalytic site. Mutations in the PTPN11 gene that affect the N—SH2 or PTP domain residues involved in basal inhibition of SHP2 result in more readily activatable forms of SHP2 protein, which can lead to unregulated or increased SHP2 activity. Such activated mutants of SHP2 have been associated with developmental disorders such as Noonan syndrome, where nearly all mutated forms of SHP2 demonstrate increased PTP activity. Thus, there is a need for SHP2 phosphatase inhibitor compounds and methods for treating cancer and other disorders with these compounds.

### SUMMARY

It is understood that any of the embodiments described below can be combined in any desired way, and that any embodiment or combination of embodiments can be applied to each of the aspects described below, unless the context indicates otherwise.

For example, provided herein is a compound represented by Formula i, ii, or iii:

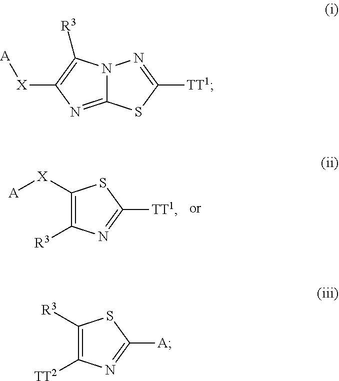

or a pharmaceutically acceptable salt or stereoisomer thereof, wherein:

TT¹ is selected from the group consisting of:

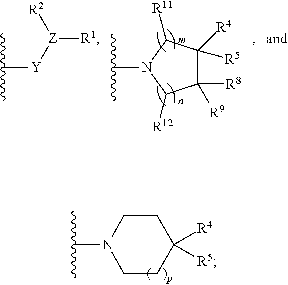

TT² is selected from the group consisting of:

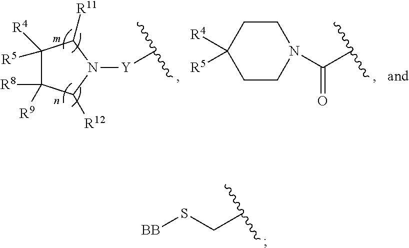

X is selected from the group consisting of a bond, —O—, —NR^{X1}—, —C(O)NR^{X11}—, —S(O)_{w}— (wherein w is 0, 1 or 2), —C(O)—, —C(R^{X2}R^{X3})—, —O—C(R^{X4}R^{X5}), —C(R^{X4}R^{X5})—O—, and —C═C(R^{X6}R^{X7})—;

Y is selected from the group consisting of a bond, —C(O)—, —NR^{Y}—, —C₁₋₃alkylene-NR^{Y}—, and —NR^{Y}—C₁₋₃alkylene-;

Z is selected from the group consisting of N and C(R^{Z});

wherein if Y is a bond or —C(O)—, Z is N; and wherein if Y is —NR^{Y}—, —C₁₋₃alkylene-NR^{Y}—, or —NR^{Y}—C₁₋₃alkylene-, Z is C(R^{Z});

R^{Z} is selected from the group consisting of hydrogen and C₁₋₆alkyl; wherein C₁₋₆alkyl may optionally be substituted by halogen, hydroxyl, and —NR^{a}R^{b},

A is selected from the group consisting of a 6-10 membered monocyclic or bicyclic aryl, a 5-7 membered monocyclic heteroaryl having one or more heteroatoms each independently selected from O, S, or N, a 8-10 membered bicyclic heteroaryl having one or more heteroatoms each independently selected from O, S, or N; and a 4-7 membered heterocyclyl, wherein A may optionally be substituted by one, two, three or more substituents each independently selected from the group consisting of —R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R¹⁰ is independently selected from the group consisting of hydrogen, C₁₋₆alkyl, C₁₋₆haloalkyl, C₃₋₆cycloalkyl, heterocycloalkyl, aryl, and heteroaryl; wherein R¹⁰ may optionally be substituted by one, two, three or more substituents each independently selected from the group consisting of halogen, —C(O)R²⁰, C₁₋₆alkyl and C₁₋₆haloalkyl;

R²⁰ is selected from the group consisting of hydroxyl, halogen, and C₁₋₆alkyl;

R¹ and R² are each selected from the group consisting of hydrogen and C₁₋₁₀alkyl; wherein C₁₋₁₀alkyl may optionally be substituted by one, two, three or more substituents each independently selected from R^{P};

or R¹ and R², together with carbon or nitrogen to which they are attached, form a 3-7 membered saturated carbocyclic or 4-7 membered saturated heterocyclic ring B having one or two heteroatoms each independently selected from the group consisting of 0, S(O), (wherein w is 0, 1, or 2), and NR^{h}; wherein carbocyclic or heterocyclic ring B may be monocyclic or bicyclic;

carbocyclic or heterocyclic ring B may optionally be substituted on one or more available carbons by one, two, three or more substituents each independently selected from the group consisting of halogen, hydroxyl, cyano, oxo, —NR^{a}R^{b}, —CO₂H, —C(O)—NR^{a}R^{b}, —S(O)₂—NR^{a}R^{b}, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})—, C₁₋₆alkoxycarbonyl-N(R^{a})—, aryl and heteroaryl; wherein C₁₋₆alkyl, C₂₋₆alkenyl, C₂₋₆alkynyl, C₃₋₆cycloalkyl, C₁₋₆alkoxy, C₃₋₆ alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})— and C₁₋₆ alkoxycarbonyl-N(R^{a})— may optionally be substituted by one or more substituents each independently selected from R^{P}; and wherein aryl and heteroaryl may optionally be substituted by one or more substituents each independently selected from R^{f};

R^{X1} is selected from the group consisting of hydrogen, C₁₋₆alkyl and phenyl, or R^{X1} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X11} is selected from the group consisting of hydrogen, C₁₋₆alkyl and phenyl, or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X2} and R^{X3} are each independently selected from the group consisting of hydrogen, halogen, hydroxyl, —NR^{a}R^{b}, —C(O)—NR^{a}R^{b}, C₁₋₆alkyl and C₁₋₆alkoxy; wherein C₁₋₆alkyl and C₁₋₆alkoxy may optionally be substituted by one or more substituents each independently selected from R^{P};

or R^{X2} and R^{X3} together with the carbon to which they are attached form a 3-6 membered carbocycle optionally substituted by one or more substituents each independently selected from the group consisting of hydrogen, halogen, —NR^{a}R^{b}, —C(O)—NR^{a}R^{b}, oxo, C₁₋₆ alkyl and C₁₋₆alkoxy;

R^{X4} and R^{X5} are each independently selected from the group consisting of hydrogen and C₁₋₆alkyl; wherein C₁₋₆alkyl may optionally be substituted by one or more substituents each independently selected from R^{P};

R^{X6} and R^{X7} are each independently selected from the group consisting of hydrogen, halogen, —C(O)—NR^{a}R^{b}, cyano and C₁₋₆alkyl; wherein C₁₋₆alkyl may optionally be substituted by one or more substituents each independently selected from R^{P};

R^{Y} is selected from the group consisting of hydrogen, C₁₋₆alkyl and phenyl;

R³ is selected from the group consisting of hydrogen, halogen, hydroxyl, C₁₋₆alkyl and —NR^{a}R^{b};

R⁴ and R⁵ are each independently selected from the group consisting of hydrogen, hydroxyl, C₁₋₆alkyl, C₁₋₆alkoxy, C₁₋₆alkyl-O—R⁶, —C(O)N(R⁶)₂, —N(R⁶)₂, halogen, C₁₋₆alkyl-N(R⁶)₂, and cyano; wherein C₁₋₆alkyl, C₁₋₆alkoxy, C₁₋₆alkyl-O—R⁶, and C₁₋₆alkyl-N(R⁶)₂ may optionally be substituted by one or more substituents each independently selected from the group consisting of hydroxyl, —NH₂, oxo, and halogen,

or R⁴ and R⁵, taken together with the atoms to which they are attached, form a 3-7 membered carbocyclic or heterocyclic saturated or partially unsaturated ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;

or R⁴ and R⁸, taken together with the atoms to which they are attached, form a 4-7 membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;

or R⁴ is absent, and R⁴ and R⁸, taken together with the atoms to which they are attached, form a 3-membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;

R⁶ is independently for each occurrence selected from the group consisting of hydrogen, C₁₋₆alkyl and phenyl;

R¹¹ and R¹² are each independently selected from the group consisting of hydrogen, hydroxyl, C₁₋₆alkyl, C₁₋₆alkoxy, C₁₋₆alkyl-O—R⁶, aryl, arylalkylene, heteroaryl, heteroarylalkylene, —C(O)N(R⁶)₂, —N(R⁶)₂, halogen, C₁₋₆alkyl-N(R⁶)₂, —CO₂H, and cyano; wherein C₁₋₆alkyl, C₁₋₆alkoxy, C₁₋₆alkyl-O—R⁶, aryl, arylalkylene, heteroaryl, heteroarylalkylene, and C₁₋₆alkyl-N(R⁶)₂ may optionally be substituted by one or more substituents each independently selected from the group consisting of hydroxyl, —NH₂, and halogen;

or R¹¹ and R¹², taken together with the atoms to which they are attached, form a 5-7 membered heterocyclic ring;

or R⁴ and R¹², taken together with the atoms to which they are attached, form a 5-7 membered carbocyclic or heterocyclic ring;

or R⁸ and R¹¹, taken together with the atoms to which they are attached, form a 5-7 membered carbocyclic or heterocyclic ring;

R⁸ and R⁹ are each independently selected from the group consisting of hydrogen, C₁₋₆)alkyl, C₁₋₆alkyl-N(R⁶)₂, —OR⁶, C₁₋₆alkyl-O—R⁶, —C(O)NH₂, —N(R⁶)₂, halogen, and cyano;

each of m and n is, independently, 0, 1, 2, or 3, with m+n being at least 2 and no more than 4;

q is 0 or 1;

R^{f} is independently selected, for each occurrence, from the group consisting of R^{P}, C₁₋₆ alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, (wherein wherein w is 0, 1 or 2), C₁₋₆alkylcarbonyl-N(R^{a})— and C₁₋₆alkoxycarbonyl-N(R^{a})—; wherein C₁₋₆alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, C₁₋₆alkylcarbonyl-N(R^{a})—, and C₁₋₆alkoxycarbonyl-N(R^{a})— may be optionally substituted by one or more substituents selected from R^{P};

R^{h} is independently selected, for each occurrence, from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆ alkylcarbonyl-, C₁₋₆alkoxycarbonyl-, R^{a}R^{b}N-carbonyl- and R^{a}R^{b}N—SO₂—; wherein C₁₋₆alkyl, C₃₋₆alkenyl, C₃₋₆alkynyl, C₃₋₆cycloalkyl, C₁₋₆alkyl-S(O)₂—, C₁₋₆alkylcarbonyl- and C₁₋₆ alkoxycarbonyl- may optionally be substituted by one or more substituents selected from R^{P};

R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl;

or R^{a} and R^{b}, together with the nitrogen to which they are attached, may form a 4-6 membered monocyclic heterocyclic ring, which may have an additional heteroatom selected from the group consisting of O, S, and N; wherein the 4-6 membered heterocyclic ring may optionally be substituted by one or more substituents selected from the group consisting of halogen, cyano, oxo or hydroxyl;

BB is optionally substituted monocyclic or bicyclic heteroaryl; and

R^{P} is independently selected, for each occurrence, from the group consisting of halogen, hydroxyl, cyano, C₁₋₆alkoxy, R^{a}R^{b}N—, R^{a}R^{b}N-carbonyl-, R^{a}R^{b}N—SO₂—, and R^{a}R^{b}N-carbonyl-N(R^{a})—.

In an embodiment, the present disclosure provides a compound of Formula I, or a pharmaceutically acceptable salt or stereoisomer thereof, wherein Formula I is represented by:

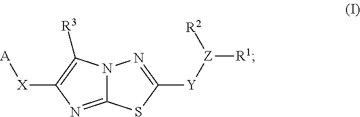

wherein

X is selected from the group consisting of a bond, —O—, —NR^{X1}—, —C(O)NR^{X11}—, —S(O)_{w}— (wherein w is 0, 1 or 2), —C(O)—, —C(R^{X2}R^{X3})—, —O—C(R^{X4}R^{X5}), —C(R^{X4}R^{X5})—O—, and —C═C(R^{X6}R^{X7})—;

Y is selected from the group consisting of a bond, —C(O)—, —NR^{Y}—, —C₁₋₃alkylene-NR^{Y}—, and —NR^{Y}—C₁₋₃alkylene-;

Z is selected from the group consisting of N and C(R^{Z});

wherein if Y is a bond or —C(O)—, Z is N; and wherein if Y is —NR^{Y}—, —C₁₋₃alkylene-NR^{Y}—, or —NR^{Y}—C₁₋₃alkylene-, Z is C(R^{Z});

R^{Z} is selected from the group consisting of hydrogen and C₁₋₆alkyl; wherein C₁₋₆alkyl may optionally be substituted by halogen, hydroxyl, and —NR^{a}R^{b},

A is selected from the group consisting of a 6-10 membered monocyclic or bicyclic aryl, a 5-7 membered monocyclic heteroaryl having one or more heteroatoms each independently selected from O, S, or N, a 8-10 membered bicyclic heteroaryl having one or more heteroatoms each independently selected from O, S, or N; and a 4-7 membered heterocyclyl, wherein A may optionally be substituted by one, two, three or more substituents each independently selected from the group consisting of —R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R¹⁰ is independently selected from the group consisting of hydrogen, C₁₋₆alkyl, C₁₋₆haloalkyl, C₃₋₆cycloalkyl, heterocycloalkyl, aryl, and heteroaryl; wherein R¹⁰ may optionally be substituted by one, two, three or more substituents each independently selected from the group consisting of halogen, —C(O)R²⁰, C₁₋₆alkyl and C₁₋₆haloalkyl;

R²⁰ is selected from the group consisting of hydroxyl, halogen, and C₁₋₆alkyl;

R¹ and R² are each selected from the group consisting of hydrogen and C₁₋₁₀alkyl; wherein C₁₋₁₀alkyl may optionally be substituted by one, two, three or more substituents each independently selected from R^{P};

or R¹ and R², together with carbon or nitrogen to which they are attached, form a 3-7 membered saturated carbocyclic or 4-7 membered saturated heterocyclic ring B having one or two heteroatoms each independently selected from the group consisting of 0, S(O), (wherein w is 0, 1, or 2), and NR^{h}; wherein carbocyclic or heterocyclic ring B may be monocyclic or bicyclic;

carbocyclic or heterocyclic ring B may optionally be substituted on one or more available carbons by one, two, three or more substituents each independently selected from the group consisting of halogen, hydroxyl, cyano, oxo, —NR^{a}R^{b}, —CO₂H, —C(O)—NR^{a}R^{b}, —S(O)₂—NR^{a}R^{b}, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})—, C₁₋₆alkoxycarbonyl-N(R^{a})—, aryl and heteroaryl; wherein C₁₋₆alkyl, C₂₋₆alkenyl, C₂₋₆alkynyl, C₃₋₆cycloalkyl, C₁₋₆alkoxy, C₃₋₆ alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})— and C₁₋₆alkoxycarbonyl-N(R^{a})— may optionally be substituted by one or more substituents each independently selected from R^{P}; and wherein aryl and heteroaryl may optionally be substituted by one or more substituents each independently selected from R^{f};

R^{X1} is selected from the group consisting of hydrogen, C₁₋₆alkyl and phenyl, or R^{X1} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X11} is selected from the group consisting of hydrogen, C₁₋₆alkyl and phenyl, or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X2} and R^{X3} are each independently selected from the group consisting of hydrogen, halogen, hydroxyl, —NR^{a}R^{b}, —C(O)—NR^{a}R^{b}, C₁₋₆alkyl and C₁₋₆alkoxy; wherein C₁₋₆alkyl and C₁₋₆alkoxy may optionally be substituted by one or more substituents each independently selected from R^{P};

or R^{X2} and R^{X3} together with the carbon to which they are attached form a 3-6 membered carbocycle optionally substituted by one or more substituents each independently selected from the group consisting of hydrogen, halogen, —NR^{a}R^{b}, —C(O)—NR^{a}R^{b}, oxo, C₁₋₆ alkyl and C₁₋₆alkoxy;

R^{X4} and R^{X5} are each independently selected from the group consisting of hydrogen and C₁₋₆alkyl; wherein C₁₋₆alkyl may optionally be substituted by one or more substituents each independently selected from R^{P};

R^{X6} and R^{X7} are each independently selected from the group consisting of hydrogen, halogen, —C(O)—NR^{a}R^{b}, cyano and C₁₋₆alkyl; wherein C₁₋₆alkyl may optionally be substituted by one or more substituents each independently selected from R^{P};

R^{Y} is selected from the group consisting of hydrogen, C₁₋₆alkyl and phenyl;

R³ is selected from the group consisting of hydrogen, halogen, hydroxyl, C₁₋₆alkyl and —NR^{a}R^{b};

R^{f} is independently selected, for each occurrence, from the group consisting of R^{P}, C₁₋₆ alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, (wherein wherein w is 0, 1 or 2), C₁₋₆alkylcarbonyl-N(R^{a})— and C₁₋₆alkoxycarbonyl-N(R^{a})—; wherein C₁₋₆alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, C₁₋₆alkylcarbonyl-N(R^{a})—, and C₁₋₆alkoxycarbonyl-N(R^{a})— may be optionally substituted by one or more substituents selected from R^{P};

R^{h} is independently selected, for each occurrence, from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆ alkylcarbonyl-, C₁₋₆alkoxycarbonyl-, R^{a}R^{b}N-carbonyl- and R^{a}R^{b}N—SO₂—; wherein C₁₋₆alkyl, C₃₋₆alkenyl, C₃₋₆alkynyl, C₃₋₆cycloalkyl, C₁₋₆alkyl-S(O)₂—, C₁₋₆alkylcarbonyl- and C₁₋₆ alkoxycarbonyl- may optionally be substituted by one or more substituents selected from R^{P};

R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl;

or R^{a} and R^{b}, together with the nitrogen to which they are attached, may form a 4-6 membered monocyclic heterocyclic ring, which may have an additional heteroatom selected from the group consisting of O, S, and N; wherein the 4-6 membered heterocyclic ring may optionally be substituted by one or more substituents selected from the group consisting of halogen, cyano, oxo or hydroxyl; and

R^{P} is independently selected, for each occurrence, from the group consisting of halogen, hydroxyl, cyano, C₁₋₆alkoxy, R^{a}R^{b}N—, R^{a}R^{b}N-carbonyl-, R^{a}R^{b}N—SO₂—, and R^{a}R^{b}N-carbonyl-N(R^{a})—.

Also provided herein is a compound of Formula IV, or a pharmaceutically acceptable salt or stereoisomer thereof, wherein Formula IV is represented by:

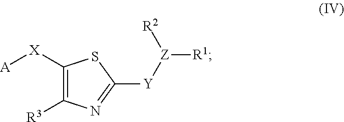

wherein

X is selected from the group consisting of a bond, —O—, —NR^{X1}—, —C(O)NR^{X11}—, —S(O)_{w}— (wherein w is 0, 1 or 2), —C(O)—, —C(R^{X2}R^{X3})—, —O—C(R^{X4}R^{X5}), —C(R^{X4}R^{X5})—O—, and —C═C(R^{X6}R^{X7})—;

Y is selected from the group consisting of a bond, —C(O)—, —NR^{Y}—, —C₁₋₃alkylene-NR^{Y}—, and —NR^{Y}—C₁₋₃alkylene-;

Z is selected from the group consisting of N and C(R^{Z});

wherein if Y is a bond or —C(O)—, Z is N; and wherein if Y is —NR^{Y}—, —C₁₋₃alkylene-NR^{Y}—, or —NR^{Y}—C₁₋₃alkylene-, Z is C(R^{Z});

R^{Z} is selected from the group consisting of hydrogen and C₁₋₆alkyl; wherein C₁₋₆alkyl may optionally be substituted by halogen, hydroxyl, and —NR^{a}R^{b},

A is selected from the group consisting of a 6-10 membered monocyclic or bicyclic aryl, a 5-7 membered monocyclic heteroaryl having one or more heteroatoms each independently selected from O, S, or N, a 8-10 membered bicyclic heteroaryl having one or more heteroatoms each independently selected from O, S, or N; and a 4-7 membered heterocyclyl, wherein A may optionally be substituted by one, two, three or more substituents each independently selected from the group consisting of —R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R¹⁰ is independently selected from the group consisting of hydrogen, C₁₋₆alkyl, C₁₋₆haloalkyl, C₃₋₆cycloalkyl, heterocycloalkyl, aryl, and heteroaryl; wherein R¹⁰ may optionally be substituted by one, two, three or more substituents each independently selected from the group consisting of halogen, —C(O)R²⁰, C₁₋₆alkyl and C₁₋₆haloalkyl;

R²⁰ is selected from the group consisting of hydroxyl, halogen, and C₁₋₆alkyl;

R¹ and R² are each selected from the group consisting of hydrogen and C₁₋₁₀alkyl; wherein C₁₋₁₀alkyl may optionally be substituted by one, two, three or more substituents each independently selected from R^{P};

or R¹ and R², together with carbon or nitrogen to which they are attached, form a 3-7 membered saturated carbocyclic or 4-7 membered saturated heterocyclic ring B having one or two heteroatoms each independently selected from the group consisting of 0, S(O), (wherein w is 0, 1, or 2), and NR^{h}; wherein carbocyclic or heterocyclic ring B may be monocyclic or bicyclic;

carbocyclic or heterocyclic ring B may optionally be substituted on one or more available carbons by one, two, three or more substituents each independently selected from the group consisting of halogen, hydroxyl, cyano, oxo, —NR^{a}R^{b}, —CO₂H, —C(O)—NR^{a}R^{b}, —S(O)₂—NR^{a}R^{b}, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})—, C₁₋₆alkoxycarbonyl-N(R^{a})—, aryl and heteroaryl; wherein C₁₋₆alkyl, C₂₋₆alkenyl, C₂₋₆alkynyl, C₃₋₆cycloalkyl, C₁₋₆alkoxy, C₃₋₆ alkenyloxy, C₃₋₆alkynyloxy, C₃₋₆cycloalkoxy, C₁₋₆alkylcarbonyl-, C₁₋₆alkoxycarbonyl, C₁₋₆ alkyl-S(O)_{w}— (wherein w is 0, 1 or 2), C₁₋₆alkylcarbonyl-N(R^{a})— and C₁₋₆alkoxycarbonyl-N(R^{a})— may optionally be substituted by one or more substituents each independently selected from R^{P}; and wherein aryl and heteroaryl may optionally be substituted by one or more substituents each independently selected from R^{f};

R^{X1} is selected from the group consisting of hydrogen, C₁₋₆alkyl and phenyl, or R^{X1} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X11} is selected from the group consisting of hydrogen, C₁₋₆alkyl and phenyl, or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X2} and R^{X3} are each independently selected from the group consisting of hydrogen, halogen, hydroxyl, —NR^{a}R^{b}, —C(O)—NR^{a}R^{b}, C₁₋₆alkyl and C₁₋₆alkoxy; wherein C₁₋₆alkyl and C₁₋₆alkoxy may optionally be substituted by one or more substituents each independently selected from R^{P};

or R^{X2} and R^{X3} together with the carbon to which they are attached form a 3-6 membered carbocycle optionally substituted by one or more substituents each independently selected from the group consisting of hydrogen, halogen, —NR^{a}R^{b}, —C(O)—NR^{a}R^{b}, oxo, C₁₋₆ alkyl and C₁₋₆alkoxy;

R^{X4} and R^{X5} are each independently selected from the group consisting of hydrogen and C₁₋₆alkyl; wherein C₁₋₆alkyl may optionally be substituted by one or more substituents each independently selected from R^{P};

R^{X6} and R^{X7} are each independently selected from the group consisting of hydrogen, halogen, —C(O)—NR^{a}R^{b}, cyano and C₁₋₆alkyl; wherein C₁₋₆alkyl may optionally be substituted by one or more substituents each independently selected from R^{P};

R^{Y} is selected from the group consisting of hydrogen, C₁₋₆alkyl and phenyl;

R³ is selected from the group consisting of hydrogen, halogen, hydroxyl, C₁₋₆alkyl and —NR^{a}R^{b};

R^{f} is independently selected, for each occurrence, from the group consisting of R^{P}, C₁₋₆ alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, (wherein wherein w is 0, 1 or 2), C₁₋₆alkylcarbonyl-N(R^{a})— and C₁₋₆alkoxycarbonyl-N(R^{a})—; wherein C₁₋₆alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, C₁₋₆alkylcarbonyl-N(R^{a})—, and C₁₋₆alkoxycarbonyl-N(R^{a})— may be optionally substituted by one or more substituents selected from R^{P};

R^{h} is independently selected, for each occurrence, from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆ alkylcarbonyl-, C₁₋₆alkoxycarbonyl-, R^{a}R^{b}N-carbonyl- and R^{a}R^{b}N—SO₂—; wherein C₁₋₆alkyl, C₃₋₆alkenyl, C₃₋₆alkynyl, C₃₋₆cycloalkyl, C₁₋₆alkyl-S(O)₂—, C₁₋₆alkylcarbonyl- and C₁₋₆ alkoxycarbonyl- may optionally be substituted by one or more substituents selected from R^{P};

R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl;

or R^{a} and R^{b}, together with the nitrogen to which they are attached, may form a 4-6 membered monocyclic heterocyclic ring, which may have an additional heteroatom selected from the group consisting of O, S, and N; wherein the 4-6 membered heterocyclic ring may optionally be substituted by one or more substituents selected from the group consisting of halogen, cyano, oxo or hydroxyl; and

R^{P} is independently selected, for each occurrence, from the group consisting of halogen, hydroxyl, cyano, C₁₋₆alkoxy, R^{a}R^{b}N—, R^{a}R^{b}N-carbonyl-, R^{a}R^{b}N—SO₂—, and R^{a}R^{b}N-carbonyl-N(R^{a})—.

Also disclosed herein is a method of treating a disorder in a patient in need thereof, comprising administering to the patient an effective amount of a compound of Formula XII, or a pharmaceutically acceptable salt, stereoisomer or N-oxide thereof, wherein Formula XII is represented by:

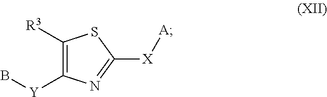

wherein

X is selected from the group consisting of a bond, —O—, —NR^{X1}—, —C(O)NR^{X11}—, —S(O)_{w}— (wherein w is 0, 1 or 2), —C(O)—, —C(R^{X4}R^{X3})—, —O—C(R^{X4}R^{X5}), —C(R^{X4}R^{X5})—O—, and —C═C(R^{X6}R^{X7})—;

A is selected from the group consisting of a 6-10 membered monocyclic or bicyclic aryl, a 5-7 membered monocyclic heteroaryl having one or more heteroatoms each independently selected from O, S, or N, a 8-10 membered bicyclic heteroaryl having one or more heteroatoms each independently selected from O, S, or N; and a 4-7 membered heterocyclyl, wherein A may optionally be substituted by one, two, three or more substituents each independently selected from the group consisting of —R¹⁰, —OR¹⁰, methylenedioxy, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R¹⁰ is independently selected from the group consisting of hydrogen, C₁₋₆alkyl, C₁₋₆haloalkyl, C₃₋₆cycloalkyl, heterocycloalkyl, aryl, and heteroaryl; wherein R¹⁰ may optionally be substituted by one, two, three or more substituents each independently selected from the group consisting of halogen, —C(O)R²⁰, C₁₋₆alkyl and C₁₋₆haloalkyl;

R²⁰ is selected from the group consisting of hydroxyl, halogen, and C₁₋₆alkyl;

Y is selected from the group consisting of C₁₋₆alkylene and C₁₋₆alkenylene; wherein C₁₋₆alkylene and C₁₋₆alkenylene may optionally be substituted by one or two substituents each independently selected from the group consisting of halogen and hydroxyl; and wherein one or two methylene units of L may optionally and independently be replaced by a moiety selected from the group consisting of a bond, —O—, —C(O)—, —O—C(O)—, —C(O)—O—, —NR^{a}—, —C(O)—NR^{a}—, —NR^{a}—C(O)—, —O—C(O)—NR^{a}, R^{a}—C(O)—O—, —S(O)_{w}— (wherein w is 0, 1, or 2), —S(O)_{w}—NR^{a}—, and —NR^{a}—S(O)_{w}—;

B is selected from the group consisting of aryl, heteroaryl, heterocyclyl, and hydrogen; wherein aryl and heteroaryl may optionally be substituted by one, two, three or more substituents each independently selected from R^{e}; and wherein heterocyclyl is bound to L through a ring carbon or ring nitrogen and may optionally be substituted by one, two, three or more substituents each independently selected from R^{g}; and wherein if said heterocyclyl contains a —NH moiety that nitrogen may optionally be substituted by R^{h};

R^{X1} is selected from the group consisting of hydrogen, C₁₋₆alkyl and phenyl; or R and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X11} is selected from the group consisting of hydrogen, C₁₋₆alkyl and phenyl; or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X2} and R^{X3} are each independently selected from the group consisting of hydrogen, halogen, hydroxyl, —NR^{a}R^{b}, —C(O)—NR^{a}R^{b}, C₁₋₆alkyl and C₁₋₆alkoxy; wherein C₁₋₆alkyl and C₁₋₆alkoxy may optionally be substituted by one or more substituents each independently selected from R^{P};

or R^{X2} and R^{X3} together with the carbon to which they are attached form a 3-6 membered carbocycle optionally substituted by one or more substituents each independently selected from the group consisting of hydrogen, halogen, —NR^{a}R^{b}, —C(O)—NR^{a}R^{b}, oxo, C₁₋₆ alkyl and C₁₋₆alkoxy;

R^{X4} and R^{X5} are each independently selected from the group consisting of hydrogen and C₁₋₆alkyl; wherein C₁₋₆alkyl may optionally be substituted by one or more substituents each independently selected from R^{P};

R^{X6} and R^{X7} are each independently selected from the group consisting of hydrogen, halogen, —C(O)—NR^{a}R^{b}, cyano and C₁₋₆alkyl; wherein C₁₋₆alkyl may optionally be substituted by one or more substituents each independently selected from R^{P};

R³ is selected from the group consisting of hydrogen, halogen, hydroxyl, C₁₋₆alkyl and —NR^{a}R^{b};

R^{e} is independently selected, for each occurrence, from the group consisting of halogen, hydroxyl, cyano, oxo, thio, —NR^{a}R^{b}, —CO₂H, —C(O)—NR^{a}R^{b}, —S(O)₂—NR^{a}R^{b}, C₁₋₆ alkyl, C₂₋₆alkenyl, C₂₋₆alkynyl, C₃₋₆cycloalkyl, C₁₋₆alkoxy, —S—C₁₋₆alkyl, —S—C₂₋₆alkenyl, C₁₋₆ alkyl-O—C₁₋₆alkyl, C₃₋₆alkenyloxy, C₃₋₆alkynyloxy, C₃₋₆cycloalkoxy, C₁₋₆alkylcarbonyl-, C₁₋₆ alkoxycarbonyl, C₁₋₆alkyl-S(O)_{w}— (wherein w is 0, 1 or 2), C₁₋₆alkylcarbonyl-N(R^{a})—, C₁₋₆ alkoxycarbonyl-N(R^{a})—, aryl and heteroaryl; wherein C₁₋₆alkyl, C₂₋₆alkenyl, C₂₋₆alkynyl, C₃₋₆ cycloalkyl, C₁₋₆alkoxy, —S—C₁₋₆alkyl, —S—C₂₋₆alkenyl, C₁₋₆alkyl-O—C₁₋₆alkyl, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})— and C₁₋₆alkoxycarbonyl-N(R^{a})— may optionally be substituted by one or more substituents each independently selected from R^{P}; and wherein aryl and heteroaryl may optionally be substituted by one or more substituents each independently selected from R^{f};

R^{f} is independently selected, for each occurrence, from the group consisting of R^{P}, C₁₋₆ alkyl, C₃₋₆cycloalkyl, C₂₋₆alkenyl, C₂₋₆alkynyl, C₁₋₆alkoxy, C₁₋₆ alkylcarbonyl-, C₁₋₆alkoxycarbonyl, C₁₋₆alkyl-S(O)_{w}—, (wherein w is 0, 1 or 2), C₁₋₆alkylcarbonyl-N(R^{a})— and C₁₋₆ alkoxycarbonyl-N(R^{a})—; wherein C₁₋₆alkyl, C₃₋₆cycloalkyl, C₂₋₆alkenyl, C₂₋₆alkynyl, C₁₋₆ alkoxy, C₁₋₆alkylcarbonyl-, C₁₋₆alkoxycarbonyl, C₁₋₆ alkyl-S(O)_{w}—, C₁₋₆alkylcarbonyl-N(R^{a})—, and C₁₋₆alkoxycarbonyl-N(R^{a})— may be optionally substituted by one or more substituents selected from R^{P};

R^{g} is independently selected, for each occurrence, from the group consisting of halogen, hydroxyl, cyano, oxo, thio, —NR^{a}R^{b}, —CO₂H, —C(O)—NR^{a}R^{b}, —S(O)₂—NR^{a}R^{b}, C₁₋₆ alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})—, C₁₋₆alkoxycarbonyl-N(R^{a})—, aryl and heteroaryl; wherein C₁₋₆ alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})— and C₁₋₆alkoxycarbonyl-N(R^{a})— may optionally be substituted by one or more substituents each independently selected from R^{P}; and wherein aryl and heteroaryl may optionally be substituted by one or more substituents each independently selected from R^{f};

R^{h} is independently selected, for each occurrence, from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆ alkylcarbonyl-, C₁₋₆alkoxycarbonyl-, R^{a}R^{b}N-carbonyl-, R^{a}R^{b}N—SO₂— and aryl; wherein C₁₋₆ alkyl, C₃₋₆alkenyl, C₃₋₆alkynyl, C₃₋₆cycloalkyl, C₁₋₆alkyl-S(O)₂—, C₁₋₆alkylcarbonyl- and C₁₋₆ alkoxycarbonyl- may optionally be substituted by one or more substituents selected from R^{P}; and wherein aryl may optionally be substituted by one or more substituents each independently selected from R^{f};

R^{a} and R^{b} are independently selected, for each occurrence, from the group consisting of hydrogen, C₁₋₆alkyl, C₃₋₆cycloalkyl, and phenyl; wherein C₁₋₆alkyl may optionally be substituted by one or more substituents selected from halogen, cyano, oxo, heterocyclyl and hydroxyl; and wherein phenyl may optionally be substituted by C₁₋₃alkyl, C₁₋₃alkoxy, halogen, hydroxyl;

or R^{a} and R^{b}, together with the nitrogen to which they are attached, may form a 4-6 membered monocyclic heterocyclic ring, which may have an additional heteroatom selected from the group consisting of O, S, and N; wherein the 4-6 membered heterocyclic ring may optionally be substituted by one or more substituents selected from the group consisting of halogen, cyano, oxo, hydroxyl and C₁₋₃alkyl (optionally substituted with —NH₂); and

R^{P} is independently selected, for each occurrence, from the group consisting of halogen, hydroxyl, cyano, C₁₋₆alkoxy, R^{a}R^{b}N—, R^{a}R^{b}N-carbonyl-, R^{a}R^{b}N—SO₂—, and R^{a}R^{b}N-carbonyl-N(R^{a})—.

Also disclosed herein is a compound of Formula XIII, or a pharmaceutically acceptable salt or stereoisomer thereof, wherein Formula XIII is represented by:

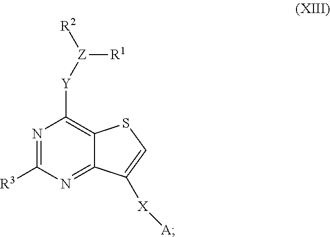

wherein

X is selected from the group consisting of a bond, —O—, —NR^{X1}—, —C(O)NR^{X11}—, —S(O)_{w}— (wherein w is 0, 1 or 2), —C(O)—, —C(R^{X2}R^{X3})—, —O—C(R^{X4}R^{X5}), —C(R^{X4}R^{X5})—O—, and —C═C(R^{X6}R^{X7})—;

Y is selected from the group consisting of a bond, —NR^{Y}—, —C₁₋₃alkylene-NR^{Y}—, and —NR^{Y}—C₁₋₃alkylene-;

Z is selected from the group consisting of N, C(R^{Z}) and a bond;

wherein if Y is a bond, Z is N or a bond; and wherein if Y is —NR^{Y}—, —C₁₋₃alkylene-NR^{Y}—, or —NR^{Y}—C₁₋₃alkylene-, Z is C(R^{Z});

R^{Z} is selected from the group consisting of hydrogen and C₁₋₆alkyl; wherein C₁₋₆alkyl may optionally be substituted by halogen, hydroxyl, and —NR^{a}R^{b},

A is selected from the group consisting of a 6-10 membered monocyclic or bicyclic aryl, a 5-7 membered monocyclic heteroaryl having one or more heteroatoms each independently selected from O, S, or N, a 8-10 membered bicyclic heteroaryl having one or more heteroatoms each independently selected from O, S, or N; and a 4-7 membered heterocyclyl, wherein A may optionally be substituted by one, two, three or more substituents each independently selected from the group consisting of —R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano, or when X is a bond, A may also be selected from the group consisting of H and halogen;

R¹⁰ is independently selected from the group consisting of hydrogen, C₁₋₆alkyl, C₁₋₆haloalkyl, C₃₋₆cycloalkyl, heterocycloalkyl, aryl, and heteroaryl; wherein R¹⁰ may optionally be substituted by one, two, three or more substituents each independently selected from the group consisting of halogen, —C(O)R²⁰, C₁₋₆alkyl and C₁₋₆haloalkyl;

R²⁰ is selected from the group consisting of hydroxyl, halogen, and C₁₋₆alkyl;

R¹ and R² are each selected from the group consisting of hydrogen, C₁₋₁₀alkyl and phenyl; wherein C₁₋₁₀alkyl and phenyl may optionally be substituted by one, two, three or more substituents each independently selected from R^{P}, and wherein when Z and Y are each a bond R² is not present;

or R¹ and R², together with carbon or nitrogen to which they are attached, form a 3-7 membered saturated carbocyclic or 4-7 membered saturated heterocyclic ring B having one or two heteroatoms each independently selected from the group consisting of O, S(O), (wherein w is 0, 1, or 2), and NR^{h}; wherein carbocyclic or heterocyclic ring B may be monocyclic or bicyclic;

carbocyclic or heterocyclic ring B may optionally be substituted on one or more available carbons by one, two, three or more substituents each independently selected from the group consisting of halogen, hydroxyl, cyano, oxo, —NR^{a}R^{b}, —CO₂H, —C(O)—NR^{a}R^{b}, —S(O)₂—NR^{a}R^{b}, C₁₋₆alkyl, C₂₋₆alkenyl, C₂₋₆alkynyl, C₃₋₆cycloalkyl, C₁₋₆ alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})—, C₁₋₆alkoxycarbonyl-N(R^{a})—, aryl and heteroaryl; wherein C₁₋₆alkyl, C₂₋₆alkenyl, C₂₋₆alkynyl, C₃₋₆cycloalkyl, C₁₋₆alkoxy, C₃₋₆ alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})— and C₁₋₆alkoxycarbonyl-N(R^{a})— may optionally be substituted by one or more substituents each independently selected from R^{P}; and wherein aryl and heteroaryl may optionally be substituted by one or more substituents each independently selected from R^{f};

R^{X1} is selected from the group consisting of hydrogen, C₁₋₆alkyl and phenyl, or R^{X1} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X11} is selected from the group consisting of hydrogen, C₁₋₆alkyl and phenyl, or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X2} and R^{X3} are each independently selected from the group consisting of hydrogen, halogen, hydroxyl, —NR^{a}R^{b}, —C(O)—NR^{a}R^{b}, C₁₋₆alkyl and C₁₋₆alkoxy; wherein C₁₋₆alkyl and C₁₋₆alkoxy may optionally be substituted by one or more substituents each independently selected from R^{P};

or R^{X2} and R^{X3} together with the carbon to which they are attached form a 3-6 membered carbocycle optionally substituted by one or more substituents each independently selected from the group consisting of hydrogen, halogen, —NR^{a}R^{b}, —C(O)—NR^{a}R^{b}, oxo, C₁₋₆ alkyl and C₁₋₆alkoxy;

R^{X4} and R^{X5} are each independently selected from the group consisting of hydrogen and C₁₋₆alkyl; wherein C₁₋₆alkyl may optionally be substituted by one or more substituents each independently selected from R^{P};

R^{X6} and R^{X7} are each independently selected from the group consisting of hydrogen, halogen, —C(O)—NR^{a}R^{b}, cyano and C₁₋₆alkyl; wherein C₁₋₆alkyl may optionally be substituted by one or more substituents each independently selected from R^{P};

R^{Y} is selected from the group consisting of hydrogen, C₁₋₆alkyl and phenyl;

R³ is selected from the group consisting of hydrogen, halogen, hydroxyl, C₁₋₆alkyl and —NR^{a}R^{b};

R^{f} is independently selected, for each occurrence, from the group consisting of R^{P}, C₁₋₆ alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, (wherein wherein w is 0, 1 or 2), C₁₋₆alkylcarbonyl-N(R^{a})— and C₁₋₆alkoxycarbonyl-N(R^{a})—; wherein C₁₋₆alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, C₁₋₆alkylcarbonyl-N(R^{a})—, and C₁₋₆alkoxycarbonyl-N(R^{a})— may be optionally substituted by one or more substituents selected from R^{P};

R^{h} is independently selected, for each occurrence, from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆ alkylcarbonyl-, C₁₋₆alkoxycarbonyl-, R^{a}R^{b}N-carbonyl- and R^{a}R^{b}N—SO₂—; wherein C₁₋₆alkyl, C₃₋₆alkenyl, C₃₋₆alkynyl, C₃₋₆cycloalkyl, C₁₋₆alkyl-S(O)₂—, C₁₋₆alkylcarbonyl- and C₁₋₆ alkoxycarbonyl- may optionally be substituted by one or more substituents selected from R^{P};

R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl;

or R^{a} and R^{b}, together with the nitrogen to which they are attached, may form a 4-6 membered monocyclic heterocyclic ring, which may have an additional heteroatom selected from the group consisting of O, S, and N; wherein the 4-6 membered heterocyclic ring may optionally be substituted by one or more substituents selected from the group consisting of halogen, cyano, oxo or hydroxyl; and

R^{P} is independently selected, for each occurrence, from the group consisting of halogen, hydroxyl, cyano, C₁₋₆alkoxy, R^{a}R^{b}N—, R^{a}R^{b}N-carbonyl-, R^{a}R^{b}N—SO₂—, and R^{a}R^{b}N-carbonyl-N(R^{a})—.

Further disclosed herein is a compound of Formula IX, or a pharmaceutically acceptable salt or stereoisomer thereof, wherein Formula IX is represented by:

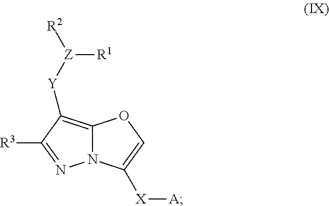

wherein

X is selected from the group consisting of a bond, —O—, —NR^{X1}—, —C(O)NR^{X11}—, —S(O)_{w}— (wherein w is 0, 1 or 2), —C(O)—, —C(R^{X2}R^{X3})—, —O—C(R^{X4}R^{X5}), —C(R^{X4}R^{X5})—O—, and —C═C(R^{X6}R^{X7})—;

Y is selected from the group consisting of a bond, —NR^{Y}—, —C₁₋₃alkylene-NR^{Y}—, and —NR^{Y}—C₁₋₃alkylene-;

Z is selected from the group consisting of N and C(R^{Z}), or Z and R¹ are absent;

wherein if Y is a bond, Z is N or absent; and wherein if Y is —NR^{Y}—, —C₁₋₃alkylene-NR^{Y}—, or —NR^{Y}—C₁₋₃alkylene-, Z is C(R^{Z});

R^{Z} is selected from the group consisting of hydrogen and C₁₋₆alkyl; wherein C₁₋₆alkyl may optionally be substituted by halogen, hydroxyl, and —NR^{a}R^{b},

A is selected from the group consisting of a 6-10 membered monocyclic or bicyclic aryl, a 5-7 membered monocyclic heteroaryl having one or more heteroatoms each independently selected from O, S, or N, a 8-10 membered bicyclic heteroaryl having one or more heteroatoms each independently selected from O, S, or N; and a 4-7 membered heterocyclyl, wherein A may optionally be substituted by one, two, three or more substituents each independently selected from the group consisting of —R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R¹⁰ is independently selected from the group consisting of hydrogen, C₁₋₆alkyl, C₁₋₆haloalkyl, C₃₋₆cycloalkyl, heterocycloalkyl, aryl, and heteroaryl; wherein R¹⁰ may optionally be substituted by one, two, three or more substituents each independently selected from the group consisting of halogen, —C(O)R²⁰, C₁₋₆alkyl and C₁₋₆haloalkyl;

R²⁰ is selected from the group consisting of hydroxyl, halogen, and C₁₋₆alkyl;

R¹ and R² are each selected from the group consisting of hydrogen and C₁₋₁₀alkyl; wherein C₁₋₁₀alkyl may optionally be substituted by one, two, three or more substituents each independently selected from R^{P};

or R¹ and R², together with carbon or nitrogen to which they are attached, form a 3-7 membered saturated carbocyclic or 4-7 membered saturated heterocyclic ring B having one or two heteroatoms each independently selected from the group consisting of 0, S(O), (wherein w is 0, 1, or 2), and NR^{h}; wherein carbocyclic or heterocyclic ring B may be monocyclic or bicyclic;

carbocyclic or heterocyclic ring B may optionally be substituted on one or more available carbons by one, two, three or more substituents each independently selected from the group consisting of halogen, hydroxyl, cyano, oxo, —NR^{a}R^{b}, —CO₂H, —C(O)—NR^{a}R^{b}, —S(O)₂—NR^{a}R^{b}, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})—, C₁₋₆alkoxycarbonyl-N(R^{a})—, aryl and heteroaryl; wherein C₁₋₆alkyl, C₂₋₆alkenyl, C₂₋₆alkynyl, C₃₋₆cycloalkyl, C₁₋₆alkoxy, C₃₋₆ alkenyloxy, C₃₋₆alkynyloxy, C₃₋₆cycloalkoxy, C₁₋₆alkylcarbonyl-, C₁₋₆alkoxycarbonyl, C₁₋₆ alkyl-S(O)_{w}— (wherein w is 0, 1 or 2), C₁₋₆alkylcarbonyl-N(R^{a})— and C₁₋₆alkoxycarbonyl-N(R^{a})— may optionally be substituted by one or more substituents each independently selected from R^{P}; and wherein aryl and heteroaryl may optionally be substituted by one or more substituents each independently selected from R^{f};

R^{X1} is selected from the group consisting of hydrogen, C₁₋₆alkyl and phenyl, or R^{X1} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X11} is selected from the group consisting of hydrogen, C₁₋₆alkyl and phenyl, or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X2} and R^{X3} are each independently selected from the group consisting of hydrogen, halogen, hydroxyl, —NR^{a}R^{b}, —C(O)—NR^{a}R^{b}, C₁₋₆alkyl and C₁₋₆alkoxy; wherein C₁₋₆alkyl and C₁₋₆alkoxy may optionally be substituted by one or more substituents each independently selected from R^{P};

or R^{X2} and R^{X3} together with the carbon to which they are attached form a 3-6 membered carbocycle optionally substituted by one or more substituents each independently selected from the group consisting of hydrogen, halogen, —NR^{a}R^{b}, —C(O)—NR^{a}R^{b}, oxo, C₁₋₆ alkyl and C₁₋₆alkoxy;

R^{X4} and R^{X5} are each independently selected from the group consisting of hydrogen and C₁₋₆alkyl; wherein C₁₋₆alkyl may optionally be substituted by one or more substituents each independently selected from R^{P};

R^{X6} and R^{X7} are each independently selected from the group consisting of hydrogen, halogen, —C(O)—NR^{a}R^{b}, cyano and C₁₋₆alkyl; wherein C₁₋₆alkyl may optionally be substituted by one or more substituents each independently selected from R^{P};

R^{Y} is selected from the group consisting of hydrogen, C₁₋₆alkyl and phenyl;

R³ is selected from the group consisting of hydrogen, halogen, hydroxyl, C₁₋₆ alkyl and —NR^{a}R^{b};

R is independently selected, for each occurrence, from the group consisting of R^{P}, C₁₋₆ alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, (wherein wherein w is 0, 1 or 2), C₁₋₆alkylcarbonyl-N(R^{a})— and C₁₋₆alkoxycarbonyl-N(R^{a})—; wherein C₁₋₆alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, C₁₋₆alkylcarbonyl-N(R^{a})—, and C₁₋₆alkoxycarbonyl-N(R^{a})— may be optionally substituted by one or more substituents selected from R^{P};

R^{h} is independently selected, for each occurrence, from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆ alkylcarbonyl-, C₁₋₆alkoxycarbonyl-, R^{a}R^{b}N-carbonyl- and R^{a}R^{b}N—SO₂—; wherein C₁₋₆alkyl, C₃₋₆alkenyl, C₃₋₆alkynyl, C₃₋₆cycloalkyl, C₁₋₆alkyl-S(O)₂—, C₁₋₆alkylcarbonyl- and C₁₋₆ alkoxycarbonyl- may optionally be substituted by one or more substituents selected from R^{P};

R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl;

or R^{a} and R^{b}, together with the nitrogen to which they are attached, may form a 4-6 membered monocyclic heterocyclic ring, which may have an additional heteroatom selected from the group consisting of O, S, and N; wherein the 4-6 membered heterocyclic ring may optionally be substituted by one or more substituents selected from the group consisting of halogen, cyano, oxo or hydroxyl; and

R^{P} is independently selected, for each occurrence, from the group consisting of halogen, hydroxyl, cyano, C₁₋₆alkoxy, R^{a}R^{b}N—, R^{a}R^{b}N-carbonyl-, R^{a}R^{b}N—SO₂—, and R^{a}R^{b}N-carbonyl-N(R^{a})—.

Further disclosed herein a compound of Formula X, or a pharmaceutically acceptable salt or stereoisomer thereof, wherein Formula X is represented by:

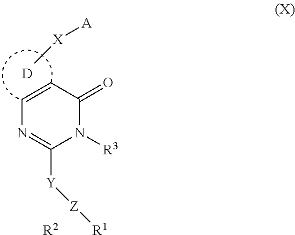

wherein

Ring D is absent or together with carbons to which it is attached, is a 5 membered heteroaryl having one or two heteroatoms each selected from S, O, N, or NR^{a};

X is bound to a free carbon on the pyrimidinone when D is absent, or on a free carbon of ring D, and selected from the group consisting of a bond, —O—, —NR^{X1}—, —C(O)NR^{X11}—, —S(O)_{w}— (wherein w is 0, 1 or 2), —C(O)—, —C(R^{X2}R^{X3})—, —O—C(R^{X4}R^{X5}), —C(R^{X4}R^{X5})—O—, and —C═C(R^{X6}R^{X7})—;

Y is selected from the group consisting of a bond, —NR^{Y}—, —C₁₋₃alkylene-NR^{Y}—, and —NR^{Y}—C₁₋₃alkylene-;

Z is selected from the group consisting of N and C(R^{Z}), or Z and R¹ are absent;

wherein if Y is a bond, Z is N or is absent, and wherein if Y is —NR^{Y}—, —C₁₋₃alkylene-NR^{Y}—, or —NR^{Y}—C₁₋₃alkylene-, Z is C(R^{Z});

R^{Z} is selected from the group consisting of hydrogen and C₁₋₆alkyl; wherein C₁₋₆alkyl may optionally be substituted by halogen, hydroxyl, and —NR^{a}R^{b},

A is selected from the group consisting of a 6-10 membered monocyclic or bicyclic aryl, a 5-7 membered monocyclic heteroaryl having one or more heteroatoms each independently selected from O, S, or N, a 8-10 membered bicyclic heteroaryl having one or more heteroatoms each independently selected from O, S, or N; and a 4-7 membered heterocyclyl, wherein A may optionally be substituted by one, two, three or more substituents each independently selected from the group consisting of —R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R¹⁰ is independently selected from the group consisting of hydrogen, C₁₋₆alkyl, C₁₋₆haloalkyl, C₃₋₆cycloalkyl, heterocycloalkyl, aryl, and heteroaryl; wherein R¹⁰ may optionally be substituted by one, two, three or more substituents each independently selected from the group consisting of halogen, —C(O)R²⁰, C₁₋₆alkyl and C₁₋₆haloalkyl;

R²⁰ is selected from the group consisting of hydroxyl, halogen, and C₁₋₆alkyl;

R¹ and R² are each selected from the group consisting of hydrogen and C₁₋₁₀alkyl; wherein C₁₋₁₀alkyl may optionally be substituted by one, two, three or more substituents each independently selected from R^{P};

or R¹ and R², together with carbon or nitrogen to which they are attached, form a 3-7 membered saturated carbocyclic or 4-7 membered saturated heterocyclic ring B having one or two heteroatoms each independently selected from the group consisting of 0, S(O), (wherein w is 0, 1, or 2), and NR^{h}; wherein carbocyclic or heterocyclic ring B may be monocyclic or bicyclic;

carbocyclic or heterocyclic ring B may optionally be substituted on one or more available carbons by one, two, three or more substituents each independently selected from the group consisting of halogen, hydroxyl, cyano, oxo, —NR^{a}R^{b}, —CO₂H, —C(O)—NR^{a}R^{b}, —S(O)₂—NR^{a}R^{b}, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})—, C₁₋₆alkoxycarbonyl-N(R^{a})—, aryl and heteroaryl; wherein C₁₋₆alkyl, C₂₋₆alkenyl, C₂₋₆alkynyl, C₃₋₆cycloalkyl, C₁₋₆alkoxy, C₃₋₆ alkenyloxy, C₃₋₆alkynyloxy, C₃₋₆cycloalkoxy, C₁₋₆alkylcarbonyl-, C₁₋₆alkoxycarbonyl, C₁₋₆ alkyl-S(O)_{w}— (wherein w is 0, 1 or 2), C₁₋₆alkylcarbonyl-N(R^{a})— and C₁₋₆alkoxycarbonyl-N(R^{a})— may optionally be substituted by one or more substituents each independently selected from R^{P}; and wherein aryl and heteroaryl may optionally be substituted by one or more substituents each independently selected from R^{f};

R^{X1} is selected from the group consisting of hydrogen, C₁₋₆alkyl and phenyl, or R^{X1} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X11} is selected from the group consisting of hydrogen, C₁₋₆alkyl and phenyl, or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X2} and R^{X3} are each independently selected from the group consisting of hydrogen, halogen, hydroxyl, —NR^{a}R^{b}, —C(O)—NR^{a}R^{b}, C₁₋₆alkyl and C₁₋₆alkoxy; wherein C₁₋₆alkyl and C₁₋₆alkoxy may optionally be substituted by one or more substituents each independently selected from R^{P};

or R^{X2} and R^{X3} together with the carbon to which they are attached form a 3-6 membered carbocycle optionally substituted by one or more substituents each independently selected from the group consisting of hydrogen, halogen, —NR^{a}R^{b}, —C(O)—NR^{a}R^{b}, oxo, C₁₋₆ alkyl and C₁₋₆alkoxy;

R^{X4} and R^{X5} are each independently selected from the group consisting of hydrogen and C₁₋₆alkyl; wherein C₁₋₆alkyl may optionally be substituted by one or more substituents each independently selected from R^{P};

R^{X6} and R^{X7} are each independently selected from the group consisting of hydrogen, halogen, —C(O)—NR^{a}R^{b}, cyano and C₁₋₆alkyl; wherein C₁₋₆alkyl may optionally be substituted by one or more substituents each independently selected from R^{P};

R^{Y} is selected from the group consisting of hydrogen, C₁₋₆alkyl and phenyl;

R³ is selected from the group consisting of hydrogen, C₁₋₆alkyl, C₃₋₆alkyl, phenyl, and heterocycle;

R^{f} is independently selected, for each occurrence, from the group consisting of R^{P}, C₁₋₆ alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, (wherein wherein w is 0, 1 or 2), C₁₋₆alkylcarbonyl-N(R^{a})— and C₁₋₆alkoxycarbonyl-N(R^{a})—; wherein C₁₋₆alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, C₁₋₆alkylcarbonyl-N(R^{a})—, and C₁₋₆alkoxycarbonyl-N(R^{a})— may be optionally substituted by one or more substituents selected from R^{P};

R^{h} is independently selected, for each occurrence, from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆ alkylcarbonyl-, C₁₋₆alkoxycarbonyl-, R^{a}R^{b}N-carbonyl- and R^{a}R^{b}N—SO₂—; wherein C₁₋₆alkyl, C₃₋₆alkenyl, C₃₋₆alkynyl, C₃₋₆cycloalkyl, C₁₋₆alkyl-S(O)₂—, C₁₋₆alkylcarbonyl- and C₁₋₆ alkoxycarbonyl- may optionally be substituted by one or more substituents selected from R^{P};

R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl;

or R^{a} and R^{b}, together with the nitrogen to which they are attached, may form a 4-6 membered monocyclic heterocyclic ring, which may have an additional heteroatom selected from the group consisting of O, S, and N; wherein the 4-6 membered heterocyclic ring may optionally be substituted by one or more substituents selected from the group consisting of halogen, cyano, oxo or hydroxyl; and

R^{P} is independently selected, for each occurrence, from the group consisting of halogen, hydroxyl, cyano, C₁₋₆alkoxy, R^{a}R^{b}N—, R^{a}R^{b}N-carbonyl-, R^{a}R^{b}N—SO₂—, and R^{a}R^{b}N-carbonyl-N(R^{a})—.

The disclosure also provides, in an embodiment, pharmaceutical compositions that include one or more compounds described herein. Further, the disclosure provides a method of inhibiting SHP2 phosphatase activity in a subject by administering a therapeutically effective amount of a compound or composition described herein, to a subject, e.g., a human, in need.

The disclosure further provides a method of treating a disorder in a subject by administering a therapeutically effective amount of a compound or composition described herein, to a subject in need thereof. Examples of disorders include Noonan syndrome, neutropenia, diabetes, neuroblastoma, melanoma, acute myeloid leukemia, juvenile leukemia, juvenile myelomonocytic leukemia, breast cancer, lung cancer, and colorectal cancer. In addition to the compound or composition described herein, such method may include administration of a therapeutically effective amount of an antibody, an antibody-drug conjugate, an immunomodulator, or a histone deacetylase inhibitor.

The present disclosure is based, in part, on certain discoveries which are described more fully in the Examples section of the present application. For example, the present disclosure is based, in part, on the discovery of disclosed compounds and the SHP2 phosphatase inhibition exhibited by such compounds.

These and other embodiments of the disclosure are further described in the following sections of the application, including the Detailed Description, Examples, and Claims. Still other objects and advantages of the disclosure will become apparent by those of skill in the art from the disclosure herein, which are simply illustrative and not restrictive. Thus, other embodiments will be recognized by the ordinarily skilled artisan without departing from the spirit and scope of the disclosure.

### DETAILED DESCRIPTION

The compounds and/or compositions of the disclosure, alone or in combination with other treatments, may be effective in treating, reducing, and/or suppressing disorders related to SHP2 phosphatase activity such as, e.g., Noonan syndrome, Leopard Syndrome, diabetes, neuroblastoma, melanoma, juvenile leukemia, juvenile myelomonocytic leukemia (JMML), chronic myelomonocytic leukemia, acute myeloid leukemia, HER2-positive breast cancer, triple-negative breast cancer, ductal carcinoma of the breast, invasive ductal carcinoma of the breast, non-small cell lung cancer (including adenocarcinoma of the lung), colorectal cancer, esophageal cancer, gastric cancer, squamous-cell carcinoma of the head and neck (SCCHN), neutropenia (Kostmann's syndrome), and systemic lupus erythematosus.

In some embodiments, methods described herein may also include additionally administering a therapeutically effective amount of an antibody, an antibody-drug conjugate, an immunomodulator, or a histone deacetylase inhibitor.

### Abbreviations and Definitions

The term “compound of the disclosure” as used herein means a compound described herein. The term is also intended to encompass salts, hydrates, tautomers, stereoisomers, and isotopic substitutions thereof.

The term “composition(s) of the disclosure” as used herein means compositions comprising a disclosed compound, and salts thereof. The compositions of the disclosure may further comprise other agents such as, e.g., excipients, stabilants, lubricants, solvents, and the like.

The term “isomer” as used herein refers to a compound having the identical chemical formula but different structural or optical configurations. The term “stereoisomer” as used herein refers to and includes isomeric molecules that have the same molecular formula but differ in positioning of atoms and/or functional groups in the space. All stereoisomers of the present compounds (e.g., those which may exist due to asymmetric carbons on various substituents), including enantiomeric forms and diastereomeric forms, are contemplated within the scope of the disclosure.

The term “tautomer” as used herein refers to one of two or more structural isomers which exist in equilibrium and which are readily converted from one isomeric form to another. It is understood that tautomers encompass valence tautomers and proton tautomers (also known as prototropic tautomers). Valence tautomers include interconversions by reorganization of some of the bonding electrons. Proton tautomers include interconversions via migration of a proton, such as keto-enol and imine-enamine isomerizations.

The term “isotopic substitution” as used herein refers to the substitution of an atom with its isotope. The term “isotope” as used herein refers to an atom having the same atomic number as that of atoms dominant in nature but having a mass number (neutron number) different from the mass number of the atoms dominant in nature. It is understood that a compound with an isotopic substitution refers to a compound in which at least one atom contained therein is substituted with its isotope. Atoms that can be substituted with its isotope include, but are not limited to, hydrogen, carbon, and oxygen. Examples of the isotope of a hydrogen atom include ²H (also represented as D) and ³H. Examples of the isotope of a carbon atom include ¹³C and ¹⁴C. Examples of the isotope of an oxygen atom include ¹⁸O.

The term “alkyl”, as used herein, unless otherwise indicated, refers to a monovalent aliphatic hydrocarbon radical having a straight chain, branched chain, monocyclic moiety, or polycyclic moiety or combinations thereof, wherein the radical is optionally substituted at one or more carbons of the straight chain, branched chain, monocyclic moiety, or polycyclic moiety or combinations thereof with one or more substituents at each carbon, wherein the one or more substituents are independently C₁-C₁₀ alkyl. Examples of “alkyl” groups include methyl, ethyl, propyl, isopropyl, butyl, iso-butyl, sec-butyl, tert-butyl, pentyl, hexyl, heptyl, cyclopropyl, cyclobutyl, cyclopentyl, cyclohexyl, cycloheptyl, norbornyl, and the like.

The terms “heteroaryl” or “heteroaromatic group” as used herein refers to a monocyclic aromatic 5-6 membered ring system containing one or more heteroatoms, for example one to three heteroatoms, such as nitrogen, oxygen, and sulfur, or a 8-10 membered bicyclic unsaturated or partially unsaturated ring system containing one or more heteroatoms, for example one to three heteroatoms, such as nitrogen, oxygen, and sulfur. Where possible, said heteroaryl ring may be linked to the adjacent radical though carbon or nitrogen. Examples of heteroaryl rings include but are not limited to furan, thiophene, pyrrole, thiazole, oxazole, isothiazole, isoxazole, imidazole, pyrazole, triazole, pyridine or pyrimidine, tetrahydroquinoline, etc.

The terms “heterocyclyl” or “heterocyclic group” are art-recognized and refer to saturated 4-10 membered monocyclic and bicyclic ring structures, including bridged or fused rings, and whose ring structures include one to three heteroatoms, such as nitrogen, oxygen, and sulfur. Where possible, heterocyclyl rings may be linked to the adjacent radical through carbon or nitrogen.

The term “pharmaceutically acceptable salt” is intended to include salts derived from inorganic or organic acids including, e.g., hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluroacetic, trichloroacetic, naphthalene-2 sulfonic and other acids; and salts derived from inorganic or organic bases including, e.g., sodium, potassium, calcium, magnesium, zinc, ammonia, lysine, arginine, histidine, polyhydroxylated amines or tetrafluoroborate. Exemplary pharmaceutically acceptable salts are found, e.g., in Berge, et al. (J. Pharm. Sci. 1977, 66(1), 1; and Gould, P. L., Int. J. Pharmaceutics 1986, 33, 201-217; (each hereby incorporated by reference in its entirety). Pharmaceutically acceptable salts are also intended to encompass hemi-salts, wherein the ratio of compound:acid is respectively 2:1. Exemplary hemi-salts are those salts derived from acids comprising two carboxylic acid groups, such as malic acid, fumaric acid, maleic acid, succinic acid, tartaric acid, glutaric acid, oxalic acid, adipic acid and citric acid. Other exemplary hemi-salts are those salts derived from diprotic mineral acids such as sulfuric acid. Exemplary preferred hemi-salts include, but are not limited to, hemimaleate, hemifumarate, and hemisuccinate.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

An “effective amount”, “sufficient amount” or “therapeutically effective amount” as used herein is an amount of a compound that is sufficient to effect beneficial or desired results, including clinical results. As such, the effective amount may be sufficient, e.g., to reduce or ameliorate the severity and/or duration of afflictions related to SHP2 phosphatase, or one or more symptoms thereof, prevent the advancement of conditions or symptoms related to afflictions related to SHP2 phosphatase, or enhance or otherwise improve the prophylactic or therapeutic effect(s) of another therapy. An effective amount also includes the amount of the compound that avoids or substantially attenuates undesirable side effects.

As used herein and as well understood in the art, “treatment” is an approach for obtaining beneficial or desired results, including clinical results. Beneficial or desired clinical results may include, but are not limited to, alleviation or amelioration of one or more symptoms or conditions, diminution of extent of disease or affliction, a stabilized (i.e., not worsening) state of disease or affliction, preventing spread of disease or affliction, delay or slowing of disease or affliction progression, amelioration or palliation of the disease or affliction state and remission (whether partial or total), whether detectable or undetectable. “Treatment” can also mean prolonging survival as compared to expected survival if not receiving treatment.

The phrase “in need thereof” refers to the need for symptomatic or asymptomatic relief from conditions related to SHP2 phosphatase activity or that may otherwise be relieved by the compounds and/or compositions of the disclosure.

For example, provided herein is a compound represented by Formula i, ii, or iii:

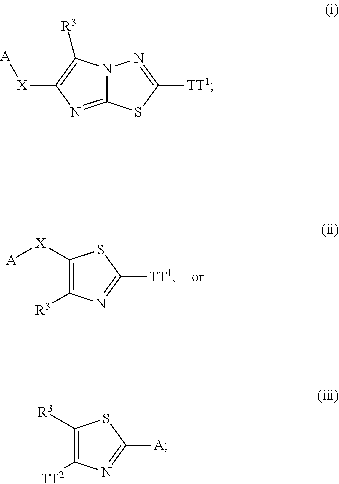

or a pharmaceutically acceptable salt or stereoisomer thereof, wherein:

TT¹ is selected from the group consisting of:

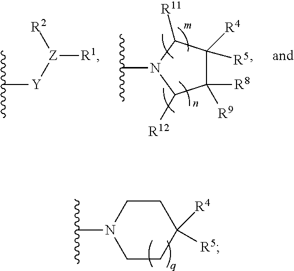

TT² is selected from the group consisting of:

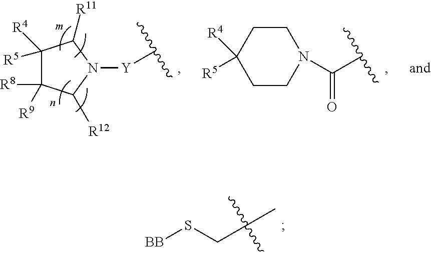

X is selected from the group consisting of a bond, —O—, —NR^{X1}—, —C(O)NR^{X11}—, —S(O)_{w}— (wherein w is 0, 1 or 2), —C(O)—, —C(R^{X2}R^{X3})—, —O—C(R^{X4}R^{X5}), —C(R^{X4}R^{X5})—O—, and —C═C(R^{X6}R^{X7})—;

Y is selected from the group consisting of a bond, —C(O)—, —NR^{Y}—, —C₁₋₃alkylene-NR^{Y}—, and —NR^{Y}—C₁₋₃alkylene-;

Z is selected from the group consisting of N and C(R^{Z});

wherein if Y is a bond or —C(O)—, Z is N; and wherein if Y is —NR^{Y}—, —C₁₋₃alkylene-NR^{Y}—, or —NR^{Y}—C₁₋₃alkylene-, Z is C(R^{Z});

R^{Z} is selected from the group consisting of hydrogen and C₁₋₆alkyl; wherein C₁₋₆alkyl may optionally be substituted by halogen, hydroxyl, and —NR^{a}R^{b},

A is selected from the group consisting of a 6-10 membered monocyclic or bicyclic aryl, a 5-7 membered monocyclic heteroaryl having one or more heteroatoms each independently selected from O, S, or N, a 8-10 membered bicyclic heteroaryl having one or more heteroatoms each independently selected from O, S, or N; and a 4-7 membered heterocyclyl, wherein A may optionally be substituted by one, two, three or more substituents each independently selected from the group consisting of —R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R¹⁰ is independently selected from the group consisting of hydrogen, C₁₋₆alkyl, C₁₋₆haloalkyl, C₃₋₆cycloalkyl, heterocycloalkyl, aryl, and heteroaryl; wherein R¹⁰ may optionally be substituted by one, two, three or more substituents each independently selected from the group consisting of halogen, —C(O)R²⁰, C₁₋₆alkyl and C₁₋₆haloalkyl;

R²⁰ is selected from the group consisting of hydroxyl, halogen, and C₁₋₆alkyl;

R¹ and R² are each selected from the group consisting of hydrogen and C₁₋₁₀alkyl; wherein C₁₋₁₀alkyl may optionally be substituted by one, two, three or more substituents each independently selected from R^{P};

or R¹ and R², together with carbon or nitrogen to which they are attached, form a 3-7 membered saturated carbocyclic or 4-7 membered saturated heterocyclic ring B having one or two heteroatoms each independently selected from the group consisting of 0, S(O), (wherein w is 0, 1, or 2), and NR^{h}; wherein carbocyclic or heterocyclic ring B may be monocyclic or bicyclic;

carbocyclic or heterocyclic ring B may optionally be substituted on one or more available carbons by one, two, three or more substituents each independently selected from the group consisting of halogen, hydroxyl, cyano, oxo, —NR^{a}R^{b}, —CO₂H, —C(O)—NR^{a}R^{b}, —S(O)₂—NR^{a}R^{b}, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})—, C₁₋₆alkoxycarbonyl-N(R^{a})—, aryl and heteroaryl; wherein C₁₋₆alkyl, C₂₋₆alkenyl, C₂₋₆alkynyl, C₃₋₆cycloalkyl, C₁₋₆alkoxy, C₃₋₆ alkenyloxy, C₃₋₆alkynyloxy, C₃₋₆cycloalkoxy, C₁₋₆alkylcarbonyl-, C₁₋₆alkoxycarbonyl, C₁₋₆ alkyl-S(O)_{w}— (wherein w is 0, 1 or 2), C₁₋₆alkylcarbonyl-N(R^{a})— and C₁₋₆alkoxycarbonyl-N(R^{a})— may optionally be substituted by one or more substituents each independently selected from R^{P}; and wherein aryl and heteroaryl may optionally be substituted by one or more substituents each independently selected from R^{f};

R^{X1} is selected from the group consisting of hydrogen, C₁₋₆alkyl and phenyl, or R^{X1} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X11} is selected from the group consisting of hydrogen, C₁₋₆alkyl and phenyl, or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X2} and R^{X3} are each independently selected from the group consisting of hydrogen, halogen, hydroxyl, —NR^{a}R^{b}, —C(O)—NR^{a}R^{b}, C₁₋₆alkyl and C₁₋₆alkoxy; wherein C₁₋₆alkyl and C₁₋₆alkoxy may optionally be substituted by one or more substituents each independently selected from R^{P};

or R^{X2} and R^{X3} together with the carbon to which they are attached form a 3-6 membered carbocycle optionally substituted by one or more substituents each independently selected from the group consisting of hydrogen, halogen, —NR^{a}R^{b}, —C(O)—NR^{a}R^{b}, oxo, C₁₋₆ alkyl and C₁₋₆alkoxy;

R^{X4} and R^{X5} are each independently selected from the group consisting of hydrogen and C₁₋₆alkyl; wherein C₁₋₆alkyl may optionally be substituted by one or more substituents each independently selected from R^{P};

R^{X6} and R^{X7} are each independently selected from the group consisting of hydrogen, halogen, —C(O)—NR^{a}R^{b}, cyano and C₁₋₆alkyl; wherein C₁₋₆alkyl may optionally be substituted by one or more substituents each independently selected from R^{P};

R^{Y} is selected from the group consisting of hydrogen, C₁₋₆alkyl and phenyl;

R³ is selected from the group consisting of hydrogen, halogen, hydroxyl, C₁₋₆alkyl and —NR^{a}R^{b};

R⁴ and R⁵ are each independently selected from the group consisting of hydrogen, hydroxyl, C₁₋₆alkyl, C₁₋₆alkoxy, C₁₋₆alkyl-O—R⁶, —C(O)N(R⁶)₂, —N(R⁶)₂, halogen, C₁₋₆alkyl-N(R⁶)₂, and cyano; wherein C₁₋₆alkyl, C₁₋₆alkoxy, C₁₋₆alkyl-O—R⁶, and C₁₋₆alkyl-N(R⁶)₂ may optionally be substituted by one or more substituents each independently selected from the group consisting of hydroxyl, —NH₂, oxo, and halogen,

or R⁴ and R⁵, taken together with the atoms to which they are attached, form a 3-7 membered carbocyclic or heterocyclic saturated or partially unsaturated ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;

or R⁴ and R⁸, taken together with the atoms to which they are attached, form a 4-7 membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;

or R⁴ is absent, and R⁴ and R⁸, taken together with the atoms to which they are attached, form a 3-membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;

R⁶ is independently for each occurrence selected from the group consisting of hydrogen, C₁₋₆alkyl and phenyl;

R¹¹ and R¹² are each independently selected from the group consisting of hydrogen, hydroxyl, C₁₋₆alkyl, C₁₋₆alkoxy, C₁₋₆alkyl-O—R⁶, aryl, arylalkylene, heteroaryl, heteroarylalkylene, —C(O)N(R⁶)₂, —N(R⁶)₂, halogen, C₁₋₆alkyl-N(R⁶)₂, —CO₂H, and cyano; wherein C₁₋₆alkyl, C₁₋₆alkoxy, C₁₋₆alkyl-O—R⁶, aryl, arylalkylene, heteroaryl, heteroarylalkylene, and C₁₋₆alkyl-N(R⁶)₂ may optionally be substituted by one or more substituents each independently selected from the group consisting of hydroxyl, —NH₂, and halogen;

or R¹¹ and R¹², taken together with the atoms to which they are attached, form a 5-7 membered heterocyclic ring;

or R⁴ and R¹², taken together with the atoms to which they are attached, form a 5-7 membered carbocyclic or heterocyclic ring;

or R⁸ and R¹¹, taken together with the atoms to which they are attached, form a 5-7 membered carbocyclic or heterocyclic ring;

R⁸ and R⁹ are each independently selected from the group consisting of hydrogen, C₁₋₆)alkyl, C₁₋₆alkyl-N(R⁶)₂, —OR⁶, C₁₋₆alkyl-O—R⁶, —C(O)NH₂, —N(R⁶)₂, halogen, and cyano;

each of m and n is, independently, 0, 1, 2, or 3, with m+n being at least 2 and no more than 4;

q is 0 or 1;

R^{f} is independently selected, for each occurrence, from the group consisting of R^{P}, C₁₋₆ alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, (wherein wherein w is 0, 1 or 2), C₁₋₆alkylcarbonyl-N(R^{a})— and C₁₋₆alkoxycarbonyl-N(R^{a})—; wherein C₁₋₆alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, C₁₋₆alkylcarbonyl-N(R^{a})—, and C₁₋₆alkoxycarbonyl-N(R^{a})— may be optionally substituted by one or more substituents selected from R^{P};

R^{h} is independently selected, for each occurrence, from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆ alkylcarbonyl-, C₁₋₆alkoxycarbonyl-, R^{a}R^{b}N-carbonyl- and R^{a}R^{b}N—SO₂—; wherein C₁₋₆alkyl, C₃₋₆alkenyl, C₃₋₆alkynyl, C₃₋₆cycloalkyl, C₁₋₆alkyl-S(O)₂—, C₁₋₆alkylcarbonyl- and C₁₋₆ alkoxycarbonyl- may optionally be substituted by one or more substituents selected from R^{P};

R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl;

or R^{a} and R^{b}, together with the nitrogen to which they are attached, may form a 4-6 membered monocyclic heterocyclic ring, which may have an additional heteroatom selected from the group consisting of O, S, and N; wherein the 4-6 membered heterocyclic ring may optionally be substituted by one or more substituents selected from the group consisting of halogen, cyano, oxo or hydroxyl;

BB is optionally substituted monocyclic or bicyclic heteroaryl; and

R^{P} is independently selected, for each occurrence, from the group consisting of halogen, hydroxyl, cyano, C₁₋₆alkoxy, R^{a}R^{b}N—, R^{a}R^{b}N-carbonyl-, R^{a}R^{b}N—SO₂—, and R^{a}R^{b}N-carbonyl-N(R^{a})—.

In some embodiments, SHP2 phosphatase inhibitors described herein encompass compounds of Formula I, or a pharmaceutically acceptable salt or stereoisomer thereof, wherein Formula I is represented by:

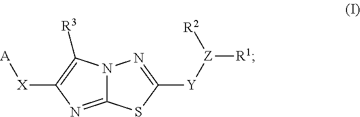

wherein

X is selected from the group consisting of a bond, —O—, —NR^{X1}—, —C(O)NR^{X11}—, —S(O)_{w}— (wherein w is 0, 1 or 2), —C(O)—, —C(R^{X2}R^{X3})—, —O—C(R^{X4}R^{X5}), —C(R^{X4}R^{X5})—O—, and —C═C(R^{X6}R^{X7})—;

Y is selected from the group consisting of a bond, —C(O)—, —NR^{Y}—, —C₁₋₃alkylene-NR^{Y}—, and —NR^{Y}—C₁₋₃alkylene-;

Z is selected from the group consisting of N and C(R^{Z});

wherein if Y is a bond or —C(O)—, Z is N; and wherein if Y is —NR^{Y}—, —C₁₋₃alkylene-NR^{Y}—, or —NR^{Y}—C₁₋₃alkylene-, Z is C(R^{Z});

R^{Z} is selected from the group consisting of hydrogen and C₁₋₆alkyl; wherein C₁₋₆alkyl may optionally be substituted by halogen, hydroxyl, and —NR^{a}R^{b},

A is selected from the group consisting of a 6-10 membered monocyclic or bicyclic aryl, a 5-7 membered monocyclic heteroaryl having one or more heteroatoms each independently selected from O, S, or N, a 8-10 membered bicyclic heteroaryl having one or more heteroatoms each independently selected from O, S, or N; and a 4-7 membered heterocyclyl, wherein A may optionally be substituted by one, two, three or more substituents each independently selected from the group consisting of —R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R¹⁰ is independently selected from the group consisting of hydrogen, C₁₋₆alkyl, C₁₋₆haloalkyl, C₃₋₆cycloalkyl, heterocycloalkyl, aryl, and heteroaryl; wherein R¹⁰ may optionally be substituted by one, two, three or more substituents each independently selected from the group consisting of halogen, —C(O)R²⁰, C₁₋₆alkyl and C₁₋₆haloalkyl;

R²⁰ is selected from the group consisting of hydroxyl, halogen, and C₁₋₆alkyl;

R¹ and R² are each selected from the group consisting of hydrogen and C₁₋₁₀alkyl; wherein C₁₋₁₀alkyl may optionally be substituted by one, two, three or more substituents each independently selected from R^{P};

or R¹ and R², together with carbon or nitrogen to which they are attached, form a 3-7 membered saturated carbocyclic or 4-7 membered saturated heterocyclic ring B having one or two heteroatoms each independently selected from the group consisting of 0, S(O), (wherein w is 0, 1, or 2), and NR^{h}; wherein carbocyclic or heterocyclic ring B may be monocyclic or bicyclic;

carbocyclic or heterocyclic ring B may optionally be substituted on one or more available carbons by one, two, three or more substituents each independently selected from the group consisting of halogen, hydroxyl, cyano, oxo, —NR^{a}R^{b}, —CO₂H, —C(O)—NR^{a}R^{b}, —S(O)₂—NR^{a}R^{b}, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})—, C₁₋₆alkoxycarbonyl-N(R^{a})—, aryl and heteroaryl; wherein C₁₋₆alkyl, C₂₋₆alkenyl, C₂₋₆alkynyl, C₃₋₆cycloalkyl, C₁₋₆alkoxy, C₃₋₆ alkenyloxy, C₃₋₆alkynyloxy, C₃₋₆cycloalkoxy, C₁₋₆alkylcarbonyl-, C₁₋₆alkoxycarbonyl, C₁₋₆ alkyl-S(O)_{w}— (wherein w is 0, 1 or 2), C₁₋₆alkylcarbonyl-N(R^{a})— and C₁₋₆alkoxycarbonyl-N(R^{a})— may optionally be substituted by one or more substituents each independently selected from R^{P}; and wherein aryl and heteroaryl may optionally be substituted by one or more substituents each independently selected from R^{f};

R^{X1} is selected from the group consisting of hydrogen, C₁₋₆alkyl and phenyl, or R^{X1} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X11} is selected from the group consisting of hydrogen, C₁₋₆alkyl and phenyl, or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X2} and R^{X3} are each independently selected from the group consisting of hydrogen, halogen, hydroxyl, —NR^{a}R^{b}, —C(O)—NR^{a}R^{b}, C₁₋₆alkyl and C₁₋₆alkoxy; wherein C₁₋₆alkyl and C₁₋₆alkoxy may optionally be substituted by one or more substituents each independently selected from R^{P};

or R^{X2} and R^{X3} together with the carbon to which they are attached form a 3-6 membered carbocycle optionally substituted by one or more substituents each independently selected from the group consisting of hydrogen, halogen, —NR^{a}R^{b}, —C(O)—NR^{a}R^{b}, oxo, C₁₋₆ alkyl and C₁₋₆alkoxy;

R^{X4} and R^{X5} are each independently selected from the group consisting of hydrogen and C₁₋₆alkyl; wherein C₁₋₆alkyl may optionally be substituted by one or more substituents each independently selected from R^{P};

R^{X6} and R^{X7} are each independently selected from the group consisting of hydrogen, halogen, —C(O)—NR^{a}R^{b}, cyano and C₁₋₆alkyl; wherein C₁₋₆alkyl may optionally be substituted by one or more substituents each independently selected from R^{P};

R^{Y} is selected from the group consisting of hydrogen, C₁₋₆alkyl and phenyl;

R³ is selected from the group consisting of hydrogen, halogen, hydroxyl, C₁₋₆alkyl and —NR^{a}R^{b};

R^{f} is independently selected, for each occurrence, from the group consisting of R^{P}, C₁₋₆ alkyl, C₃₋₆cycloalkyl, C₂₋₆alkenyl, C₂₋₆alkynyl, C₁₋₆alkoxy, C₁₋₆ alkylcarbonyl-, C₁₋₆alkoxycarbonyl, C₁₋₆alkyl-S(O)_{w}—, (wherein wherein w is 0, 1 or 2), C₁₋₆alkylcarbonyl-N(R^{a})— and C₁₋₆alkoxycarbonyl-N(R^{a})—; wherein C₁₋₆alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, C₁₋₆alkylcarbonyl-N(R^{a})—, and C₁₋₆alkoxycarbonyl-N(R^{a})— may be optionally substituted by one or more substituents selected from R^{P};

R^{h} is independently selected, for each occurrence, from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆ alkylcarbonyl-, C₁₋₆alkoxycarbonyl-, R^{a}R^{b}N-carbonyl- and R^{a}R^{b}N—SO₂—; wherein C₁₋₆alkyl, C₃₋₆alkenyl, C₃₋₆alkynyl, C₃₋₆cycloalkyl, C₁₋₆alkyl-S(O)₂—, C₁₋₆alkylcarbonyl- and C₁₋₆ alkoxycarbonyl- may optionally be substituted by one or more substituents selected from R^{P};

R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl;

or R^{a} and R^{b}, together with the nitrogen to which they are attached, may form a 4-6 membered monocyclic heterocyclic ring, which may have an additional heteroatom selected from the group consisting of O, S, and N; wherein the 4-6 membered heterocyclic ring may optionally be substituted by one or more substituents selected from the group consisting of halogen, cyano, oxo or hydroxyl; and

R^{P} is independently selected, for each occurrence, from the group consisting of halogen, hydroxyl, cyano, C₁₋₆alkoxy, R^{a}R^{b}N—, R^{a}R^{b}N-carbonyl-, R^{a}R^{b}N—SO₂—, and R^{a}R^{b}N-carbonyl-N(R^{a})—.

In some embodiments, X is selected from the group consisting of a bond, —O—, —NR^{X1}— and —NR^{X11}—.

In some embodiments, A is selected from the group consisting of phenyl, pyridyl, quinolinyl, indolyl, and indolinyl, wherein phenyl, pyridyl, quinolinyl, indolyl and indolinyl may optionally be substituted by one, two or three substituents each independently selected from the group consisting of —R¹⁰, —OR¹⁰, —SR¹⁰, —N(R¹⁰)₂, —OSO₂R¹⁰, —SO₂R¹⁰, —C(O)N(R¹⁰)₂, halogen, and cyano. In certain embodiments, A is phenyl; wherein phenyl may optionally be substituted by one, two, or three substituents each independently selected from the group consisting of —OR¹⁰, halogen, and cyano. In certain other embodiments, A is pyridyl; wherein pyridyl may optionally be substituted by one, two, or three substituents each independently selected from the group consisting of —OR¹⁰, halogen, and cyano.

In some embodiments, R^{X1} and ring A together with the nitrogen to which they are attached form a 1,2,3,4-tetrahydro-1,5-naphthyridine moiety. In some embodiments, R^{X11} and ring A together with the nitrogen to which they are attached form a 1,2,3,4-tetrahydro-1,5-naphthyridine moiety. In some embodiments, R^{X1} and ring A together with the nitrogen to which they are attached form a 1,2,3,4-tetrahydroquinoline moiety. In some embodiments, R^{X11} and ring A together with the nitrogen to which they are attached form a 1,2,3,4-tetrahydroquinoline moiety.

In some embodiments, A is phenyl, pyridyl, indolyl, or indolinyl, wherein said phenyl, pyridyl, indolyl or indolinyl is optionally substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano.

In some embodiments, A is phenyl, pyridyl, imidazolyl, indolyl, indolinyl, naphthyl, quinolinyl, isoquinolinyl, benzimidazolyl, indazolyl, benzotriazolyl, benzothiazolyl, benzofuranyl, pyrazolopyridyl, quinazolinyl, quinoxalinyl, or cinnolinyl. In some embodiments, A is phenyl. In some embodiments, A is pyridyl. In some embodiments, A is 2-pyridyl. In some embodiments, A is 3-pyridyl. In some embodiments, A is 4-pyridyl. In some embodiments, A is naphthyl. In some embodiments, A is 1-naphthyl. In some embodiments, A is 2-naphthyl. In some embodiments, A is quinolinyl. In some embodiments, A is 2-quinolinyl. In some embodiments, A is 3-quinolinyl. In some embodiments, A is 4-quinolinyl. In some embodiments, A is 5-quinolinyl. In some embodiments, A is 6-quinolinyl. In some embodiments, A is 7-quinolinyl. In some embodiments, A is 8-quinolinyl. In some embodiments, A is isoquinolinyl. In some embodiments, A is 1-isoquinolinyl. In some embodiments, A is 3-isoquinolinyl. In some embodiments, A is 4-isoquinolinyl. In some embodiments, A is 5-isoquinolinyl. In some embodiments, A is 6-isoquinolinyl. In some embodiments, A is 7-isoquinolinyl. In some embodiments, A is 8-isoquinolinyl. In some embodiments, A is indolyl. In some embodiments, A is 2-indolyl. In some embodiments, A is 3-indolyl. In some embodiments, A is 4-indolyl. In some embodiments, A is 5-indolyl. In some embodiments, A is 6-indolyl. In some embodiments, A is 7-indolyl. In some embodiments, A is aryl. In some embodiments, A is bicyclic aryl. In some embodiments, A is heteroaryl. In some embodiments, A is fused bicyclic heteroaryl.

In some embodiments, A is phenyl, pyridyl, or indolyl, wherein said phenyl, pyridyl, or indolyl is optionally substituted with —OR¹⁰, halogen, or cyano.

In some embodiments, A is phenyl, wherein said phenyl is optionally substituted with —OR¹⁰, halogen, or cyano.

In some embodiments, A is pyridyl, wherein said pyridyl is optionally substituted with —OR¹⁰, halogen, or cyano.

In some embodiments, A is indolyl or indolinyl, wherein said indolyl or indolinyl is optionally substituted with —OR¹⁰, halogen, or cyano.

In some embodiments, A is phenyl, pyridyl, indolyl, or indolinyl, wherein said phenyl, pyridyl, indolyl or indolinyl is optionally substituted with —OR¹⁰ or halogen.

In some embodiments, A is phenyl, pyridyl, indolyl, or indolinyl, wherein said phenyl, pyridyl, indolyl or indolinyl is optionally substituted with halogen.

In some embodiments, A is phenyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is phenyl substituted with —R¹⁰. In some embodiments, A is phenyl substituted with —OR °. In some embodiments, A is phenyl substituted with —SR¹⁰. In some embodiments, A is phenyl substituted with —N(R¹⁰)₂. In some embodiments, A is phenyl substituted with —OSO₂R¹⁰. In some embodiments, A is phenyl substituted with —SO₂R¹⁰. In some embodiments, A is phenyl substituted with —C(O)N(R¹⁰)₂. In some embodiments, A is phenyl substituted with halogen. In some embodiments, A is phenyl substituted with cyano.

In some embodiments, A is pyridyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is pyridyl substituted with —R¹⁰. In some embodiments, A is pyridyl substituted with —OR¹⁰. In some embodiments, A is pyridyl substituted with —SR¹⁰. In some embodiments, A is pyridyl substituted with —N(R¹⁰)₂. In some embodiments, A is pyridyl substituted with —OSO₂R¹⁰. In some embodiments, A is pyridyl substituted with —SO₂R¹⁰. In some embodiments, A is pyridyl substituted with —C(O)N(R¹⁰)₂. In some embodiments, A is pyridyl substituted with halogen. In some embodiments, A is pyridyl substituted with cyano.

In some embodiments, A is 3-pyridyl substituted with —OR¹⁰. In some embodiments, A is 3-pyridyl substituted with —OCH₃. In some embodiments, A is 6-methoxypyrid-3-yl. In some embodiments, A is 3-pyridyl substituted with halogen. In some embodiments, A is 3-pyridyl substituted with Cl. In some embodiments, A is 4-chloropyrid-3-yl. In some embodiments, A is 2,3-dichloropyrid-4-yl. In some embodiments, A is 3-chloropyrid-2-yl.

In some embodiments, A is naphthyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is naphthyl substituted with —R¹⁰. In some embodiments, A is naphthyl substituted with —OR¹⁰. In some embodiments, A is naphthyl substituted with —SR¹⁰. In some embodiments, A is naphthyl substituted with —N(R¹⁰)₂. In some embodiments, A is naphthyl substituted with —OSO₂R¹⁰. In some embodiments, A is naphthyl substituted with —SO₂R¹⁰. In some embodiments, A is naphthyl substituted with —C(O)N(R¹⁰)₂. In some embodiments, A is naphthyl substituted with halogen. In some embodiments, A is naphthyl substituted with cyano.

In some embodiments, A is quinolinyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is quinolinyl substituted with —R¹⁰. In some embodiments, A is quinolinyl substituted with —OR¹⁰. In some embodiments, A is quinolinyl substituted with —SR¹⁰. In some embodiments, A is quinolinyl substituted with —N(R¹⁰)₂. In some embodiments, A is quinolinyl substituted with —OSO₂R¹⁰. In some embodiments, A is quinolinyl substituted with —SO₂R¹⁰. In some embodiments, A is quinolinyl substituted with —C(O)N(R¹⁰)₂. In some embodiments, A is quinolinyl substituted with halogen. In some embodiments, A is quinolinyl substituted with cyano.

In some embodiments, A is isoquinolinyl substituted with —R¹⁰, —OR, —SR¹⁰, —N(R¹⁰)₂, —OSO₂R¹⁰, —SO₂R¹⁰, —C(O)N(R¹⁰)₂, halogen, or cyano. In some embodiments, A is isoquinolinyl substituted with —R¹⁰. In some embodiments, A is isoquinolinyl substituted with —OR¹⁰. In some embodiments, A is isoquinolinyl substituted with —SR¹⁰. In some embodiments, A is isoquinolinyl substituted with —N(R¹⁰)₂. In some embodiments, A is isoquinolinyl substituted with —OSO₂R¹⁰. In some embodiments, A is isoquinolinyl substituted with —SO₂R¹⁰. In some embodiments, A is isoquinolinyl substituted with —C(O)N(R¹⁰)₂. In some embodiments, A is isoquinolinyl substituted with halogen. In some embodiments, A is isoquinolinyl substituted with cyano.

In some embodiments, A is indolyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is indolyl substituted with —R¹⁰. In some embodiments, A is indolyl substituted with —OR¹⁰. In some embodiments, A is indolyl substituted with —SR¹⁰. In some embodiments, A is indolyl substituted with —N(R¹⁰)₂. In some embodiments, A is indolyl substituted with —OSO₂R¹⁰. In some embodiments, A is indolyl substituted with —SO₂R¹⁰. In some embodiments, A is indolyl substituted with —C(O)N(R¹⁰)₂. In some embodiments, A is indolyl substituted with halogen. In some embodiments, A is indolyl substituted with cyano.

In some embodiments, A is 1-methyl-1H-indol-4-yl. In some embodiments, A is 1-methyl-1H-indol-3-yl.

In some embodiments, A is imidazol-2-yl. In some embodiments, A is 1-methyl-imidazol-2-yl.

In some embodiments, A is selected from the group consisting of 1,2,3,4-tetrahydroquinoline; 1,2,3,4-tetrahydro-1,5,napthyridine, dihydropyridopyrazine, benzoimidazole, benzoazepine, and dihydropyridooxazine, wherein A is bound to X through an available nitrogen on X; and where A is optionally substituted with one or two substituents each independently selected from halo, CF₃, phenyl (optionally substituted with one, two or three halo, C₁₋₃alkyl or C₁₋₃haloalkyl), C₁₋₃alkyl, C₁₋₃haloalkyl, and heteroaryl (optionally substituted with one, two or three halo, C₁₋₃alkyl or C₁₋₃haloalkyl).

In some embodiments, A is selected from the group consisting of

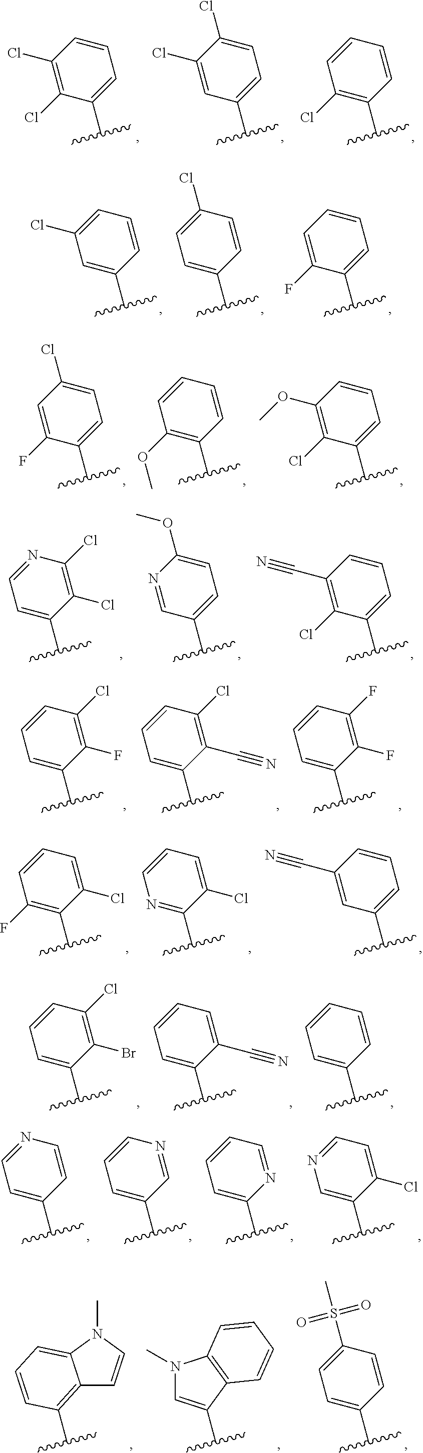

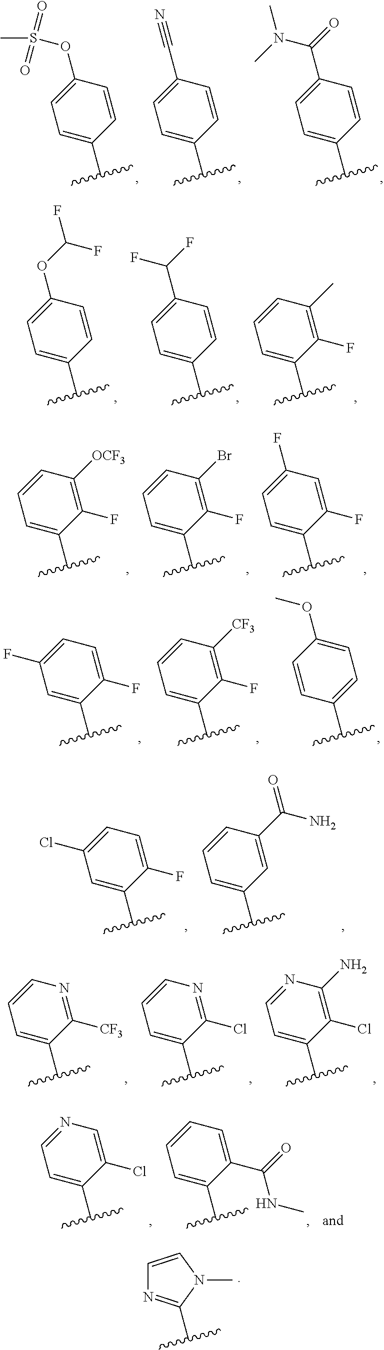

In some embodiments, A is

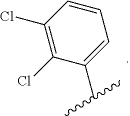 [SMILES: *c1cccc(Cl)c1Cl]

In some embodiments, A is

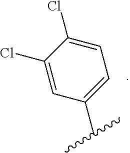 [SMILES: *c1ccc(Cl)c(Cl)c1]

In some embodiments, A is

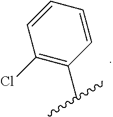 [SMILES: *c1ccccc1Cl]

In some embodiments, A is

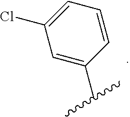 [SMILES: *c1cccc(Cl)c1]

In some embodiments, A is

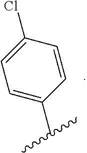 [SMILES: *c1ccc(Cl)cc1]

In some embodiments, A is

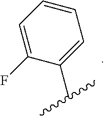 [SMILES: *c1ccccc1F]

In some embodiments, A is

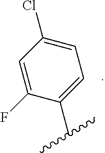 [SMILES: *c1ccc(Cl)cc1F]

In some embodiments, A is

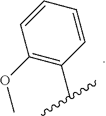 [SMILES: *c1ccccc1OC]

In some embodiments, A is

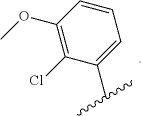 [SMILES: *c1cccc(OC)c1Cl]

In some embodiments, A is

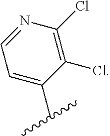 [SMILES: *c1ccnc(Cl)c1Cl]

In some embodiments, A is

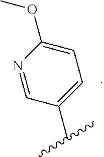 [SMILES: *c1ccc(OC)nc1]

In some embodiments, A is

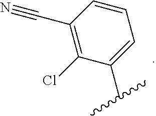 [SMILES: *c1cccc(C#N)c1Cl]

In some embodiments, A is

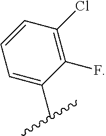 [SMILES: *c1cccc(Cl)c1F]

In some embodiments, A is

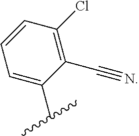 [SMILES: *c1cccc(Cl)c1C#N]

In some embodiments, A is

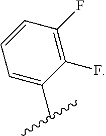 [SMILES: *c1cccc(F)c1F]

In some embodiments, A is

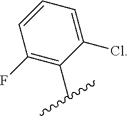 [SMILES: *c1c(F)cccc1Cl]

In some embodiments, A is

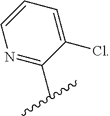 [SMILES: *c1ncccc1Cl]

In some embodiments, A is

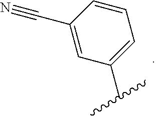 [SMILES: *c1cccc(C#N)c1]

In some embodiments, A is

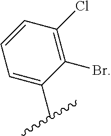 [SMILES: *c1cccc(Cl)c1Br]

In some embodiments, A is

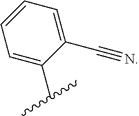 [SMILES: *c1ccccc1C#N]

In some embodiments, A is

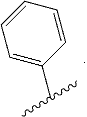 [SMILES: *c1ccccc1]

In some embodiments, A is

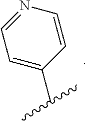 [SMILES: *c1ccncc1]

In some embodiments, A is

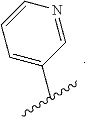 [SMILES: *c1cccnc1]

In some embodiments, A is

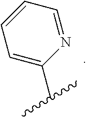 [SMILES: *c1ccccn1]

In some embodiments, A is

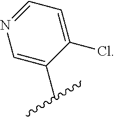 [SMILES: *c1cnccc1Cl]

In some embodiments, A is

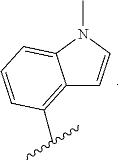 [SMILES: *c1cccc2c1ccn2C]

In some embodiments, A is

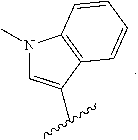 [SMILES: *c1cn(C)c2ccccc12]

In some embodiments, A is

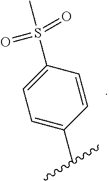 [SMILES: *c1ccc(S(C)(=O)=O)cc1]

In some embodiments, A is

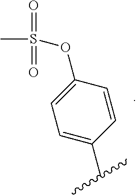 [SMILES: *c1ccc(OS(C)(=O)=O)cc1]

In some embodiments, A is

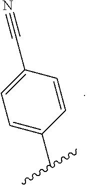 [SMILES: *c1ccc(C#N)cc1]

In some embodiments, A is

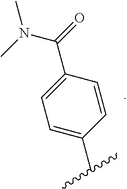 [SMILES: *c1ccc(C(=O)N(C)C)cc1]

In some embodiments, A is

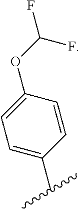 [SMILES: *c1ccc(OC(F)F)cc1]

In some embodiments, A is

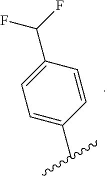 [SMILES: *c1ccc(C(F)F)cc1]

In some embodiments, A is

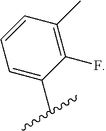 [SMILES: *c1cccc(C)c1F]

In some embodiments, A is

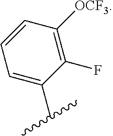 [SMILES: *c1cccc(OC(F)(F)F)c1F]

In some embodiments, A is

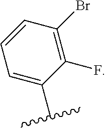 [SMILES: *c1cccc(Br)c1F]

In some embodiments, A is

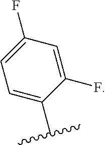 [SMILES: *c1ccc(F)cc1F]

In some embodiments, A is

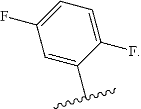 [SMILES: *c1cc(F)ccc1F]

In some embodiments, A is

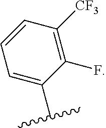 [SMILES: *c1cccc(C(F)(F)F)c1F]

In some embodiments, A is

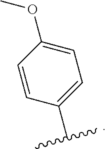 [SMILES: *c1ccc(OC)cc1 ; "."]

In some embodiments, A is

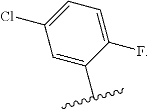 [SMILES: *c1cc(Cl)ccc1F]

In some embodiments, A is

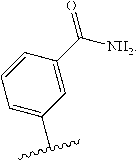 [SMILES: *c1cccc(C(N)=O)c1]

In some embodiments, A is

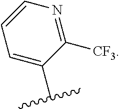 [SMILES: *c1cccnc1C(F)(F)F]

In some embodiments, A is

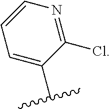 [SMILES: *c1cccnc1Cl]

In some embodiments, A is

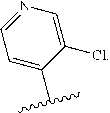 [SMILES: *c1ccncc1Cl]

In some embodiments, A is

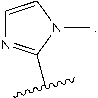 [SMILES: *c1nccn1C ; "."]

In some embodiments, A is

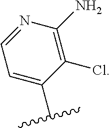 [SMILES: *c1ccnc(N)c1Cl]

In some embodiments, A is

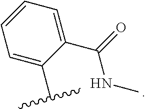 [SMILES: *c1ccccc1C(=O)NC ; "."]

In certain embodiments, R³ is selected from the group consisting of hydrogen, CH₃ and —NH₂. In some embodiments, Y is a bond and Z is N.

In some embodiments, R¹ and R² together with the nitrogen to which they are attached form heterocyclic ring B; wherein ring B is selected from the group consisting of pyrrolidinyl, piperidinyl, azepanyl and morpholinyl; wherein pyrrolidinyl, piperidinyl, azepanyl and morpholinyl may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃alkoxy; wherein C₁₋₃alkyl and C₁₋₃alkoxy may optionally be substituted by halogen, hydroxyl and —NR^{a}R^{b}.

In some embodiments, Y is —NH—, —N(CH₃)—, —N(H)—CH₂—, or —N(CH₃)—CH₂—; and Z is C(H).

In certain embodiments, R¹ and R² together with the carbon to which they are attached form carbocyclic ring B; wherein ring B is selected from the group consisting of cyclopentyl and cyclohexyl, and wherein cyclopentyl and cyclohexyl may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃ alkoxy; wherein C₁₋₃alkyl and C₁₋₃alkoxy may optionally be substituted by halogen, hydroxyl and —NR^{a}R^{b}.

In certain other embodiments, R¹ and R² together with the carbon to which they are attached form heterocyclic ring B; wherein ring B is selected from the group consisting of tetrahydrofuranyl and tetrahydropyranyl, wherein tetrahydrofuranyl and tetrahydropyranyl may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃alkoxy; wherein C₁₋₃alkyl and C₁₋₃alkoxy may optionally be substituted by halogen, hydroxyl and —NR^{a}R^{b}.

For example, in some embodiments, ring B is substituted on an available carbon by a substituent selected from the group consisting of —NH₂, —CH₃, and —CH₂NH₂.

In certain embodiments, R¹ is hydrogen and R² is branched C₁₋₁₀alkyl; wherein C₁₋₁₀alkyl may optionally be substituted by one, two, three or more substituents each independently selected from the group consisting of halogen and —NH₂.

In some embodiments, SHP2 phosphatase inhibitors described herein encompass compounds of Formula II, or a pharmaceutically acceptable salt or stereoisomer thereof, wherein Formula II is represented by:

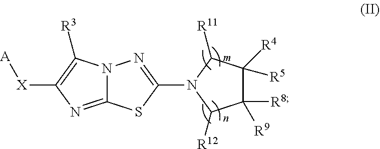

wherein

X is selected from the group consisting of a bond, —O—, —NR^{X1}—, —C(O)NR^{X11}—, —S(O)_{w}— (wherein w is 0, 1 or 2), —C(O)— and —C(R^{X2}R^{X3})—;

A is selected from the group consisting of phenyl, a 5-7 membered monocyclic heteroaryl having one or more heteroatoms each independently selected from O, S, and N, a 8-10 membered bicyclic heteroaryl having one or more heteroatoms each independently selected from the group consisting of O, S, and N, and a 4-7 membered heterocyclyl; wherein A may optionally be substituted by one, two, three or more substituents each independently selected from the group consisting of —R¹⁰, —OR¹⁰, —N(R¹⁰)₂, —C(O)N(R¹⁰)₂, oxo, halogen and cyano;

R¹⁰ is independently selected from the group consisting of hydrogen, C₁₋₆alkyl, C₁₋₆haloalkyl, C₃₋₆cycloalkyl, heterocycloalkyl, aryl, and heteroaryl, where R¹⁰ may optionally be substituted by one, two, three or more substituents each independently selected from the group consisting of halogen, —C(O)R²⁰, C₁₋₆alkyl and C₁₋₆haloalkyl;

R²⁰ is selected from the group consisting of hydroxyl, halogen, and C₁₋₆alkyl;

R^{X1} is selected from the group consisting of hydrogen, C₁₋₆alkyl and phenyl; or R and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —N(R¹⁰)₂, —C(O)N(R¹⁰)₂, oxo, halogen and cyano;

R^{X11} is selected from the group consisting of hydrogen, C₁₋₆alkyl and phenyl; or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —N(R¹⁰)₂, —C(O)N(R¹⁰)₂, oxo, halogen and cyano;

R^{X2} and R^{X3} are each independently selected from the group consisting of hydrogen, halogen, hydroxyl, —NR^{a}R^{b}, —C(O)—NR^{a}R^{b}, C₁₋₆alkyl and C₁₋₆alkoxy; or R^{X2} and R^{X3} together with the carbon to which they are attached form a 3-6 membered carbocyclyl which may optionally be substituted by one or more substituents each independently selected from the group consisting of hydrogen, halogen, —NR^{a}R^{b}, —C(O)—NR^{a}R^{b}, oxo, C₁₋₆alkyl and C₁₋₆ alkoxy;

R³ is selected from the group consisting of hydrogen, halogen, hydroxyl, C₁₋₆alkyl and —NR^{a}R^{b};

R⁴ and R⁵ are each independently selected from the group consisting of hydrogen, hydroxyl, C₁₋₆alkyl, C₁₋₆alkoxy, C₁₋₆alkyl-O—R⁶, —C(O)N(R⁶)₂, —N(R⁶)₂, halogen, C₁₋₆alkyl-N(R⁶)₂, and cyano; wherein C₁₋₆alkyl, C₁₋₆alkoxy, C₁₋₆alkyl-O—R⁶, and C₁₋₆alkyl-N(R⁶)₂ may optionally be substituted by one or more substituents each independently selected from the group consisting of hydroxyl, —NH₂, oxo, and halogen,

or R⁴ and R⁵, taken together with the atoms to which they are attached, form a 3-7 membered carbocyclic or heterocyclic saturated or partially unsaturated ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;

or R⁴ and R⁸, taken together with the atoms to which they are attached, form a 4-7 membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;

or R⁴ is absent, and R⁴ and R⁸, taken together with the atoms to which they are attached, form a 3-membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;

R⁶ is independently for each occurrence selected from the group consisting of hydrogen, C₁₋₆alkyl and phenyl;

R¹¹ and R¹² are each independently selected from the group consisting of hydrogen, hydroxyl, C₁₋₆alkyl, C₁₋₆alkoxy, C₁₋₆alkyl-O—R⁶, aryl, arylalkylene, heteroaryl, heteroarylalkylene, —C(O)N(R⁶)₂, —N(R⁶)₂, halogen, C₁₋₆alkyl-N(R⁶)₂, —CO₂H, and cyano; wherein C₁₋₆alkyl, C₁₋₆alkoxy, C₁₋₆alkyl-O—R⁶, aryl, arylalkylene, heteroaryl, heteroarylalkylene, and C₁₋₆alkyl-N(R⁶)₂ may optionally be substituted by one or more substituents each independently selected from the group consisting of hydroxyl, —NH₂, and halogen;

or R¹¹ and R¹², taken together with the atoms to which they are attached, form a 5-7 membered heterocyclic ring;

or R⁴ and R¹², taken together with the atoms to which they are attached, form a 5-7 membered carbocyclic or heterocyclic ring;

or R⁸ and R¹¹, taken together with the atoms to which they are attached, form a 5-7 membered carbocyclic or heterocyclic ring;

R⁸ and R⁹ are each independently selected from the group consisting of hydrogen, C₁₋₆)alkyl, C₁₋₆alkyl-N(R⁶)₂, —OR⁶, C₁₋₆alkyl-O—R⁶, —C(O)NH₂, —N(R⁶)₂, halogen, and cyano; and

each of m and n is, independently, 0, 1, 2, or 3, with m+n being at least 2 and no more than 4.

In some embodiments, A is phenyl; wherein phenyl may optionally be substituted by one, two, or three substituents each independently selected from the group consisting of —OR¹⁰, halogen, and cyano. In some embodiments, A is pyridyl; wherein pyridyl may optionally be substituted by one, two, or three substituents each independently selected from the group consisting of —OR¹⁰, halogen, and cyano.

In some embodiments, R^{X1} and ring A together with the nitrogen to which they are attached form a 1,2,3,4-tetrahydro-1,5-naphthyridine moiety. In some embodiments, R^{X11} and ring A together with the nitrogen to which they are attached form a 1,2,3,4-tetrahydro-1,5-naphthyridine moiety. In some embodiments, R^{X1} and ring A together with the nitrogen to which they are attached form a 1,2,3,4-tetrahydroquinoline moiety. In some embodiments, R^{X11} and ring A together with the nitrogen to which they are attached form a 1,2,3,4-tetrahydroquinoline moiety.

In certain embodiments, R³ is selected from the group consisting of hydrogen, CH₃ and —NH₂. In certain other embodiments, m is an integer selected from 1 or 2; and n is 1.

In some embodiments, R⁴ and R⁵ are each independently selected from the group consisting of hydrogen, hydroxyl, C₁₋₃alkyl, C₁₋₃alkoxy, C₁₋₃alkyl-O—R⁶, —C(O)NH₂, —N(R⁶)₂, halogen, C₁₋₃alkyl-N(R⁶)₂, and cyano; wherein C₁₋₃alkyl, C₁₋₃alkoxy, C₁₋₃alkyl-O—R⁶ and C₁₋₃alkyl-N(R⁶)₂ may optionally be substituted by one or more substituents each independently selected from the group consisting of hydroxyl, —N(R⁶)₂, and halogen;

or R⁴ and R⁵, taken together with the atoms to which they are attached, form a 3-7 membered carbocyclic or heterocyclic ring;

or R⁴ and R⁸, taken together with the atoms to which they are attached, form a 4-7 membered carbocyclic or heterocyclic ring;

or R⁴ is a bond, and R⁴ and R⁸, taken together with the atoms to which they are attached, form a 3-membered carbocyclic or heterocyclic ring; and

each R⁶ is independently hydrogen or C₁₋₃alkyl.

For example, in some embodiments, R⁴ and R⁵ are each independently selected from the group consisting of C₁₋₃alkyl and —NR^{a}R^{b}; wherein C₁₋₃alkyl may optionally be substituted by one, two or three substituents each independently selected from the group consisting of halogen, hydroxyl and —NR^{a}R^{b}. In certain embodiments, R⁴ and R⁵ are each independently selected from the group consisting of —NH₂, —CH₃, and —CH₂NH₂.

In some embodiments, SHP2 phosphatase inhibitors described herein encompass compounds of Formula III, or a pharmaceutically acceptable salt or stereoisomer thereof, wherein Formula III is represented by:

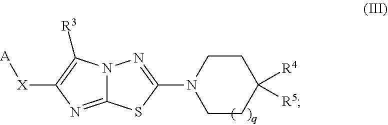

wherein

X is selected from the group consisting of a bond, —NR^{X1}—, and —C(O)NR^{X11}—;

A is selected from the group consisting of phenyl, a 5-7 membered monocyclic heteroaryl having one or more heteroatoms each independently selected from O, S, and N, a 8-10 membered bicyclic heteroaryl having one or more heteroatoms each independently selected from the group consisting of O, S, and N, and a 4-7 membered heterocyclyl; wherein A may optionally be substituted by one, two, three or more substituents each independently selected from the group consisting of —R¹⁰, —OR¹⁰, —N(R¹⁰)₂, —C(O)N(R¹⁰)₂, oxo, halogen and cyano;

R¹⁰ is independently selected from the group consisting of hydrogen, C₁₋₆alkyl, C₁₋₆haloalkyl, C₃₋₆cycloalkyl, heterocycloalkyl, aryl, and heteroaryl, where R¹⁰ may optionally be substituted by one, two, three or more substituents each independently selected from the group consisting of halogen, —C(O)R²⁰, C₁₋₆alkyl and C₁₋₆haloalkyl;

R²⁰ is selected from the group consisting of hydroxyl, halogen, and C₁₋₆alkyl;

R^{X1} is selected from the group consisting of hydrogen, C₁₋₆alkyl and phenyl, or R^{X1} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —N(R¹⁰)₂, —C(O)N(R¹⁰)₂, oxo, halogen and cyano;

R^{X11} is selected from the group consisting of hydrogen, C₁₋₆alkyl and phenyl, or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —N(R¹⁰)₂, —C(O)N(R¹⁰)₂, oxo, halogen and cyano;

R³ is selected from the group consisting of hydrogen, halogen, hydroxyl, C₁₋₆alkyl and —NR^{a}R^{b};

R⁴ and R⁵ are each independently selected from the group consisting of hydrogen, halogen, cyano, —NR^{a}R^{b}, —C(O)—NR^{a}R^{b} and C₁₋₆alkyl, wherein C₁₋₆alkyl may optionally be substituted by one, two or three or more substituents each independently selected from the group consisting of halogen, hydroxyl, —NR^{a}R^{b} and oxo;

R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl; and q is 0 or 1.

In some embodiments, A is phenyl; wherein phenyl may optionally be substituted by one, two, or three substituents each independently selected from the group consisting of —OR¹⁰, halogen, and cyano. In some embodiments, A is pyridyl; wherein pyridyl may optionally be substituted by one, two, or three substituents each independently selected from the group consisting of —OR¹⁰, halogen, and cyano.

In some embodiments, R^{X1} and ring A together with the nitrogen to which they are attached form a 1,2,3,4-tetrahydro-1,5-naphthyridine moiety. In some embodiments, R^{X11} and ring A together with the nitrogen to which they are attached form a 1,2,3,4-tetrahydro-1,5-naphthyridine moiety. In some embodiments, R^{X1} and ring A together with the nitrogen to which they are attached form a 1,2,3,4-tetrahydroquinoline moiety. In some embodiments, R^{X11} and ring A together with the nitrogen to which they are attached form a 1,2,3,4-tetrahydroquinoline moiety.

In some embodiments, R³ is selected from the group consisting of hydrogen, CH₃ and —NH₂. In some embodiments, R⁴ and R⁵ are each independently selected from the group consisting of C₁₋₃alkyl and —NR^{a}R^{b}; wherein C₁₋₃alkyl may optionally be substituted by one, two or three substituents each independently selected from the group consisting of halogen, hydroxyl and —NR^{a}R^{b}. For example, in some embodiments, R⁴ and R⁵ are each independently selected from the group consisting of —NH₂, —CH₃, and —CH₂NH₂.

Examples of compounds of the present disclosure include:

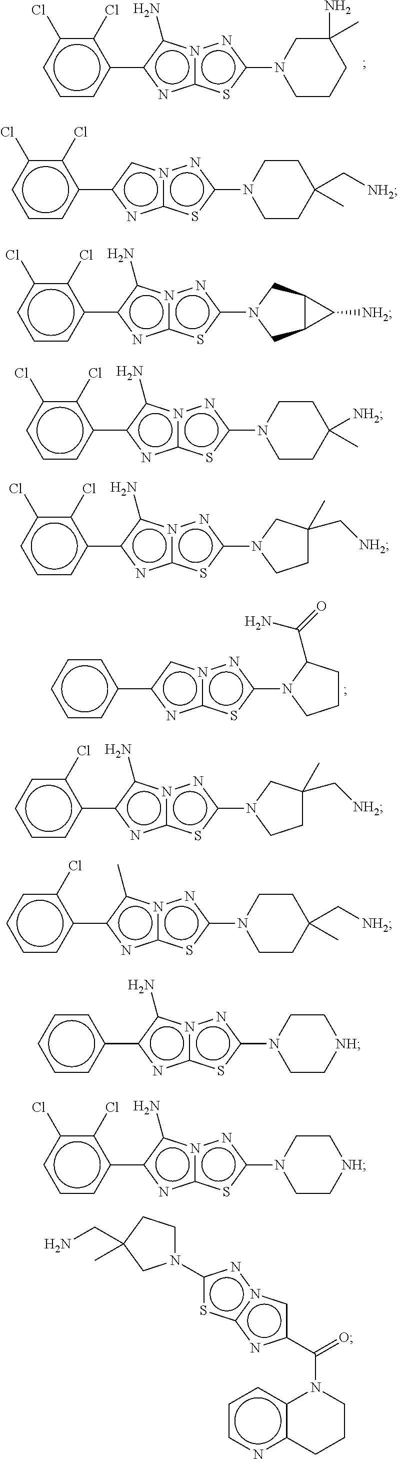

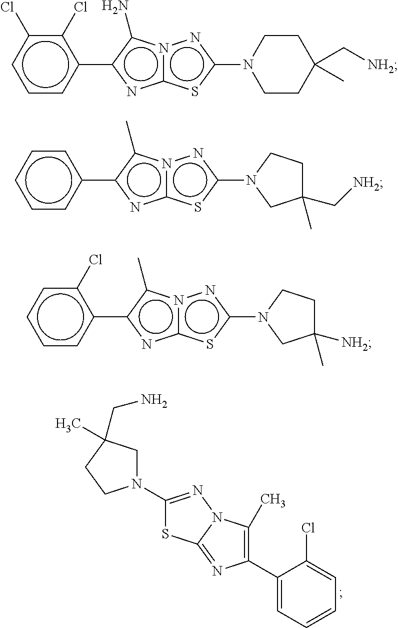

and a pharmaceutically acceptable salt or stereoisomer thereof.

In some embodiments, SHP2 phosphatase inhibitors described herein encompass compounds of Formula IV, or a pharmaceutically acceptable salt or stereoisomer thereof, wherein Formula IV is represented by:

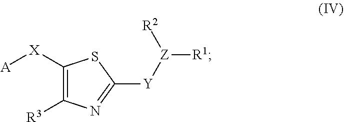

wherein

X is selected from the group consisting of a bond, —O—, —NR^{X1}—, —C(O)NR^{X11}—, —S(O)_{w}— (wherein w is 0, 1 or 2), —C(O)—, —C(R^{X2}R^{X3})—, —O—C(R^{X4}R^{X5})—, —C(R^{X4}R^{X5})—O—, and —C═C(R^{X6}R^{X7})—;

Y is selected from the group consisting of a bond, —C(O)—, —NR^{Y}—, —C₁₋₃alkylene-NR^{Y}—, and —NR^{Y}—C₁₋₃alkylene-;

Z is selected from the group consisting of N and C(R^{Z});

wherein if Y is a bond or —C(O)—, Z is N; and wherein if Y is —NR^{Y}—, —C₁₋₃alkylene-NR^{Y}—, or —NR^{Y}—C₁₋₃alkylene-, Z is C(R^{Z});

R^{Z} is selected from the group consisting of hydrogen and C₁₋₆alkyl; wherein C₁₋₆alkyl may optionally be substituted by halogen, hydroxyl, and —NR^{a}R^{b},

A is selected from the group consisting of a 6-10 membered monocyclic or bicyclic aryl, a 5-7 membered monocyclic heteroaryl having one or more heteroatoms each independently selected from O, S, or N, a 8-10 membered bicyclic heteroaryl having one or more heteroatoms each independently selected from O, S, or N; and a 4-7 membered heterocyclyl, wherein A may optionally be substituted by one, two, three or more substituents each independently selected from the group consisting of —R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R¹⁰ is independently selected from the group consisting of hydrogen, C₁₋₆alkyl, C₁₋₆haloalkyl, C₃₋₆cycloalkyl, heterocycloalkyl, aryl, and heteroaryl; wherein R¹⁰ may optionally be substituted by one, two, three or more substituents each independently selected from the group consisting of halogen, —C(O)R²⁰, C₁₋₆alkyl and C₁₋₆haloalkyl;

R²⁰ is selected from the group consisting of hydroxyl, halogen, and C₁₋₆alkyl;

R¹ and R² are each selected from the group consisting of hydrogen and C₁₋₁₀alkyl; wherein C₁₋₁₀alkyl may optionally be substituted by one, two, three or more substituents each independently selected from R^{P};

or R¹ and R², together with carbon or nitrogen to which they are attached, form a 3-7 membered saturated carbocyclic or 4-7 membered saturated heterocyclic ring B having one or two heteroatoms each independently selected from the group consisting of O, S(O)_{w} (wherein w is 0, 1, or 2), and NR^{h}; wherein carbocyclic or heterocyclic ring B may be monocyclic or bicyclic;

carbocyclic or heterocyclic ring B may optionally be substituted on one or more available carbons by one, two, three or more substituents each independently selected from the group consisting of halogen, hydroxyl, cyano, oxo, —NR^{a}R^{b}, —CO₂H, —C(O)—NR^{a}R^{b}, —S(O)₂—NR^{a}R^{b}, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})—, C₁₋₆alkoxycarbonyl-N(R^{a})—, aryl and heteroaryl; wherein C₁₋₆alkyl, C₂₋₆alkenyl, C₂₋₆alkynyl, C₃₋₆cycloalkyl, C₁₋₆alkoxy, C₃₋₆ alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})— and C₁₋₆alkoxycarbonyl-N(R^{a})— may optionally be substituted by one or more substituents each independently selected from R^{P}; and wherein aryl and heteroaryl may optionally be substituted by one or more substituents each independently selected from R^{f};

R^{X1} is selected from the group consisting of hydrogen, C₁₋₆alkyl and phenyl, or R^{X1} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X11} is selected from the group consisting of hydrogen, C₁₋₆alkyl and phenyl, or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X2} and R^{X3} are each independently selected from the group consisting of hydrogen, halogen, hydroxyl, —NR^{a}R^{b}, —C(O)—NR^{a}R^{b}, C₁₋₆alkyl and C₁₋₆alkoxy; wherein C₁₋₆alkyl and C₁₋₆alkoxy may optionally be substituted by one or more substituents each independently selected from R^{P};

or R^{X2} and R^{X3} together with the carbon to which they are attached form a 3-6 membered carbocycle optionally substituted by one or more substituents each independently selected from the group consisting of hydrogen, halogen, —NR^{a}R^{b}, —C(O)—NR^{a}R^{b}, oxo, C₁₋₆ alkyl and C₁₋₆alkoxy;

R^{X4} and R^{X5} are each independently selected from the group consisting of hydrogen and C₁₋₆alkyl; wherein C₁₋₆alkyl may optionally be substituted by one or more substituents each independently selected from R^{P};

R^{X6} and R^{X7} are each independently selected from the group consisting of hydrogen, halogen, —C(O)—NR^{a}R^{b}, cyano and C₁₋₆alkyl; wherein C₁₋₆alkyl may optionally be substituted by one or more substituents each independently selected from R^{P};

R^{Y} is selected from the group consisting of hydrogen, C₁₋₆alkyl and phenyl;

R³ is selected from the group consisting of hydrogen, halogen, hydroxyl, C₁₋₆ alkyl and —NR^{a}R^{b};

R^{f} is independently selected, for each occurrence, from the group consisting of R^{P}, C₁₋₆ alkyl, C₃₋₆cycloalkyl, C₂₋₆alkenyl, C₂₋₆alkynyl, C₁₋₆alkoxy, C₁₋₆ alkylcarbonyl-, C₁₋₆alkoxycarbonyl, C₁₋₆alkyl-S(O)_{w}—, (wherein wherein w is 0, 1 or 2), C₁₋₆alkylcarbonyl-N(R^{a})— and C₁₋₆alkoxycarbonyl-N(R^{a})—; wherein C₁₋₆alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, C₁₋₆alkylcarbonyl-N(R^{a})—, and C₁₋₆alkoxycarbonyl-N(R^{a})— may be optionally substituted by one or more substituents selected from R^{P};

R^{h} is independently selected, for each occurrence, from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆ alkylcarbonyl-, C₁₋₆alkoxycarbonyl-, R^{a}R^{b}N-carbonyl- and R^{a}R^{b}N—SO₂—; wherein C₁₋₆alkyl, C₃₋₆alkenyl, C₃₋₆alkynyl, C₃₋₆cycloalkyl, C₁₋₆alkyl-S(O)₂—, C₁₋₆alkylcarbonyl- and C₁₋₆ alkoxycarbonyl- may optionally be substituted by one or more substituents selected from R^{P};

R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl;

or R^{a} and R^{b}, together with the nitrogen to which they are attached, may form a 4-6 membered monocyclic heterocyclic ring, which may have an additional heteroatom selected from the group consisting of O, S, and N; wherein the 4-6 membered heterocyclic ring may optionally be substituted by one or more substituents selected from the group consisting of halogen, cyano, oxo or hydroxyl; and

R^{P} is independently selected, for each occurrence, from the group consisting of halogen, hydroxyl, cyano, C₁₋₆alkoxy, R^{a}R^{b}N—, R^{a}R^{b}N-carbonyl-, R^{a}R^{b}N—SO₂—, and R^{a}R^{b}N-carbonyl-N(R^{a})—.

In some embodiments, X is selected from the group consisting of a bond, —O—, —NR^{X1}— and —NR^{X11}.

In some embodiments, A is selected from the group consisting of phenyl, pyridyl, quinolinyl, indolyl, and indolinyl, wherein phenyl, pyridyl, quinolinyl, indolyl and indolinyl may optionally be substituted by one, two or three substituents each independently selected from the group consisting of —R¹⁰, —OR¹⁰, —SR¹⁰, —N(R¹⁰)₂, —OSO₂R¹⁰, —SO₂R¹⁰, —C(O)N(R¹⁰)₂, halogen, and cyano. In certain embodiments, A is phenyl; wherein phenyl may optionally be substituted by one, two, or three substituents each independently selected from the group consisting of —OR¹⁰, halogen, and cyano. In certain other embodiments, A is pyridyl; wherein pyridyl may optionally be substituted by one, two, or three substituents each independently selected from the group consisting of —OR¹⁰, halogen, and cyano.

In some embodiments, R^{X1} and ring A together with the nitrogen to which they are attached form a 1,2,3,4-tetrahydro-1,5-naphthyridine moiety. In some embodiments, R^{X11} and ring A together with the nitrogen to which they are attached form a 1,2,3,4-tetrahydro-1,5-naphthyridine moiety. In some embodiments, R^{X1} and ring A together with the nitrogen to which they are attached form a 1,2,3,4-tetrahydroquinoline moiety. In some embodiments, R^{X11} and ring A together with the nitrogen to which they are attached form a 1,2,3,4-tetrahydroquinoline moiety.

In some embodiments, A is phenyl, pyridyl, indolyl, or indolinyl, wherein said phenyl, pyridyl, indolyl or indolinyl is optionally substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano.

In some embodiments, A is phenyl, pyridyl, imidazolyl, indolyl, indolinyl, naphthyl, quinolinyl, isoquinolinyl, benzimidazolyl, indazolyl, benzotriazolyl, benzothiazolyl, benzofuranyl, pyrazolopyridyl, quinazolinyl, quinoxalinyl, or cinnolinyl. In some embodiments, A is phenyl. In some embodiments, A is pyridyl. In some embodiments, A is 2-pyridyl. In some embodiments, A is 3-pyridyl. In some embodiments, A is 4-pyridyl. In some embodiments, A is naphthyl. In some embodiments, A is 1-naphthyl. In some embodiments, A is 2-naphthyl. In some embodiments, A is quinolinyl. In some embodiments, A is 2-quinolinyl. In some embodiments, A is 3-quinolinyl. In some embodiments, A is 4-quinolinyl. In some embodiments, A is 5-quinolinyl. In some embodiments, A is 6-quinolinyl. In some embodiments, A is 7-quinolinyl. In some embodiments, A is 8-quinolinyl. In some embodiments, A is isoquinolinyl. In some embodiments, A is 1-isoquinolinyl. In some embodiments, A is 3-isoquinolinyl. In some embodiments, A is 4-isoquinolinyl. In some embodiments, A is 5-isoquinolinyl. In some embodiments, A is 6-isoquinolinyl. In some embodiments, A is 7-isoquinolinyl. In some embodiments, A is 8-isoquinolinyl. In some embodiments, A is indolyl. In some embodiments, A is 2-indolyl. In some embodiments, A is 3-indolyl. In some embodiments, A is 4-indolyl. In some embodiments, A is 5-indolyl. In some embodiments, A is 6-indolyl. In some embodiments, A is 7-indolyl. In some embodiments, A is aryl. In some embodiments, A is bicyclic aryl. In some embodiments, A is heteroaryl. In some embodiments, A is fused bicyclic heteroaryl.

In some embodiments, A is phenyl, pyridyl, or indolyl, wherein said phenyl, pyridyl, or indolyl is optionally substituted with —OR¹⁰, halogen, or cyano.

In some embodiments, A is phenyl, wherein said phenyl is optionally substituted with —OR¹⁰, halogen, or cyano.

In some embodiments, A is pyridyl, wherein said pyridyl is optionally substituted with —OR¹⁰, halogen, or cyano.

In some embodiments, A is indolyl or indolinyl, wherein said indolyl or indolinyl is optionally substituted with —OR¹⁰, halogen, or cyano.

In some embodiments, A is phenyl, pyridyl, indolyl, or indolinyl, wherein said phenyl, pyridyl, indolyl or indolinyl is optionally substituted with —OR¹⁰ or halogen.

In some embodiments, A is phenyl, pyridyl, indolyl, or indolinyl, wherein said phenyl, pyridyl, indolyl or indolinyl is optionally substituted with halogen.

In some embodiments, A is phenyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is phenyl substituted with —R¹⁰. In some embodiments, A is phenyl substituted with —OR¹⁰. In some embodiments, A is phenyl substituted with —SR¹⁰. In some embodiments, A is phenyl substituted with —N(R¹⁰)₂. In some embodiments, A is phenyl substituted with —OSO₂R¹⁰. In some embodiments, A is phenyl substituted with —SO₂R¹⁰. In some embodiments, A is phenyl substituted with —C(O)N(R¹⁰)₂. In some embodiments, A is phenyl substituted with halogen. In some embodiments, A is phenyl substituted with cyano.

In some embodiments, A is pyridyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is pyridyl substituted with —R¹⁰. In some embodiments, A is pyridyl substituted with —OR¹⁰. In some embodiments, A is pyridyl substituted with —SR¹⁰. In some embodiments, A is pyridyl substituted with —N(R¹⁰)₂. In some embodiments, A is pyridyl substituted with —OSO₂R¹⁰. In some embodiments, A is pyridyl substituted with —SO₂R¹⁰. In some embodiments, A is pyridyl substituted with —C(O)N(R¹⁰)₂. In some embodiments, A is pyridyl substituted with halogen. In some embodiments, A is pyridyl substituted with cyano.

In some embodiments, A is 3-pyridyl substituted with —OR¹⁰. In some embodiments, A is 3-pyridyl substituted with —OCH₃. In some embodiments, A is 6-methoxypyrid-3-yl. In some embodiments, A is 3-pyridyl substituted with halogen. In some embodiments, A is 3-pyridyl substituted with Cl. In some embodiments, A is 4-chloropyrid-3-yl. In some embodiments, A is 2,3-dichloropyrid-4-yl. In some embodiments, A is 3-chloropyrid-2-yl.

In some embodiments, A is naphthyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is naphthyl substituted with —R¹⁰. In some embodiments, A is naphthyl substituted with —OR¹⁰ In some embodiments, A is naphthyl substituted with —SR¹⁰. In some embodiments, A is naphthyl substituted with —N(R¹⁰)₂. In some embodiments, A is naphthyl substituted with —OSO₂R¹⁰. In some embodiments, A is naphthyl substituted with —SO₂R¹⁰. In some embodiments, A is naphthyl substituted with —C(O)N(R¹⁰)₂. In some embodiments, A is naphthyl substituted with halogen. In some embodiments, A is naphthyl substituted with cyano.

In some embodiments, A is quinolinyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is quinolinyl substituted with —R¹⁰. In some embodiments, A is quinolinyl substituted with —OR¹⁰. In some embodiments, A is quinolinyl substituted with —SR¹⁰. In some embodiments, A is quinolinyl substituted with —N(R¹⁰)₂. In some embodiments, A is quinolinyl substituted with —OSO₂R¹⁰. In some embodiments, A is quinolinyl substituted with —SO₂R¹⁰. In some embodiments, A is quinolinyl substituted with —C(O)N(R¹⁰)₂. In some embodiments, A is quinolinyl substituted with halogen. In some embodiments, A is quinolinyl substituted with cyano.

In some embodiments, A is isoquinolinyl substituted with —R¹⁰, —OR, —SR¹⁰, —N(R¹⁰)₂, —OSO₂R¹⁰, —SO₂R¹⁰, —C(O)N(R¹⁰)₂, halogen, or cyano. In some embodiments, A is isoquinolinyl substituted with —R¹⁰. In some embodiments, A is isoquinolinyl substituted with —OR¹⁰. In some embodiments, A is isoquinolinyl substituted with —SR¹⁰. In some embodiments, A is isoquinolinyl substituted with —N(R¹⁰)₂. In some embodiments, A is isoquinolinyl substituted with —OSO₂R¹⁰. In some embodiments, A is isoquinolinyl substituted with —SO₂R¹⁰. In some embodiments, A is isoquinolinyl substituted with —C(O)N(R¹⁰)₂. In some embodiments, A is isoquinolinyl substituted with halogen. In some embodiments, A is isoquinolinyl substituted with cyano.

In some embodiments, A is indolyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is indolyl substituted with —R¹⁰. In some embodiments, A is indolyl substituted with —OR¹⁰. In some embodiments, A is indolyl substituted with —SR¹⁰. In some embodiments, A is indolyl substituted with —N(R¹⁰)₂. In some embodiments, A is indolyl substituted with —OSO₂R¹⁰. In some embodiments, A is indolyl substituted with —SO₂R¹⁰. In some embodiments, A is indolyl substituted with —C(O)N(R¹⁰)₂. In some embodiments, A is indolyl substituted with halogen. In some embodiments, A is indolyl substituted with cyano.

In some embodiments, A is 1-methyl-1H-indol-4-yl. In some embodiments, A is 1-methyl-1H-indol-3-yl.

In some embodiments, A is imidazol-2-yl. In some embodiments, A is 1-methyl-imidazol-2-yl.

In some embodiments, A is selected from the group consisting of 1,2,3,4-tetrahydroquinoline; 1,2,3,4-tetrahydro-1,5,napthyridine, dihydropyridopyrazine, benzoimidazole, benzoazepine, and dihydropyridooxazine, wherein A is bound to X through an available nitrogen on X; and where A is optionally substituted with one or two substituents each independently selected from halo, CF₃, phenyl (optionally substituted with one, two or three halo, C₁₋₃alkyl or C₁₋₃haloalkyl), C₁₋₃alkyl, C₁₋₃haloalkyl, and heteroaryl (optionally substituted with one, two or three halo, C₁₋₃alkyl or C₁₋₃haloalkyl).

In some embodiments, A is selected from the group consisting of

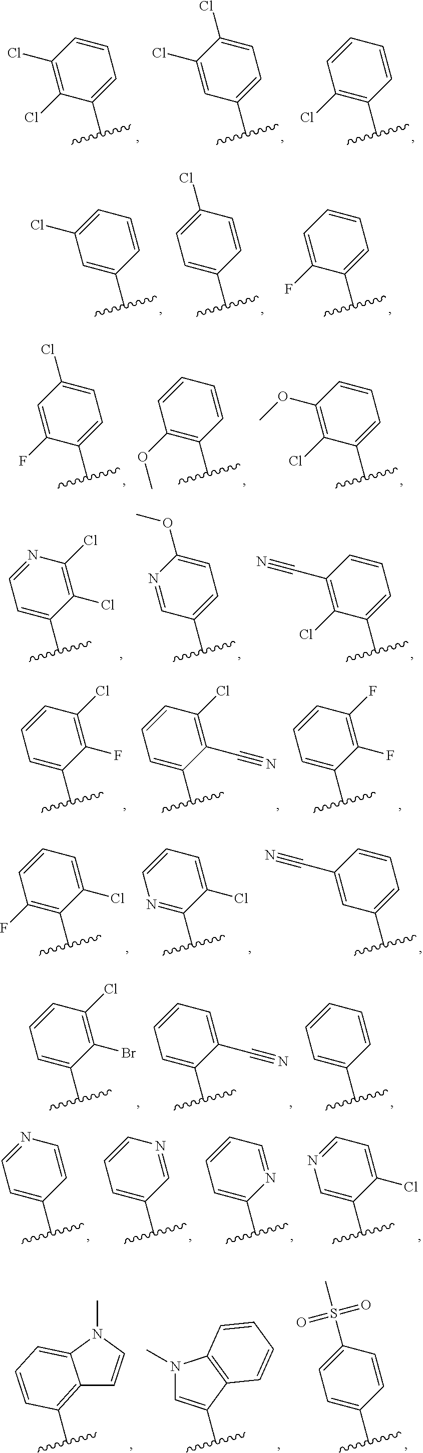

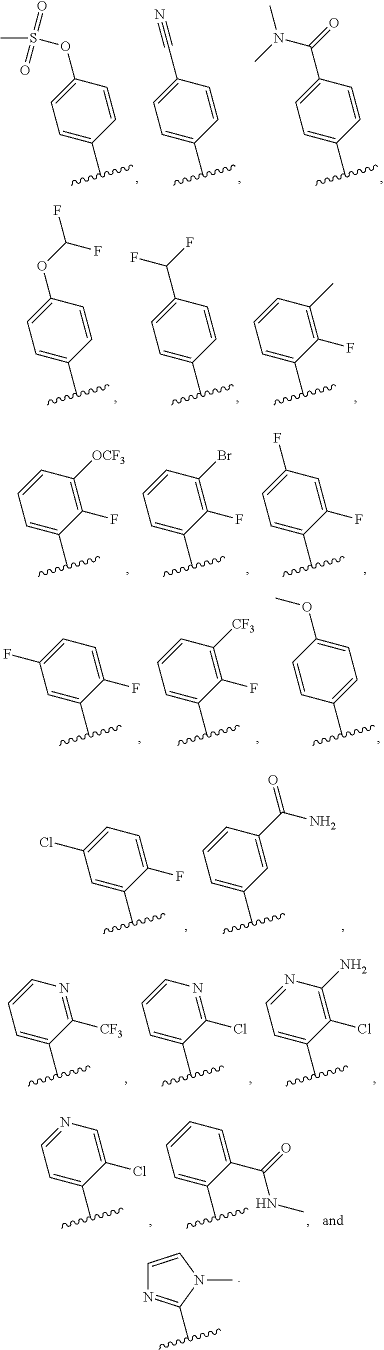

In some embodiments, A is

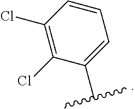 [SMILES: *c1cccc(Cl)c1Cl ; "."]

In some embodiments, A is

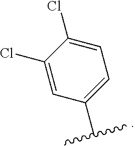 [SMILES: *c1ccc(Cl)c(Cl)c1 ; "."]

In some embodiments, A is

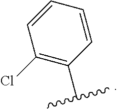 [SMILES: *c1ccccc1Cl ; "."]

In some embodiments, A is

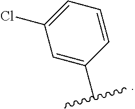 [SMILES: *c1cccc(Cl)c1 ; "."]

In some embodiments, A is

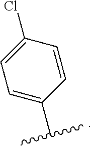 [SMILES: *c1ccc(Cl)cc1 ; "."]

In some embodiments, A is

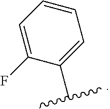 [SMILES: *c1ccccc1F ; "."]

In some embodiments, A is

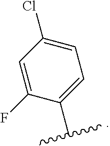 [SMILES: *c1ccc(Cl)cc1F ; "."]

In some embodiments, A is

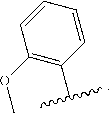 [SMILES: *c1ccccc1OC ; "."]

In some embodiments, A is

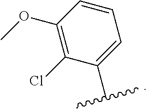 [SMILES: *c1cccc(OC)c1Cl ; "."]

In some embodiments, A is

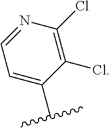 [SMILES: *c1ccnc(Cl)c1Cl]

In some embodiments, A is

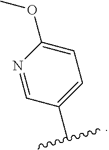 [SMILES: *c1ccc(OC)nc1 ; "."]

In some embodiments, A is

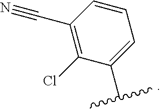 [SMILES: *c1cccc(C#N)c1Cl ; "."]

In some embodiments, A is

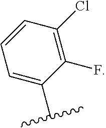 [SMILES: *c1cccc(Cl)c1F]

In some embodiments, A is

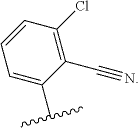 [SMILES: *c1cccc(Cl)c1C#N]

In some embodiments, A is

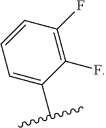 [SMILES: *c1cccc(F)c1F]

In some embodiments, A is

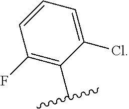 [SMILES: *c1c(F)cccc1Cl]

In some embodiments, A is

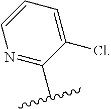 [SMILES: *c1ncccc1Cl]

In some embodiments, A is

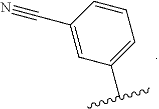 [SMILES: *c1cccc(C#N)c1]

In some embodiments, A is

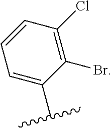 [SMILES: *c1cccc(Cl)c1Br]

In some embodiments, A is

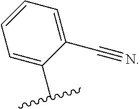 [SMILES: *c1ccccc1C#N]

In some embodiments, A is

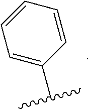 [SMILES: *c1ccccc1]

In some embodiments, A is

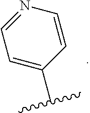 [SMILES: *c1ccncc1]

In some embodiments, A is

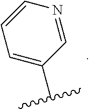 [SMILES: *c1cccnc1]

In some embodiments, A is

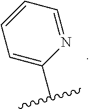 [SMILES: *c1ccccn1]

In some embodiments, A is

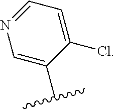 [SMILES: *c1cnccc1Cl]

In some embodiments, A is

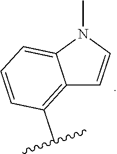 [SMILES: *c1cccc2c1ccn2C]

In some embodiments, A is

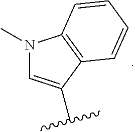 [SMILES: *c1cn(C)c2ccccc12]

In some embodiments, A is

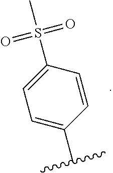 [SMILES: *c1ccc(S(C)(=O)=O)cc1]

In some embodiments, A is

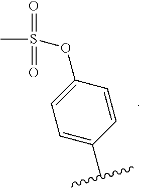 [SMILES: *c1ccc(OS(C)(=O)=O)cc1]

In some embodiments, A is

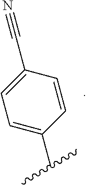 [SMILES: *c1ccc(C#N)cc1]

In some embodiments, A is

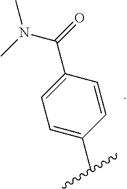 [SMILES: *c1ccc(C(=O)N(C)C)cc1]

In some embodiments, A is

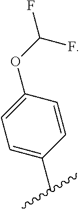 [SMILES: *c1ccc(OC(F)F)cc1]

In some embodiments, A is

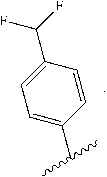 [SMILES: *c1ccc(C(F)F)cc1]

In some embodiments, A is

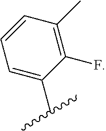 [SMILES: *c1cccc(C)c1F]

In some embodiments, A is

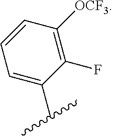 [SMILES: *c1cccc(OC(F)(F)F)c1F]

In some embodiments, A is

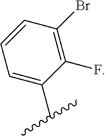 [SMILES: *c1cccc(Br)c1F]

In some embodiments, A is

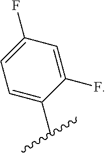 [SMILES: *c1ccc(F)cc1F]

In some embodiments, A is

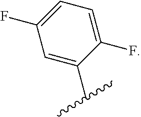 [SMILES: *c1cc(F)ccc1F]

In some embodiments, A is

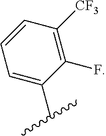 [SMILES: *c1cccc(C(F)(F)F)c1F]

In some embodiments, A is

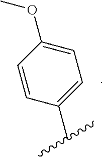 [SMILES: *c1ccc(OC)cc1]

In some embodiments, A is

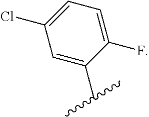 [SMILES: *c1cc(Cl)ccc1F]

In some embodiments, A is

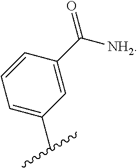 [SMILES: *c1cccc(C(N)=O)c1]

In some embodiments, A is

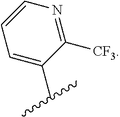 [SMILES: *c1cccnc1C(F)(F)F]

In some embodiments, A is

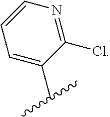 [SMILES: *c1cccnc1Cl]

In some embodiments, A is

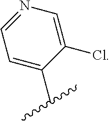 [SMILES: *c1ccncc1Cl]

In some embodiments, A is

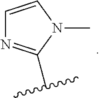 [SMILES: *c1nccn1C]

In some embodiments, A is

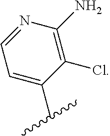 [SMILES: *c1ccnc(N)c1Cl]

In some embodiments, A is

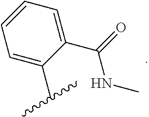 [SMILES: *c1ccccc1C(=O)NC]

In certain embodiments, R³ is selected from the group consisting of hydrogen, CH₃ and —NH₂. In some embodiments, Y is a bond and Z is N.

In some embodiments, R¹ and R² together with the nitrogen to which they are attached form heterocyclic ring B; wherein ring B is selected from the group consisting of pyrrolidinyl, piperidinyl, azepanyl and morpholinyl; wherein pyrrolidinyl, piperidinyl, azepanyl and morpholinyl may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃alkoxy; wherein C₁₋₃alkyl and C₁₋₃alkoxy may optionally be substituted by halogen, hydroxyl and —NR^{a}R^{b}.

In some embodiments, Y is —NH—, —N(CH₃)—, —N(H)—CH₂—, or —N(CH₃)—CH₂—; and Z is C(H).

In certain embodiments, R¹ and R² together with the carbon to which they are attached form carbocyclic ring B; wherein ring B is selected from the group consisting of cyclopentyl and cyclohexyl, and wherein cyclopentyl and cyclohexyl may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃ alkoxy; wherein C₁₋₃alkyl and C₁₋₃alkoxy may optionally be substituted by halogen, hydroxyl and —NR^{a}R^{b}.

In certain other embodiments, R¹ and R² together with the carbon to which they are attached form heterocyclic ring B; wherein ring B is selected from the group consisting of tetrahydrofuranyl and tetrahydropyranyl, wherein tetrahydrofuranyl and tetrahydropyranyl may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃alkoxy; wherein C₁₋₃alkyl and C₁₋₃alkoxy may optionally be substituted by halogen, hydroxyl and —NR^{a}R^{b}.

For example, in some embodiments, ring B is substituted on an available carbon by a substituent selected from the group consisting of —NH₂, —CH₃, and —CH₂NH₂.

In certain embodiments, R¹ is hydrogen and R² is branched C₁₋₁₀alkyl; wherein C₁₋₁₀alkyl may optionally be substituted by one, two, three or more substituents each independently selected from the group consisting of halogen and —NH₂.

In some embodiments, SHP2 phosphatase inhibitors described herein encompass compounds of Formula V, or a pharmaceutically acceptable salt or stereoisomer thereof, wherein Formula V is represented by:

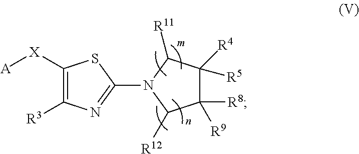

wherein

X is selected from the group consisting of a bond, —O—, —NR^{X1}—, —C(O)NR^{X11}—, —S(O)_{w}— (wherein w is 0, 1 or 2), —C(O)— and —C(R^{X2}R^{X3});

A is selected from the group consisting of phenyl, a 5-7 membered monocyclic heteroaryl having one or more heteroatoms each independently selected from O, S, and N, a 8-10 membered bicyclic heteroaryl having one or more heteroatoms each independently selected from the group consisting of O, S, and N, and a 4-7 membered heterocyclyl; wherein A may optionally be substituted by one, two, three or more substituents each independently selected from the group consisting of —R¹⁰, —OR¹⁰, —N(R¹⁰)₂, —C(O)N(R¹⁰)₂, oxo, halogen and cyano;

R¹⁰ is independently selected from the group consisting of hydrogen, C₁₋₆alkyl, C₁₋₆haloalkyl, C₃₋₆cycloalkyl, heterocycloalkyl, aryl, and heteroaryl, where R¹⁰ may optionally be substituted by one, two, three or more substituents each independently selected from the group consisting of halogen, —C(O)R²⁰, C₁₋₆alkyl and C₁₋₆haloalkyl;

R²⁰ is selected from the group consisting of hydroxyl, halogen, and C₁₋₆alkyl;

R^{X1} is selected from the group consisting of hydrogen, C₁₋₆alkyl and phenyl; or R^{X1} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —N(R¹⁰)₂, —C(O)N(R¹⁰)₂, oxo, halogen and cyano;

R^{X11} is selected from the group consisting of hydrogen, C₁₋₆alkyl and phenyl; or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —N(R¹⁰)₂, —C(O)N(R¹⁰)₂, oxo, halogen and cyano;

R^{X2} and R^{X3} are each independently selected from the group consisting of hydrogen, halogen, hydroxyl, —NR^{a}R^{b}, —C(O)—NR^{a}R^{b}, C₁₋₆alkyl and C₁₋₆alkoxy; or R^{X2} and R^{X3} together with the carbon to which they are attached form a 3-6 membered carbocyclyl which may optionally be substituted by one or more substituents each independently selected from the group consisting of hydrogen, halogen, —NR^{a}R^{b}, —C(O)—NR^{a}R^{b}, oxo, C₁₋₆alkyl and C₁₋₆ alkoxy;

R³ is selected from the group consisting of hydrogen, halogen, hydroxyl, C₁₋₆alkyl and —NR^{a}R^{b};

R⁴ and R⁵ are each independently selected from the group consisting of hydrogen, hydroxyl, C₁₋₆alkyl, C₁₋₆alkoxy, C₁₋₆alkyl-O—R⁶, —C(O)N(R⁶)₂, —N(R⁶)₂, halogen, C₁₋₆alkyl-N(R⁶)₂, and cyano; wherein C₁₋₆alkyl, C₁₋₆alkoxy, C₁₋₆alkyl-O—R⁶, and C₁₋₆alkyl-N(R⁶)₂ may optionally be substituted by one or more substituents each independently selected from the group consisting of hydroxyl, —NH₂, oxo, and halogen,

or R⁴ and R⁵, taken together with the atoms to which they are attached, form a 3-7 membered carbocyclic or heterocyclic saturated or partially unsaturated ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;

or R⁴ and R⁸, taken together with the atoms to which they are attached, form a 4-7 membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;

or R⁴ is absent, and R⁴ and R⁸, taken together with the atoms to which they are attached, form a 3-membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;

R⁶ is independently for each occurrence selected from the group consisting of hydrogen, C₁₋₆alkyl and phenyl;

R¹¹ and R¹² are each independently selected from the group consisting of hydrogen, hydroxyl, C₁₋₆alkyl, C₁₋₆alkoxy, C₁₋₆alkyl-O—R⁶, aryl, arylalkylene, heteroaryl, heteroarylalkylene, —C(O)N(R⁶)₂, —N(R⁶)₂, halogen, C₁₋₆alkyl-N(R⁶)₂, —CO₂H, and cyano; wherein C₁₋₆alkyl, C₁₋₆alkoxy, C₁₋₆alkyl-O—R⁶, aryl, arylalkylene, heteroaryl, heteroarylalkylene, and C₁₋₆alkyl-N(R⁶)₂ may optionally be substituted by one or more substituents each independently selected from the group consisting of hydroxyl, —NH₂, and halogen;

or R¹¹ and R¹², taken together with the atoms to which they are attached, form a 5-7 membered heterocyclic ring;

or R⁴ and R¹², taken together with the atoms to which they are attached, form a 5-7 membered carbocyclic or heterocyclic ring;

or R⁸ and R¹¹, taken together with the atoms to which they are attached, form a 5-7 membered carbocyclic or heterocyclic ring;

R⁸ and R⁹ are each independently selected from the group consisting of hydrogen, C₁₋₆)alkyl, C₁₋₆alkyl-N(R⁶)₂, —OR⁶, C₁₋₆alkyl-O—R⁶, —C(O)NH₂, —N(R⁶)₂, halogen, and cyano; and

each of m and n is, independently, 0, 1, 2, or 3, with m+n being at least 2 and no more than 4.

In some embodiments, A is phenyl; wherein phenyl may optionally be substituted by one, two, or three substituents each independently selected from the group consisting of —OR¹⁰, halogen, and cyano. In some embodiments, A is pyridyl; wherein pyridyl may optionally be substituted by one, two, or three substituents each independently selected from the group consisting of —OR¹⁰, halogen, and cyano.

In some embodiments, R^{X1} and ring A together with the nitrogen to which they are attached form a 1,2,3,4-tetrahydro-1,5-naphthyridine moiety. In some embodiments, R^{X11} and ring A together with the nitrogen to which they are attached form a 1,2,3,4-tetrahydro-1,5-naphthyridine moiety. In some embodiments, R^{X1} and ring A together with the nitrogen to which they are attached form a 1,2,3,4-tetrahydroquinoline moiety. In some embodiments, R^{X11} and ring A together with the nitrogen to which they are attached form a 1,2,3,4-tetrahydroquinoline moiety.

In certain embodiments, R³ is selected from the group consisting of hydrogen, CH₃ and —NH₂. In certain other embodiments, m is an integer selected from 1 or 2; and n is 1.

In some embodiments, R⁴ and R⁵ are each independently selected from the group consisting of hydrogen, hydroxyl, C₁₋₃alkyl, C₁₋₃alkoxy, C₁₋₃alkyl-O—R⁶, —C(O)NH₂, —N(R⁶)₂, halogen, C₁₋₃alkyl-N(R⁶)₂, and cyano; wherein C₁₋₃alkyl, C₁₋₃alkoxy, C₁₋₃alkyl-O—R⁶ and C₁₋₃alkyl-N(R⁶)₂ may optionally be substituted by one or more substituents each independently selected from the group consisting of hydroxyl, —N(R⁶)₂, and halogen;

or R⁴ and R⁵, taken together with the atoms to which they are attached, form a 3-7 membered carbocyclic or heterocyclic ring;

or R⁴ and R⁸, taken together with the atoms to which they are attached, form a 4-7 membered carbocyclic or heterocyclic ring;

or R⁴ is a bond, and R⁴ and R⁸, taken together with the atoms to which they are attached, form a 3-membered carbocyclic or heterocyclic ring; and

each R⁶ is independently hydrogen or C₁₋₃alkyl.

For example, in some embodiments, R⁴ and R⁵ are each independently selected from the group consisting of C₁₋₃alkyl and —NR^{a}R^{b}; wherein C₁₋₃alkyl may optionally be substituted by one, two or three substituents each independently selected from the group consisting of halogen, hydroxyl and —NR^{a}R^{b}. In certain embodiments, R⁴ and R⁵ are each independently selected from the group consisting of —NH₂, —CH₃, and —CH₂NH₂.

In some embodiments, SHP2 phosphatase inhibitors described herein encompass compounds of Formula VI, or a pharmaceutically acceptable salt or stereoisomer thereof, wherein Formula VI is represented by:

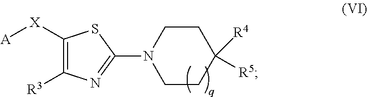

wherein

X is selected from the group consisting of a bond, —NR^{X1}—, and —C(O)NR^{X11}—;

A is selected from the group consisting of phenyl, a 5-7 membered monocyclic heteroaryl having one or more heteroatoms each independently selected from O, S, and N, a 8-10 membered bicyclic heteroaryl having one or more heteroatoms each independently selected from the group consisting of O, S, and N, and a 4-7 membered heterocyclyl; wherein A may optionally be substituted by one, two, three or more substituents each independently selected from the group consisting of —R¹⁰, —OR¹⁰, —N(R¹⁰)₂, —C(O)N(R¹⁰)₂, oxo, halogen and cyano;

R¹⁰ is independently selected from the group consisting of hydrogen, C₁₋₆alkyl, C₁₋₆haloalkyl, C₃₋₆cycloalkyl, heterocycloalkyl, aryl, and heteroaryl, where R¹⁰ may optionally be substituted by one, two, three or more substituents each independently selected from the group consisting of halogen, —C(O)R²⁰, C₁₋₆alkyl and C₁₋₆haloalkyl;

R²⁰ is selected from the group consisting of hydroxyl, halogen, and C₁₋₆alkyl;

R^{X1} is selected from the group consisting of hydrogen, C₁₋₆alkyl and phenyl, or R^{X1} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —N(R¹⁰)₂, —C(O)N(R¹⁰)₂, oxo, halogen and cyano;

R^{X11} is selected from the group consisting of hydrogen, C₁₋₆alkyl and phenyl, or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —N(R¹⁰)₂, —C(O)N(R¹⁰)₂, oxo, halogen and cyano;

R³ is selected from the group consisting of hydrogen, halogen, hydroxyl, C₁₋₆alkyl and —NR^{a}R^{b};

R⁴ and R⁵ are each independently selected from the group consisting of hydrogen, halogen, cyano, —NR^{a}R^{b}, —C(O)—NR^{a}R^{b} and C₁₋₆alkyl, wherein C₁₋₆alkyl may optionally be substituted by one, two or three or more substituents each independently selected from the group consisting of halogen, hydroxyl, —NR^{a}R^{b} and oxo;

R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl; and q is 0 or 1.

In some embodiments, A is phenyl; wherein phenyl may optionally be substituted by one, two, or three substituents each independently selected from the group consisting of —OR¹⁰, halogen, and cyano. In some embodiments, A is pyridyl; wherein pyridyl may optionally be substituted by one, two, or three substituents each independently selected from the group consisting of —OR¹⁰, halogen, and cyano.

In some embodiments, R^{X1} and ring A together with the nitrogen to which they are attached form a 1,2,3,4-tetrahydro-1,5-naphthyridine moiety. In some embodiments, R^{X11} and ring A together with the nitrogen to which they are attached form a 1,2,3,4-tetrahydro-1,5-naphthyridine moiety. In some embodiments, R^{X1} and ring A together with the nitrogen to which they are attached form a 1,2,3,4-tetrahydroquinoline moiety. In some embodiments, R^{X11} and ring A together with the nitrogen to which they are attached form a 1,2,3,4-tetrahydroquinoline moiety.

In some embodiments, R³ is selected from the group consisting of hydrogen, CH₃ and —NH₂. In some embodiments, R⁴ and R⁵ are each independently selected from the group consisting of C₁₋₃alkyl and —NR^{a}R^{b}; wherein C₁₋₃alkyl may optionally be substituted by one, two or three substituents each independently selected from the group consisting of halogen, hydroxyl and —NR^{a}R^{b}. For example, in some embodiments, R⁴ and R⁵ are each independently selected from the group consisting of —NH₂, —CH₃, and —CH₂NH₂.

Examples of compounds of the present disclosure include:

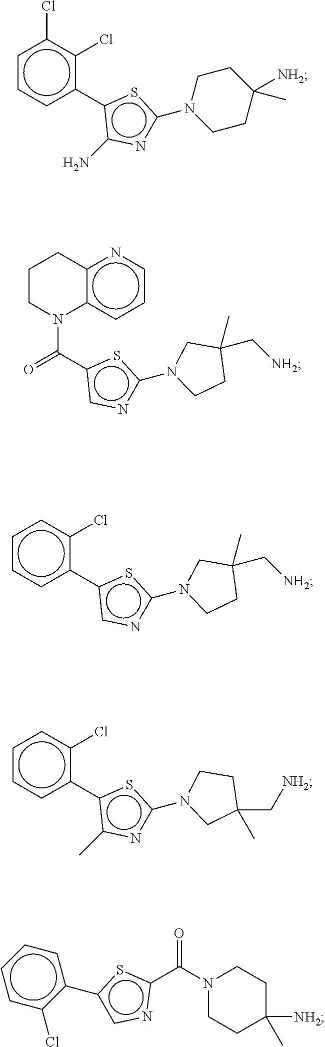

and a pharmaceutically acceptable salt or stereoisomer thereof.

Also provided herein is a compound of Formula VII, or a pharmaceutically acceptable salt, stereoisomer or N-oxide thereof, wherein Formula VII is represented by:

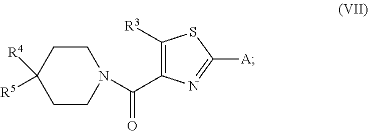

wherein

A is phenyl, wherein A may optionally be substituted by one, two, three or more substituents each independently selected from the group consisting of —R¹⁰, —OR¹⁰, methylenedioxy, —N(R¹⁰)₂, —C(O)N(R¹⁰)₂, halogen and cyano;

R¹⁰ is independently selected from the group consisting of hydrogen, C₁₋₆alkyl, C₁₋₆haloalkyl, C₃₋₆cycloalkyl, heterocycloalkyl, aryl, and heteroaryl, where R¹⁰ may optionally be substituted one, two, three or more substituents each independently selected from the group consisting of halogen, —C(O)R²⁰, C₁₋₆alkyl and C₁₋₆haloalkyl;

R²⁰ is selected from the group consisting of hydroxyl, halogen, and C₁₋₆alkyl;

R³ is selected from the group consisting of hydrogen, halogen, hydroxyl, C₁₋₆alkyl and —NR^{a}R^{b};

R⁴ and R⁵ are each independently selected from the group consisting of hydrogen, halogen, cyano, —NR^{a}R^{b}, —C(O)—NR^{a}R^{b} and C₁₋₆alkyl, wherein C₁₋₆alkyl may optionally be substituted by one, two or three or more substituents each independently selected from the group consisting of halogen, hydroxyl, —NR^{a}R^{b} and oxo; and

R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl.

In some embodiments, R⁴ and R⁵ are each independently selected from the group consisting of C₁₋₃alkyl and —NR^{a}R^{b}; wherein C₁₋₃alkyl may optionally be substituted by one, two or three substituents each independently selected from the group consisting of halogen, hydroxyl and —NR^{a}R^{b}. In some embodiments, R⁴ and R⁵ are each independently selected from the group consisting of —NH₂, —CH₃, and —CH₂NH₂, —C(H)NH₂CH₃ and —CH₂NHCH₃.

In some embodiments, SHP2 phosphatase inhibitors described herein encompass compounds of Formula XIII, or a pharmaceutically acceptable salt or stereoisomer thereof, wherein Formula XIII is represented by:

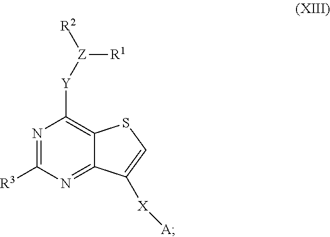

wherein

X is selected from the group consisting of a bond, —O—, —NR^{X1}—, —C(O)NR^{X11}—, —S(O)_{w}— (wherein w is 0, 1 or 2), —C(O)—, —C(R^{X2}R^{X3})—, —O—C(R^{X4}R^{X5})—, —C(R^{X4}R^{X5})—O—, and —C═C(R^{X6}R^{X7})—;

Y is selected from the group consisting of a bond, —NR^{Y}—, —C₁₋₃alkylene-NR^{Y}—, and —NR^{Y}—C₁₋₃alkylene-;

Z is selected from the group consisting of N, C(R^{Z}) and a bond;

wherein if Y is a bond, Z is N or a bond; and wherein if Y is —NR^{Y}—, —C₁₋₃alkylene-NR^{Y}—, or —NR^{Y}—C₁₋₃alkylene-, Z is C(R^{Z});

R^{Z} is selected from the group consisting of hydrogen and C₁₋₆alkyl; wherein C₁₋₆alkyl may optionally be substituted by halogen, hydroxyl, and —NR^{a}R^{b},

A is selected from the group consisting of a 6-10 membered monocyclic or bicyclic aryl, a 5-7 membered monocyclic heteroaryl having one or more heteroatoms each independently selected from O, S, or N, a 8-10 membered bicyclic heteroaryl having one or more heteroatoms each independently selected from O, S, or N; and a 4-7 membered heterocyclyl, wherein A may optionally be substituted by one, two, three or more substituents each independently selected from the group consisting of —R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano, or when X is a bond, A may also be selected from the group consisting of H and halogen;

R¹⁰ is independently selected from the group consisting of hydrogen, C₁₋₆alkyl, C₁₋₆haloalkyl, C₃₋₆cycloalkyl, heterocycloalkyl, aryl, and heteroaryl; wherein R¹⁰ may optionally be substituted by one, two, three or more substituents each independently selected from the group consisting of halogen, —C(O)R²⁰, C₁₋₆alkyl and C₁₋₆haloalkyl;

R²⁰ is selected from the group consisting of hydroxyl, halogen, and C₁₋₆alkyl;

R¹ and R² are each selected from the group consisting of hydrogen, C₁₋₁₀alkyl and phenyl; wherein C₁₋₁₀alkyl and phenyl may optionally be substituted by one, two, three or more substituents each independently selected from R^{P}, and wherein when Z and Y are each a bond R² is not present;

or R¹ and R², together with carbon or nitrogen to which they are attached, form a 3-7 membered saturated carbocyclic or 4-7 membered saturated heterocyclic ring B having one or two heteroatoms each independently selected from the group consisting of O, S(O)_{w} (wherein w is 0, 1, or 2), and NR^{h}; wherein carbocyclic or heterocyclic ring B may be monocyclic or bicyclic;

carbocyclic or heterocyclic ring B may optionally be substituted on one or more available carbons by one, two, three or more substituents each independently selected from the group consisting of halogen, hydroxyl, cyano, oxo, —NR^{a}R^{b}, —CO₂H, —C(O)—NR^{a}R^{b}, —S(O)₂—NR^{a}R^{b}, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})—, C₁₋₆alkoxycarbonyl-N(R^{a})—, aryl and heteroaryl; wherein C₁₋₆alkyl, C₂₋₆alkenyl, C₂₋₆alkynyl, C₃₋₆cycloalkyl, C₁₋₆alkoxy, C₃₋₆ alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})— and C₁₋₆alkoxycarbonyl-N(R^{a})— may optionally be substituted by one or more substituents each independently selected from R^{P}; and wherein aryl and heteroaryl may optionally be substituted by one or more substituents each independently selected from R^{f};

R^{X1} is selected from the group consisting of hydrogen, C₁₋₆alkyl and phenyl, or R^{X1} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X11} is selected from the group consisting of hydrogen, C₁₋₆alkyl and phenyl, or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X2} and R^{X3} are each independently selected from the group consisting of hydrogen, halogen, hydroxyl, —NR^{a}R^{b}, —C(O)—NR^{a}R^{b}, C₁₋₆alkyl and C₁₋₆alkoxy; wherein C₁₋₆alkyl and C₁₋₆alkoxy may optionally be substituted by one or more substituents each independently selected from R^{P};

or R^{X2} and R^{X3} together with the carbon to which they are attached form a 3-6 membered carbocycle optionally substituted by one or more substituents each independently selected from the group consisting of hydrogen, halogen, —NR^{a}R^{b}, —C(O)—NR^{a}R^{b}, oxo, C₁₋₆ alkyl and C₁₋₆alkoxy;

R^{X4} and R^{X5} are each independently selected from the group consisting of hydrogen and C₁₋₆alkyl; wherein C₁₋₆alkyl may optionally be substituted by one or more substituents each independently selected from R^{P};

R^{X6} and R^{X7} are each independently selected from the group consisting of hydrogen, halogen, —C(O)—NR^{a}R^{b}, cyano and C₁₋₆alkyl; wherein C₁₋₆alkyl may optionally be substituted by one or more substituents each independently selected from R^{P};

R^{Y} is selected from the group consisting of hydrogen, C₁₋₆alkyl and phenyl;

R³ is selected from the group consisting of hydrogen, halogen, hydroxyl, C₁₋₆alkyl and —NR^{a}R^{b};

R^{f} is independently selected, for each occurrence, from the group consisting of R^{P}, C₁₋₆ alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, (wherein wherein w is 0, 1 or 2), C₁₋₆alkylcarbonyl-N(R^{a})— and C₁₋₆alkoxycarbonyl-N(R^{a})—; wherein C₁₋₆alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, C₁₋₆alkylcarbonyl-N(R^{a})—, and C₁₋₆alkoxycarbonyl-N(R^{a})— may be optionally substituted by one or more substituents selected from R^{P};

R^{h} is independently selected, for each occurrence, from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆ alkylcarbonyl-, C₁₋₆alkoxycarbonyl-, R^{a}R^{b}N-carbonyl- and R^{a}R^{b}N—SO₂—; wherein C₁₋₆alkyl, C₃₋₆alkenyl, C₃₋₆alkynyl, C₃₋₆cycloalkyl, C₁₋₆alkyl-S(O)₂—, C₁₋₆alkylcarbonyl- and C₁₋₆ alkoxycarbonyl- may optionally be substituted by one or more substituents selected from R^{P};

R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl;

or R^{a} and R^{b}, together with the nitrogen to which they are attached, may form a 4-6 membered monocyclic heterocyclic ring, which may have an additional heteroatom selected from the group consisting of O, S, and N; wherein the 4-6 membered heterocyclic ring may optionally be substituted by one or more substituents selected from the group consisting of halogen, cyano, oxo or hydroxyl; and

R^{P} is independently selected, for each occurrence, from the group consisting of halogen, hydroxyl, cyano, C₁₋₆alkoxy, R^{a}R^{b}N—, R^{a}R^{b}N-carbonyl-, R^{a}R^{b}N—SO₂—, and R^{a}R^{b}N-carbonyl-N(R^{a})—.

In some embodiments, X is selected from the group consisting of a bond, —O—, —NR^{X1}— and —NR^{X11}.

In some embodiments, A is selected from the group consisting of phenyl, pyridyl, quinolinyl, indolyl, and indolinyl, wherein phenyl, pyridyl, quinolinyl, indolyl and indolinyl may optionally be substituted by one, two or three substituents each independently selected from the group consisting of —R¹⁰, —OR¹⁰, —SR¹⁰, —N(R¹⁰)₂, —OSO₂R¹⁰, —SO₂R¹⁰, —C(O)N(R¹⁰)₂, halogen, and cyano. In certain embodiments, A is phenyl; wherein phenyl may optionally be substituted by one, two, or three substituents each independently selected from the group consisting of —OR¹⁰, halogen, and cyano. In certain other embodiments, A is pyridyl; wherein pyridyl may optionally be substituted by one, two, or three substituents each independently selected from the group consisting of —OR¹⁰, halogen, and cyano.

In some embodiments, R^{X1} and ring A together with the nitrogen to which they are attached form a 1,2,3,4-tetrahydro-1,5-naphthyridine moiety. In some embodiments, R^{X11} and ring A together with the nitrogen to which they are attached form a 1,2,3,4-tetrahydro-1,5-naphthyridine moiety. In some embodiments, R^{X1} and ring A together with the nitrogen to which they are attached form a 1,2,3,4-tetrahydroquinoline moiety. In some embodiments, R^{X11} and ring A together with the nitrogen to which they are attached form a 1,2,3,4-tetrahydroquinoline moiety.

In some embodiments, A is phenyl, pyridyl, indolyl, or indolinyl, wherein said phenyl, pyridyl, indolyl or indolinyl is optionally substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano.

In some embodiments, A is phenyl, pyridyl, imidazolyl, indolyl, indolinyl, naphthyl, quinolinyl, isoquinolinyl, benzimidazolyl, indazolyl, benzotriazolyl, benzothiazolyl, benzofuranyl, pyrazolopyridyl, quinazolinyl, quinoxalinyl, or cinnolinyl. In some embodiments, A is phenyl. In some embodiments, A is pyridyl. In some embodiments, A is 2-pyridyl. In some embodiments, A is 3-pyridyl. In some embodiments, A is 4-pyridyl. In some embodiments, A is naphthyl. In some embodiments, A is 1-naphthyl. In some embodiments, A is 2-naphthyl. In some embodiments, A is quinolinyl. In some embodiments, A is 2-quinolinyl. In some embodiments, A is 3-quinolinyl. In some embodiments, A is 4-quinolinyl. In some embodiments, A is 5-quinolinyl. In some embodiments, A is 6-quinolinyl. In some embodiments, A is 7-quinolinyl. In some embodiments, A is 8-quinolinyl. In some embodiments, A is isoquinolinyl. In some embodiments, A is 1-isoquinolinyl. In some embodiments, A is 3-isoquinolinyl. In some embodiments, A is 4-isoquinolinyl. In some embodiments, A is 5-isoquinolinyl. In some embodiments, A is 6-isoquinolinyl. In some embodiments, A is 7-isoquinolinyl. In some embodiments, A is 8-isoquinolinyl. In some embodiments, A is indolyl. In some embodiments, A is 2-indolyl. In some embodiments, A is 3-indolyl. In some embodiments, A is 4-indolyl. In some embodiments, A is 5-indolyl. In some embodiments, A is 6-indolyl. In some embodiments, A is 7-indolyl. In some embodiments, A is aryl. In some embodiments, A is bicyclic aryl. In some embodiments, A is heteroaryl. In some embodiments, A is fused bicyclic heteroaryl.

In some embodiments, A is phenyl, pyridyl, or indolyl, wherein said phenyl, pyridyl, or indolyl is optionally substituted with —OR¹⁰, halogen, or cyano.

In some embodiments, A is phenyl, wherein said phenyl is optionally substituted with —OR¹⁰, halogen, or cyano.

In some embodiments, A is pyridyl, wherein said pyridyl is optionally substituted with —OR¹⁰, halogen, or cyano.

In some embodiments, A is indolyl or indolinyl, wherein said indolyl or indolinyl is optionally substituted with —OR¹⁰, halogen, or cyano.

In some embodiments, A is phenyl, pyridyl, indolyl, or indolinyl, wherein said phenyl, pyridyl, indolyl or indolinyl is optionally substituted with —OR¹⁰ or halogen.

In some embodiments, A is phenyl, pyridyl, indolyl, or indolinyl, wherein said phenyl, pyridyl, indolyl or indolinyl is optionally substituted with halogen.

In some embodiments, A is phenyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is phenyl substituted with —R¹⁰. In some embodiments, A is phenyl substituted with —OR¹⁰. In some embodiments, A is phenyl substituted with —SR¹⁰. In some embodiments, A is phenyl substituted with —N(R¹⁰)₂. In some embodiments, A is phenyl substituted with —OSO₂R¹⁰. In some embodiments, A is phenyl substituted with —SO₂R¹⁰. In some embodiments, A is phenyl substituted with —C(O)N(R¹⁰)₂. In some embodiments, A is phenyl substituted with halogen. In some embodiments, A is phenyl substituted with cyano.

In some embodiments, A is pyridyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is pyridyl substituted with —R¹⁰. In some embodiments, A is pyridyl substituted with —OR¹⁰. In some embodiments, A is pyridyl substituted with —SR¹⁰. In some embodiments, A is pyridyl substituted with —N(R¹⁰)₂. In some embodiments, A is pyridyl substituted with —OSO₂R¹⁰. In some embodiments, A is pyridyl substituted with —SO₂R¹⁰. In some embodiments, A is pyridyl substituted with —C(O)N(R¹⁰)₂. In some embodiments, A is pyridyl substituted with halogen. In some embodiments, A is pyridyl substituted with cyano.

In some embodiments, A is 3-pyridyl substituted with —OR¹⁰. In some embodiments, A is 3-pyridyl substituted with —OCH₃. In some embodiments, A is 6-methoxypyrid-3-yl. In some embodiments, A is 3-pyridyl substituted with halogen. In some embodiments, A is 3-pyridyl substituted with Cl. In some embodiments, A is 4-chloropyrid-3-yl. In some embodiments, A is 2,3-dichloropyrid-4-yl. In some embodiments, A is 3-chloropyrid-2-yl.

In some embodiments, A is naphthyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is naphthyl substituted with —R¹⁰. In some embodiments, A is naphthyl substituted with —OR¹⁰. In some embodiments, A is naphthyl substituted with —SR¹⁰. In some embodiments, A is naphthyl substituted with —N(R¹⁰)₂. In some embodiments, A is naphthyl substituted with —OSO₂R¹⁰. In some embodiments, A is naphthyl substituted with —SO₂R¹⁰. In some embodiments, A is naphthyl substituted with —C(O)N(R¹⁰)₂. In some embodiments, A is naphthyl substituted with halogen. In some embodiments, A is naphthyl substituted with cyano.

In some embodiments, A is quinolinyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is quinolinyl substituted with —R¹⁰. In some embodiments, A is quinolinyl substituted with —OR¹⁰. In some embodiments, A is quinolinyl substituted with —SR¹⁰. In some embodiments, A is quinolinyl substituted with —N(R¹⁰)₂. In some embodiments, A is quinolinyl substituted with —OSO₂R¹⁰. In some embodiments, A is quinolinyl substituted with —SO₂R¹⁰. In some embodiments, A is quinolinyl substituted with —C(O)N(R¹⁰)₂. In some embodiments, A is quinolinyl substituted with halogen. In some embodiments, A is quinolinyl substituted with cyano.

In some embodiments, A is isoquinolinyl substituted with —R¹⁰, —OR, —SR¹⁰, —N(R¹⁰)₂, —OSO₂R¹⁰, —SO₂R¹⁰, —C(O)N(R¹⁰)₂, halogen, or cyano. In some embodiments, A is isoquinolinyl substituted with —R¹⁰. In some embodiments, A is isoquinolinyl substituted with —OR¹⁰. In some embodiments, A is isoquinolinyl substituted with —SR¹⁰. In some embodiments, A is isoquinolinyl substituted with —N(R¹⁰)₂. In some embodiments, A is isoquinolinyl substituted with —OSO₂R¹⁰. In some embodiments, A is isoquinolinyl substituted with —SO₂R¹⁰. In some embodiments, A is isoquinolinyl substituted with —C(O)N(R¹⁰)₂. In some embodiments, A is isoquinolinyl substituted with halogen. In some embodiments, A is isoquinolinyl substituted with cyano.

In some embodiments, A is indolyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is indolyl substituted with —R¹⁰. In some embodiments, A is indolyl substituted with —OR¹⁰. In some embodiments, A is indolyl substituted with —SR¹⁰. In some embodiments, A is indolyl substituted with —N(R¹⁰)₂. In some embodiments, A is indolyl substituted with —OSO₂R¹⁰. In some embodiments, A is indolyl substituted with —SO₂R¹⁰. In some embodiments, A is indolyl substituted with —C(O)N(R¹⁰)₂. In some embodiments, A is indolyl substituted with halogen. In some embodiments, A is indolyl substituted with cyano.

In some embodiments, A is 1-methyl-1H-indol-4-yl. In some embodiments, A is 1-methyl-1H-indol-3-yl.

In some embodiments, A is imidazol-2-yl. In some embodiments, A is 1-methyl-imidazol-2-yl.

In some embodiments, A is selected from the group consisting of 1,2,3,4-tetrahydroquinoline; 1,2,3,4-tetrahydro-1,5,napthyridine, dihydropyridopyrazine, benzoimidazole, benzoazepine, and dihydropyridooxazine, wherein A is bound to X through an available nitrogen on X; and where A is optionally substituted with one or two substituents each independently selected from halo, CF₃, phenyl (optionally substituted with one, two or three halo, C₁₋₃alkyl or C₁₋₃haloalkyl), C₁₋₃alkyl, C₁₋₃haloalkyl, and heteroaryl (optionally substituted with one, two or three halo, C₁₋₃alkyl or C₁₋₃haloalkyl).

In some embodiments, A is selected from the group consisting of

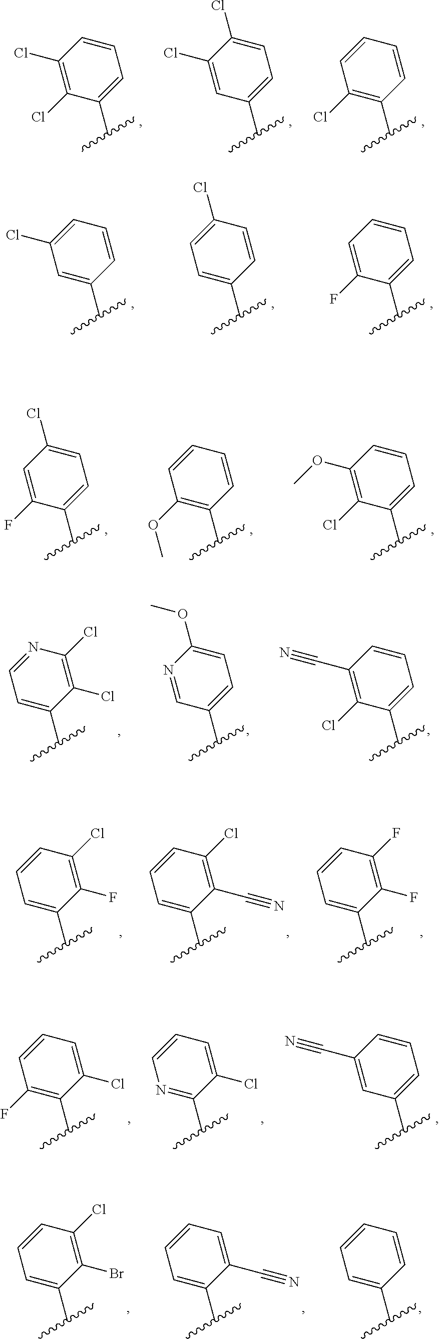

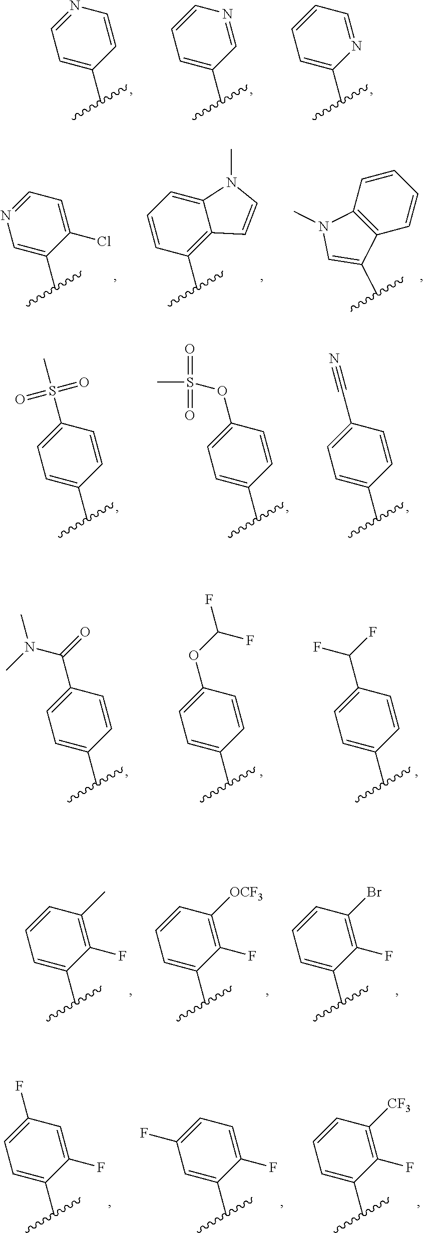

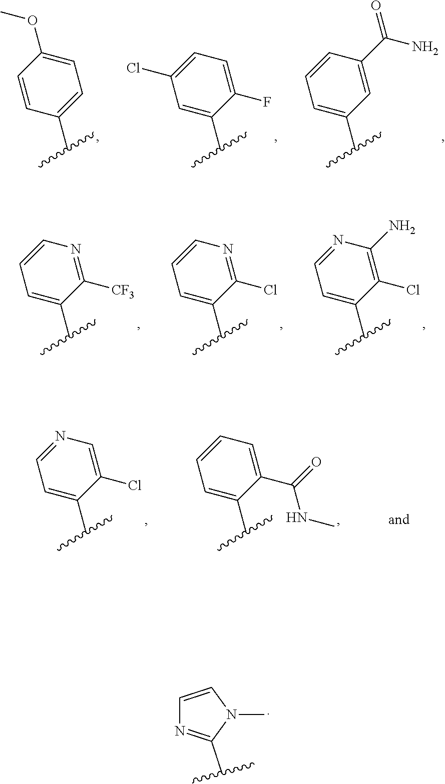

In some embodiments, A is

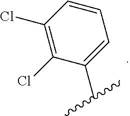 [SMILES: *c1cccc(Cl)c1Cl]

In some embodiments, A is

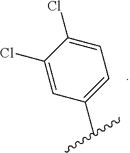 [SMILES: *c1ccc(Cl)c(Cl)c1]

In some embodiments, A is

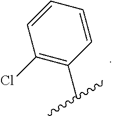 [SMILES: *c1ccccc1Cl]

In some embodiments, A is

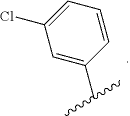 [SMILES: *c1cccc(Cl)c1]

In some embodiments, A is

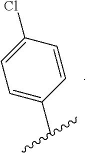 [SMILES: *c1ccc(Cl)cc1]

In some embodiments, A is

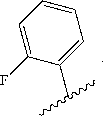 [SMILES: *c1ccccc1F]

In some embodiments, A is

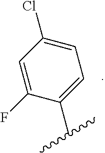 [SMILES: *c1ccc(Cl)cc1F]

In some embodiments, A is

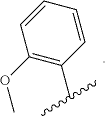 [SMILES: *c1ccccc1OC]

In some embodiments, A is

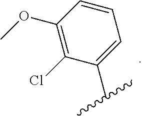 [SMILES: *c1cccc(OC)c1Cl]

In some embodiments, A is

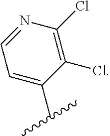 [SMILES: *c1ccnc(Cl)c1Cl]

In some embodiments, A is

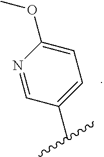 [SMILES: *c1ccc(OC)nc1]

In some embodiments, A is

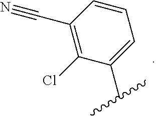 [SMILES: *c1cccc(C#N)c1Cl]

In some embodiments, A is

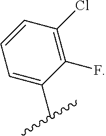 [SMILES: *c1cccc(Cl)c1F]

In some embodiments, A is

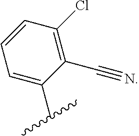 [SMILES: *c1cccc(Cl)c1C#N]

In some embodiments, A is

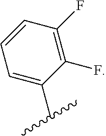 [SMILES: *c1cccc(F)c1F]

In some embodiments, A is

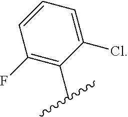 [SMILES: *c1c(F)cccc1Cl]

In some embodiments, A is

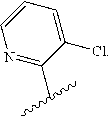 [SMILES: *c1ncccc1Cl]

In some embodiments, A is

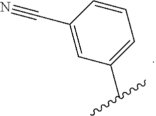 [SMILES: *c1cccc(C#N)c1]

In some embodiments, A is

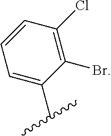 [SMILES: *c1cccc(Cl)c1Br]

In some embodiments, A is

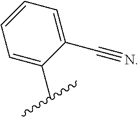 [SMILES: *c1ccccc1C#N]

In some embodiments, A is

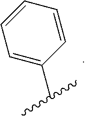 [SMILES: *c1ccccc1]

In some embodiments, A is

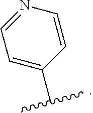 [SMILES: *c1ccncc1]

In some embodiments, A is

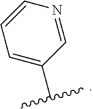 [SMILES: *c1cccnc1]

In some embodiments, A is

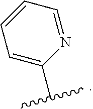 [SMILES: *c1ccccn1]

In some embodiments, A is

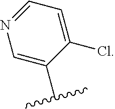 [SMILES: *c1cnccc1Cl]

In some embodiments, A is

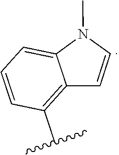 [SMILES: *c1cccc2c1ccn2C]

In some embodiments, A is

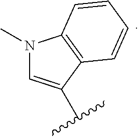 [SMILES: *c1cn(C)c2ccccc12]

In some embodiments, A is

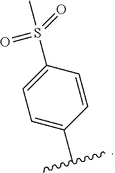 [SMILES: *c1ccc(S(C)(=O)=O)cc1]

In some embodiments, A is

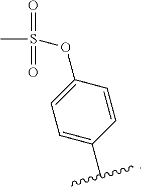 [SMILES: *c1ccc(OS(C)(=O)=O)cc1]

In some embodiments, A is

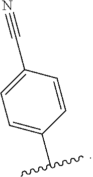 [SMILES: *c1ccc(C#N)cc1]

In some embodiments, A is

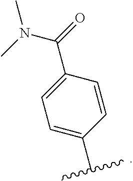 [SMILES: *c1ccc(C(=O)N(C)C)cc1]

In some embodiments, A is

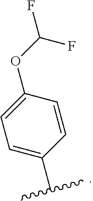 [SMILES: *c1ccc(OC(F)F)cc1]

In some embodiments, A is

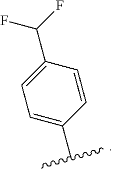 [SMILES: *c1ccc(C(F)F)cc1]

In some embodiments, A is

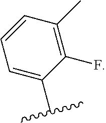 [SMILES: *c1cccc(C)c1F]

In some embodiments, A is

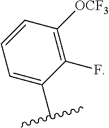 [SMILES: *c1cccc(OC(F)(F)F)c1F]

In some embodiments, A is

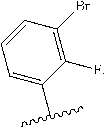 [SMILES: *c1cccc(Br)c1F]

In some embodiments, A is

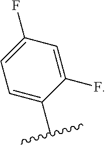 [SMILES: *c1ccc(F)cc1F]

In some embodiments, A is

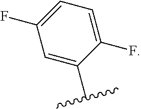 [SMILES: *c1cc(F)ccc1F]

In some embodiments, A is

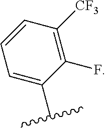 [SMILES: *c1cccc(C(F)(F)F)c1F]

In some embodiments, A is

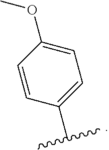 [SMILES: *c1ccc(OC)cc1 ; "."]

In some embodiments, A is

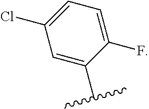 [SMILES: *c1cc(Cl)ccc1F]

In some embodiments, A is

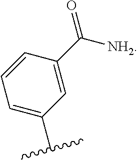 [SMILES: *c1cccc(C(N)=O)c1]

In some embodiments, A is

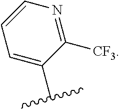 [SMILES: *c1cccnc1C(F)(F)F]

In some embodiments, A is

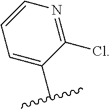 [SMILES: *c1cccnc1Cl]

In some embodiments, A is

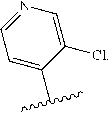 [SMILES: *c1ccncc1Cl]

In some embodiments, A is

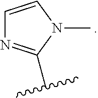 [SMILES: *c1nccn1C ; "."]

In some embodiments, A is

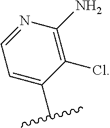 [SMILES: *c1ccnc(N)c1Cl]

In some embodiments, A is

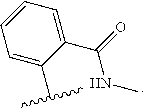 [SMILES: *c1ccccc1C(=O)NC ; "."]

In certain embodiments, R³ is selected from the group consisting of hydrogen, CH₃ and —NH₂. In some embodiments, Y is a bond and Z is N.

In some embodiments, R¹ and R² together with the nitrogen to which they are attached form heterocyclic ring B; wherein ring B is selected from the group consisting of pyrrolidinyl, piperidinyl, azepanyl and morpholinyl; wherein pyrrolidinyl, piperidinyl, azepanyl and morpholinyl may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃alkoxy; wherein C₁₋₃alkyl and C₁₋₃alkoxy may optionally be substituted by halogen, hydroxyl and —NR^{a}R^{b}.

In some embodiments, Y is —NH—, —N(CH₃)—, —N(H)—CH₂—, or —N(CH₃)—CH₂—; and Z is C(H).

In certain embodiments, R¹ and R² together with the carbon to which they are attached form carbocyclic ring B; wherein ring B is selected from the group consisting of cyclopentyl and cyclohexyl, and wherein cyclopentyl and cyclohexyl may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃ alkoxy; wherein C₁₋₃alkyl and C₁₋₃alkoxy may optionally be substituted by halogen, hydroxyl and —NR^{a}R^{b}.

In certain other embodiments, R¹ and R² together with the carbon to which they are attached form heterocyclic ring B; wherein ring B is selected from the group consisting of tetrahydrofuranyl and tetrahydropyranyl, wherein tetrahydrofuranyl and tetrahydropyranyl may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃alkoxy; wherein C₁₋₃alkyl and C₁₋₃alkoxy may optionally be substituted by halogen, hydroxyl and —NR^{a}R^{b}.

For example, in some embodiments, ring B is substituted on an available carbon by a substituent selected from the group consisting of —NH₂, —CH₃, and —CH₂NH₂.

In certain embodiments, R¹ is hydrogen and R² is branched C₁₋₁₀alkyl; wherein C₁₋₁₀alkyl may optionally be substituted by one, two, three or more substituents each independently selected from the group consisting of halogen and —NH₂.

In some embodiments, —Y—Z—(R¹)(R²) is:

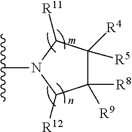

wherein

R⁴ and R⁵ are each independently selected from the group consisting of hydrogen, hydroxyl, C₁₋₆alkyl, C₁₋₆alkoxy, C₁₋₆alkyl-O—R⁶, —C(O)N(R⁶)₂, —N(R⁶)₂, halogen, C₁₋₆alkyl-N(R⁶)₂, and cyano; wherein C₁₋₆alkyl, C₁₋₆alkoxy, C₁₋₆alkyl-O—R⁶, and C₁₋₆alkyl-N(R⁶)₂ may optionally be substituted by one or more substituents each independently selected from the group consisting of hydroxyl, —NH₂, oxo, and halogen,

or R⁴ and R⁵, taken together with the atoms to which they are attached, form a 3-7 membered carbocyclic or heterocyclic saturated or partially unsaturated ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;

or R⁴ and R⁸, taken together with the atoms to which they are attached, form a 4-7 membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;

or R⁴ is absent, and R⁴ and R⁸, taken together with the atoms to which they are attached, form a 3-membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;

R⁶ is independently for each occurrence selected from the group consisting of hydrogen, C₁₋₆alkyl and phenyl;

R¹¹ and R¹² are each independently selected from the group consisting of hydrogen, hydroxyl, C₁₋₆alkyl, C₁₋₆alkoxy, C₁₋₆alkyl-O—R⁶, aryl, arylalkylene, heteroaryl, heteroarylalkylene, —C(O)N(R⁶)₂, —N(R⁶)₂, halogen, C₁₋₆alkyl-N(R⁶)₂, —CO₂H, and cyano; wherein C₁₋₆alkyl, C₁₋₆alkoxy, C₁₋₆alkyl-O—R⁶, aryl, arylalkylene, heteroaryl, heteroarylalkylene, and C₁₋₆alkyl-N(R⁶)₂ may optionally be substituted by one or more substituents each independently selected from the group consisting of hydroxyl, —NH₂, and halogen;

or R¹¹ and R¹², taken together with the atoms to which they are attached, form a 5-7 membered heterocyclic ring;

or R⁴ and R¹², taken together with the atoms to which they are attached, form a 5-7 membered carbocyclic or heterocyclic ring;

or R⁸ and R¹¹, taken together with the atoms to which they are attached, form a 5-7 membered carbocyclic or heterocyclic ring;

R⁸ and R⁹ are each independently selected from the group consisting of hydrogen, C₁₋₆)alkyl, C₁₋₆alkyl-N(R⁶)₂, —OR⁶, C₁₋₆alkyl-O—R⁶, —C(O)NH₂, —N(R⁶)₂, halogen, and cyano; and

each of m and n is, independently, 0, 1, 2, or 3, with m+n being at least 2 and no more than 4

In some embodiments, R⁴ and R⁵ are each independently selected from the group consisting of C₁₋₃alkyl and —NR^{a}R^{b}; wherein C₁₋₃alkyl may optionally be substituted by one, two or three substituents each independently selected from the group consisting of halogen, hydroxyl and —NR^{a}R^{b}. In certain embodiments, R⁴ and R⁵ are each independently selected from the group consisting of —NH₂, —CH₃, and —CH₂NH₂.

In some embodiments, —Y—Z—(R¹)(R²) is represented by:

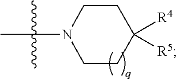

wherein

R⁴ and R⁵ are each independently selected from the group consisting of hydrogen, halogen, cyano, —NR^{a}R^{b}, —C(O)—NR^{a}R^{b} and C₁₋₆alkyl, wherein C₁₋₆alkyl may optionally be substituted by one, two or three or more substituents each independently selected from the group consisting of halogen, hydroxyl, —NR^{a}R^{b} and oxo;

R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl; and q is 0 or 1.

Examples of compounds of the present disclosure include:

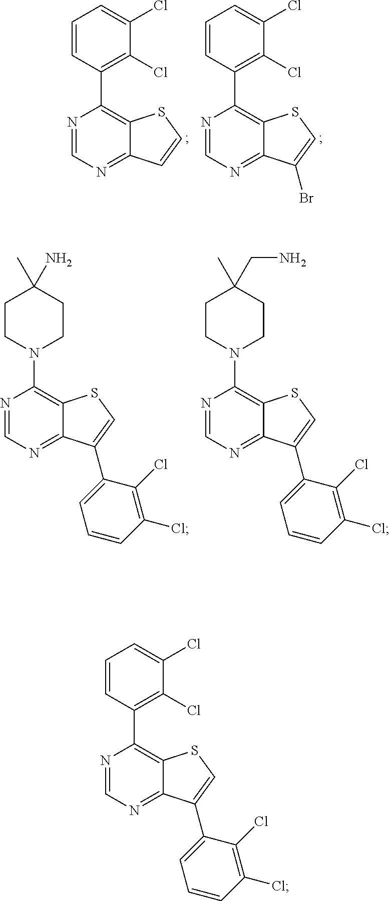

and a pharmaceutically acceptable salt or stereoisomer thereof.

In some embodiments, SHP2 phosphatase inhibitors described herein encompass compounds of Formula IX, or a pharmaceutically acceptable salt or stereoisomer thereof, wherein Formula IX is represented by:

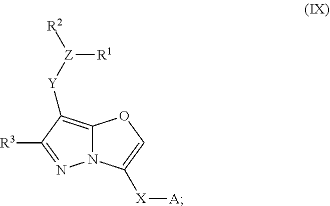

wherein

X is selected from the group consisting of a bond, —O—, —NR^{X1}—, —C(O)NR^{X11}—, —S(O)_{w}— (wherein w is 0, 1 or 2), —C(O)—, —C(R^{X2}R^{X3})—, —O—C(R^{X4}R^{X5})—, —C(R^{X4}R^{X5})—O—, and —C═C(R^{X6}R^{X7})—;

Y is selected from the group consisting of a bond, —NR^{Y}—, —C₁₋₃alkylene-NR^{Y}—, and —NR^{Y}—C₁₋₃alkylene-;

Z is selected from the group consisting of N and C(R^{Z}), or Z and R¹ are absent;

wherein if Y is a bond, Z is N or absent; and wherein if Y is —NR^{Y}—, —C₁₋₃alkylene-NR^{Y}—, or —NR^{Y}—C₁₋₃alkylene-, Z is C(R^{Z});

R^{Z} is selected from the group consisting of hydrogen and C₁₋₆alkyl; wherein C₁₋₆alkyl may optionally be substituted by halogen, hydroxyl, and —NR^{a}R^{b},

A is selected from the group consisting of a 6-10 membered monocyclic or bicyclic aryl, a 5-7 membered monocyclic heteroaryl having one or more heteroatoms each independently selected from O, S, or N, a 8-10 membered bicyclic heteroaryl having one or more heteroatoms each independently selected from O, S, or N; and a 4-7 membered heterocyclyl, wherein A may optionally be substituted by one, two, three or more substituents each independently selected from the group consisting of —R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R¹⁰ is independently selected from the group consisting of hydrogen, C₁₋₆alkyl, C₁₋₆haloalkyl, C₃₋₆cycloalkyl, heterocycloalkyl, aryl, and heteroaryl; wherein R¹⁰ may optionally be substituted by one, two, three or more substituents each independently selected from the group consisting of halogen, —C(O)R²⁰, C₁₋₆alkyl and C₁₋₆haloalkyl;

R²⁰ is selected from the group consisting of hydroxyl, halogen, and C₁₋₆alkyl;

R¹ and R² are each selected from the group consisting of hydrogen and C₁₋₁₀alkyl; wherein C₁₋₁₀alkyl may optionally be substituted by one, two, three or more substituents each independently selected from R^{P};

or R¹ and R², together with carbon or nitrogen to which they are attached, form a 3-7 membered saturated carbocyclic or 4-7 membered saturated heterocyclic ring B having one or two heteroatoms each independently selected from the group consisting of O, S(O)_{w} (wherein w is 0, 1, or 2), and NR^{h}; wherein carbocyclic or heterocyclic ring B may be monocyclic or bicyclic;

carbocyclic or heterocyclic ring B may optionally be substituted on one or more available carbons by one, two, three or more substituents each independently selected from the group consisting of halogen, hydroxyl, cyano, oxo, —NR^{a}R^{b}, —CO₂H, —C(O)—NR^{a}R^{b}, —S(O)₂—NR^{a}R^{b}, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})—, C₁₋₆alkoxycarbonyl-N(R^{a})—, aryl and heteroaryl; wherein C₁₋₆alkyl, C₂₋₆alkenyl, C₂₋₆alkynyl, C₃₋₆cycloalkyl, C₁₋₆alkoxy, C₃₋₆ alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})— and C₁₋₆alkoxycarbonyl-N(R^{a})_{w}— may optionally be substituted by one or more substituents each independently selected from R^{P}; and wherein aryl and heteroaryl may optionally be substituted by one or more substituents each independently selected from R^{f};

R^{X1} is selected from the group consisting of hydrogen, C₁₋₆alkyl and phenyl, or R^{X1} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X11} is selected from the group consisting of hydrogen, C₁₋₆alkyl and phenyl, or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X2} and R^{X3} are each independently selected from the group consisting of hydrogen, halogen, hydroxyl, —NR^{a}R^{b}, —C(O)—NR^{a}R^{b}, C₁₋₆alkyl and C₁₋₆alkoxy; wherein C₁₋₆alkyl and C₁₋₆alkoxy may optionally be substituted by one or more substituents each independently selected from R^{P};

or R^{X2} and R^{X3} together with the carbon to which they are attached form a 3-6 membered carbocycle optionally substituted by one or more substituents each independently selected from the group consisting of hydrogen, halogen, —NR^{a}R^{b}, —C(O)—NR^{a}R^{b}, oxo, C₁₋₆ alkyl and C₁₋₆alkoxy;

R^{X4} and R^{X5} are each independently selected from the group consisting of hydrogen and C₁₋₆alkyl; wherein C₁₋₆alkyl may optionally be substituted by one or more substituents each independently selected from R^{P};

R^{X6} and R^{X7} are each independently selected from the group consisting of hydrogen, halogen, —C(O)—NR^{a}R^{b}, cyano and C₁₋₆alkyl; wherein C₁₋₆alkyl may optionally be substituted by one or more substituents each independently selected from R^{P};

R^{Y} is selected from the group consisting of hydrogen, C₁₋₆alkyl and phenyl;

R³ is selected from the group consisting of hydrogen, halogen, hydroxyl, C₁₋₆ alkyl and —NR^{a}R^{b};

R^{f} is independently selected, for each occurrence, from the group consisting of R^{P}, C₁₋₆ alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, (wherein wherein w is 0, 1 or 2), C₁₋₆alkylcarbonyl-N(R^{a})— and C₁₋₆alkoxycarbonyl-N(R^{a})—; wherein C₁₋₆alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, C₁₋₆alkylcarbonyl-N(R^{a})—, and C₁₋₆alkoxycarbonyl-N(R^{a})— may be optionally substituted by one or more substituents selected from R^{P};

R^{h} is independently selected, for each occurrence, from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆ alkylcarbonyl-, C₁₋₆alkoxycarbonyl-, R^{a}R^{b}N-carbonyl- and R^{a}R^{b}N—SO₂—; wherein C₁₋₆alkyl, C₃₋₆alkenyl, C₃₋₆alkynyl, C₃₋₆cycloalkyl, C₁₋₆alkyl-S(O)₂—, C₁₋₆alkylcarbonyl- and C₁₋₆ alkoxycarbonyl- may optionally be substituted by one or more substituents selected from R^{P};

R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl;

or R^{a} and R^{b}, together with the nitrogen to which they are attached, may form a 4-6 membered monocyclic heterocyclic ring, which may have an additional heteroatom selected from the group consisting of O, S, and N; wherein the 4-6 membered heterocyclic ring may optionally be substituted by one or more substituents selected from the group consisting of halogen, cyano, oxo or hydroxyl; and

R^{P} is independently selected, for each occurrence, from the group consisting of halogen, hydroxyl, cyano, C₁₋₆alkoxy, R^{a}R^{b}N—, R^{a}R^{b}N-carbonyl-, R^{a}R^{b}N—SO₂—, and R^{a}R^{b}N-carbonyl-N(R^{a})—.

In some embodiments, X is selected from the group consisting of a bond, —O—, —NR^{X1}— and —NR^{X11}—.

In some embodiments, A is selected from the group consisting of phenyl, pyridyl, quinolinyl, indolyl, and indolinyl, wherein phenyl, pyridyl, quinolinyl, indolyl and indolinyl may optionally be substituted by one, two or three substituents each independently selected from the group consisting of —R¹⁰, —OR¹⁰, —SR¹⁰, —N(R¹⁰)₂, —OSO₂R¹⁰, —SO₂R¹⁰, —C(O)N(R¹⁰)₂, halogen, and cyano. In certain embodiments, A is phenyl; wherein phenyl may optionally be substituted by one, two, or three substituents each independently selected from the group consisting of —OR¹⁰, halogen, and cyano. In certain other embodiments, A is pyridyl; wherein pyridyl may optionally be substituted by one, two, or three substituents each independently selected from the group consisting of —OR¹⁰, halogen, and cyano.

In some embodiments, R^{X1} and ring A together with the nitrogen to which they are attached form a 1,2,3,4-tetrahydro-1,5-naphthyridine moiety. In some embodiments, R^{X11} and ring A together with the nitrogen to which they are attached form a 1,2,3,4-tetrahydro-1,5-naphthyridine moiety. In some embodiments, R^{X1} and ring A together with the nitrogen to which they are attached form a 1,2,3,4-tetrahydroquinoline moiety. In some embodiments, R^{X11} and ring A together with the nitrogen to which they are attached form a 1,2,3,4-tetrahydroquinoline moiety.

In some embodiments, A is phenyl, pyridyl, indolyl, or indolinyl, wherein said phenyl, pyridyl, indolyl or indolinyl is optionally substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano.

In some embodiments, A is phenyl, pyridyl, imidazolyl, indolyl, indolinyl, naphthyl, quinolinyl, isoquinolinyl, benzimidazolyl, indazolyl, benzotriazolyl, benzothiazolyl, benzofuranyl, pyrazolopyridyl, quinazolinyl, quinoxalinyl, or cinnolinyl. In some embodiments, A is phenyl. In some embodiments, A is pyridyl. In some embodiments, A is 2-pyridyl. In some embodiments, A is 3-pyridyl. In some embodiments, A is 4-pyridyl. In some embodiments, A is naphthyl. In some embodiments, A is 1-naphthyl. In some embodiments, A is 2-naphthyl. In some embodiments, A is quinolinyl. In some embodiments, A is 2-quinolinyl. In some embodiments, A is 3-quinolinyl. In some embodiments, A is 4-quinolinyl. In some embodiments, A is 5-quinolinyl. In some embodiments, A is 6-quinolinyl. In some embodiments, A is 7-quinolinyl. In some embodiments, A is 8-quinolinyl. In some embodiments, A is isoquinolinyl. In some embodiments, A is 1-isoquinolinyl. In some embodiments, A is 3-isoquinolinyl. In some embodiments, A is 4-isoquinolinyl. In some embodiments, A is 5-isoquinolinyl. In some embodiments, A is 6-isoquinolinyl. In some embodiments, A is 7-isoquinolinyl. In some embodiments, A is 8-isoquinolinyl. In some embodiments, A is indolyl. In some embodiments, A is 2-indolyl. In some embodiments, A is 3-indolyl. In some embodiments, A is 4-indolyl. In some embodiments, A is 5-indolyl. In some embodiments, A is 6-indolyl. In some embodiments, A is 7-indolyl. In some embodiments, A is aryl. In some embodiments, A is bicyclic aryl. In some embodiments, A is heteroaryl. In some embodiments, A is fused bicyclic heteroaryl.

In some embodiments, A is phenyl, pyridyl, or indolyl, wherein said phenyl, pyridyl, or indolyl is optionally substituted with —OR¹⁰, halogen, or cyano.

In some embodiments, A is phenyl, wherein said phenyl is optionally substituted with —OR¹⁰, halogen, or cyano.

In some embodiments, A is pyridyl, wherein said pyridyl is optionally substituted with —OR¹⁰, halogen, or cyano.

In some embodiments, A is indolyl or indolinyl, wherein said indolyl or indolinyl is optionally substituted with —OR¹⁰, halogen, or cyano.

In some embodiments, A is phenyl, pyridyl, indolyl, or indolinyl, wherein said phenyl, pyridyl, indolyl or indolinyl is optionally substituted with —OR¹⁰ or halogen.

In some embodiments, A is phenyl, pyridyl, indolyl, or indolinyl, wherein said phenyl, pyridyl, indolyl or indolinyl is optionally substituted with halogen.

In some embodiments, A is phenyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is phenyl substituted with —R¹⁰. In some embodiments, A is phenyl substituted with —OR °. In some embodiments, A is phenyl substituted with —SR¹⁰. In some embodiments, A is phenyl substituted with —N(R¹⁰)₂. In some embodiments, A is phenyl substituted with —OSO₂R¹⁰. In some embodiments, A is phenyl substituted with —SO₂R¹⁰. In some embodiments, A is phenyl substituted with —C(O)N(R¹⁰)₂. In some embodiments, A is phenyl substituted with halogen. In some embodiments, A is phenyl substituted with cyano.

In some embodiments, A is pyridyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is pyridyl substituted with —R¹⁰. In some embodiments, A is pyridyl substituted with —OR¹⁰. In some embodiments, A is pyridyl substituted with —SR¹⁰. In some embodiments, A is pyridyl substituted with —N(R¹⁰)₂. In some embodiments, A is pyridyl substituted with —OSO₂R¹⁰. In some embodiments, A is pyridyl substituted with —SO₂R¹⁰. In some embodiments, A is pyridyl substituted with —C(O)N(R¹⁰)₂. In some embodiments, A is pyridyl substituted with halogen. In some embodiments, A is pyridyl substituted with cyano.

In some embodiments, A is 3-pyridyl substituted with —OR¹⁰. In some embodiments, A is 3-pyridyl substituted with —OCH₃. In some embodiments, A is 6-methoxypyrid-3-yl. In some embodiments, A is 3-pyridyl substituted with halogen. In some embodiments, A is 3-pyridyl substituted with Cl. In some embodiments, A is 4-chloropyrid-3-yl. In some embodiments, A is 2,3-dichloropyrid-4-yl. In some embodiments, A is 3-chloropyrid-2-yl.

In some embodiments, A is naphthyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is naphthyl substituted with —R¹⁰. In some embodiments, A is naphthyl substituted with —OR¹⁰. In some embodiments, A is naphthyl substituted with —SR¹⁰. In some embodiments, A is naphthyl substituted with —N(R¹⁰)₂. In some embodiments, A is naphthyl substituted with —OSO₂R¹⁰. In some embodiments, A is naphthyl substituted with —SO₂R¹⁰. In some embodiments, A is naphthyl substituted with —C(O)N(R¹⁰)₂. In some embodiments, A is naphthyl substituted with halogen. In some embodiments, A is naphthyl substituted with cyano.

In some embodiments, A is quinolinyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is quinolinyl substituted with —R¹⁰. In some embodiments, A is quinolinyl substituted with —OR¹⁰. In some embodiments, A is quinolinyl substituted with —SR¹⁰. In some embodiments, A is quinolinyl substituted with —N(R¹⁰)₂. In some embodiments, A is quinolinyl substituted with —OSO₂R¹⁰. In some embodiments, A is quinolinyl substituted with —SO₂R¹⁰. In some embodiments, A is quinolinyl substituted with —C(O)N(R¹⁰)₂. In some embodiments, A is quinolinyl substituted with halogen. In some embodiments, A is quinolinyl substituted with cyano.

In some embodiments, A is isoquinolinyl substituted with —R¹⁰, —OR, —SR¹⁰, —N(R¹⁰)₂, —OSO₂R¹⁰, —SO₂R¹⁰, —C(O)N(R¹⁰)₂, halogen, or cyano. In some embodiments, A is isoquinolinyl substituted with —R¹⁰. In some embodiments, A is isoquinolinyl substituted with —OR¹⁰. In some embodiments, A is isoquinolinyl substituted with —SR¹⁰. In some embodiments, A is isoquinolinyl substituted with —N(R¹⁰)₂. In some embodiments, A is isoquinolinyl substituted with —OSO₂R¹⁰. In some embodiments, A is isoquinolinyl substituted with —SO₂R¹⁰. In some embodiments, A is isoquinolinyl substituted with —C(O)N(R¹⁰)₂. In some embodiments, A is isoquinolinyl substituted with halogen. In some embodiments, A is isoquinolinyl substituted with cyano.

In some embodiments, A is indolyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is indolyl substituted with —R¹⁰. In some embodiments, A is indolyl substituted with —OR¹⁰. In some embodiments, A is indolyl substituted with —SR¹⁰. In some embodiments, A is indolyl substituted with —N(R¹⁰)₂. In some embodiments, A is indolyl substituted with —OSO₂R¹⁰. In some embodiments, A is indolyl substituted with —SO₂R¹⁰. In some embodiments, A is indolyl substituted with —C(O)N(R¹⁰)₂. In some embodiments, A is indolyl substituted with halogen. In some embodiments, A is indolyl substituted with cyano.

In some embodiments, A is 1-methyl-1H-indol-4-yl. In some embodiments, A is 1-methyl-1H-indol-3-yl.

In some embodiments, A is imidazol-2-yl. In some embodiments, A is 1-methyl-imidazol-2-yl.

In some embodiments, A is selected from the group consisting of 1,2,3,4-tetrahydroquinoline; 1,2,3,4-tetrahydro-1,5,napthyridine, dihydropyridopyrazine, benzoimidazole, benzoazepine, and dihydropyridooxazine, wherein A is bound to X through an available nitrogen on X; and where A is optionally substituted with one or two substituents each independently selected from halo, CF₃, phenyl (optionally substituted with one, two or three halo, C₁₋₃alkyl or C₁₋₃haloalkyl), C₁₋₃alkyl, C₁₋₃haloalkyl, and heteroaryl (optionally substituted with one, two or three halo, C₁₋₃alkyl or C₁₋₃haloalkyl).

In some embodiments, A is selected from the group consisting of

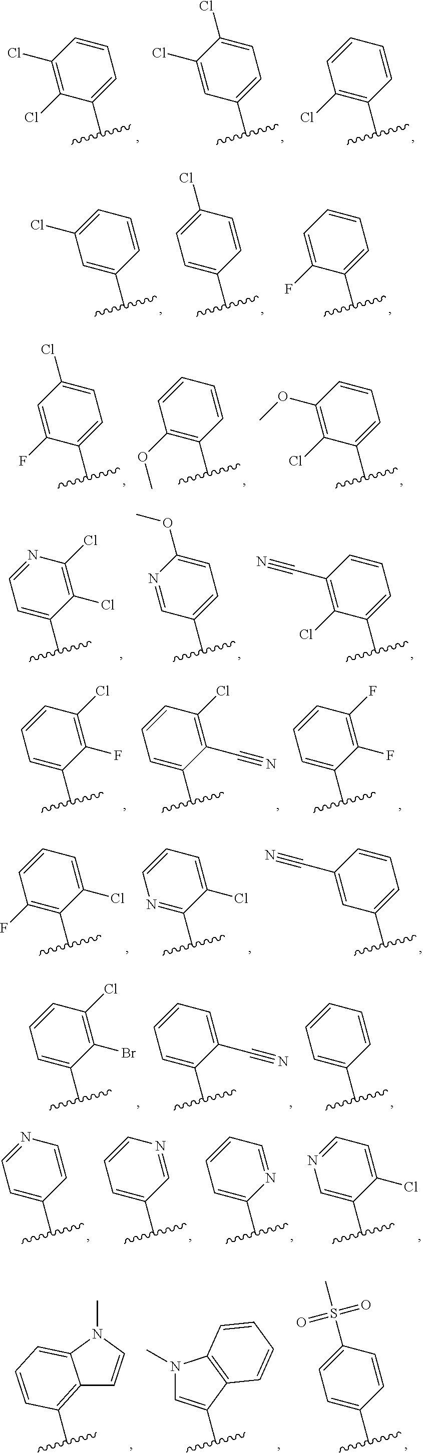

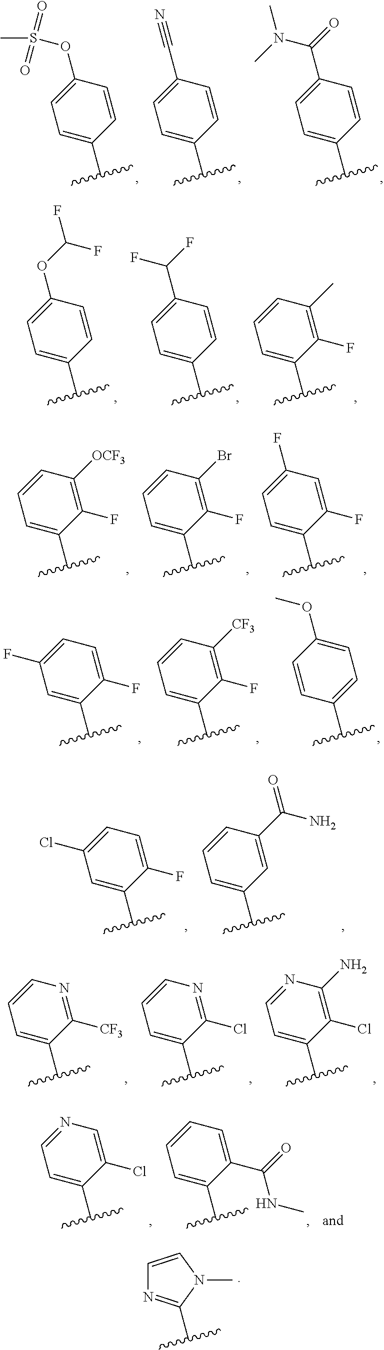

In some embodiments, A is

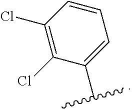 [SMILES: *c1cccc(Cl)c1Cl ; "."]

In some embodiments, A is

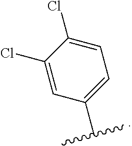 [SMILES: *c1ccc(Cl)c(Cl)c1 ; "."]

In some embodiments, A is

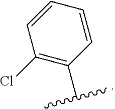 [SMILES: *c1ccccc1Cl ; "."]

In some embodiments, A is

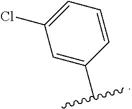 [SMILES: *c1cccc(Cl)c1 ; "."]

In some embodiments, A is

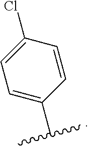 [SMILES: *c1ccc(Cl)cc1 ; "."]

In some embodiments, A is

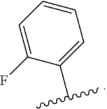 [SMILES: *c1ccccc1F ; "."]

In some embodiments, A is

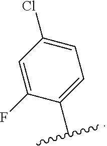 [SMILES: *c1ccc(Cl)cc1F ; "."]

In some embodiments, A is

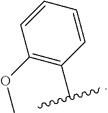 [SMILES: *c1ccccc1OC ; "."]

In some embodiments, A is

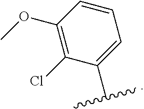 [SMILES: *c1cccc(OC)c1Cl ; "."]

In some embodiments, A is

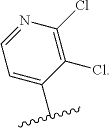 [SMILES: *c1ccnc(Cl)c1Cl]

In some embodiments, A is

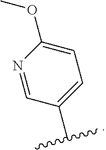 [SMILES: *c1ccc(OC)nc1 ; "."]

In some embodiments, A is

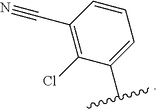 [SMILES: *c1cccc(C#N)c1Cl ; "."]

In some embodiments, A is

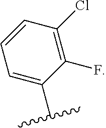 [SMILES: *c1cccc(Cl)c1F]

In some embodiments, A is

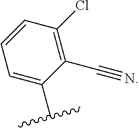 [SMILES: *c1cccc(Cl)c1C#N]

In some embodiments, A is

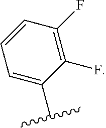 [SMILES: *c1cccc(F)c1F]

In some embodiments, A is

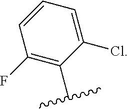 [SMILES: *c1c(F)cccc1Cl]

In some embodiments, A is

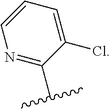 [SMILES: *c1ncccc1Cl]

In some embodiments, A is

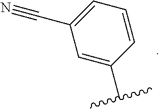 [SMILES: *c1cccc(C#N)c1]

In some embodiments, A is

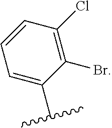 [SMILES: *c1cccc(Cl)c1Br]

In some embodiments, A is

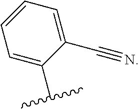 [SMILES: *c1ccccc1C#N]

In some embodiments, A is

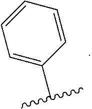 [SMILES: *c1ccccc1]

In some embodiments, A is

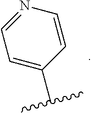 [SMILES: *c1ccncc1]

In some embodiments, A is

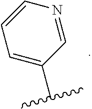 [SMILES: *c1cccnc1]

In some embodiments, A is

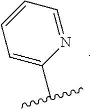 [SMILES: *c1ccccn1]

In some embodiments, A is

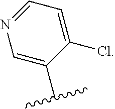 [SMILES: *c1cnccc1Cl]

In some embodiments, A is

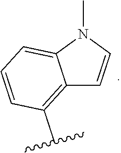 [SMILES: *c1cccc2c1ccn2C]

In some embodiments, A is

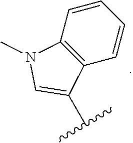 [SMILES: *c1cn(C)c2ccccc12]

In some embodiments, A is

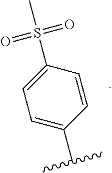 [SMILES: *c1ccc(S(C)(=O)=O)cc1]

In some embodiments, A is

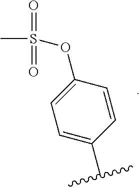 [SMILES: *c1ccc(OS(C)(=O)=O)cc1]

In some embodiments, A is

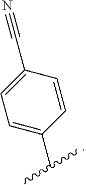 [SMILES: *c1ccc(C#N)cc1]

In some embodiments,

### A is

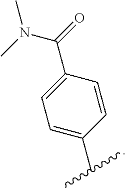 [SMILES: *c1ccc(C(=O)N(C)C)cc1]

In some embodiments, A is

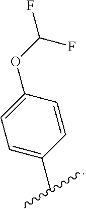 [SMILES: *c1ccc(OC(F)F)cc1]

In some embodiments, A is

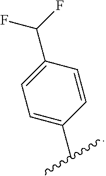 [SMILES: *c1ccc(C(F)F)cc1]

In some embodiments, A is

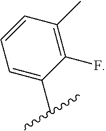 [SMILES: *c1cccc(C)c1F]

In some embodiments, A is

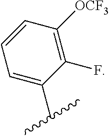 [SMILES: *c1cccc(OC(F)(F)F)c1F]

In some embodiments, A is

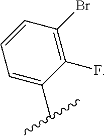 [SMILES: *c1cccc(Br)c1F]

In some embodiments, A is

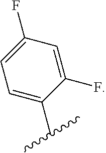 [SMILES: *c1ccc(F)cc1F]

In some embodiments, A is

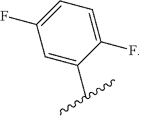 [SMILES: *c1cc(F)ccc1F]

In some embodiments, A is

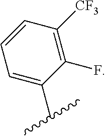 [SMILES: *c1cccc(C(F)(F)F)c1F]

In some embodiments, A is

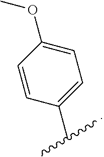 [SMILES: *c1ccc(OC)cc1]

In some embodiments, A is

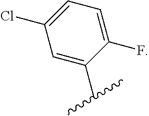 [SMILES: *c1cc(Cl)ccc1F]

In some embodiments, A is

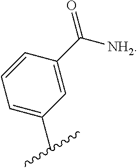 [SMILES: *c1cccc(C(N)=O)c1]

In some embodiments, A is

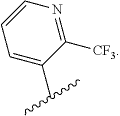 [SMILES: *c1cccnc1C(F)(F)F]

In some embodiments, A is

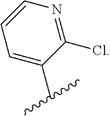 [SMILES: *c1cccnc1Cl]

In some embodiments, A is

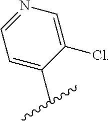 [SMILES: *c1ccncc1Cl]

In some embodiments, A is

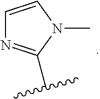 [SMILES: *c1nccn1C]

In some embodiments, A is

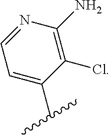 [SMILES: *c1ccnc(N)c1Cl]

In some embodiments, A is

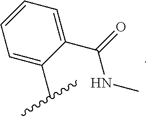 [SMILES: *c1ccccc1C(=O)NC]

In certain embodiments, R³ is selected from the group consisting of hydrogen, CH₃ and —NH₂. In some embodiments, Y is a bond and Z is N.

In some embodiments, R¹ and R² together with the nitrogen to which they are attached form heterocyclic ring B; wherein ring B is selected from the group consisting of pyrrolidinyl, piperidinyl, azepanyl and morpholinyl; wherein pyrrolidinyl, piperidinyl, azepanyl and morpholinyl may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃alkoxy; wherein C₁₋₃alkyl and C₁₋₃alkoxy may optionally be substituted by halogen, hydroxyl and —NR^{a}R^{b}.

In some embodiments, Y is —NH—, —N(CH₃)—, —N(H)—CH₂—, or —N(CH₃)—CH₂—; and Z is C(H).

In certain embodiments, R¹ and R² together with the carbon to which they are attached form carbocyclic ring B; wherein ring B is selected from the group consisting of cyclopentyl and cyclohexyl, and wherein cyclopentyl and cyclohexyl may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃alkoxy; wherein C₁₋₃alkyl and C₁₋₃alkoxy may optionally be substituted by halogen, hydroxyl and —NR^{a}R^{b}.

In certain other embodiments, R¹ and R² together with the carbon to which they are attached form heterocyclic ring B; wherein ring B is selected from the group consisting of tetrahydrofuranyl and tetrahydropyranyl, wherein tetrahydrofuranyl and tetrahydropyranyl may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃alkoxy; wherein C₁₋₃alkyl and C₁₋₃alkoxy may optionally be substituted by halogen, hydroxyl and —NR^{a}R^{b}.

For example, in some embodiments, ring B is substituted on an available carbon by a substituent selected from the group consisting of —NH₂, —CH₃, and —CH₂NH₂.

In certain embodiments, R is hydrogen and R² is branched C₁₋₁₀alkyl; wherein C₁₋₁₀alkyl may optionally be substituted by one, two, three or more substituents each independently selected from the group consisting of halogen and —NH₂.

In some embodiments, —Y—Z—(R¹)(R²) is.

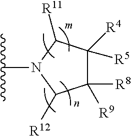

wherein

R⁴ and R⁵ are each independently selected from the group consisting of hydrogen, hydroxyl, C₁₋₆alkyl, C₁₋₆alkoxy, C₁₋₆alkyl-O—R⁶, —C(O)N(R⁶)₂, —N(R⁶)₂, halogen, C₁₋₆alkyl-N(R⁶)₂, and cyano; wherein C₁₋₆alkyl, C₁₋₆alkoxy, C₁₋₆alkyl-O—R⁶, and C₁₋₆alkyl-N(R⁶)₂ may optionally be substituted by one or more substituents each independently selected from the group consisting of hydroxyl, —NH₂, oxo, and halogen,

or R⁴ and R⁵, taken together with the atoms to which they are attached, form a 3-7 membered carbocyclic or heterocyclic saturated or partially unsaturated ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;

or R⁴ and R⁸, taken together with the atoms to which they are attached, form a 4-7 membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;

or R⁴ is absent, and R⁴ and R⁸, taken together with the atoms to which they are attached, form a 3-membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;

R⁶ is independently for each occurrence selected from the group consisting of hydrogen, C₁₋₆alkyl and phenyl;

R¹¹ and R¹² are each independently selected from the group consisting of hydrogen, hydroxyl, C₁₋₆alkyl, C₁₋₆alkoxy, C₁₋₆alkyl-O—R⁶, aryl, arylalkylene, heteroaryl, heteroarylalkylene, —C(O)N(R⁶)₂, —N(R⁶)₂, halogen, C₁₋₆alkyl-N(R⁶)₂, —CO₂H, and cyano; wherein C₁₋₆alkyl, C₁₋₆alkoxy, C₁₋₆alkyl-O—R⁶, aryl, arylalkylene, heteroaryl, heteroarylalkylene, and C₁₋₆alkyl-N(R⁶)₂ may optionally be substituted by one or more substituents each independently selected from the group consisting of hydroxyl, —NH₂, and halogen;

or R¹¹ and R¹², taken together with the atoms to which they are attached, form a 5-7 membered heterocyclic ring;

or R⁴ and R¹², taken together with the atoms to which they are attached, form a 5-7 membered carbocyclic or heterocyclic ring;

or R⁸ and R¹¹, taken together with the atoms to which they are attached, form a 5-7 membered carbocyclic or heterocyclic ring;

R⁸ and R⁹ are each independently selected from the group consisting of hydrogen, C₁₋₆)alkyl, C₁₋₆alkyl-N(R⁶)₂, —OR⁶, C₁₋₆alkyl-O—R⁶, —C(O)NH₂, —N(R⁶)₂, halogen, and cyano; and

each of m and n is, independently, 0, 1, 2, or 3, with m+n being at least 2 and no more than 4.

In some embodiments, R⁴ and R⁵ are each independently selected from the group consisting of C₁₋₃alkyl and —NR^{a}R^{b}; wherein C₁₋₃alkyl may optionally be substituted by one, two or three substituents each independently selected from the group consisting of halogen, hydroxyl and —NR^{a}R^{b}. In certain embodiments, R⁴ and R⁵ are each independently selected from the group consisting of —NH₂, —CH₃, and —CH₂NH₂.

In some embodiments, —Y—Z—(R¹)(R²) is represented by:

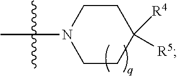

wherein

R⁴ and R⁵ are each independently selected from the group consisting of hydrogen, halogen, cyano, —NR^{a}R^{b}, —C(O)—NR^{a}R^{b} and C₁₋₆alkyl, wherein C₁₋₆alkyl may optionally be substituted by one, two or three or more substituents each independently selected from the group consisting of halogen, hydroxyl, —NR^{a}R^{b} and oxo;

R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl; and q is 0 or 1.

Examples of compounds of the present disclosure include:

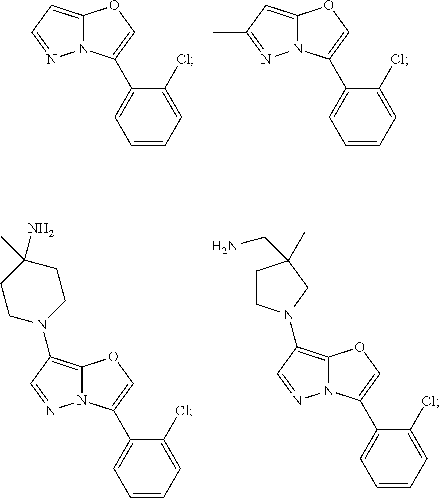

and a pharmaceutically acceptable salt or stereoisomer thereof.

In some embodiments, SHP2 phosphatase inhibitors described herein encompass compounds of Formula X, or a pharmaceutically acceptable salt or stereoisomer thereof, wherein Formula X is represented by:

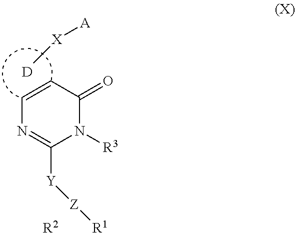

wherein

Ring D is absent or together with carbons to which it is attached, is a 5 membered heteroaryl having one or two heteroatoms each selected from S, O, N, or NR^{a};

X is bound to a free carbon on the pyrimidinone when D is absent, or on a free carbon of ring D, and selected from the group consisting of a bond, —O—, —NR^{X1}—, —C(O)NR^{X11}—, —S(O)_{w}— (wherein w is 0, 1 or 2), —C(O)—, —C(R^{X2}R^{X3})—, —O—C(R^{X4}R^{X5}), —C(R^{X4}R^{X5})—O—, and —C═C(R^{X6}R^{X7})—;

Y is selected from the group consisting of a bond, —NR^{Y}—, —C₁₋₃alkylene-NR^{Y}—, and —NR^{Y}—C₁₋₃alkylene-;

Z is selected from the group consisting of N and C(R^{Z}), or Z and R¹ are absent;

wherein if Y is a bond, Z is N or is absent, and wherein if Y is —NR^{Y}—, —C₁₋₃alkylene-NR^{Y}—, or —NR^{Y}—C₁₋₃alkylene-, Z is C(R^{Z});

R^{Z} is selected from the group consisting of hydrogen and C₁₋₆alkyl; wherein C₁₋₆alkyl may optionally be substituted by halogen, hydroxyl, and —NR^{a}R^{b},

A is selected from the group consisting of a 6-10 membered monocyclic or bicyclic aryl, a 5-7 membered monocyclic heteroaryl having one or more heteroatoms each independently selected from O, S, or N, a 8-10 membered bicyclic heteroaryl having one or more heteroatoms each independently selected from O, S, or N; and a 4-7 membered heterocyclyl, wherein A may optionally be substituted by one, two, three or more substituents each independently selected from the group consisting of —R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R¹⁰ is independently selected from the group consisting of hydrogen, C₁₋₆alkyl, C₁₋₆haloalkyl, C₃₋₆cycloalkyl, heterocycloalkyl, aryl, and heteroaryl; wherein R¹⁰ may optionally be substituted by one, two, three or more substituents each independently selected from the group consisting of halogen, —C(O)R²⁰, C₁₋₆alkyl and C₁₋₆haloalkyl;

R²⁰ is selected from the group consisting of hydroxyl, halogen, and C₁₋₆alkyl;

R¹ and R² are each selected from the group consisting of hydrogen and C₁₋₁₀alkyl; wherein C₁₋₁₀alkyl may optionally be substituted by one, two, three or more substituents each independently selected from R^{P};

or R¹ and R², together with carbon or nitrogen to which they are attached, form a 3-7 membered saturated carbocyclic or 4-7 membered saturated heterocyclic ring B having one or two heteroatoms each independently selected from the group consisting of 0, S(O)_{w} (wherein w is 0, 1, or 2), and NR^{h}; wherein carbocyclic or heterocyclic ring B may be monocyclic or bicyclic;

carbocyclic or heterocyclic ring B may optionally be substituted on one or more available carbons by one, two, three or more substituents each independently selected from the group consisting of halogen, hydroxyl, cyano, oxo, —NR^{a}R^{b}, —CO₂H, —C(O)—NR^{a}R^{b}, —S(O)₂—NR^{a}R^{b}, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})—, C₁₋₆alkoxycarbonyl-N(R^{a})—, aryl and heteroaryl; wherein C₁₋₆alkyl, C₂₋₆alkenyl, C₂₋₆alkynyl, C₃₋₆cycloalkyl, C₁₋₆alkoxy, C₃₋₆ alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})— and C₁₋₆alkoxycarbonyl-N(R^{a})— may optionally be substituted by one or more substituents each independently selected from R^{P}; and wherein aryl and heteroaryl may optionally be substituted by one or more substituents each independently selected from R^{f};

R^{X1} is selected from the group consisting of hydrogen, C₁₋₆alkyl and phenyl, or R^{X1} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X11} is selected from the group consisting of hydrogen, C₁₋₆alkyl and phenyl, or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X2} and R^{X3} are each independently selected from the group consisting of hydrogen, halogen, hydroxyl, —NR^{a}R^{b}, —C(O)—NR^{a}R^{b}, C₁₋₆alkyl and C₁₋₆alkoxy; wherein C₁₋₆alkyl and C₁₋₆alkoxy may optionally be substituted by one or more substituents each independently selected from R^{P};

or R^{X2} and R^{X3} together with the carbon to which they are attached form a 3-6 membered carbocycle optionally substituted by one or more substituents each independently selected from the group consisting of hydrogen, halogen, —NR^{a}R^{b}, —C(O)—NR^{a}R^{b}, oxo, C₁₋₆ alkyl and C₁₋₆alkoxy;

R^{X4} and R^{X5} are each independently selected from the group consisting of hydrogen and C₁₋₆alkyl; wherein C₁₋₆alkyl may optionally be substituted by one or more substituents each independently selected from R^{P};

R^{X6} and R^{X7} are each independently selected from the group consisting of hydrogen, halogen, —C(O)—NR^{a}R^{b}, cyano and C₁₋₆alkyl; wherein C₁₋₆alkyl may optionally be substituted by one or more substituents each independently selected from R^{P};

R^{Y} is selected from the group consisting of hydrogen, C₁₋₆alkyl and phenyl;

R³ is selected from the group consisting of hydrogen, C₁₋₆alkyl, C₃₋₆alkyl, phenyl, and heterocycle;

R^{f} is independently selected, for each occurrence, from the group consisting of R^{P}, C₁₋₆ alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, (wherein wherein w is 0, 1 or 2), C₁₋₆alkylcarbonyl-N(R^{a})— and C₁₋₆alkoxycarbonyl-N(R^{a})—; wherein C₁₋₆alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, C₁₋₆alkylcarbonyl-N(R^{a})—, and C₁₋₆alkoxycarbonyl-N(R^{a})— may be optionally substituted by one or more substituents selected from R^{P};

R^{h} is independently selected, for each occurrence, from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆ alkylcarbonyl-, C₁₋₆alkoxycarbonyl-, R^{a}R^{b}N-carbonyl- and R^{a}R^{b}N—SO₂—; wherein C₁₋₆alkyl, C₃₋₆alkenyl, C₃₋₆alkynyl, C₃₋₆cycloalkyl, C₁₋₆alkyl-S(O)₂—, C₁₋₆alkylcarbonyl- and C₁₋₆ alkoxycarbonyl- may optionally be substituted by one or more substituents selected from R^{P};

R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl;

or R^{a} and R^{b}, together with the nitrogen to which they are attached, may form a 4-6 membered monocyclic heterocyclic ring, which may have an additional heteroatom selected from the group consisting of O, S, and N; wherein the 4-6 membered heterocyclic ring may optionally be substituted by one or more substituents selected from the group consisting of halogen, cyano, oxo or hydroxyl; and

R^{P} is independently selected, for each occurrence, from the group consisting of halogen, hydroxyl, cyano, C₁₋₆alkoxy, R^{a}R^{b}N—, R^{a}R^{b}N-carbonyl-, R^{a}R^{b}N—SO₂—, and R^{a}R^{b}N-carbonyl-N(R^{a})—.

In some embodiments, X is selected from the group consisting of a bond, —O—, —NR^{X1}— and —NR^{X11}—.

In some embodiments, A is selected from the group consisting of phenyl, pyridyl, quinolinyl, indolyl, and indolinyl, wherein phenyl, pyridyl, quinolinyl, indolyl and indolinyl may optionally be substituted by one, two or three substituents each independently selected from the group consisting of —R¹⁰, —OR¹⁰, —SR¹⁰, —N(R¹⁰)₂, —OSO₂R¹⁰, —SO₂R¹⁰, —C(O)N(R¹⁰)₂, halogen, and cyano. In certain embodiments, A is phenyl; wherein phenyl may optionally be substituted by one, two, or three substituents each independently selected from the group consisting of —OR¹⁰, halogen, and cyano. In certain other embodiments, A is pyridyl; wherein pyridyl may optionally be substituted by one, two, or three substituents each independently selected from the group consisting of —OR¹⁰, halogen, and cyano.

In some embodiments, R^{X1} and ring A together with the nitrogen to which they are attached form a 1,2,3,4-tetrahydro-1,5-naphthyridine moiety. In some embodiments, R^{X11} and ring A together with the nitrogen to which they are attached form a 1,2,3,4-tetrahydro-1,5-naphthyridine moiety. In some embodiments, R^{X1} and ring A together with the nitrogen to which they are attached form a 1,2,3,4-tetrahydroquinoline moiety. In some embodiments, R^{X11} and ring A together with the nitrogen to which they are attached form a 1,2,3,4-tetrahydroquinoline moiety.

In some embodiments, A is phenyl, pyridyl, indolyl, or indolinyl, wherein said phenyl, pyridyl, indolyl or indolinyl is optionally substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano.

In some embodiments, A is phenyl, pyridyl, imidazolyl, indolyl, indolinyl, naphthyl, quinolinyl, isoquinolinyl, benzimidazolyl, indazolyl, benzotriazolyl, benzothiazolyl, benzofuranyl, pyrazolopyridyl, quinazolinyl, quinoxalinyl, or cinnolinyl. In some embodiments, A is phenyl. In some embodiments, A is pyridyl. In some embodiments, A is 2-pyridyl. In some embodiments, A is 3-pyridyl. In some embodiments, A is 4-pyridyl. In some embodiments, A is naphthyl. In some embodiments, A is 1-naphthyl. In some embodiments, A is 2-naphthyl. In some embodiments, A is quinolinyl. In some embodiments, A is 2-quinolinyl. In some embodiments, A is 3-quinolinyl. In some embodiments, A is 4-quinolinyl. In some embodiments, A is 5-quinolinyl. In some embodiments, A is 6-quinolinyl. In some embodiments, A is 7-quinolinyl. In some embodiments, A is 8-quinolinyl. In some embodiments, A is isoquinolinyl. In some embodiments, A is 1-isoquinolinyl. In some embodiments, A is 3-isoquinolinyl. In some embodiments, A is 4-isoquinolinyl. In some embodiments, A is 5-isoquinolinyl. In some embodiments, A is 6-isoquinolinyl. In some embodiments, A is 7-isoquinolinyl. In some embodiments, A is 8-isoquinolinyl. In some embodiments, A is indolyl. In some embodiments, A is 2-indolyl. In some embodiments, A is 3-indolyl. In some embodiments, A is 4-indolyl. In some embodiments, A is 5-indolyl. In some embodiments, A is 6-indolyl. In some embodiments, A is 7-indolyl. In some embodiments, A is aryl. In some embodiments, A is bicyclic aryl. In some embodiments, A is heteroaryl. In some embodiments, A is fused bicyclic heteroaryl.

In some embodiments, A is phenyl, pyridyl, or indolyl, wherein said phenyl, pyridyl, or indolyl is optionally substituted with —OR¹⁰, halogen, or cyano.

In some embodiments, A is phenyl, wherein said phenyl is optionally substituted with —OR¹⁰, halogen, or cyano.

In some embodiments, A is pyridyl, wherein said pyridyl is optionally substituted with —OR¹⁰, halogen, or cyano.

In some embodiments, A is indolyl or indolinyl, wherein said indolyl or indolinyl is optionally substituted with —OR¹⁰, halogen, or cyano.

In some embodiments, A is phenyl, pyridyl, indolyl, or indolinyl, wherein said phenyl, pyridyl, indolyl or indolinyl is optionally substituted with —OR¹⁰ or halogen.

In some embodiments, A is phenyl, pyridyl, indolyl, or indolinyl, wherein said phenyl, pyridyl, indolyl or indolinyl is optionally substituted with halogen.

In some embodiments, A is phenyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is phenyl substituted with —R¹⁰. In some embodiments, A is phenyl substituted with —OR °. In some embodiments, A is phenyl substituted with —SR¹⁰. In some embodiments, A is phenyl substituted with —N(R¹⁰)₂. In some embodiments, A is phenyl substituted with —OSO₂R¹⁰. In some embodiments, A is phenyl substituted with —SO₂R¹⁰. In some embodiments, A is phenyl substituted with —C(O)N(R¹⁰)₂. In some embodiments, A is phenyl substituted with halogen. In some embodiments, A is phenyl substituted with cyano.

In some embodiments, A is pyridyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is pyridyl substituted with —R¹⁰. In some embodiments, A is pyridyl substituted with —OR¹⁰. In some embodiments, A is pyridyl substituted with —SR¹⁰. In some embodiments, A is pyridyl substituted with —N(R¹⁰)₂. In some embodiments, A is pyridyl substituted with —OSO₂R¹⁰. In some embodiments, A is pyridyl substituted with —SO₂R¹⁰. In some embodiments, A is pyridyl substituted with —C(O)N(R¹⁰)₂. In some embodiments, A is pyridyl substituted with halogen. In some embodiments, A is pyridyl substituted with cyano.

In some embodiments, A is 3-pyridyl substituted with —OR¹⁰. In some embodiments, A is 3-pyridyl substituted with —OCH₃. In some embodiments, A is 6-methoxypyrid-3-yl. In some embodiments, A is 3-pyridyl substituted with halogen. In some embodiments, A is 3-pyridyl substituted with Cl. In some embodiments, A is 4-chloropyrid-3-yl. In some embodiments, A is 2,3-dichloropyrid-4-yl. In some embodiments, A is 3-chloropyrid-2-yl.

In some embodiments, A is naphthyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is naphthyl substituted with —R¹⁰. In some embodiments, A is naphthyl substituted with —OR¹⁰. In some embodiments, A is naphthyl substituted with —SR¹⁰. In some embodiments, A is naphthyl substituted with —N(R¹⁰)₂. In some embodiments, A is naphthyl substituted with —OSO₂R¹⁰. In some embodiments, A is naphthyl substituted with —SO₂R¹⁰. In some embodiments, A is naphthyl substituted with —C(O)N(R¹⁰)₂. In some embodiments, A is naphthyl substituted with halogen. In some embodiments, A is naphthyl substituted with cyano.

In some embodiments, A is quinolinyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is quinolinyl substituted with —R¹⁰. In some embodiments, A is quinolinyl substituted with —OR¹⁰. In some embodiments, A is quinolinyl substituted with —SR¹⁰. In some embodiments, A is quinolinyl substituted with —N(R¹⁰)₂. In some embodiments, A is quinolinyl substituted with —OSO₂R¹⁰. In some embodiments, A is quinolinyl substituted with —SO₂R¹⁰. In some embodiments, A is quinolinyl substituted with —C(O)N(R¹⁰)₂. In some embodiments, A is quinolinyl substituted with halogen. In some embodiments, A is quinolinyl substituted with cyano.

In some embodiments, A is isoquinolinyl substituted with —R¹⁰, —OR, —SR¹⁰, —N(R¹⁰)₂, —OSO₂R¹⁰, —SO₂R¹⁰, —C(O)N(R¹⁰)₂, halogen, or cyano. In some embodiments, A is isoquinolinyl substituted with —R¹⁰. In some embodiments, A is isoquinolinyl substituted with —OR¹⁰. In some embodiments, A is isoquinolinyl substituted with —SR¹⁰. In some embodiments, A is isoquinolinyl substituted with —N(R¹⁰)₂. In some embodiments, A is isoquinolinyl substituted with —OSO₂R¹⁰. In some embodiments, A is isoquinolinyl substituted with —SO₂R¹⁰. In some embodiments, A is isoquinolinyl substituted with —C(O)N(R¹⁰)₂. In some embodiments, A is isoquinolinyl substituted with halogen. In some embodiments, A is isoquinolinyl substituted with cyano.

In some embodiments, A is indolyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is indolyl substituted with —R¹⁰. In some embodiments, A is indolyl substituted with —OR¹⁰. In some embodiments, A is indolyl substituted with —SR¹⁰. In some embodiments, A is indolyl substituted with —N(R¹⁰)₂. In some embodiments, A is indolyl substituted with —OSO₂R¹⁰. In some embodiments, A is indolyl substituted with —SO₂R¹⁰. In some embodiments, A is indolyl substituted with —C(O)N(R¹⁰)₂. In some embodiments, A is indolyl substituted with halogen. In some embodiments, A is indolyl substituted with cyano.

In some embodiments, A is 1-methyl-1H-indol-4-yl. In some embodiments, A is 1-methyl-1H-indol-3-yl.

In some embodiments, A is imidazol-2-yl. In some embodiments, A is 1-methyl-imidazol-2-yl.

In some embodiments, A is selected from the group consisting of 1,2,3,4-tetrahydroquinoline; 1,2,3,4-tetrahydro-1,5,napthyridine, dihydropyridopyrazine, benzoimidazole, benzoazepine, and dihydropyridooxazine, wherein A is bound to X through an available nitrogen on X; and where A is optionally substituted with one or two substituents each independently selected from halo, CF₃, phenyl (optionally substituted with one, two or three halo, C₁₋₃alkyl or C₁₋₃haloalkyl), C₁₋₃alkyl, C₁₋₃haloalkyl, and heteroaryl (optionally substituted with one, two or three halo, C₁₋₃alkyl or C₁₋₃haloalkyl).

In some embodiments, A is selected from the group consisting of

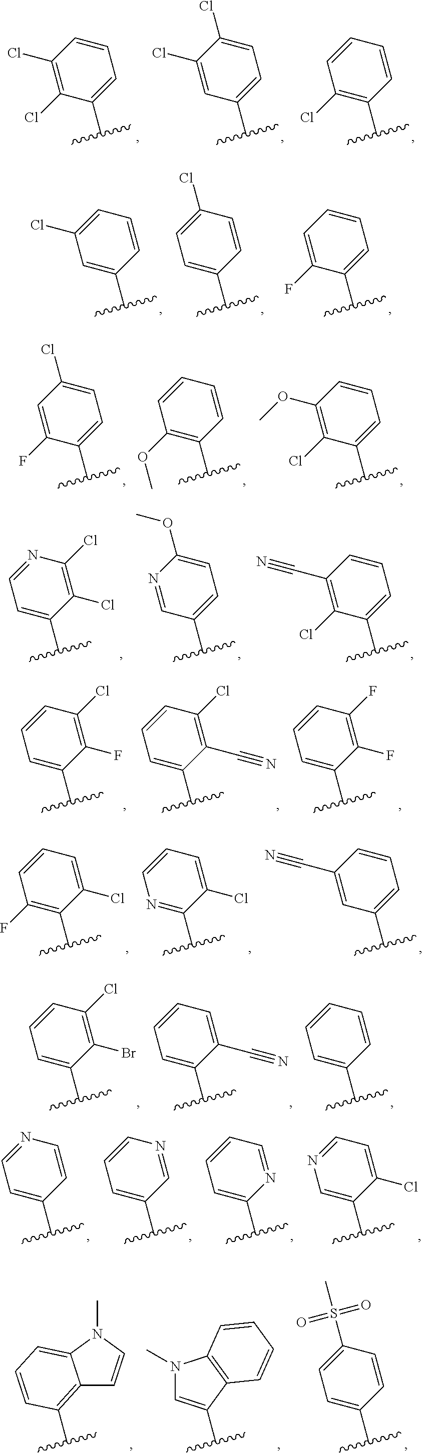

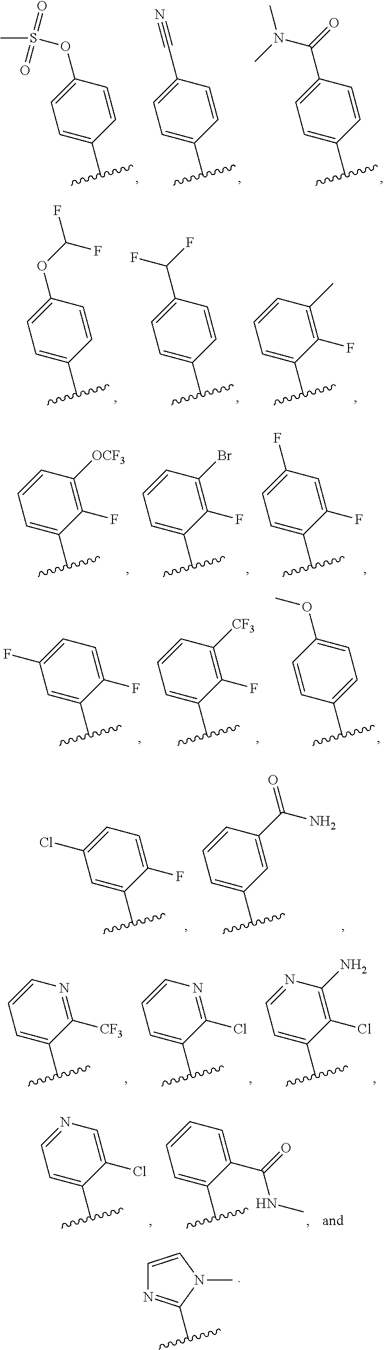

In some embodiments, A is

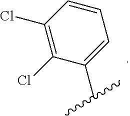 [SMILES: *c1cccc(Cl)c1Cl]

In some embodiments, A is

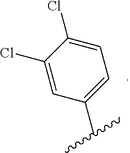 [SMILES: *c1ccc(Cl)c(Cl)c1]

In some embodiments, A is

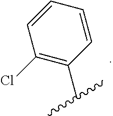 [SMILES: *c1ccccc1Cl]

In some embodiments, A is

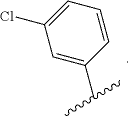 [SMILES: *c1cccc(Cl)c1]

In some embodiments, A is

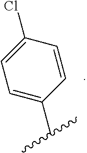 [SMILES: *c1ccc(Cl)cc1]

In some embodiments, A is

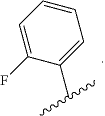 [SMILES: *c1ccccc1F]

In some embodiments, A is

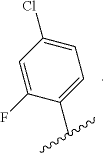 [SMILES: *c1ccc(Cl)cc1F]

In some embodiments, A is

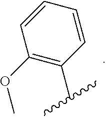 [SMILES: *c1ccccc1OC]

In some embodiments, A is

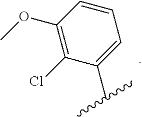 [SMILES: *c1cccc(OC)c1Cl]

In some embodiments, A is

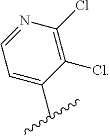 [SMILES: *c1ccnc(Cl)c1Cl]

In some embodiments, A is

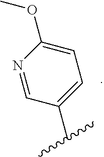 [SMILES: *c1ccc(OC)nc1]

In some embodiments, A is

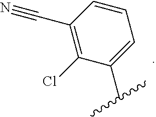 [SMILES: *c1cccc(C#N)c1Cl]

In some embodiments, A is

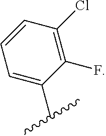 [SMILES: *c1cccc(Cl)c1F]

In some embodiments, A is

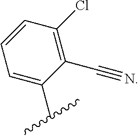 [SMILES: *c1cccc(Cl)c1C#N]

In some embodiments, A is

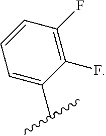 [SMILES: *c1cccc(F)c1F]

In some embodiments, A is

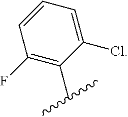 [SMILES: *c1c(F)cccc1Cl]

In some embodiments, A is

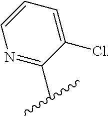 [SMILES: *c1ncccc1Cl]

In some embodiments, A is

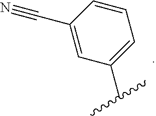 [SMILES: *c1cccc(C#N)c1]

In some embodiments, A is

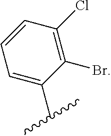 [SMILES: *c1cccc(Cl)c1Br]

In some embodiments, A is

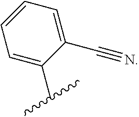 [SMILES: *c1ccccc1C#N]

In some embodiments, A is

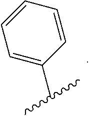 [SMILES: *c1ccccc1]

In some embodiments, A is

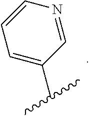 [SMILES: *c1cccnc1]

In some embodiments, A is

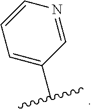 [SMILES: *c1cccnc1]

In some embodiments, A is

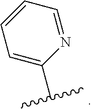 [SMILES: *c1ccccn1]

In some embodiments, A is

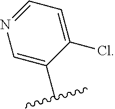 [SMILES: *c1cnccc1Cl]

In some embodiments, A is

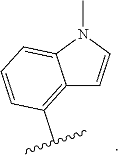 [SMILES: *c1cccc2c1ccn2C]

In some embodiments, A is

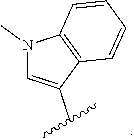 [SMILES: *c1cn(C)c2ccccc12]

In some embodiments, A is

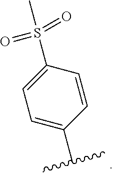 [SMILES: *c1ccc(S(C)(=O)=O)cc1]

In some embodiments, A is

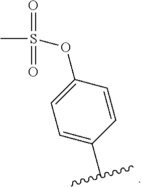 [SMILES: *c1ccc(OS(C)(=O)=O)cc1]

In some embodiments, A is

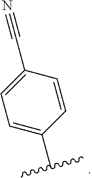 [SMILES: *c1ccc(C#N)cc1]

In some embodiments, A is

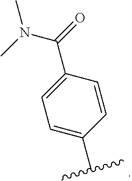 [SMILES: *c1ccc(C(=O)N(C)C)cc1]

In some embodiments, A is

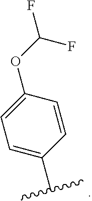 [SMILES: *c1ccc(OC(F)F)cc1]

In some embodiments, A is

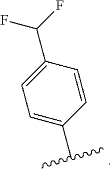 [SMILES: *c1ccc(C(F)F)cc1]

In some embodiments, A is

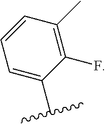 [SMILES: *c1cccc(C)c1F]

In some embodiments, A is

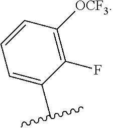 [SMILES: *c1cccc(OC(F)(F)F)c1F]

In some embodiments, A is

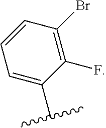 [SMILES: *c1cccc(Br)c1F]

In some embodiments, A is

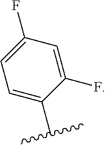 [SMILES: *c1ccc(F)cc1F]

In some embodiments, A is

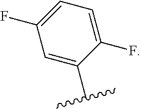 [SMILES: *c1cc(F)ccc1F]

In some embodiments, A is

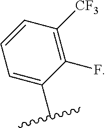 [SMILES: *c1cccc(C(F)(F)F)c1F]

In some embodiments, A is

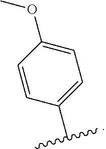 [SMILES: *c1ccc(OC)cc1 ; "."]

In some embodiments, A is

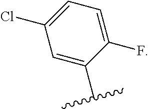 [SMILES: *c1cc(Cl)ccc1F]

In some embodiments, A is

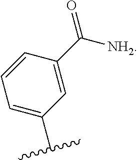 [SMILES: *c1cccc(C(N)=O)c1]

In some embodiments, A is

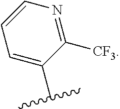 [SMILES: *c1cccnc1C(F)(F)F]

In some embodiments, A is

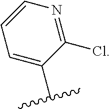 [SMILES: *c1cccnc1Cl]

In some embodiments, A is

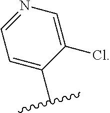 [SMILES: *c1ccncc1Cl]

In some embodiments, A is

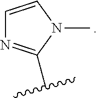 [SMILES: *c1nccn1C ; "."]

In some embodiments, A is

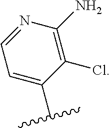 [SMILES: *c1ccnc(N)c1Cl]

In some embodiments, A is

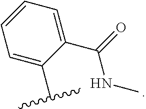 [SMILES: *c1ccccc1C(=O)NC ; "."]

In certain embodiments, R³ is selected from the group consisting of hydrogen, CH₃ and —NH₂. In some embodiments, Y is a bond and Z is N.

In some embodiments, R¹ and R² together with the nitrogen to which they are attached form heterocyclic ring B; wherein ring B is selected from the group consisting of pyrrolidinyl, piperidinyl, azepanyl and morpholinyl; wherein pyrrolidinyl, piperidinyl, azepanyl and morpholinyl may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃alkoxy; wherein C₁₋₃alkyl and C₁₋₃alkoxy may optionally be substituted by halogen, hydroxyl and —NR^{a}R^{b}.

In some embodiments, Y is —NH—, —N(CH₃)—, —N(H)—CH₂—, or —N(CH₃)—CH₂—; and Z is C(H).

In certain embodiments, R¹ and R² together with the carbon to which they are attached form carbocyclic ring B; wherein ring B is selected from the group consisting of cyclopentyl and cyclohexyl, and wherein cyclopentyl and cyclohexyl may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃ alkoxy; wherein C₁₋₃alkyl and C₁₋₃alkoxy may optionally be substituted by halogen, hydroxyl and —NR^{a}R^{b}.

In certain other embodiments, R¹ and R² together with the carbon to which they are attached form heterocyclic ring B; wherein ring B is selected from the group consisting of tetrahydrofuranyl and tetrahydropyranyl, wherein tetrahydrofuranyl and tetrahydropyranyl may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃alkoxy; wherein C₁₋₃alkyl and C₁₋₃alkoxy may optionally be substituted by halogen, hydroxyl and —NR^{a}R^{b}.

For example, in some embodiments, ring B is substituted on an available carbon by a substituent selected from the group consisting of —NH₂, —CH₃, and —CH₂NH₂.

In certain embodiments, R¹ is hydrogen and R² is branched C₁₋₁₀alkyl; wherein C₁₋₁₀alkyl may optionally be substituted by one, two, three or more substituents each independently selected from the group consisting of halogen and —NH₂.

In some embodiments, ring D is absent, and formula X is represented by formula Xa:

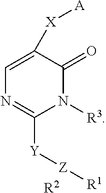

In some embodiments, ring D is present, and formula I is represented by Xb or Xc:

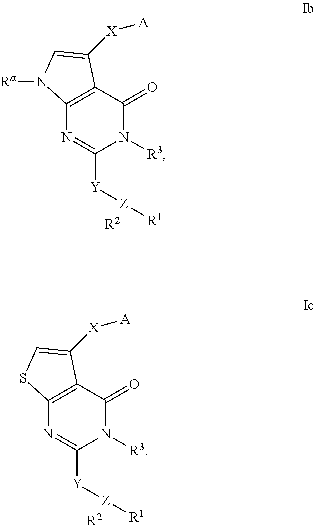

In some embodiments, SHP2 phosphatase inhibitors described herein encompass compounds of Formula XI, or a pharmaceutically acceptable salt or stereoisomer thereof, wherein Formula XI is represented by:

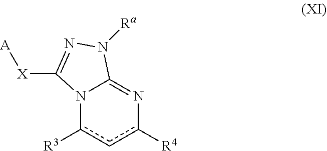

wherein

X is selected from the group consisting of a bond, —O—, —NR^{X1}—, —C(O)NR^{X11}—, —S(O)_{w}— (wherein w is 0, 1 or 2), —C(O)—, —C(R^{X2}R^{X3})—, —O—C(R^{X4}R^{X5}), —C(R^{X4}R^{X5})—O—, and —C═C(R^{X6}R^{X7})—;

R³ is —Y—Z(R¹)(R²) and R⁴ is ═O; or R³ is ═O and R⁴ is —Y—Z(R¹)(R²);

Y is selected from the group consisting of a bond, —NR^{Y}—, —C₁₋₃alkylene-NR^{Y}—, and —NR^{Y}—C₁₋₃alkylene-;

Z is selected from the group consisting of N and C(R^{Z});

wherein if Y is a bond, Z is N, and wherein if Y is —NR^{Y}—, —C₁₋₃alkylene-NR^{Y}—, or —NR^{Y}—C₁₋₃alkylene-, Z is C(R^{Z});

R^{Z} is selected from the group consisting of hydrogen and C₁₋₆alkyl; wherein C₁₋₆alkyl may optionally be substituted by halogen, hydroxyl, and —NR^{a}R^{b},

A is selected from the group consisting of a 6-10 membered monocyclic or bicyclic aryl, a 5-7 membered monocyclic heteroaryl having one or more heteroatoms each independently selected from O, S, or N, a 8-10 membered bicyclic heteroaryl having one or more heteroatoms each independently selected from O, S, or N; and a 4-7 membered heterocyclyl, wherein A may optionally be substituted by one, two, three or more substituents each independently selected from the group consisting of —R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R¹⁰ is independently selected from the group consisting of hydrogen, C₁₋₆alkyl, C₁₋₆haloalkyl, C₃₋₆cycloalkyl, heterocycloalkyl, aryl, and heteroaryl; wherein R¹⁰ may optionally be substituted by one, two, three or more substituents each independently selected from the group consisting of halogen, —C(O)R²⁰, C₁₋₆alkyl and C₁₋₆haloalkyl;

R²⁰ is selected from the group consisting of hydroxyl, halogen, and C₁₋₆alkyl;

R¹ and R² are each selected from the group consisting of hydrogen and C₁₋₁₀alkyl; wherein C₁₋₁₀alkyl may optionally be substituted by one, two, three or more substituents each independently selected from R^{P};

or R¹ and R², together with carbon or nitrogen to which they are attached, form a 3-7 membered saturated carbocyclic or 4-7 membered saturated heterocyclic ring B having one or two heteroatoms each independently selected from the group consisting of 0, S(O)_{w} (wherein w is 0, 1, or 2), and NR^{h}; wherein carbocyclic or heterocyclic ring B may be monocyclic or bicyclic;

carbocyclic or heterocyclic ring B may optionally be substituted on one or more available carbons by one, two, three or more substituents each independently selected from the group consisting of halogen, hydroxyl, cyano, oxo, —NR^{a}R^{b}, —CO₂H, —C(O)—NR^{a}R^{b}, —S(O)₂—NR^{a}R^{b}, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})—, C₁₋₆alkoxycarbonyl-N(R^{a})—, aryl and heteroaryl; wherein C₁₋₆alkyl, C₂₋₆alkenyl, C₂₋₆alkynyl, C₃₋₆cycloalkyl, C₁₋₆alkoxy, C₃₋₆ alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})— and C₁₋₆alkoxycarbonyl-N(R^{a})— may optionally be substituted by one or more substituents each independently selected from R^{P}; and wherein aryl and heteroaryl may optionally be substituted by one or more substituents each independently selected from R^{f};

R^{X1} is selected from the group consisting of hydrogen, C₁₋₆alkyl and phenyl, or R^{X1} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X11} is selected from the group consisting of hydrogen, C₁₋₆alkyl and phenyl, or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X2} and R^{X3} are each independently selected from the group consisting of hydrogen, halogen, hydroxyl, —NR^{a}R^{b}, —C(O)—NR^{a}R^{b}, C₁₋₆alkyl and C₁₋₆alkoxy; wherein C₁₋₆alkyl and C₁₋₆alkoxy may optionally be substituted by one or more substituents each independently selected from R^{P};

or R^{X2} and R^{X3} together with the carbon to which they are attached form a 3-6 membered carbocycle optionally substituted by one or more substituents each independently selected from the group consisting of hydrogen, halogen, —NR^{a}R^{b}, —C(O)—NR^{a}R^{b}, oxo, C₁₋₆ alkyl and C₁₋₆alkoxy;

R^{X4} and R^{X5} are each independently selected from the group consisting of hydrogen and C₁₋₆alkyl; wherein C₁₋₆alkyl may optionally be substituted by one or more substituents each independently selected from R^{P};

R^{X6} and R^{X7} are each independently selected from the group consisting of hydrogen, halogen, —C(O)—NR^{a}R^{b}, cyano and C₁₋₆alkyl; wherein C₁₋₆alkyl may optionally be substituted by one or more substituents each independently selected from R^{P};

R^{Y} is selected from the group consisting of hydrogen, C₁₋₆alkyl and phenyl;

R³ is selected from the group consisting of hydrogen, C₁₋₆alkyl, C₃₋₆alkyl, phenyl, and heterocycle;

R^{f} is independently selected, for each occurrence, from the group consisting of R^{P}, C₁₋₆ alkyl, C₃₋₆cycloalkyl, C₂₋₆alkenyl, C₂₋₆alkynyl, C₁₋₆alkoxy, C₁₋₆ alkylcarbonyl-, C₁₋₆alkoxycarbonyl, C₁₋₆alkyl-S(O)_{w}—, (wherein wherein w is 0, 1 or 2), C₁₋₆alkylcarbonyl-N(R^{a})— and C₁₋₆alkoxycarbonyl-N(R^{a})—; wherein C₁₋₆alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, C₁₋₆alkylcarbonyl-N(R^{a})—, and C₁₋₆alkoxycarbonyl-N(R^{a})— may be optionally substituted by one or more substituents selected from R^{P};

R^{h} is independently selected, for each occurrence, from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆ alkylcarbonyl-, C₁₋₆alkoxycarbonyl-, R^{a}R^{b}N-carbonyl- and R^{a}R^{b}N—SO₂—; wherein C₁₋₆alkyl, C₃₋₆alkenyl, C₃₋₆alkynyl, C₃₋₆cycloalkyl, C₁₋₆alkyl-S(O)₂—, C₁₋₆alkylcarbonyl- and C₁₋₆ alkoxycarbonyl- may optionally be substituted by one or more substituents selected from R^{P};

R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl;

or R^{a} and R^{b}, together with the nitrogen to which they are attached, may form a 4-6 membered monocyclic heterocyclic ring, which may have an additional heteroatom selected from the group consisting of O, S, and N; wherein the 4-6 membered heterocyclic ring may optionally be substituted by one or more substituents selected from the group consisting of halogen, cyano, oxo or hydroxyl; and

R^{P} is independently selected, for each occurrence, from the group consisting of halogen, hydroxyl, cyano, C₁₋₆alkoxy, R^{a}R^{b}N—, R^{a}R^{b}N-carbonyl-, R^{a}R^{b}N—SO₂—, and R^{a}R^{b}N-carbonyl-N(R^{a})—.

In some embodiments, the compound is represented by XIb or XIc:

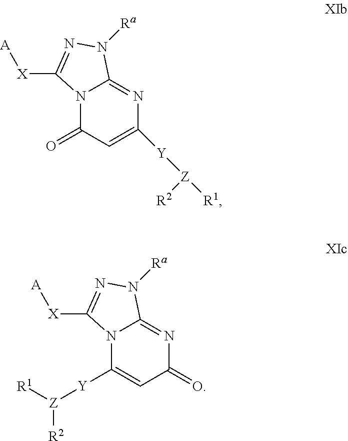

In some embodiments, —Y—Z—(R¹)(R²) is:

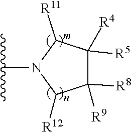

wherein

R⁴ and R⁵ are each independently selected from the group consisting of hydrogen, hydroxyl, C₁₋₆alkyl, C₁₋₆alkoxy, C₁₋₆alkyl-O—R⁶, —C(O)N(R⁶)₂, —N(R⁶)₂, halogen, C₁₋₆alkyl-N(R⁶)₂, and cyano; wherein C₁₋₆alkyl, C₁₋₆alkoxy, C₁₋₆alkyl-O—R⁶, and C₁₋₆alkyl-N(R⁶)₂ may optionally be substituted by one or more substituents each independently selected from the group consisting of hydroxyl, —NH₂, oxo, and halogen,

or R⁴ and R⁵, taken together with the atoms to which they are attached, form a 3-7 membered carbocyclic or heterocyclic saturated or partially unsaturated ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;

or R⁴ and R⁸, taken together with the atoms to which they are attached, form a 4-7 membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;

or R⁴ is absent, and R⁴ and R⁸, taken together with the atoms to which they are attached, form a 3-membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;

R⁶ is independently for each occurrence selected from the group consisting of hydrogen, C₁₋₆alkyl and phenyl;

R¹¹ and R¹² are each independently selected from the group consisting of hydrogen, hydroxyl, C₁₋₆alkyl, C₁₋₆alkoxy, C₁₋₆alkyl-O—R⁶, aryl, arylalkylene, heteroaryl, heteroarylalkylene, —C(O)N(R⁶)₂, —N(R⁶)₂, halogen, C₁₋₆alkyl-N(R⁶)₂, —CO₂H, and cyano; wherein C₁₋₆alkyl, C₁₋₆alkoxy, C₁₋₆alkyl-O—R⁶, aryl, arylalkylene, heteroaryl, heteroarylalkylene, and C₁₋₆alkyl-N(R⁶)₂ may optionally be substituted by one or more substituents each independently selected from the group consisting of hydroxyl, —NH₂, and halogen;

or R¹¹ and R¹², taken together with the atoms to which they are attached, form a 5-7 membered heterocyclic ring;

or R⁴ and R¹², taken together with the atoms to which they are attached, form a 5-7 membered carbocyclic or heterocyclic ring;

or R⁸ and R¹¹, taken together with the atoms to which they are attached, form a 5-7 membered carbocyclic or heterocyclic ring;

R⁸ and R⁹ are each independently selected from the group consisting of hydrogen, C₁₋₆)alkyl, C₁₋₆alkyl-N(R⁶)₂, —OR⁶, C₁₋₆alkyl-O—R⁶, —C(O)NH₂, —N(R⁶)₂, halogen, and cyano; and

each of m and n is, independently, 0, 1, 2, or 3, with m+n being at least 2 and no more than 4.

In some embodiments, R⁴ and R⁵ are each independently selected from the group consisting of C₁₋₃alkyl and —NR^{a}R^{b}; wherein C₁₋₃alkyl may optionally be substituted by one, two or three substituents each independently selected from the group consisting of halogen, hydroxyl and —NR^{a}R^{b}. In certain embodiments, R⁴ and R⁵ are each independently selected from the group consisting of —NH₂, —CH₃, and —CH₂NH₂.

In some embodiments, —Y—Z—(R¹)(R²) is represented by:

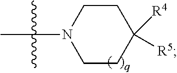

wherein

R⁴ and R⁵ are each independently selected from the group consisting of hydrogen, halogen, cyano, —NR^{a}R^{b}, —C(O)—NR^{a}R^{b} and C₁₋₆alkyl, wherein C₁₋₆alkyl may optionally be substituted by one, two or three or more substituents each independently selected from the group consisting of halogen, hydroxyl, —NR^{a}R^{b} and oxo;

R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl; and q is 0 or 1.

Examples of compounds of the present disclosure include:

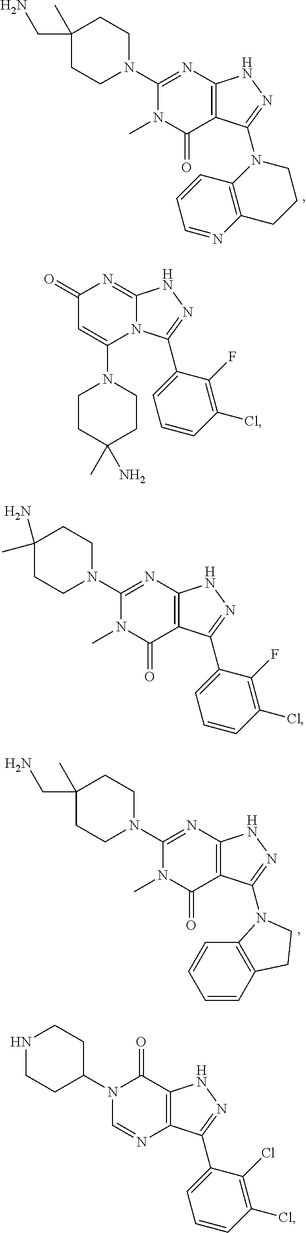 [SMILES: Cn1c(N2CCC(C)(CN)CC2)nc2[nH]nc(N3CCCc4ncccc43)c2c1=O ; CC1(N)CCN(c2cc(=O)nc3[nH]nc(-c4cccc(Cl)c4F)n23)CC1 ; Cn1c(N2CCC(C)(N)CC2)nc2[nH]nc(-c3cccc(Cl)c3F)c2c1=O ; Cn1c(N2CCC(C)(CN)CC2)nc2[nH]nc(N3CCc4ccccc43)c2c1=O ; O=c1c2[nH]nc(-c3cccc(Cl)c3Cl)c2ncn1C1CCNCC1]

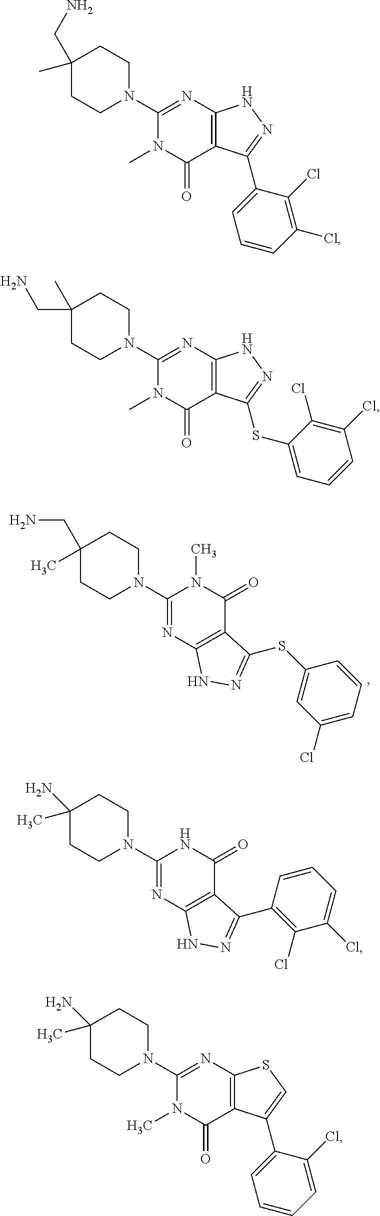 [SMILES: Cn1c(N2CCC(C)(CN)CC2)nc2[nH]nc(-c3cccc(Cl)c3Cl)c2c1=O ; Cn1c(N2CCC(C)(CN)CC2)nc2[nH]nc(Sc3cccc(Cl)c3Cl)c2c1=O ; Cn1c(N2CCC(C)(CN)CC2)nc2[nH]nc(Sc3cccc(Cl)c3)c2c1=O ; CC1(N)CCN(c2nc3[nH]nc(-c4cccc(Cl)c4Cl)c3c(=O)[nH]2)CC1 ; Cn1c(N2CCC(C)(N)CC2)nc2scc(-c3ccccc3Cl)c2c1=O]

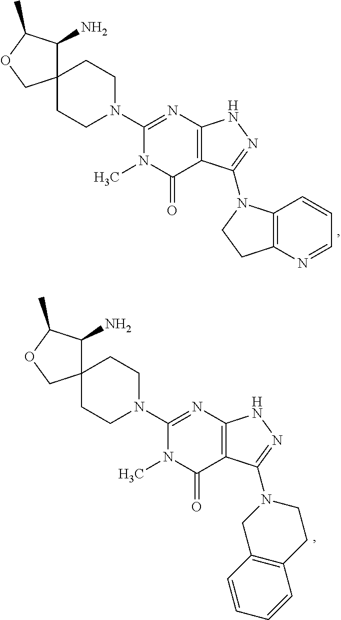 [SMILES: C[C@@H]1OCC2(CCN(c3nc4[nH]nc(N5CCc6ncccc65)c4c(=O)n3C)CC2)[C@@H]1N ; C[C@@H]1OCC2(CCN(c3nc4[nH]nc(N5CCc6ccccc6C5)c4c(=O)n3C)CC2)[C@@H]1N]

and a pharmaceutically acceptable salt or stereoisomer thereof.

Also disclosed herein is a method of treating a disorder in a patient in need thereof, comprising administering to the patient an effective amount of a compound of Formula XII, or a pharmaceutically acceptable salt, stereoisomer or N-oxide thereof, wherein Formula XII is represented by:

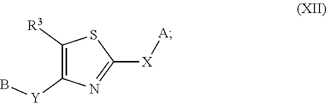

wherein

X is selected from the group consisting of a bond, —O—, —NR^{X1}—, —C(O)NR^{X11}—, —S(O)_{w}— (wherein w is 0, 1 or 2), —C(O)—, —C(R^{X2}R^{X3})—, —O—C(R^{X4}R^{X5}), —C(R^{X4}R^{X5})—O—, and —C═C(R^{X6}R^{X7})—;

A is selected from the group consisting of a 6-10 membered monocyclic or bicyclic aryl, a 5-7 membered monocyclic heteroaryl having one or more heteroatoms each independently selected from O, S, or N, a 8-10 membered bicyclic heteroaryl having one or more heteroatoms each independently selected from O, S, or N; and a 4-7 membered heterocyclyl, wherein A may optionally be substituted by one, two, three or more substituents each independently selected from the group consisting of —R¹⁰, —OR¹⁰, methylenedioxy, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R¹⁰ is independently selected from the group consisting of hydrogen, C₁₋₆alkyl, C₁₋₆haloalkyl, C₃₋₆cycloalkyl, heterocycloalkyl, aryl, and heteroaryl; wherein R¹⁰ may optionally be substituted by one, two, three or more substituents each independently selected from the group consisting of halogen, —C(O)R²⁰, C₁₋₆alkyl and C₁₋₆haloalkyl;

R²⁰ is selected from the group consisting of hydroxyl, halogen, and C₁₋₆alkyl;

Y is selected from the group consisting of C₁₋₆alkylene and C₁₋₆alkenylene; wherein C₁₋₆alkylene and C₁₋₆alkenylene may optionally be substituted by one or two substituents each independently selected from the group consisting of halogen and hydroxyl; and wherein one or two methylene units of L may optionally and independently be replaced by a moiety selected from the group consisting of a bond, —O—, —C(O)—, —O—C(O)—, —C(O)—O—, —NR^{a}—, —C(O)—NR^{a}—, —NR^{a}—C(O)—, —O—C(O)—NR^{a}, R^{a}—C(O)—O—, —S(O)_{w}— (wherein w is 0, 1, or 2), —S(O)_{w}—NR^{a}—, and —NR^{a}—S(O)_{w}—;

B is selected from the group consisting of aryl, heteroaryl, heterocyclyl, and hydrogen; wherein aryl and heteroaryl may optionally be substituted by one, two, three or more substituents each independently selected from R^{e}; and wherein heterocyclyl is bound to L through a ring carbon or ring nitrogen and may optionally be substituted by one, two, three or more substituents each independently selected from R^{g}; and wherein if said heterocyclyl contains a —NH moiety that nitrogen may optionally be substituted by R^{h};

R^{X1} is selected from the group consisting of hydrogen, C₁₋₆alkyl and phenyl; or R^{X1} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X11} is selected from the group consisting of hydrogen, C₁₋₆alkyl and phenyl; or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;

R^{X2} and R^{X3} are each independently selected from the group consisting of hydrogen, halogen, hydroxyl, —NR^{a}R^{b}, —C(O)—NR^{a}R^{b}, C₁₋₆alkyl and C₁₋₆alkoxy; wherein C₁₋₆alkyl and C₁₋₆alkoxy may optionally be substituted by one or more substituents each independently selected from R^{P};

or R^{X2} and R^{X3} together with the carbon to which they are attached form a 3-6 membered carbocycle optionally substituted by one or more substituents each independently selected from the group consisting of hydrogen, halogen, —NR^{a}R^{b}, —C(O)—NR^{a}R^{b}, oxo, C₁₋₆ alkyl and C₁₋₆alkoxy;

R^{X4} and R^{X5} are each independently selected from the group consisting of hydrogen and C₁₋₆alkyl; wherein C₁₋₆alkyl may optionally be substituted by one or more substituents each independently selected from R^{P};

R^{X6} and R^{X7} are each independently selected from the group consisting of hydrogen, halogen, —C(O)—NR^{a}R^{b}, cyano and C₁₋₆alkyl; wherein C₁₋₆alkyl may optionally be substituted by one or more substituents each independently selected from R^{P};

R³ is selected from the group consisting of hydrogen, halogen, hydroxyl, C₁₋₆ alkyl and —NR^{a}R^{b};

R^{e} is independently selected, for each occurrence, from the group consisting of halogen, hydroxyl, cyano, oxo, thio, —NR^{a}R^{b}, —CO₂H, —C(O)—NR^{a}R^{b}, —S(O)₂—NR^{a}R^{b}, C₁₋₆alkyl, C₂₋₆alkenyl, C₂₋₆alkynyl, C₃₋₆cycloalkyl, C₁₋₆alkoxy, —S—C₁₋₆alkyl, —S—C₂₋₆alkenyl, C₁₋₆ alkyl-O—C₁₋₆alkyl, C₃₋₆alkenyloxy, C₃₋₆alkynyloxy, C₃₋₆cycloalkoxy, C₁₋₆alkylcarbonyl-, C₁₋₆ alkoxycarbonyl, C₁₋₆alkyl-S(O)_{w}— (wherein w is 0, 1 or 2), C₁₋₆alkylcarbonyl-N(R^{a})—, C₁₋₆ alkoxycarbonyl-N(R^{a})—, aryl and heteroaryl; wherein C₁₋₆alkyl, C₂₋₆alkenyl, C₂₋₆alkynyl, C₃₋₆ cycloalkyl, C₁₋₆alkoxy, —S—C₁₋₆alkyl, —S—C₂₋₆alkenyl, C₁₋₆alkyl-O—C₁₋₆alkyl, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})— and C₁₋₆alkoxycarbonyl-N(R^{a})— may optionally be substituted by one or more substituents each independently selected from R^{P}; and wherein aryl and heteroaryl may optionally be substituted by one or more substituents each independently selected from R^{f};

R^{f} is independently selected, for each occurrence, from the group consisting of R^{P}, C₁₋₆ alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, (wherein w is 0, 1 or 2), C₁₋₆alkylcarbonyl-N(R^{a})— and C₁₋₆ alkoxycarbonyl-N(R^{a})—; wherein C₁₋₆alkyl, C₃₋₆cycloalkyl, C₂₋₆alkenyl, C₂₋₆alkynyl, C₁₋₆ alkoxy, C₁₋₆alkylcarbonyl-, C₁₋₆alkoxycarbonyl, C₁₋₆ alkyl-S(O)_{w}—, C₁₋₆alkylcarbonyl-N(R^{a})—, and C₁₋₆alkoxycarbonyl-N(R^{a})— may be optionally substituted by one or more substituents selected from R^{P};

R^{g} is independently selected, for each occurrence, from the group consisting of halogen, hydroxyl, cyano, oxo, thio, —NR^{a}R^{b}, —CO₂H, —C(O)—NR^{a}R^{b}, —S(O)₂—NR^{a}R^{b}, C₁₋₆ alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})—, C₁₋₆alkoxycarbonyl-N(R^{a})—, aryl and heteroaryl; wherein C₁₋₆ alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})— and C₁₋₆alkoxycarbonyl-N(R^{a})— may optionally be substituted by one or more substituents each independently selected from R^{P}; and wherein aryl and heteroaryl may optionally be substituted by one or more substituents each independently selected from R;

R^{h} is independently selected, for each occurrence, from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆ alkylcarbonyl-, C₁₋₆alkoxycarbonyl-, R^{a}R^{b}N-carbonyl-, R^{a}R^{b}N—SO₂— and aryl; wherein C₁₋₆ alkyl, C₃₋₆alkenyl, C₃₋₆alkynyl, C₃₋₆cycloalkyl, C₁₋₆alkyl-S(O)₂—, C₁₋₆alkylcarbonyl- and C₁₋₆ alkoxycarbonyl- may optionally be substituted by one or more substituents selected from R^{P}; and wherein aryl may optionally be substituted by one or more substituents each independently selected from R^{f};

R^{a} and R^{b} are independently selected, for each occurrence, from the group consisting of hydrogen, C₁₋₆alkyl, C₃₋₆cycloalkyl, and phenyl; wherein C₁₋₆alkyl may optionally be substituted by one or more substituents selected from halogen, cyano, oxo, heterocyclyl and hydroxyl; and wherein phenyl may optionally be substituted by C₁₋₃alkyl, C₁₋₃alkoxy, halogen, hydroxyl;

or R^{a} and R^{b}, together with the nitrogen to which they are attached, may form a 4-6 membered monocyclic heterocyclic ring, which may have an additional heteroatom selected from the group consisting of O, S, and N; wherein the 4-6 membered heterocyclic ring may optionally be substituted by one or more substituents selected from the group consisting of halogen, cyano, oxo, hydroxyl and C₁₋₃alkyl (optionally substituted with —NH₂); and

R^{P} is independently selected, for each occurrence, from the group consisting of halogen, hydroxyl, cyano, C₁₋₆alkoxy, R^{a}R^{b}N—, R^{a}R^{b}N-carbonyl-, R^{a}R^{b}N—SO₂—, and R^{a}R^{b}N-carbonyl-N(R^{a})—.

In some embodiments, X is selected from the group consisting of a bond, —O—, —NR^{X1}— and —NR^{X11}—. In some embodiments, A is selected from the group consisting of phenyl, pyridyl, quinolinyl, indolyl, and indolinyl, wherein phenyl, pyridyl, quinolinyl, indolyl and indolinyl may optionally be substituted by one, two or three substituents each independently selected from the group consisting of —R¹⁰, —OR¹⁰, methylenedioxy, —SR¹⁰, —N(R¹⁰)₂, —OSO₂R¹⁰, —SO₂R¹⁰, —C(O)N(R¹⁰)₂, halogen, and cyano. In some embodiments, A is phenyl; wherein phenyl may optionally be substituted by one, two, or three substituents each independently selected from the group consisting of —R¹⁰, —OR¹⁰, methylenedioxy, halogen, and cyano. In some embodiments, A is pyridyl; wherein pyridyl may optionally be substituted by one, two, or three substituents each independently selected from the group consisting of —R¹⁰, —OR¹⁰, halogen, and cyano.

In some embodiments, R^{X1} and ring A together with the nitrogen to which they are attached form a 1,2,3,4-tetrahydro-1,5-naphthyridine moiety. In some embodiments, R^{X11} and ring A together with the nitrogen to which they are attached form a 1,2,3,4-tetrahydro-1,5-naphthyridine moiety. In some embodiments, R^{X1} and ring A together with the nitrogen to which they are attached form a 1,2,3,4-tetrahydroquinoline moiety. In some embodiments, R^{X11} and ring A together with the nitrogen to which they are attached form a 1,2,3,4-tetrahydroquinoline moiety.

In some embodiments, A is phenyl, pyridyl, indolyl, or indolinyl, wherein said phenyl, pyridyl, indolyl or indolinyl is optionally substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano.

In some embodiments, A is phenyl, pyridyl, imidazolyl, indolyl, indolinyl, naphthyl, quinolinyl, isoquinolinyl, benzimidazolyl, indazolyl, benzotriazolyl, benzothiazolyl, benzofuranyl, pyrazolopyridyl, quinazolinyl, quinoxalinyl, or cinnolinyl. In some embodiments, A is phenyl. In some embodiments, A is pyridyl. In some embodiments, A is 2-pyridyl. In some embodiments, A is 3-pyridyl. In some embodiments, A is 4-pyridyl. In some embodiments, A is naphthyl. In some embodiments, A is 1-naphthyl. In some embodiments, A is 2-naphthyl. In some embodiments, A is quinolinyl. In some embodiments, A is 2-quinolinyl. In some embodiments, A is 3-quinolinyl. In some embodiments, A is 4-quinolinyl. In some embodiments, A is 5-quinolinyl. In some embodiments, A is 6-quinolinyl. In some embodiments, A is 7-quinolinyl. In some embodiments, A is 8-quinolinyl. In some embodiments, A is isoquinolinyl. In some embodiments, A is 1-isoquinolinyl. In some embodiments, A is 3-isoquinolinyl. In some embodiments, A is 4-isoquinolinyl. In some embodiments, A is 5-isoquinolinyl. In some embodiments, A is 6-isoquinolinyl. In some embodiments, A is 7-isoquinolinyl. In some embodiments, A is 8-isoquinolinyl. In some embodiments, A is indolyl. In some embodiments, A is 2-indolyl. In some embodiments, A is 3-indolyl. In some embodiments, A is 4-indolyl. In some embodiments, A is 5-indolyl. In some embodiments, A is 6-indolyl. In some embodiments, A is 7-indolyl. In some embodiments, A is aryl. In some embodiments, A is bicyclic aryl. In some embodiments, A is heteroaryl. In some embodiments, A is fused bicyclic heteroaryl.

In some embodiments, A is phenyl, pyridyl, or indolyl, wherein said phenyl, pyridyl, or indolyl is optionally substituted with —OR¹⁰, halogen, or cyano.

In some embodiments, A is phenyl, wherein said phenyl is optionally substituted with —OR¹⁰, halogen, or cyano.

In some embodiments, A is pyridyl, wherein said pyridyl is optionally substituted with —OR¹⁰, halogen, or cyano.

In some embodiments, A is indolyl or indolinyl, wherein said indolyl or indolinyl is optionally substituted with —OR¹⁰, halogen, or cyano.

In some embodiments, A is phenyl, pyridyl, indolyl, or indolinyl, wherein said phenyl, pyridyl, indolyl or indolinyl is optionally substituted with —OR¹⁰ or halogen.

In some embodiments, A is phenyl, pyridyl, indolyl, or indolinyl, wherein said phenyl, pyridyl, indolyl or indolinyl is optionally substituted with halogen.

In some embodiments, A is phenyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is phenyl substituted with —R¹⁰. In some embodiments, A is phenyl substituted with —OR¹⁰. In some embodiments, A is phenyl substituted with —SR¹⁰. In some embodiments, A is phenyl substituted with —N(R¹⁰)₂. In some embodiments, A is phenyl substituted with —OSO₂R¹⁰. In some embodiments, A is phenyl substituted with —SO₂R¹⁰. In some embodiments, A is phenyl substituted with —C(O)N(R¹⁰)₂. In some embodiments, A is phenyl substituted with halogen. In some embodiments, A is phenyl substituted with cyano.

In some embodiments, A is pyridyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is pyridyl substituted with —R¹⁰. In some embodiments, A is pyridyl substituted with —OR¹⁰. In some embodiments, A is pyridyl substituted with —SR¹⁰. In some embodiments, A is pyridyl substituted with —N(R¹⁰)₂. In some embodiments, A is pyridyl substituted with —OSO₂R¹⁰. In some embodiments, A is pyridyl substituted with —SO₂R¹⁰. In some embodiments, A is pyridyl substituted with —C(O)N(R¹⁰)₂. In some embodiments, A is pyridyl substituted with halogen. In some embodiments, A is pyridyl substituted with cyano.

In some embodiments, A is 3-pyridyl substituted with —OR¹⁰. In some embodiments, A is 3-pyridyl substituted with —OCH₃. In some embodiments, A is 6-methoxypyrid-3-yl. In some embodiments, A is 3-pyridyl substituted with halogen. In some embodiments, A is 3-pyridyl substituted with Cl. In some embodiments, A is 4-chloropyrid-3-yl. In some embodiments, A is 2,3-dichloropyrid-4-yl. In some embodiments, A is 3-chloropyrid-2-yl.

In some embodiments, A is naphthyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is naphthyl substituted with —R¹⁰. In some embodiments, A is naphthyl substituted with —OR¹⁰. In some embodiments, A is naphthyl substituted with —SR¹⁰. In some embodiments, A is naphthyl substituted with —N(R¹⁰)₂. In some embodiments, A is naphthyl substituted with —OSO₂R¹⁰. In some embodiments, A is naphthyl substituted with —SO₂R¹⁰. In some embodiments, A is naphthyl substituted with —C(O)N(R¹⁰)₂. In some embodiments, A is naphthyl substituted with halogen. In some embodiments, A is naphthyl substituted with cyano.

In some embodiments, A is quinolinyl substituted with —R¹⁰, —OR¹⁰, —SR¹⁰, —N(R¹⁰)₂, —OSO₂R¹⁰, —SO₂R¹⁰, —C(O)N(R¹⁰)₂, halogen, or cyano. In some embodiments, A is quinolinyl substituted with —R¹⁰. In some embodiments, A is quinolinyl substituted with —OR¹⁰. In some embodiments, A is quinolinyl substituted with —SR¹⁰. In some embodiments, A is quinolinyl substituted with —N(R¹⁰)₂. In some embodiments, A is quinolinyl substituted with —OSO₂R¹⁰. In some embodiments, A is quinolinyl substituted with —SO₂R¹⁰. In some embodiments, A is quinolinyl substituted with —C(O)N(R¹⁰)₂. In some embodiments, A is quinolinyl substituted with halogen. In some embodiments, A is quinolinyl substituted with cyano.

In some embodiments, A is isoquinolinyl substituted with —R¹⁰, —OR, —SR¹⁰, —N(R¹⁰)₂, —OSO₂R¹⁰, —SO₂R¹⁰, —C(O)N(R¹⁰)₂, halogen, or cyano. In some embodiments, A is isoquinolinyl substituted with —R¹⁰. In some embodiments, A is isoquinolinyl substituted with —OR¹⁰. In some embodiments, A is isoquinolinyl substituted with —SR¹⁰. In some embodiments, A is isoquinolinyl substituted with —N(R¹⁰)₂. In some embodiments, A is isoquinolinyl substituted with —OSO₂R¹⁰. In some embodiments, A is isoquinolinyl substituted with —SO₂R¹⁰. In some embodiments, A is isoquinolinyl substituted with —C(O)N(R¹⁰)₂. In some embodiments, A is isoquinolinyl substituted with halogen. In some embodiments, A is isoquinolinyl substituted with cyano.

In some embodiments, A is indolyl substituted with —R¹⁰, —OR¹⁰, —SR¹⁰, —N(R¹⁰)₂, —OSO₂R¹⁰, —SO₂R¹, —C(O)N(R¹⁰)₂, halogen, or cyano. In some embodiments, A is indolyl substituted with —R¹⁰. In some embodiments, A is indolyl substituted with —OR¹⁰. In some embodiments, A is indolyl substituted with —SR¹⁰. In some embodiments, A is indolyl substituted with —N(R¹⁰)₂. In some embodiments, A is indolyl substituted with —OSO₂R¹⁰. In some embodiments, A is indolyl substituted with —SO₂R¹⁰. In some embodiments, A is indolyl substituted with —C(O)N(R¹⁰)₂. In some embodiments, A is indolyl substituted with halogen. In some embodiments, A is indolyl substituted with cyano.

In some embodiments, A is 1-methyl-1H-indol-4-yl. In some embodiments, A is 1-methyl-1H-indol-3-yl.

In some embodiments, A is imidazol-2-yl. In some embodiments, A is 1-methyl-imidazol-2-yl.

In some embodiments, A is selected from the group consisting of 1,2,3,4-tetrahydroquinoline; 1,2,3,4-tetrahydro-1,5,napthyridine, dihydropyridopyrazine, benzoimidazole, benzoazepine, and dihydropyridooxazine, wherein A is bound to X through an available nitrogen on X; and where A is optionally substituted with one or two substituents each independently selected from halo, CF₃, phenyl (optionally substituted with one, two or three halo, C₁₋₃alkyl or C₁₋₃haloalkyl), C₁₋₃alkyl, C₁₋₃haloalkyl, and heteroaryl (optionally substituted with one, two or three halo, C₁₋₃alkyl or C₁₋₃haloalkyl).

In some embodiments, A is selected from the group consisting of

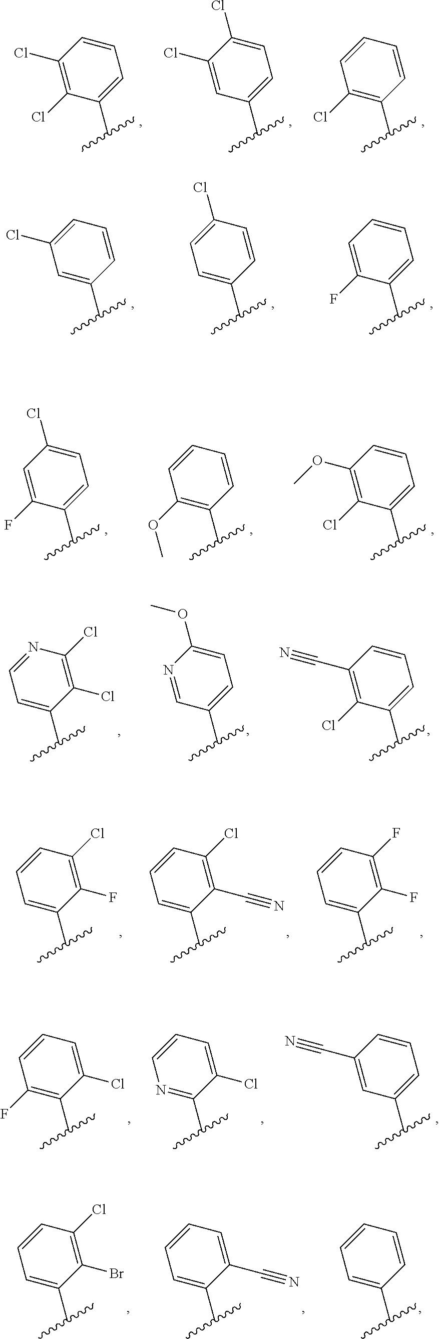

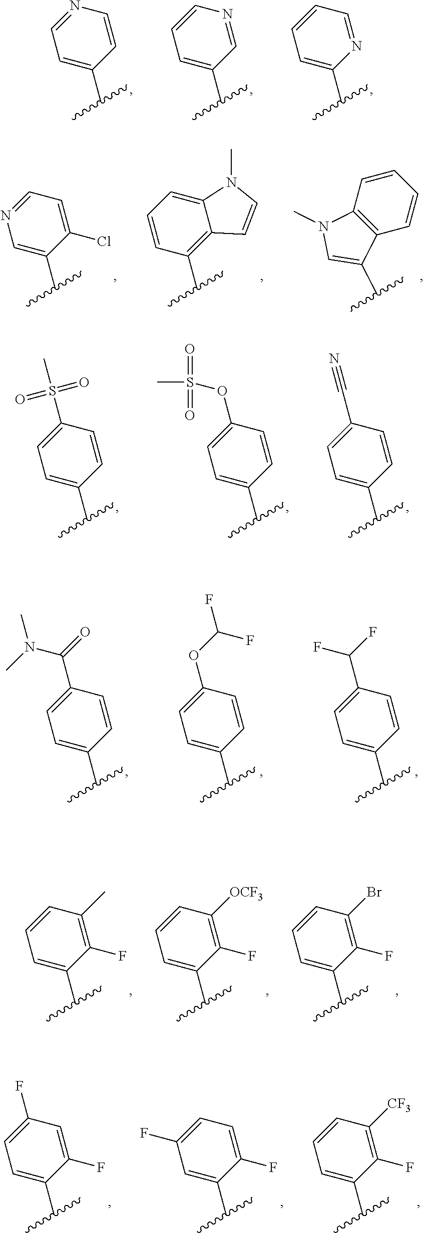

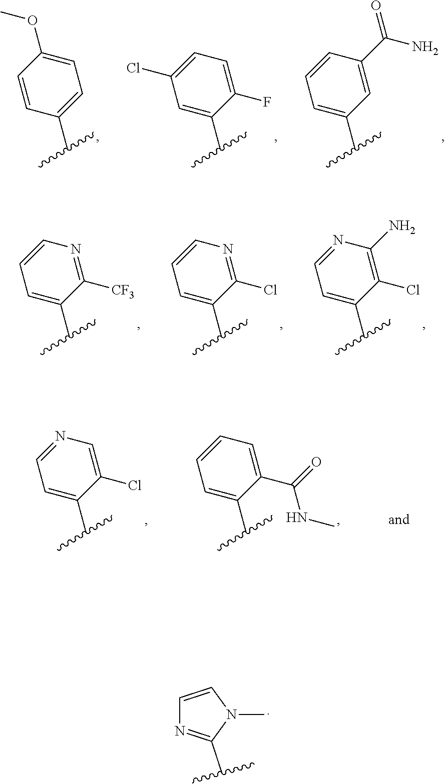

In some embodiments, A is

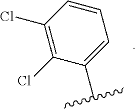 [SMILES: *c1cccc(Cl)c1Cl]

In some embodiments, A is

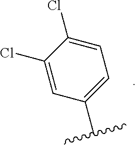 [SMILES: *c1ccc(Cl)c(Cl)c1]

In some embodiments, A is

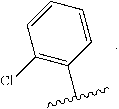 [SMILES: *c1ccccc1Cl]

In some embodiments, A is

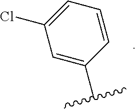 [SMILES: *c1cccc(Cl)c1]

In some embodiments, A is

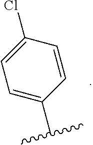 [SMILES: *c1ccc(Cl)cc1]

In some embodiments, A is

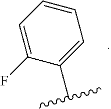 [SMILES: *c1ccccc1F]

In some embodiments, A is

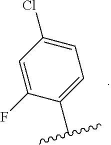 [SMILES: *c1ccc(Cl)cc1F]

In some embodiments, A is

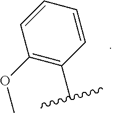 [SMILES: *c1ccccc1OC]

In some embodiments, A is

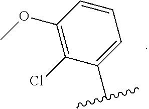 [SMILES: *c1cccc(OC)c1Cl]

In some embodiments, A is

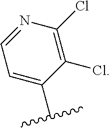 [SMILES: *c1ccnc(Cl)c1Cl]

In some embodiments, A is

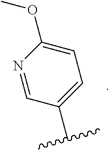 [SMILES: *c1ccc(OC)nc1]

In some embodiments, A is

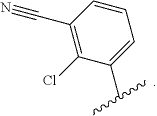 [SMILES: *c1cccc(C#N)c1Cl]

In some embodiments, A is

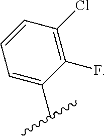 [SMILES: *c1cccc(Cl)c1F]

In some embodiments, A is

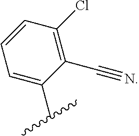 [SMILES: *c1cccc(Cl)c1C#N]

In some embodiments, A is

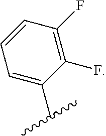 [SMILES: *c1cccc(F)c1F]

In some embodiments, A is

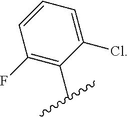 [SMILES: *c1c(F)cccc1Cl]

In some embodiments, A is

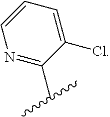 [SMILES: *c1ncccc1Cl]

In some embodiments, A is

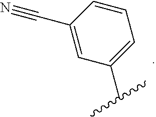 [SMILES: *c1cccc(C#N)c1]

In some embodiments, A is

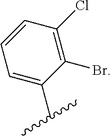 [SMILES: *c1cccc(Cl)c1Br]

In some embodiments, A is

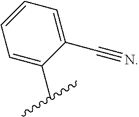 [SMILES: *c1ccccc1C#N]

In some embodiments, A is

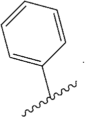 [SMILES: *c1ccccc1]

In some embodiments, A is

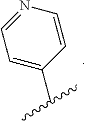 [SMILES: *c1ccncc1]

In some embodiments, A is

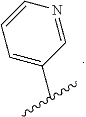 [SMILES: *c1cccnc1]

In some embodiments, A is

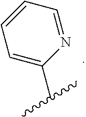 [SMILES: *c1ccccn1]

In some embodiments, A is

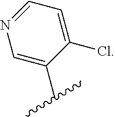 [SMILES: *c1cnccc1Cl]

In some embodiments, A is

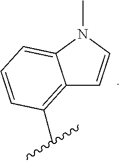 [SMILES: *c1cccc2c1ccn2C]

In some embodiments, A is

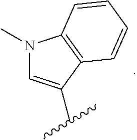 [SMILES: *c1cn(C)c2ccccc12]

In some embodiments, A is

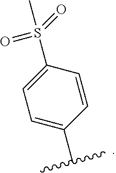 [SMILES: *c1ccc(S(C)(=O)=O)cc1]

In some embodiments, A is

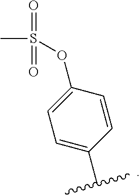 [SMILES: *c1ccc(OS(C)(=O)=O)cc1]

In some embodiments, A is

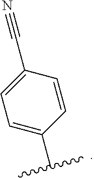 [SMILES: *c1ccc(C#N)cc1]

In some embodiments, A is

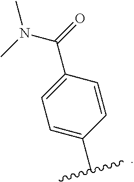 [SMILES: *c1ccc(C(=O)N(C)C)cc1]

In some embodiments, A is

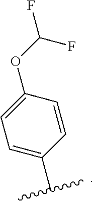 [SMILES: *c1ccc(OC(F)F)cc1]

In some embodiments, A is

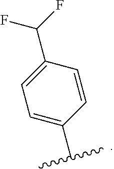 [SMILES: *c1ccc(C(F)F)cc1]

In some embodiments, A is

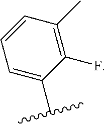 [SMILES: *c1cccc(C)c1F]

In some embodiments, A is

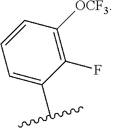 [SMILES: *c1cccc(OC(F)(F)F)c1F]

In some embodiments, A is

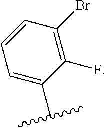 [SMILES: *c1cccc(Br)c1F]

In some embodiments, A is

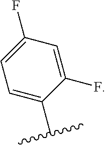 [SMILES: *c1ccc(F)cc1F]

In some embodiments, A is

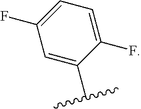 [SMILES: *c1cc(F)ccc1F]

In some embodiments, A is

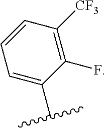 [SMILES: *c1cccc(C(F)(F)F)c1F]

In some embodiments, A is

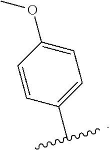 [SMILES: *c1ccc(OC)cc1]

In some embodiments, A is

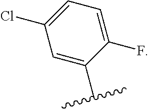 [SMILES: *c1cc(Cl)ccc1F]

In some embodiments, A is

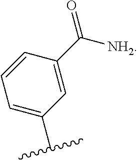 [SMILES: *c1cccc(C(N)=O)c1]

In some embodiments, A is

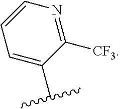 [SMILES: *c1cccnc1C(F)(F)F]

In some embodiments, A is

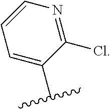 [SMILES: *c1cccnc1Cl]

In some embodiments, A is

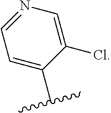 [SMILES: *c1ccncc1Cl]

In some embodiments, A is

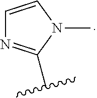 [SMILES: *c1nccn1C]

In some embodiments, A is

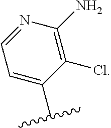 [SMILES: *c1ccnc(N)c1Cl]

In some embodiments, A is

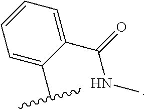 [SMILES: *c1ccccc1C(=O)NC]

In some embodiments, R³ is selected from the group consisting of hydrogen, CH₃ and —NH₂. In some embodiments, Y is selected from the group consisting of —C(O)—N(R^{a})—, —C(O)—O—, —CH₂—C(O)—O—, —C(O)—O—CH₂—CH₂—, —C(O)—, —CH₂—, —CH₂C(O)—, —CH₂—S(O)_{w}—, —CH₂—N(R^{a})—CH₂—, and —CH₂—N(R^{a})—. For example, Y is selected from the group consisting of —CH₂—S—, —CH₂—N(Me)-CH₂— and —CH₂—NH—CH₂—.

In some embodiments, B is selected from the group consisting of

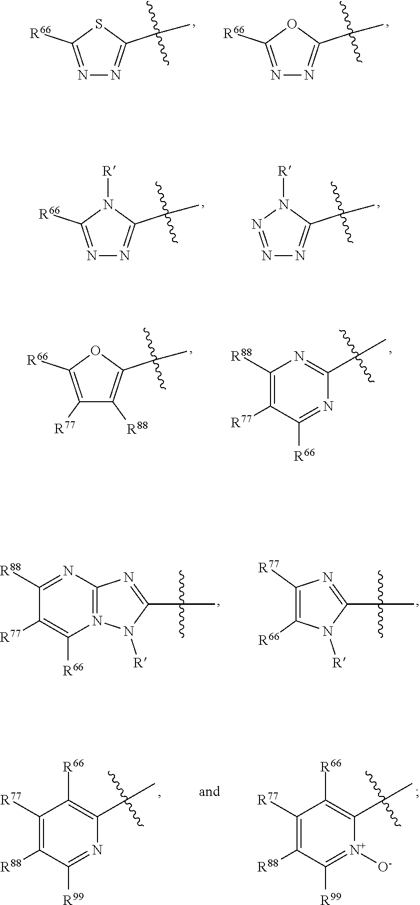

wherein

R⁶⁶, R⁷⁷, R⁸⁸ and R⁹⁹ are each independently selected from the group consisting of is selected from the group of hydrogen, halogen, —NR^{a}R^{b}, C₁₋₆alkyl, C₃₋₆cycloalkyl, C₁₋₆alkoxy, —S—C₂₋₆alkenyl, C₁₋₆alkylcarbonyl-N(R^{a})—, heteroaryl and phenyl (optionally substituted by one, two or three hydroxyl, halogen, C₁₋₃alkyl or C₁₋₃alkoxy groups); and

R′ is selected from the group consisting of hydrogen, —NR^{a}R^{b}, C₁₋₆alkyl, C₃₋₆ cycloalkyl, C₁₋₃alkyl-O—C₁₋₃alkyl, and phenyl (optionally substituted by hydroxyl or C₁₋₃ alkoxy).

For example, in some embodiments, B is selected from the group consisting of

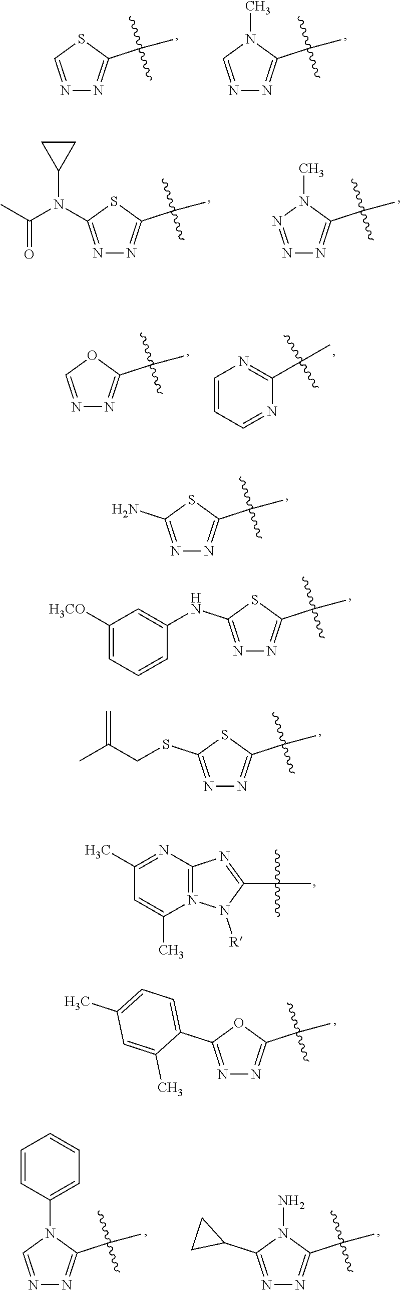

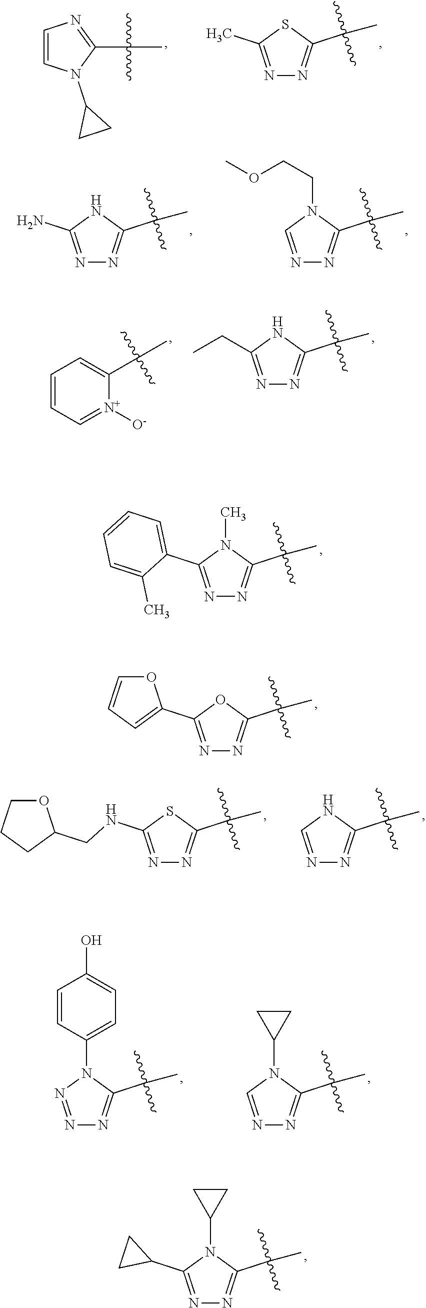

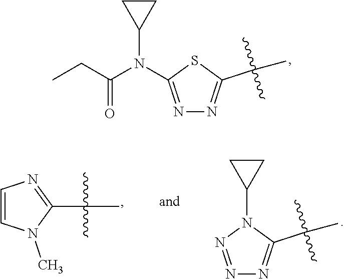

For example, in some embodiments, B is selected from the group consisting of

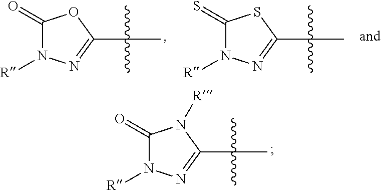

wherein

R″ and R″′ are each independently selected from the group consisting of hydrogen, C₁₋₆alkyl, C₃₋₆cycloalkyl, and phenyl; wherein phenyl may optionally be substituted by one or two substituents each independently selected from the group consisting of halogen, hydroxyl, C₁₋₃alkyl and C₁₋₃alkoxy.

For example, in some embodiments, B is selected from the group consisting of

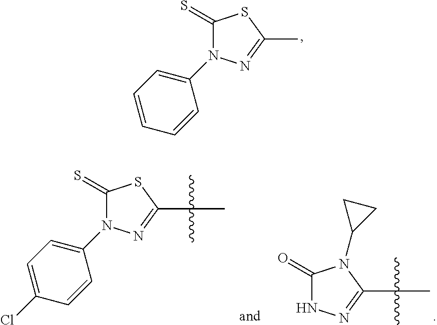

In some embodiments, Y is selected from the group consisting of a bond, —C(O)—, —CH₂—, and —CH₂C(O)—. In some embodiments, B is represented by:

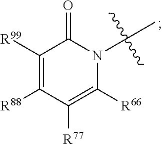

wherein

R⁶⁶, R⁷⁷, R⁸⁸ and R⁹⁹ are each independently selected from the group consisting of is selected from the group of hydrogen, halogen, C₁₋₃alkyl, C₃₋₆cycloalkyl, and C₁₋₃alkoxy; wherein C₁₋₃alkyl, C₃₋₆cycloalkyl, and C₁₋₃alkoxy may optionally be substituted by one, two or three halogens.

For example, in some embodiments, B is represented by:

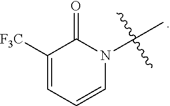 [SMILES: *n1cccc(C(F)(F)F)c1=O]

In some embodiments, B is selected from the group consisting of pyrrolidinyl, piperidinyl, azepanyl and morpholinyl; wherein pyrrolidinyl, piperidinyl, azepanyl and morpholinyl are bound to L through a ring nitrogen and may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃alkoxy; wherein C₁₋₃ alkyl and C₁₋₃alkoxy may optionally be substituted by halogen, hydroxyl and —NR^{a}R^{b}. In some embodiments, B is substituted by one, two, three or more substituents each independently selected from the group consisting of —NH₂, —CH₃, and —CH₂NH₂, —C(H)NH₂CH₃ and —CH₂NHCH₃.

In some embodiments, Y is selected from the group consisting of —C(O)—NH—, —C(O)—NMe-, —CH₂—NH—, and, —CH₂—NMe-. In some embodiments, B is phenyl; wherein phenyl may optionally be substituted by one, two or three substituents each independently selected from the group consisting of halogen, hydroxyl, methylenedioxy, C₁₋₃alkyl, and C₁₋₃ alkoxy; and wherein C₁₋₃alkyl, and C₁₋₃alkoxy may optionally be substituted by one or more fluorine atoms.

In some embodiments, Y is selected from the group consisting of —C(O)—O—, —CH₂—C(O)—O— and —C(O)—O—CH₂—CH₂—. In some embodiments, B is hydrogen.

For example, provided herein is a method of treating a disorder in a patient in need thereof, comprising administering to the patient an effective amount of a compound of Formula XIII, or a pharmaceutically acceptable salt or stereoisomer thereof, wherein Formula XIII is represented by:

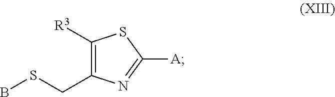

wherein

A is selected from the group consisting of phenyl, a 5-7 membered monocyclic heteroaryl having one or more heteroatoms each independently selected from O, S, and N, a 8-10 membered bicyclic heteroaryl having one or more heteroatoms each independently selected from the group consisting of O, S, and N, and a 4-7 membered heterocyclyl; wherein A may optionally be substituted by one, two, three or more substituents each independently selected from the group consisting of —R¹⁰, —OR¹⁰, methylenedioxy, —N(R¹⁰)₂, —C(O)N(R¹⁰)₂, oxo, halogen and cyano;

R¹⁰ is independently selected from the group consisting of hydrogen, C₁₋₆alkyl, C₁₋₆haloalkyl, C₃₋₆cycloalkyl, heterocycloalkyl, aryl, and heteroaryl, where R¹⁰ may optionally be substituted by one, two, three or more substituents each independently selected from the group consisting of halogen, —C(O)R²⁰, C₁₋₆alkyl and C₁₋₆haloalkyl;

R²⁰ is selected from the group consisting of hydroxyl, halogen, and C₁₋₆alkyl;

B is selected from the group consisting of:

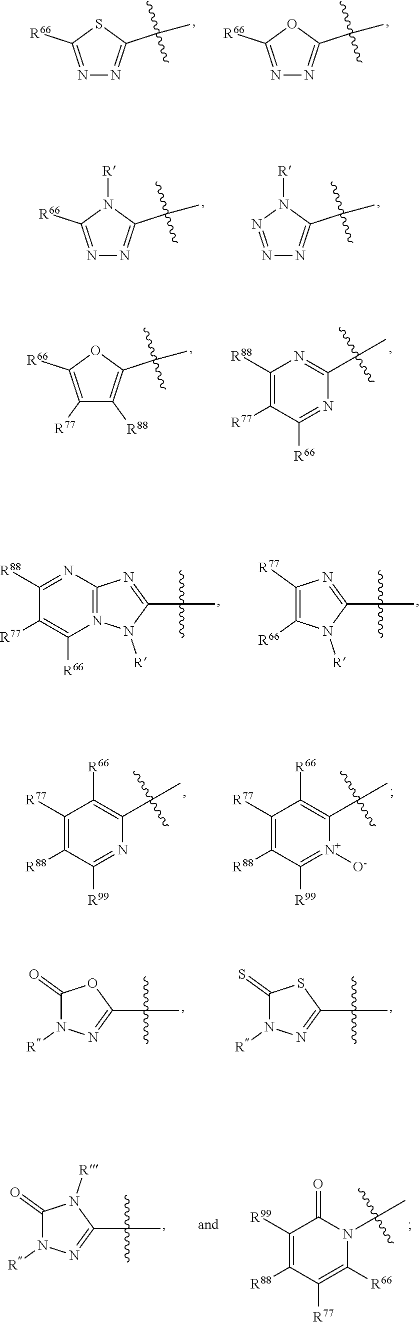

R³ is selected from the group consisting of hydrogen, halogen, hydroxyl, C₁₋₆alkyl and —NR^{a}R^{b};

R⁶⁶, R⁷⁷, R⁸⁸ and R⁹⁹ are each independently selected from the group consisting of hydrogen, halogen, —NR^{a}R^{b}, C₁₋₆alkyl, C₃₋₆cycloalkyl, C₁₋₆alkoxy, —S—C₂₋₆alkenyl, C₁₋₆ alkylcarbonyl-N(R^{a})—, heteroaryl and phenyl; wherein heteroaryl and phenyl may optionally be substituted by one, two or three substituents each independently selected from the group consisting of hydroxyl, halogen, C₁₋₃alkyl and C₁₋₃alkoxy; and wherein C₁₋₆alkyl, C₃₋₆ cycloalkyl, and C₁₋₆alkoxy may optionally be substituted by one, two or three halogens; and

R^{a} and R^{b} are independently selected, for each occurrence, from the group consisting of hydrogen, C₁₋₆alkyl, C₃₋₆cycloalkyl, and phenyl; wherein C₁₋₆alkyl may optionally be substituted by one or more substituents selected from halogen, cyano, oxo, heterocyclyl and hydroxyl; and wherein phenyl may optionally be substituted by C₁₋₃alkyl, C₁₋₃alkoxy, halogen, hydroxyl;

or R^{a} and R^{b}, together with the nitrogen to which they are attached, may form a 4-6 membered monocyclic heterocyclic ring, which may have an additional heteroatom selected from the group consisting of O, S, and N; wherein the 4-6 membered heterocyclic ring may optionally be substituted by one or more substituents selected from the group consisting of halogen, cyano, oxo, hydroxyl and C₁₋₃alkyl (optionally substituted with —NH₂).

Also provided herein is a method of treating a disorder in a patient in need thereof, comprising administering to the patient an effective amount of a compound of Formula XIV, or a pharmaceutically acceptable salt or stereoisomer thereof, wherein Formula XIV is represented by:

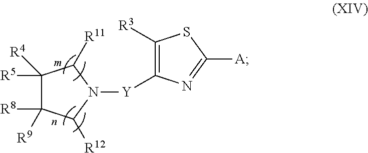

wherein

A is selected from the group consisting of phenyl, a 5-7 membered monocyclic heteroaryl having one or more heteroatoms each independently selected from O, S, and N, a 8-10 membered bicyclic heteroaryl having one or more heteroatoms each independently selected from the group consisting of O, S, and N, and a 4-7 membered heterocyclyl; wherein A may optionally be substituted by one, two, three or more substituents each independently selected from the group consisting of —R¹⁰, —OR¹⁰, methylenedioxy, —N(R¹⁰)₂, —C(O)N(R¹⁰)₂, oxo, halogen and cyano;

R¹⁰ is independently selected from the group consisting of hydrogen, C₁₋₆alkyl, C₁₋₆haloalkyl, C₃₋₆cycloalkyl, heterocycloalkyl, aryl, and heteroaryl, where R¹⁰ may optionally be substituted by one, two, three or more substituents each independently selected from the group consisting of halogen, —C(O)R²⁰, C₁₋₆alkyl and C₁₋₆haloalkyl;

R²⁰ is selected from the group consisting of hydroxyl, halogen, and C₁₋₆alkyl;

R³ is selected from the group consisting of hydrogen, halogen, hydroxyl, C₁₋₆alkyl and —NR^{a}R^{b};

Y is selected from the group consisting of a bond, —C(O)—, —CH₂—, and —CH₂C(O)—;

R⁴ and R⁵ are each independently selected from the group consisting of hydrogen, hydroxyl, C₁₋₆alkyl, C₁₋₆alkoxy, C₁₋₆alkyl-O—R⁶, —C(O)N(R⁶)₂, —N(R⁶)₂, halogen, C₁₋₆alkyl-N(R⁶)₂, and cyano; wherein C₁₋₆alkyl, C₁₋₆alkoxy, C₁₋₆alkyl-O—R⁶, and C₁₋₆alkyl-N(R⁶)₂ may optionally be substituted by one or more substituents each independently selected from the group consisting of hydroxyl, —NH₂, oxo, and halogen,

or R⁴ and R⁵, taken together with the atoms to which they are attached, form a 3-7 membered carbocyclic or heterocyclic saturated or partially unsaturated ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;

or R⁴ and R⁸, taken together with the atoms to which they are attached, form a 4-7 membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;

or R⁴ is absent, and R⁴ and R⁸, taken together with the atoms to which they are attached, form a 3-membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;

R⁶ is independently for each occurrence selected from the group consisting of hydrogen, C₁₋₆alkyl and phenyl;

R¹¹ and R¹² are each independently selected from the group consisting of hydrogen, hydroxyl, C₁₋₆alkyl, C₁₋₆alkoxy, C₁₋₆alkyl-O—R⁶, aryl, arylalkylene, heteroaryl, heteroarylalkylene, —C(O)N(R⁶)₂, —N(R⁶)₂, halogen, C₁₋₆alkyl-N(R⁶)₂, —CO₂H, and cyano; wherein C₁₋₆alkyl, C₁₋₆alkoxy, C₁₋₆alkyl-O—R⁶, aryl, arylalkylene, heteroaryl, heteroarylalkylene, and C₁₋₆alkyl-N(R⁶)₂ may optionally be substituted by one or more substituents each independently selected from the group consisting of hydroxyl, —NH₂, and halogen;

or R¹¹ and R¹², taken together with the atoms to which they are attached, form a 5-7 membered heterocyclic ring;

or R⁴ and R¹², taken together with the atoms to which they are attached, form a 5-7 membered carbocyclic or heterocyclic ring;

or R⁸ and R¹¹, taken together with the atoms to which they are attached, form a 5-7 membered carbocyclic or heterocyclic ring;

R⁸ and R⁹ are each independently selected from the group consisting of hydrogen, C₁₋₆)alkyl, C₁₋₆alkyl-N(R⁶)₂, —OR⁶, C₁₋₆alkyl-O—R⁶, —C(O)NH₂, —N(R⁶)₂, halogen, and cyano; and

each of m and n is, independently, 0, 1, 2, or 3, with m+n being at least 2 and no more than 4.

In some embodiments, A is phenyl; wherein phenyl may optionally be substituted by one, two, or three substituents each independently selected from the group consisting of —OR¹⁰, methylenedioxy, halogen, and cyano. In some embodiments, R³ is selected from the group consisting of hydrogen, CH₃ and —NH₂. In some embodiments, m is an integer selected from 1 or 2; and n is 1.

In some embodiments, R⁴ and R⁵ are each independently selected from the group consisting of hydrogen, hydroxyl, C₁₋₃alkyl, C₁₋₃alkoxy, C₁₋₃alkyl-O—R⁶, —C(O)NH₂, —N(R⁶)₂, halogen, C₁₋₃alkyl-N(R⁶)₂, and cyano; wherein C₁₋₃alkyl, C₁₋₃alkoxy, C₁₋₃alkyl-O—R⁶ and C₁₋₃alkyl-N(R⁶)₂ may optionally be substituted by one or more substituents each independently selected from the group consisting of hydroxyl, —N(R⁶)₂, and halogen;

or R⁴ and R⁵, taken together with the atoms to which they are attached, form a 3-7 membered carbocyclic or heterocyclic ring;

or R⁴ and R⁸, taken together with the atoms to which they are attached, form a 4-7 membered carbocyclic or heterocyclic ring;

or R⁴ is a bond, and R⁴ and R⁸, taken together with the atoms to which they are attached, form a 3-membered carbocyclic or heterocyclic ring; and

each R⁶ is independently hydrogen or C₁₋₃alkyl.

In some embodiments, R⁴ and R⁵ are each independently selected from the group consisting of C₁₋₃alkyl and —NR^{a}R^{b}; wherein C₁₋₃alkyl may optionally be substituted by one, two or three substituents each independently selected from the group consisting of halogen, hydroxyl and —NR^{a}R^{b}. For example, R⁴ and R⁵ are each independently selected from the group consisting of —NH₂, —CH₃, and —CH₂NH₂, —C(H)NH₂CH₃ and —CH₂NHCH₃.

In some embodiments, the compound to be administered may be selected from the group consisting of:

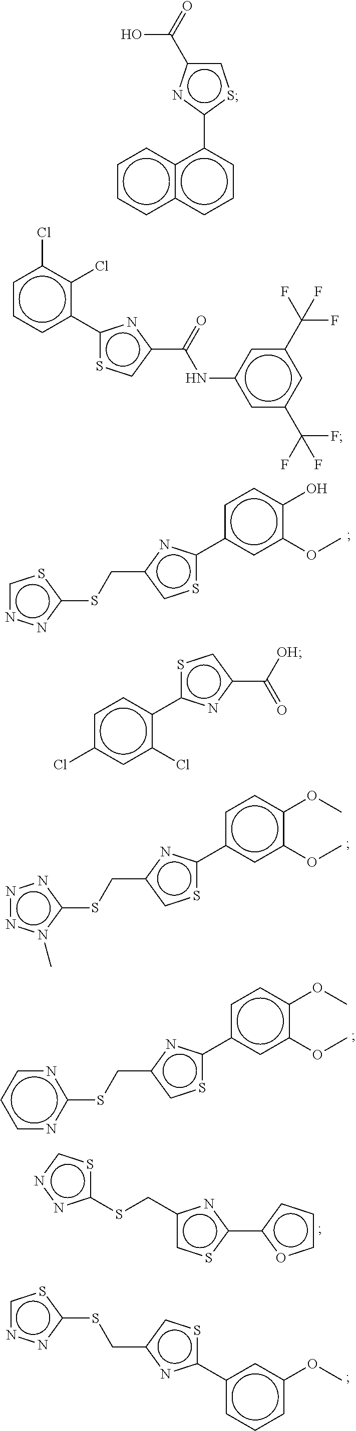

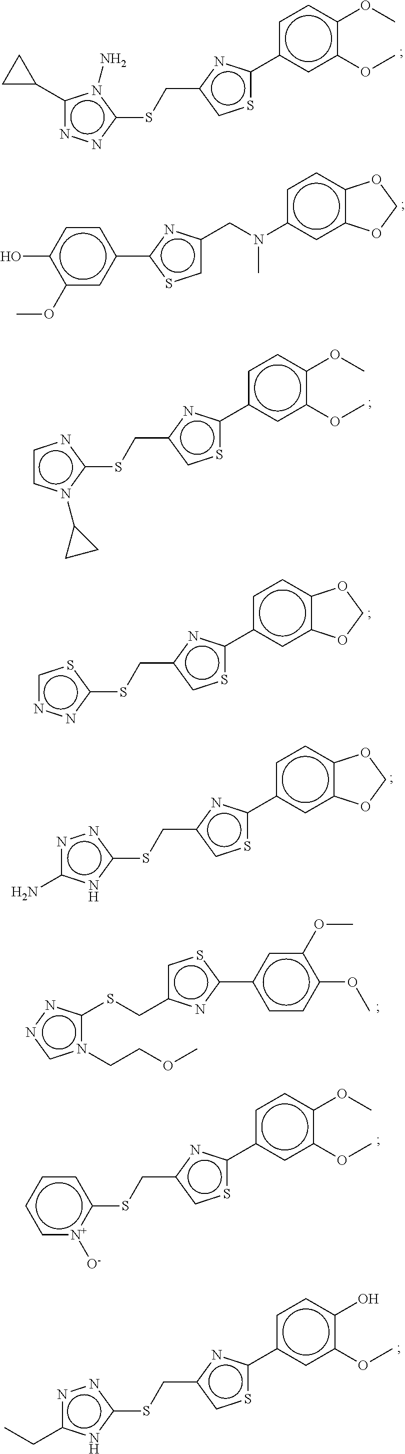

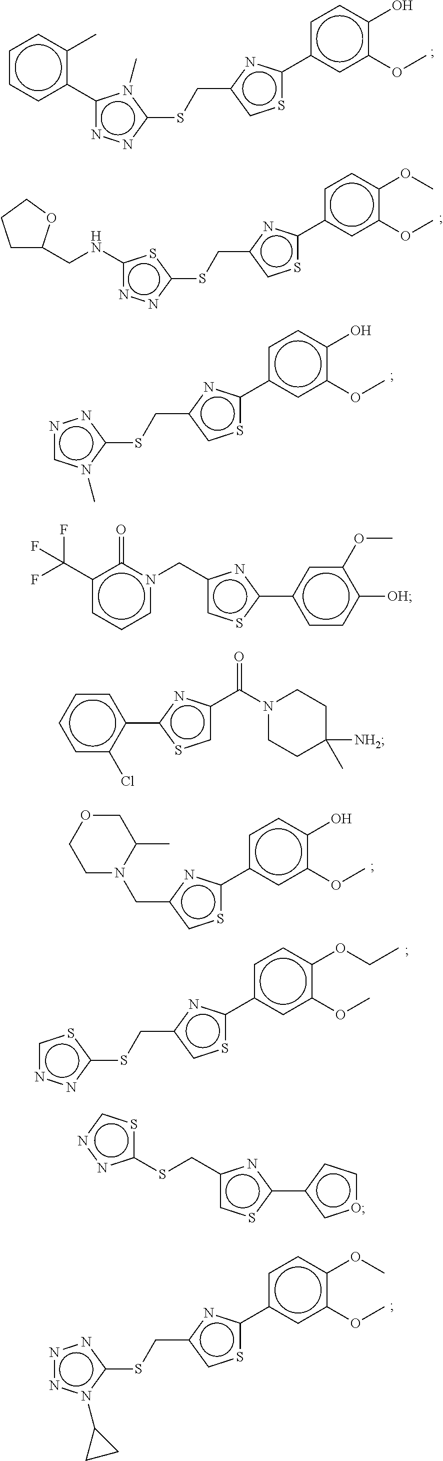

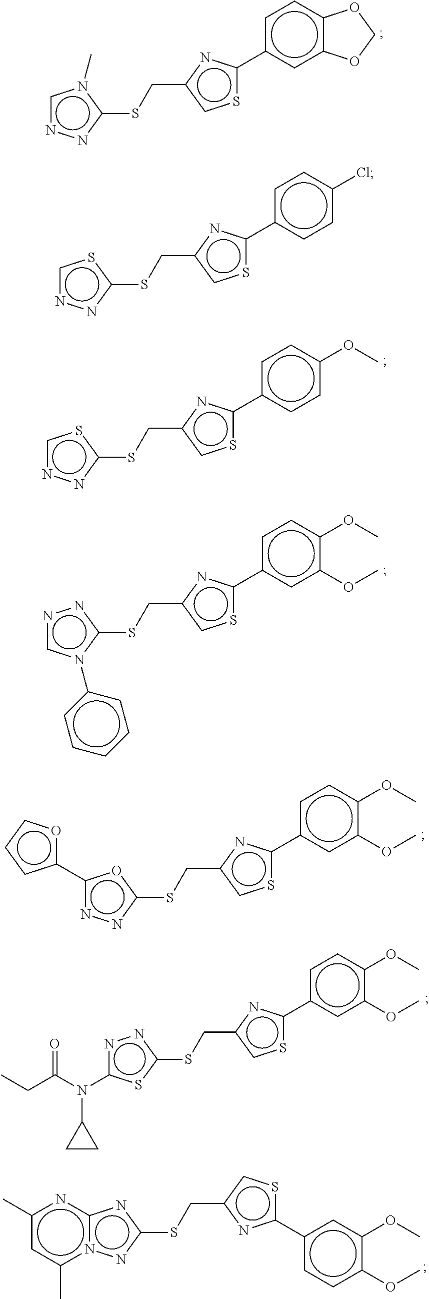

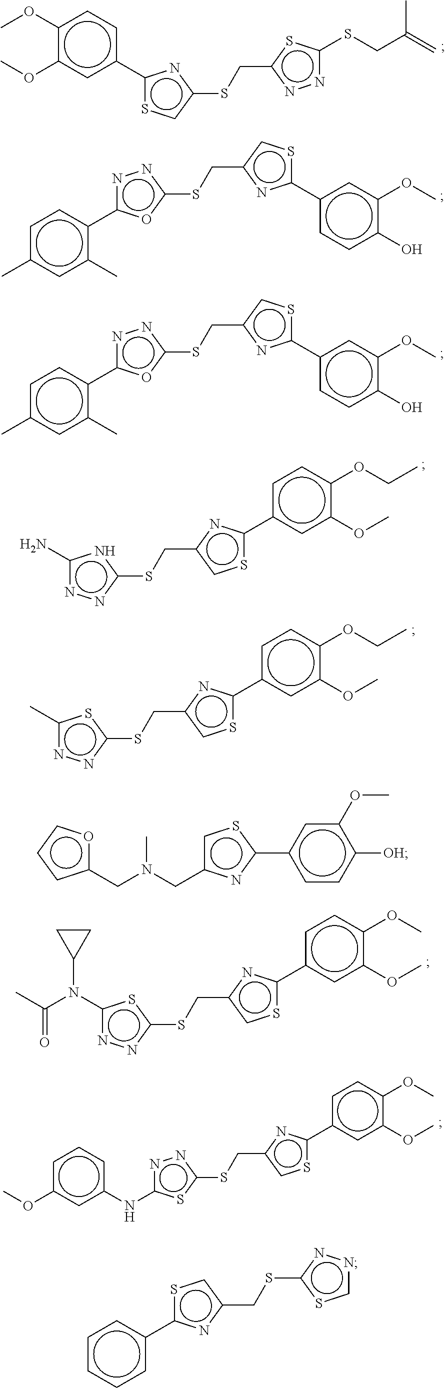

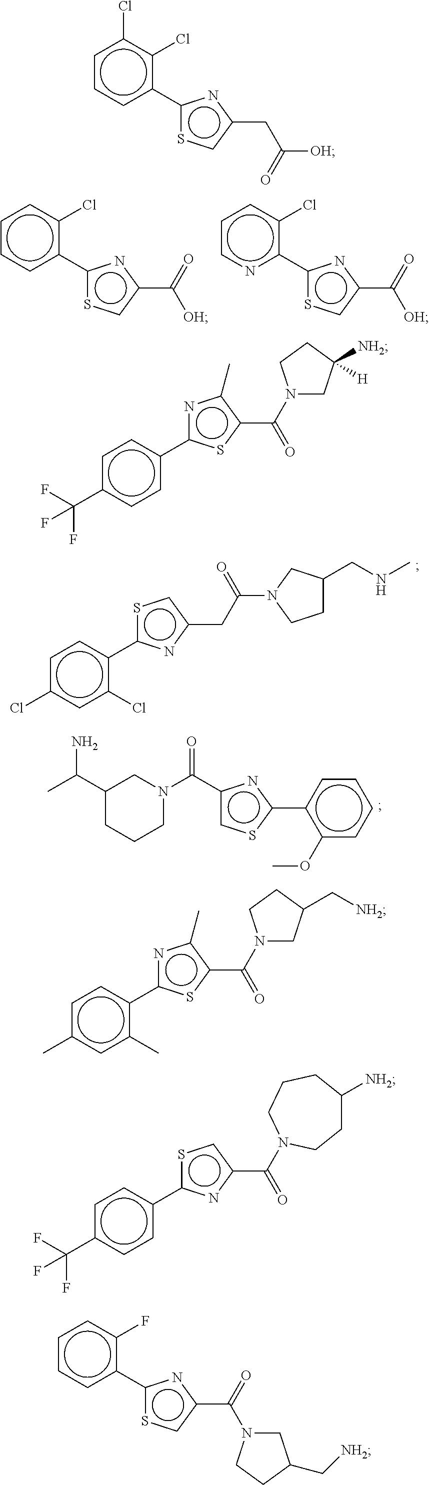

and a pharmaceutically acceptable salt or stereoisomer thereof.

In some embodiments, the methods disclosed herein may further comprise administration of a therapeutically effective amount of an antibody, an antibody-drug conjugate, an immunomodulator, or a histone deacetylase inhibitor. In some embodiments, the disorder to be treated is Noonan syndrome. In some embodiments, the disorder to be treated is neutropenia. In some embodiments, the disorder to be treated is diabetes. In some embodiments, the disorder to be treated is neuroblastoma. In some embodiments, the disorder to be treated is melanoma. In some embodiments, the disorder to be treated is acute myeloid leukemia. In some embodiments, the disorder to be treated is juvenile leukemia. In some embodiments, the disorder to be treated is juvenile myelomonocytic leukemia. In some embodiments, the disorder to be treated is breast cancer. In some embodiments, the disorder to be treated is lung cancer. In some embodiments, the disorder to be treated is colorectal cancer.

Disclosed compounds or compositions can be useful in applications that benefit from inhibition of SHP2 phosphatase enzymes. For example, inhibition of SHP2 phosphatase may offer a therapeutic approach for the treatment of cancer. (See, e.g., Y.-N. P. Chen et al., in Nature, 2016, doi: 10.1038/nature18621; and references cited therein; each of which hereby incorporated by reference in its entirety.) Inhibition of SHP2 phosphatase also has been found to ameliorate the pathogensis of systemic lupus erythematosus. (See, e.g., J. Wang et al., in J. Clin. Invest. 2016, 126, 2077-2092; and references cited therein; each of which hereby incorporated by reference in its entirety.)

In some embodiments, compounds or compositions of the disclosure can be useful in suppressing tumor cell growth. In some embodiments, compounds or compositions of the disclosure can be useful in ameliorating the pathogenesis of systemic lupus erythematosus. In some embodiments, compounds or compositions of the disclosure can be useful in the treatment of various other disorders, including Noonan syndrome (NS), diabetes, neuroblastoma, melanoma, juvenile leukemia, juvenile myelomonocytic leukemia (JMML), chronic myelomonocytic leukemia, acute myeloid leukemia, HER2-positive breast cancer, triple-negative breast cancer, ductal carcinoma of the breast, invasive ductal carcinoma of the breast, non-small cell lung cancer (including adenocarcinoma of the lung), colorectal cancer (SW480, SW620, CACO2, HCT116, HT29 colon cancer cell lines), esophageal cancer, gastric cancer, squamous-cell carcinoma of the head and neck (SCCHN), and neutropenia (Kostmann's syndrome).

In some embodiments, compounds or compositions of the disclosure can be used in combination with other treatments and/or cancer therapies. For example, compounds or compositions of the disclosure can be used in combination with, but are not limited to, antibodies, antibody-drug conjugates, kinase inhibitors, immunomodulators, and histone deacetylase inhibitors. The compounds or compositions of the disclosure can also be used in combination with other treatments and/or cancer therapies as disclosed in WO 2015/107495; and references cited therein; each of which is hereby incorporated by reference in its entirety. For example, the compounds disclosed herein (or pharmaceutical compositions containing them) can be used in the treatment of one or more of the diseases mentioned herein, alone or in combination with another therapeutic agent. For example, a compound of Formula I, Formula II or Formula III can be used in combination with the following agents: BCR-ABL inhibitors: imatinib mesylate; inilotinib hydrochloride; nilotinib; dasatinib; bosutinib; ponatinib; bafetinib; danusertib; saracatinib; N-[2-[(1S,4R)-6-[[4-(Cyclobutylamino)-5-(tjifluoromethyl)-2-pyrimidinyl]amino]-1,2,3,4-tetrahydronaphthalen-1,4-imin-9-yl]-2-oxoethyl]-acetamide. ALK inhibitors: crizotinib; 5-chloro-N4-(2-(isopropylsulfonyl)phenyl)-N2-(2-methoxy-4-(4-(4-methylpiperazin-1-yl)piperidin-1-yl)phenyl)pyrimidine-2,4-diamine, ceritinib, alectinib, brigatinib, entrecinib. BRAF inhibitors: vemurafenib and dabrafenib. FGFR inhibitors: infigratinib, dovitinib, erdafitinib, BLU-554, AZD4547. FLT3 inhibitors: sunitinib malate; midostaurin; tanutinib; sorafenib, lestaurtinib, quizartinib and crenolanib. MEK Inhibitors—trametinib, combimetinib, binimetinib, selumetinib. VEGF receptor inhibitors: bevacizumab, axitinib, Aflibercept, (N-methyl-2-[[3-[(E)-2-pyridin-2-ylethenyl]-1H-indazol-6-yl]sulfanyl]benzamide, brivanib alaninate ((S)—((R)-1-(4-(4-Fluoro-2-methyl-1H-indol-5-yloxy)-5-methylpyrrolo[2,1-f][1,2,4]triazin-6-yloxy)propan-2-yl)2-aminopropanoate, motesanib (N-(2,3-dihydro-3,3-dimethyl-1H-indol-6-yl)-2-[(4-pyridinylmethyl)amino]-3-pyridinecarboxamide, pasireotide, sorafenib. Tyrosine kinase inhibitors: erlotinib hydrochloride, linifanib, sunitinib malate, pazopanib. Epidermal growth factor receptor (EGFR) inhibitors: Gefitnib, osimertinib, cetuximab, panitumumab. HER2 receptor inhibitors: trastuzumab, neratinib, lapatinib or lapatinib ditosylate. MET inhibitors: crizotinib, cabozantinib. CD20 antibodies: rituximab, tositumomab, ofatumumab. DNA Synthesis inhibitors: capecitabine, gemcitabine hydrochloride, nelarabine, hydroxycarbamide. Antineoplastic agents: oxaliplatin. HER dimerization inhibitors: pertuzumab. Human Granulocyte colony-stimulating factor (G-CSF) modulators: Filgrastim. Immunomodulators: Afutuzumab, lenalidomide, thalidomide. CD40 inhibitors: Dacetuzumab. Pro-apoptotic receptor agonists (PARAs): Dulanermin. Heat Shock Protein (HSP) inhibitors: Tanespimycin (17-allylamino-17-demethoxygeldanamycin). Hedgehog antagonists: 2-chloro-N-[4-chloro-3-(2-pyridinyl)phenyl]-4-(methylsulfonyl)-benzamide. Proteasome inhibitors: Bortezomib. PI3K inhibitors: 4-[2-(H-Indazol-4-yl)-6-[[4-(methylsulfonyl)piperazin-1-yl]methyl]thieno[3,2-d]pyrimidin-4-yl]mo choline, 2-Methyl-2-[4-[3-methyl-2-oxo-8-(quinolin-3-yl)-2,3-dihydroimidazo[4,5-c]quinolin-1-yl]phenyl]propiocyano, buparlisib, taselisib, idelalisib, duvelisib, TGR 1202. Phospholipase A2 inhibitors: Anagrelide. BCL-2 inhibitors: 4-[4-[[2-(4-chlorophenyl)-5,5-dimethyl-1-cyclohexen-1-yl]methyl]-1-piperazinyl]-N-[[4-[[(1R)-3-(4-morpholinyl)-1-[(phenylthio)methyl]propyl] amino]-3-[(trifluoromethyl)sulfonyl]phenyl]sulfonyl]benzamide. Mitogen-activated protein kinase kinase (MEK) inhibitors: XL-518. Aromatase inhibitors: Exemestane, letrozole, anastrozole, faslodex, tamoxifen. Topoisomerase I inhibitors: Irinotecan, topotecan hydrochloride. Topoisomerase II inhibitors: etoposide, teniposide. mTOR inhibitors: Temsirolimus, ridaforolimus, everolimus. Osteoclastic bone resorption inhibitors: 1-Hydroxy-2-imidazol-1-yl-phosphonoethyl) phosphonic acid monohydrate. CD33 Antibody Drug Conjugates: Gemtuzumab ozogamicin. CD22 Antibody Drug Conjugates: Inotuzumab ozogamicin. CD20 Antibody Drug Conjugates: Ibritumomab tiuxetan. Somatostain analogs: octreotide. Synthetic Interleukin-11 (IL-11): oprelvekin. Synthetic erythropoietin: Darbepoetin alfa. Receptor Activator for Nuclear Factor κ B (RANK) inhibitors: Denosumab. Thrombopoietin mimetic peptides: Romiplostim. Cell growth stimulators: Palifermin. Anti-Insulin-like Growth Factor-1 receptor (IGF-1R) antibodies: Figitumumab. Anti-CS1 antibodies: Elotuzumab. CD52 antibodies: Alemtuzumab. CTLA-4 inhibitors: Tremelimumab, ipilimumab. PD1 inhibitors: Nivolumab; pembrolizumab; an immunoadhesin; Pidilizumab; and AMP-224. PDL1 inhibitors: MSB0010718C; YW243.55.S70, MPDL3280A; MEDI-4736, MSB-0010718C, or MDX-1105. LAG-3 inhibitors: BMS-986016. GITR agonists: GITR fusion proteins and anti-GITR antibodies. Histone deacetylase inhibitors (HDI): Voninostat. Anti-CTLA4 antibodies: Tremelimumab; and Ipilimumab. Alkylating agents: Temozolomide, dactinomycin, melphalan, altretamine carmustine, bendamustine, busulfan, carboplatin, lomustine, cisplatin, chlorambucil, cyclophosphamide, dacarbazine, altretamine, ifosfamide, procarbazine, mechlorethamine, mustine and mechloroethamine hydrochloride, streptozocin, thiotepa. Biologic response modifiers: bacillus calmette-guerin, denileukin diftitox. Anti-tumor antibiotics: doxorubicin, bleomycin, daunorubicin, daunorubicin liposomal, mitoxantrone, epirubicin, idarubicin, mitomycin C. Anti-microtubule agents: Estramustine. Cathepsin K inhibitors: Odanacatib. Epothilone B analogs: Ixabepilone. TpoR agonists: Eltrombopag. Anti-mitotic agents: Docetaxel. Adrenal steroid inhibitors: aminoglutethimide. Anti-androgens: Nilutamide, Androgen Receptor inhibitors: enzalutamide, abiraterone acetate, orteronel, galeterone, and seviteronel, bicalutamide, flutamide. Androgens: Fluoxymesterone. CDK1 inhibitors: Alvocidib, palbociclib, ribociclib, trilaciclib, abemaciclib. Gonadotropin-releasing hormone (GnRH) receptor agonists: Leuprolide or leuprolide acetate. Taxane anti-neoplastic agents: Cabazitaxel (1-hydroxy, 10-dimethoxy-9-oxo-5,20-epoxytax-11-ene-2a,4,13a-triyl-4-acetate-2-benzoate-13-[(2R,3S)-3-{[(tert-butoxy)carbonyl]amino}-2-hydroxy-3-phenylpropanoate), larotaxel ((2a,3ξ,4α,5β,7α,10β,13α)-4,10-bis(acetyloxy)-13-({(2R,3S)-3-[(tert-butoxycarbonyl) amino]-2-hydroxy-3-phenylpropanoyl}oxy)-1-hydroxy-9-oxo-5,20-epoxy-7,19-cyclotax-11-en-2-yl benzoate). 5HT1a receptor agonists: Xaliproden (also known as SR57746, 1-[2-(2-naphthyl)ethyl]-4-[3-(trifluoromethyl)phenyl]-1,2,3,6-tetrahydropyridine. HPC vaccines: Cervarix® sold by GlaxoSmithKline, Gardasil® sold by Merck;

Iron Chelating agents: Deferasinox. Anti-metabolites: Claribine (2-chlorodeoxyadenosine), 5-fluorouracil, 6-thioguanine, pemetrexed, cytarabine, cytarabine liposomal, decitabine, hydroxyurea, fludarabine, floxuridine, cladribine, methotrexate, pentostatin. Bisphosphonates: Pamidronate. Demethylating agents: 5-azacitidine, decitabine. Plant Alkaloids: Paclitaxel protein-bound; vinblastine, vincristine, vinorelbine, paclitaxel. Retinoids: Alitretinoin (sold under the tradename Panretin®), tretinoin (all-trans retinoic acid, also known as ATRA, sold under the tradename Vesanoid®), Isotretinoin (13-cis-retinoic acid, sold under the tradenames Accutane®, Amnesteem®, Claravis®, Claras®, Decutan®, Isotane®, Izotech®, Oratane®, Isotret®, and Sotret®), bexarotene (sold under the tradename Targretin®). Glucocorticosteroids: Hydrocortisone (also known as cortisone, hydrocortisone sodium succinate, hydrocortisone sodium phosphate, and sold under the tradenames Ala-Cort®, Hydrocortisone Phosphate, Solu-Cortef®, Hydrocort Acetate® and Lanacort®), dexamethazone ((8S,9R,10S,1 S,13S,14S,16R,17R)-9-fluoro-11,17-dihydroxy-17-(2-hydroxy acetyl)-10,13,16-trimethyl-6,7,8,9,10,11,12,13,14,15,16,17-dodecahydro-3H-cyclopenta[a]phenanthren-3-one), prednisolone (sold under the tradenames Delta-Cortel®, Orapred®, Pediapred® and Prelone®), prednisone (sold under the tradenames Deltasone®, Liquid Red®, Meticorten® and Orasone®), methylprednisolone (also known as 6-Methylprednisolone, Methylprednisolone Acetate, Methylprednisolone Sodium Succinate, sold under the tradenames Duralone®, Medralone®, Medrol®, M-Prednisol® and Solu-Medrol®). Cytokines: interleukin-2 (also known as aldesleukin and IL-2, sold under the tradename Proleukin®), interleukin-11 (also known as oprevelkin, sold under the tradename Neumega®), alpha interferon alfa (also known as IFN-alpha, sold under the tradenames Intron® A, and Roferon-A®). Estrogen receptor downregulators: Fulvestrant (sold under the tradename Faslodex®). Anti-estrogens: tamoxifen (sold under the tradename Novaldex®). Toremifene (sold under the tradename Fareston®). Selective estrogen receptor modulators (SERMs): Raloxifene (sold under the tradename Evista®). Leutinizing hormone releasing hormone (LHRH) agonists: Goserelin (sold under the tradename Zoladex®); Progesterones: megestrol (also known as megestrol acetate, sold under the tradename Megace®); Miscellaneous cytotoxic agents: Arsenic trioxide (sold under the tradename Trisenox®), asparaginase (also known as L-asparaginase, Erwinia L-asparaginase, sold under the tradenames Elspar® and Kidrolase®). Anti-nausea drugs: NK-1 receptor antagonists: Casopitant (sold under the tradenames Rezonic® and Zunrisa® by GlaxoSmithKline); and Cytoprotective agents: Amifostine (sold under the tradename Ethyol®), leucovorin (also known as calcium leucovorin, citrovorum factor and folinic acid). Immune checkpoint inhibitors: The term “immune checkpoints” refers to a group of molecules on the cell surface of CD4 and CD8 T cells. Immune checkpoint molecules include, but are not limited to, Programmed Death 1 (PD-1), Cytotoxic T-Lymphocyte Antigen 4 (CTLA-4), B7H1, B7H4, OX-40, CD 137, CD40, and LAG3. Immunotherapeutic agents which can act as immune checkpoint inhibitors useful in the methods of the present disclosure, include, but are not limited to, inhibitors of PD-L1, PD-L2, CTLA4, TIM3, LAG3, VISTA, BTLA, TIGIT, LAIR1, CD 160, 2B4 and/or TGFR beta.

Compounds described herein can function, in certain embodiments, as allosteric inhibitors and block the activation of SHP2 by targeting the auto-inhibited conformation of SHP2.

The compounds described herein can also inhibit SHP2 function through incorporation into agents that catalyze the destruction of SHP2. For example, the compounds can be incorporated into proteolysis targeting chimeras (PROTACs). A PROTAC is a bifunctional molecule, with one portion capable of engaging an E3 ubiquitin ligase, and the other portion having the ability to bind to a target protein meant for degradation by the cellular protein quality control machinery. Recruitment of the target protein to the specific E3 ligase results in its tagging for destruction (i.e., ubiquitination) and subsequent degradation by the proteasome. Any E3 ligase can be used. The portion of the PROTAC that engages the E3 ligase is connected to the portion of the PROTAC that engages the target protein via a linker which consists of a variable chain of atoms. Recruitment of SHP2 to the E3 ligase will thus result in the destruction of the SHP2 protein. The variable chain of atoms can include, for example, rings, heteroatoms, and/or repeating polymeric units. It can be rigid or flexible. It can be attached to the two portions described above using standard techniques.

The compounds described herein can be linked to one end of a variable chain, while the other end of the variable chain can be bound to the E3 ligase. Recruitment of SHP2 to the ligase will thus result in the destruction of the SHP2 protein.

In some embodiments, compounds or compositions of the disclosure can be used in combination with an antibody. In some embodiments, compounds or compositions of the disclosure can be used in combination with an antibody-drug conjugate. In some embodiments, compounds or compositions of the disclosure can be used in combination with a kinase inhibitor. In some embodiments, compounds or compositions of the disclosure can be used in combination with an immunomodulator. In some embodiments, compounds or compositions of the disclosure can be used in combination with a histone deacetylase inhibitor.

In some embodiments, disclosed compounds can be administered to a subject in need of treatment at dosages ranging from about 0.0001 mg to about 100 mg/kg body weight of the subject to be treated per day, such as from about 1.0 to 10 mg/kg. However, additional variations are within the scope of the disclosure.

A disclosed compound can be administered alone or in combination with pharmaceutically acceptable carriers, such as diluents, fillers, aqueous solution, and even organic solvents. The compound and/or compositions of the disclosure can be administered as a tablet, powder, lozenge, syrup, injectable solution, and the like. Additional ingredients, such as flavoring, binder, excipients, and the like are within the scope of the disclosure.

In some embodiments, pharmaceutically acceptable compositions can contain a disclosed compound and/or a pharmaceutically acceptable salt thereof at a concentration ranging from about 0.01 to about 2.0 wt %, such as 0.01 to about 1 wt % or about 0.05 to about 0.5 wt %. The composition can be formulated as a solution, suspension, ointment, or a capsule, and the like. The pharmaceutical composition can be prepared as an aqueous solution and can contain additional components, such as preservatives, buffers, tonicity agents, antioxidants, stabilizers, viscosity-modifying ingredients and the like.

In some embodiments, the present disclosure provides for the use of pharmaceutical compositions and/or medicaments comprised of a compound disclosed herein, or a pharmaceutically acceptable salt thereof, in a method of treating a disease state, and/or condition caused by or related to SHP2 phosphatase. For example, provided herein are methods of treating subjects in need thereof (e.g., subjects suffering from cancer (e.g., leukemia, breast, lung and/or colorectal cancer) an effective amount of a disclosed compound, and optionally an effective amount of an additional compound (e.g., therapeutic agent) such as disclosed herein.

In some embodiments, a method of treatment comprises the steps of: i) identifying a subject in need of such treatment; (ii) providing a compound disclosed herein, or a pharmaceutically acceptable salt thereof, and (iii) administering said compound in a therapeutically effective amount to treat, suppress and/or prevent the disease state or condition in a subject in need of such treatment.

In some embodiments, a method of treatment comprises the steps of: i) identifying a subject in need of such treatment; (ii) providing a composition comprising a compound disclosed herein, or a pharmaceutically acceptable salt thereof, and (iii) administering said composition in a therapeutically effective amount to treat, suppress and/or prevent the disease state or condition in a subject in need of such treatment.

In some embodiments, the subject is an animal. Animals include all members of the animal kingdom, but are not limited to humans, mice, rats, cats, monkeys, dogs, horses, and swine. In some embodiments, the subject is a human. In some embodiments, the subject is a mouse, a rat, a cat, a monkey, a dog, a horse, or a pig.

In some embodiments, the compound or composition is administered orally. In some embodiments, the compound or composition is administered intravenously.

In some embodiments, the methods comprise administering to the subject an effective amount of a compound disclosed herein, or a pharmaceutically acceptable salt thereof, or a composition comprising a compound disclosed herein or a pharmaceutically acceptable salt thereof, and a pharmaceutically acceptable carrier.

Pharmaceutically acceptable carriers are well-known to those skilled in the art, and include, e.g., adjuvants, diluents, excipients, fillers, lubricants and vehicles. In some embodiments, the carrier is a diluent, adjuvant, excipient, or vehicle. In some embodiments, the carrier is a diluent, adjuvant, or excipient. In some embodiments, the carrier is a diluent or adjuvant. In some embodiments, the carrier is an excipient. Often, the pharmaceutically acceptable carrier is chemically inert toward the active compounds and is non-toxic under the conditions of use. Examples of pharmaceutically acceptable carriers may include, e.g., water or saline solution, polymers such as polyethylene glycol, carbohydrates and derivatives thereof, oils, fatty acids, or alcohols. Non-limiting examples of oils as pharmaceutical carriers include oils of petroleum, animal, vegetable or synthetic origin, such as peanut oil, soybean oil, mineral oil, sesame oil and the like. The pharmaceutical carriers may also be saline, gum acacia, gelatin, starch paste, talc, keratin, colloidal silica, urea, and the like. In addition, auxiliary, stabilizing, thickening, lubricating and coloring agents may be used. Other examples of suitable pharmaceutical carriers are described in e.g., Remington's: The Science and Practice of Pharmacy, 22nd Ed. (Allen, Loyd V., Jr ed., Pharmaceutical Press (2012)); Modern Pharmaceutics, 5^{th} Ed. (Alexander T. Florence, Juergen Siepmann, CRC Press (2009)); Handbook of Pharmaceutical Excipients, 7^{th} Ed. (Rowe, Raymond C.; Sheskey, Paul J.; Cook, Walter G.; Fenton, Marian E. eds., Pharmaceutical Press (2012)) (each of which hereby incorporated by reference in its entirety).

In some embodiments, the method of treatment, prevention and/or suppression of a condition related to SHP2 phosphatase comprises the steps of: i) identifying a subject in need of such treatment; (ii) providing a compound disclosed herein or a pharmaceutically acceptable salt thereof, or a composition comprising a compound disclosed herein, or a pharmaceutically acceptable salt thereof, and a pharmaceutically acceptable carrier; and (iii) administering said compound or composition in a therapeutically effective amount to treat, prevent and/or suppress the disease state or condition related to SHP2 phosphatase in a subject in need of such treatment.

In some embodiments, the compounds of the disclosure are formulated into pharmaceutical compositions for administration to subjects in a biologically compatible form suitable for administration in vivo. According to another aspect, the present disclosure provides a pharmaceutical composition comprising a disclosed compound in admixture with a pharmaceutically acceptable diluent and/or carrier. The pharmaceutically-acceptable carrier is “acceptable” in the sense of being compatible with the other ingredients of the composition and not deleterious to the recipient thereof. The pharmaceutically-acceptable carriers employed herein may be selected from various organic or inorganic materials that are used as materials for pharmaceutical formulations and which are incorporated as analgesic agents, buffers, binders, disintegrants, diluents, emulsifiers, excipients, extenders, glidants, solubilizers, stabilizers, suspending agents, tonicity agents, vehicles and viscosity-increasing agents. Pharmaceutical additives, such as antioxidants, aromatics, colorants, flavor-improving agents, preservatives, and sweeteners, may also be added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc and water, among others. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

Surfactants such as, e.g., detergents, are also suitable for use in the formulations. Specific examples of surfactants include polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others, anionic surfactants, such as alkaline stearates, in particular sodium, potassium or ammonium stearate; calcium stearate or triethanolamine stearate; alkyl sulfates, in particular sodium lauryl sufate and sodium cetyl sulfate; sodium dodecylbenzenesulphonate or sodium dioctyl sulphosuccinate; or fatty acids, in particular those derived from coconut oil, cationic surfactants, such as water-soluble quaternary ammonium salts of formula N⁺R′R″R″R″Y⁻, in which the R radicals are identical or different optionally hydroxylated hydrocarbon radicals and Y⁻ is an anion of a strong acid, such as halide, sulfate and sulfonate anions; cetyltrimethylammonium bromide is one of the cationic surfactants which can be used, amine salts of formula N⁺R′R″R″′, in which the R radicals are identical or different optionally hydroxylated hydrocarbon radicals; octadecylamine hydrochloride is one of the cationic surfactants which can be used, non-ionic surfactants, such as optionally polyoxyethylenated esters of sorbitan, in particular Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide, amphoteric surfactants, such as substituted lauryl compounds of betaine.

When administered to a subject, the disclosed compound and pharmaceutically acceptable carriers can be sterile. Suitable pharmaceutical carriers may also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, polyethylene glycol 300, water, ethanol, polysorbate 20, and the like. The present compositions, if desired, may also contain minor amounts of wetting or emulsifying agents, or pH buffering agents.

The pharmaceutical formulations of the present disclosure are prepared by methods well-known in the pharmaceutical arts. Optionally, one or more accessory ingredients (e.g., buffers, flavoring agents, surface active agents, and the like) also are added. The choice of carrier is determined by the solubility and chemical nature of the compounds, chosen route of administration and standard pharmaceutical practice.

Additionally, the compounds and/or compositions of the present disclosure are administered to a human or animal subject by known procedures including oral administration, sublingual or buccal administration. In some embodiments, the compound and/or composition is administered orally.

For oral administration, a formulation of the compounds of the disclosure may be presented in dosage forms such as capsules, tablets, powders, granules, or as a suspension or solution. Capsule formulations may be gelatin, soft-gel or solid. Tablets and capsule formulations may further contain one or more adjuvants, binders, diluents, disintegrants, excipients, fillers, or lubricants, each of which are known in the art. Examples of such include carbohydrates such as lactose or sucrose, dibasic calcium phosphate anhydrous, corn starch, mannitol, xylitol, cellulose or derivatives thereof, microcrystalline cellulose, gelatin, stearates, silicon dioxide, talc, sodium starch glycolate, acacia, flavoring agents, preservatives, buffering agents, disintegrants, and colorants. Orally administered compositions may contain one or more optional agents such as, e.g., sweetening agents such as fructose, aspartame or saccharin; flavoring agents such as peppermint, oil of wintergreen, or cherry; coloring agents; and preservative agents, to provide a pharmaceutically palatable preparation.

In some embodiments, the composition is in unit dose form such as a tablet, capsule or single-dose vial. Suitable unit doses, i.e., therapeutically effective amounts, may be determined during clinical trials designed appropriately for each of the conditions for which administration of a chosen compound is indicated and will, of course, vary depending on the desired clinical endpoint.

In accordance with the methods of the present disclosure, the compounds of the disclosure are administered to the subject in a therapeutically effective amount, e.g., to reduce or ameliorate symptoms related to SHP2 phosphatase activity in the subject. This amount is readily determined by the skilled artisan, based upon known procedures, including analysis of titration curves established in vivo and methods and assays disclosed herein.

In some embodiments, the methods comprise administration of a therapeutically effective dosage of the compounds of the disclosure. In some embodiments, the therapeutically effective dosage is at least about 0.0001 mg/kg body weight, at least about 0.001 mg/kg body weight, at least about 0.01 mg/kg body weight, at least about 0.05 mg/kg body weight, at least about 0.1 mg/kg body weight, at least about 0.25 mg/kg body weight, at least about 0.3 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 350 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight. It will be recognized that any of the dosages listed herein may constitute an upper or lower dosage range, and may be combined with any other dosage to constitute a dosage range comprising an upper and lower limit.

In some embodiments, the therapeutically effective dosage is in the range of about 0.1 mg to about 10 mg/kg body weight, about 0.1 mg to about 6 mg/kg body weight, about 0.1 mg to about 4 mg/kg body weight, or about 0.1 mg to about 2 mg/kg body weight.

In some embodiments the therapeutically effective dosage is in the range of about 1 to 500 mg, about 2 to 150 mg, about 2 to 120 mg, about 2 to 80 mg, about 2 to 40 mg, about 5 to 150 mg, about 5 to 120 mg, about 5 to 80 mg, about 10 to 150 mg, about 10 to 120 mg, about 10 to 80 mg, about 10 to 40 mg, about 20 to 150 mg, about 20 to 120 mg, about 20 to 80 mg, about 20 to 40 mg, about 40 to 150 mg, about 40 to 120 mg or about 40 to 80 mg.

In some embodiments, the methods comprise a single dosage or administration (e.g., as a single injection or deposition). Alternatively, the methods comprise administration once daily, twice daily, three times daily or four times daily to a subject in need thereof for a period of from about 2 to about 28 days, or from about 7 to about 10 days, or from about 7 to about 15 days, or longer. In some embodiments, the methods comprise chronic administration. In yet other embodiments, the methods comprise administration over the course of several weeks, months, years or decades. In still other embodiments, the methods comprise administration over the course of several weeks. In still other embodiments, the methods comprise administration over the course of several months. In still other embodiments, the methods comprise administration over the course of several years. In still other embodiments, the methods comprise administration over the course of several decades.

The dosage administered can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion. These are all readily determined and may be used by the skilled artisan to adjust or titrate dosages and/or dosing regimens.

The precise dose to be employed in the compositions will also depend on the route of administration, and should be decided according to the judgment of the practitioner and each subject's circumstances. In specific embodiments of the disclosure, suitable dose ranges for oral administration of the compounds of the disclosure are generally about 1 mg/day to about 1000 mg/day. In some embodiments, the oral dose is about 1 mg/day to about 800 mg/day. In some embodiments, the oral dose is about 1 mg/day to about 500 mg/day. In some embodiments, the oral dose is about 1 mg/day to about 250 mg/day. In some embodiments, the oral dose is about 1 mg/day to about 100 mg/day. In some embodiments, the oral dose is about 5 mg/day to about 50 mg/day. In some embodiments, the oral dose is about 5 mg/day. In some embodiments, the oral dose is about 10 mg/day. In some embodiments, the oral dose is about 20 mg/day. In some embodiments, the oral dose is about 30 mg/day. In some embodiments, the oral dose is about 40 mg/day. In some embodiments, the oral dose is about 50 mg/day. In some embodiments, the oral dose is about 60 mg/day. In some embodiments, the oral dose is about 70 mg/day. In some embodiments, the oral dose is about 100 mg/day. It will be recognized that any of the dosages listed herein may constitute an upper or lower dosage range, and may be combined with any other dosage to constitute a dosage range comprising an upper and lower limit.

Any of the compounds and/or compositions of the disclosure may be provided in a kit comprising the compounds and/or compositions. Thus, in some embodiments, the compound and/or composition of the disclosure is provided in a kit.

Those skilled in the art will recognize, or be able to ascertain using no more than routine experimentation, many equivalents to the specific embodiments of the disclosure described herein. Such equivalents are intended to be within the scope of the present disclosure.

The disclosure is further described by the following non-limiting Examples.

### EXAMPLES

Examples are provided below to facilitate a more complete understanding of the disclosure. The following examples serve to illustrate the exemplary modes of making and practicing the disclosure. However, the scope of the disclosure is not to be construed as limited to specific embodiments disclosed in these Examples, which are illustrative only.

Instrumentation and Methods:

Reactions were monitored and final products were characterized using one of the following methods. LCMS standard conditions were: Waters HPLC system equipped with an Alliance 2695 main module, Waters 996 diode array detector and ZQ micromass ESI-MS detector. Mobile phase A: H₂O (10.0 mM NH₄HCO₂), mobile phase B: CH₃CN. HPLC conditions were: XBridge C18 column, 4.6×30 mm, 3.5 m, 0.0-0.2 min. isocratic (5% B), 0.2-2.0 min. gradient (5-100% B), 3.0-3.0 min. isocratic (100% B); flow rate: 3.0 mL/min; UV channel: 254 nm.

Purification of some racemic products was performed using semi preparative HPLC A, semi preparative HPLC B, or semi preparative SFC. Semi preparative HPLC A: Gilson 215 system equipped with a Waters 996 diode array detector and a Waters 2525 pump. Semi preparative HPLC B: Waters 2767 system equipped with a Waters 996 diode array detector, 2× Waters 515 pumps, a Waters 2525 pump and a ZQ micromass ESI-MS detector. Semi preparative SFC: Mettler Toledo Minigram SFC equipped with a Knauer K-2501 UV detector and an Alcott Model 1719 Autosampler.

Product homogeneity and enantiomeric excess determination were performed using Analytical HPLC A: Agilent 1100 HPLC system equipped with an Agilent G1315A diode array detector.

### Preparation of 2-(3-amino-3-methylpiperidin-1-yl)-6-(2,3-dichlorophenyl)imidazo[2,1-b][1,3,4]thiadiazol-5-amine formate (Compound A1)

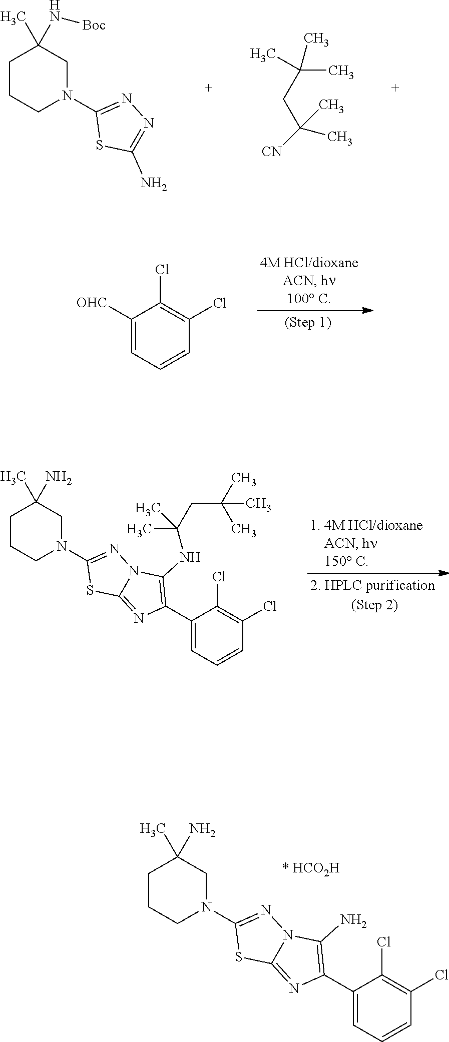

Step 1: A microwavable vial was charged with tert-butyl (1-(5-amino-1,3,4-thiadiazol-2-yl)-3-methylpiperidin-3-yl)carbamate (555 mg, 1.77 mmol) a stirbar, and MECN (5 mL). 2,3-Dichlorobenzaldehyde (339 mg, 1.94 mmol) was added, followed by the addition of 2-isocyano-2,4,4-trimethylpentane (340 μL, 1.94 mmol) and 4M hydrogen chloride in dioxane (4.42 mL, 17.7 mmol). The mixture was stirred at 100° C. for 30 min under microwave irradiation. Water (2 mL) was added to the orange solution and the mixture stirred at RT for 1 h. The solvents were removed under reduced pressure. Water (5 mL) was added and the mixture sonicated for 1 min. The oily biphasic mixture was extracted with EtOAc (3×20 mL). The combined organics were washed with brine, dried over Na₂SO₄, filtered, and concentrated under reduced pressure. The resulting crude material was purified by flash chromatography (0-100% EtOAc/heptanes) to yield intermediate 2-(3-amino-3-methylpiperidin-1-yl)-6-(2,3-dichlorophenyl)-N-(2,4,4-trimethylpentan-2-yl)imidazo[2,1-b][1,3,4]thiadiazol-5-amine as an off-white amorphous solid.

Step 2: A microwave vial was charged 2-(3-amino-3-methylpiperidin-1-yl)-6-(2,3-dichlorophenyl)-N-(2,4,4-trimethylpentan-2-yl)imidazo[2,1-b][1,3,4]thiadiazol-5-amine, hydrogen chloride (4.42 mL, 17.7 mmol) and a stirbar. The mixture was heated at 150° C. under microwave irradiation for 1 h. Water was added followed by the addition of saturated NaHCO₃. The mixture was extracted with EtOAc (3×) and the organics dried over Na₂SO₄, filtered, and concentrated under reduced pressure to give 357 mg crude product. Purification by flash chromatography (0-100% EtOAc/heptanes then 0-10% MeOH in EtOAc w/0.1% TEA yielded an impure product which was further purified by reversed-phase HPLC (0-40% MECN/0.1% formic acid). Fractions containing product were combined and lyophilized to yield Compound 1, 2-(3-amino-3-methylpiperidin-1-yl)-6-(2,3-dichlorophenyl)imidazo[2,1-b][1,3,4]thiadiazol-5-amine formate: ESMS (M+H)⁺=397.2.

### Preparation of (1-(6-(2,3-dichlorophenyl)imidazo[2,1-b][1,3,4]thiadiazol-2-yl)-4-methylpiperidin-4-yl)methanamine formate (Compound A2)

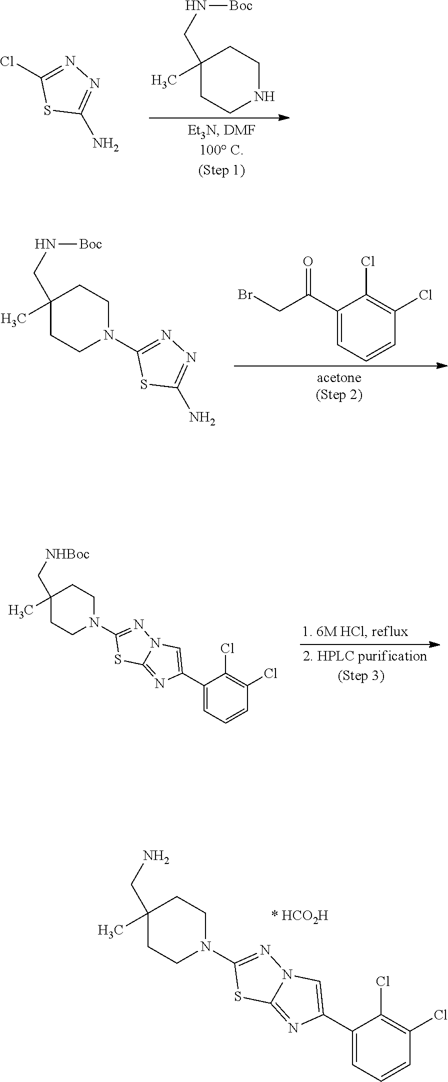 [SMILES: Nc1nnc(Cl)s1 ; CC1(CNC(=O)OC(C)(C)C)CCNCC1 ; CC1(CNC(=O)OC(C)(C)C)CCN(c2nnc(N)s2)CC1 ; O=C(CBr)c1cccc(Cl)c1Cl ; CC1(CNC(=O)OC(C)(C)C)CCN(c2nn3cc(-c4cccc(Cl)c4Cl)nc3s2)CC1 ; CC1(CN)CCN(c2nn3cc(-c4cccc(Cl)c4Cl)nc3s2)CC1]

Step 1: A disposable tube was charged with tert-butyl ((4-methylpiperidin-4-yl)methyl)carbamate hydrochloride (150 mg, 0.5664 mmol), 5-chloro-1,3,4-thiadiazol-2-amine (63.9 mg, 0.472 mmol) and a stir bar. NMP (5 mL) was added and the solution was stirred at 100° C. for 1 hour. The mixture was partitioned between EtOAc (5 mL) and water (5 mL). The aqueous phase was extracted with EtOAc (2×5 mL) and the combined organics were washed with brine, dried over sodium sulfate, filtered, and concentrated under reduced pressure. Purification by flash chromatography (0-100% EtOAc/heptanes, then 0-10% MeOH/EtOAc w/0.1% TEA) provided tert-butyl ((1-(5-amino-1,3,4-thiadiazol-2-yl)-4-methylpiperidin-4-yl)methyl)carbamate (75.0 mg, 0.2290 mmol) as an off-white solid.

Steps 2 & 3: tert-Butyl ((1-(5-amino-1,3,4-thiadiazol-2-yl)-4-methylpiperidin-4-yl)methyl)carbamate (37 mg, 0.1129 mmol) was dissolved in 2 mL acetone. 2-Bromo-1-(2,3-dichlorophenyl)ethanone (30.2 mg, 0.1129 mmol) was added and the reaction mixture was refluxed for 4 hours. After this time the mixture was cooled to 0° C. and a precipitate formed. The supernatant was removed and 6M HCl (2 mL) was added. The mixture was refluxed for 1 hour and then cooled to RT overnight. The reaction was quenched with 15% NH₄OH to a neutral pH and a precipitate formed. The mixture was extracted with EtOAc (2×20 mL) and the combined organics dried over Na₂SO₄, filtered, and concentrated under reduced pressure. The resulting crude product was taken up in a minimum of DMSO and purified by reversed phase HPLC (0-40% MECN/0.1% formic acid). Fractions containing product were combined and lyophilized to yield Compound 2 (1-(6-(2,3-dichlorophenyl)imidazo[2,1-b][1,3,4]thiadiazol-2-yl)-4-methylpiperidin-4-yl)methanamine formate (14.3 mg, 0.03608 mmol): ESMS (M+H)⁺=396; ¹H NMR (400 MHz, DMSO-d6) δ ppm 0.96 (s, 3H) 1.34-1.44 (m, 3H) 1.56 (ddd, J=13.49, 9.46, 4.15 Hz, 3H) 2.53 (s, 2H) 3.39 (ddd, J=13.12, 9.58, 3.42 Hz, 4H) 3.55 (dt, J=13.18, 4.88 Hz, 3H) 7.39 (t, J=7.93 Hz, 2H) 7.53 (dd, J=8.06, 1.46 Hz, 1H) 8.02 (dd, J=7.93, 1.59 Hz, 1H) 8.33 (s, 1H) 8.41 (s, 1H).

### Preparation of 2-((1R,5S,6r)-6-aminobicyclo[3.1.0]hexan-3-yl)-6-(2,3-dichlorophenyl) imidazo[2,1-b][1,3,4]thiadiazol-5-amine formate (Compound A3)

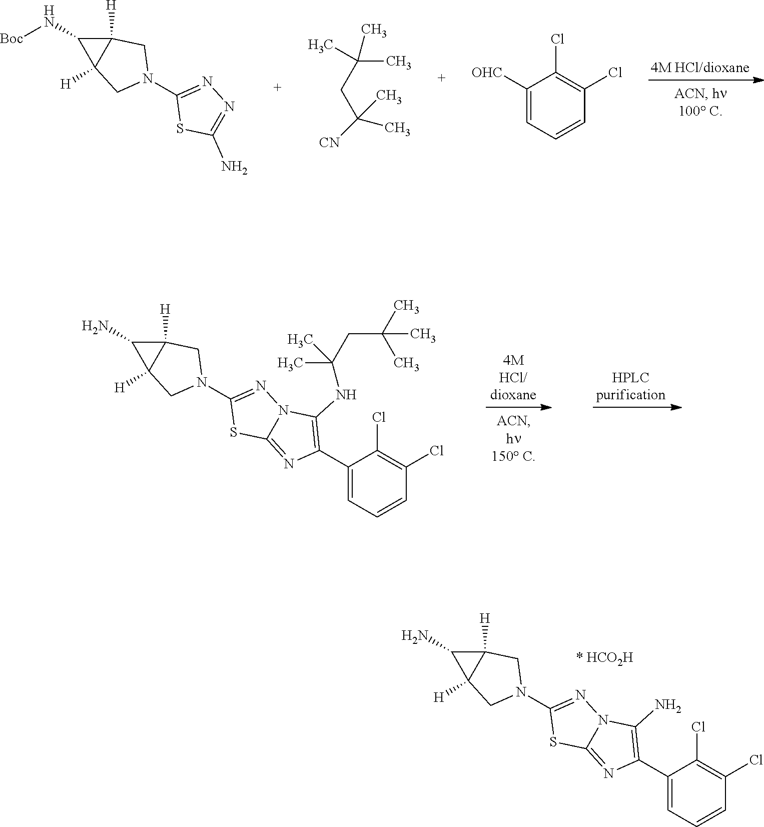

A microwavable vial was charged with tert-butyl ((1R,5S,6s)-3-(5-amino-1,3,4-thiadiazol-2-yl)-3-azabicyclo[3.1.0]hexan-6-yl)carbamate (200 mg, 0.6725 mmol), a stirbar, and MECN (5 mL). 2,3-Dichlorobenzaldehyde (129 mg, 0.7397 mmol) was added, followed by the addition of 2-isocyano-2,4,4-trimethylpentane (128 μL, 0.7397 mmol) and 4M hydrogen chloride in dioxane (16.8 μL, 0.06725 mmol). The mixture was stirred at 100° C. for 30 min under microwave irradiation. Water (2 mL) was added to the orange solution and the mixture stirred at RT for 2 h. A precipitate formed, which was a mixture of final product and 2,4,4-trimethylpentyl protected material, was separated from the solution and placed in a microwave vial. This intermediate (tert-butyl ((1R,5S,6s)-3-(6-(2,3-dichlorophenyl)-5-((2,4,4-trimethylpentan-2-yl)amino)imidazo[2,1-b][1,3,4]thiadiazol-2-yl)-3-azabicyclo[3.1.0]hexan-6-yl)carbamate) was placed in a microwavable vial and treated with 4M HCl/dioxane (269 mg, 7.39 mmol). The mixture was heated at 150° C. with stirring under microwave irradiation for 1 h. Water (5 mL) was added and the homogenous crude product was purified by reversed-phase HPLC (0-40% MECN/0.1% formic acid). Fractions containing product were combined and lyophilized to yield Compound 3, 2-((1R,5S,6r)-6-aminobicyclo[3.1.0]hexan-3-yl)-6-(2,3-dichlorophenyl)imidazo[2,1-b][1,3,4]thiadiazol-5-amine formate: ESMS (M+H)⁺=381; ¹H NMR (400 MHz, DMSO-d6) δ ppm 8.25 (s, 1H), 7.49 (dd, J=1.46, 8.06 Hz, 1H), 7.42 (dd, J=1.46, 7.81 Hz, 1H), 7.27-7.34 (m, 1H), 4.76 (s, 2H), 3.52-3.58 (m, 2H), 3.47-3.52 (m, 2H), 3.31 (br s, 2H), 2.05 (s, 1H), 1.63 (br s, 2H).

### Preparation of 2-(4-amino-4-methylpiperidin-1-yl)-6-(2,3-dichlorophenyl)imidazo[2,1-b][1,3,4]thiadiazol-5-amine formate (Compound A4)

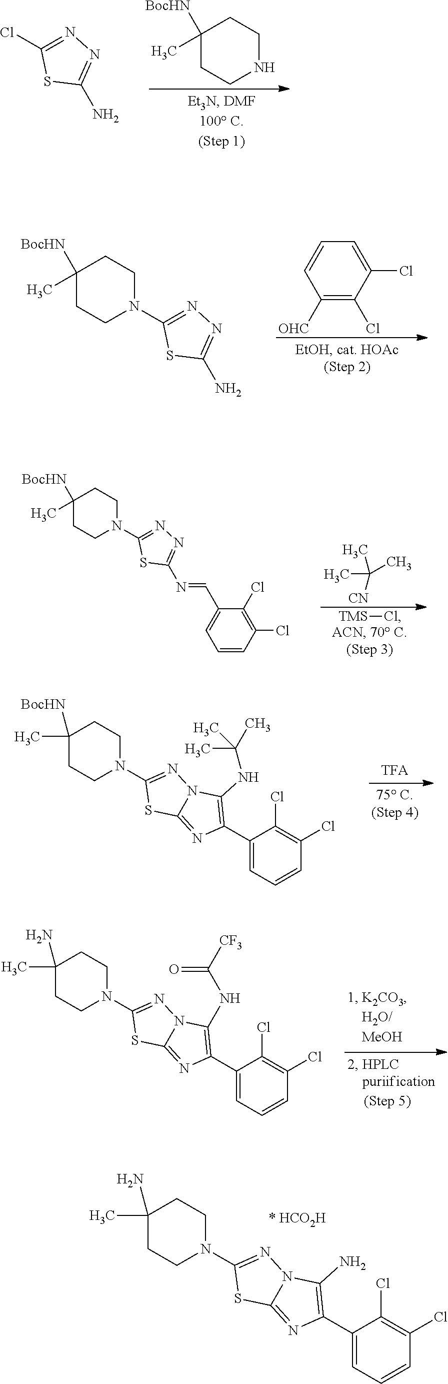

Step 1: A disposable tube was charged with tert-butyl (4-methylpiperidin-4-yl)carbamate hydrochloride (593 mg, 2.367 mmol), 5-chloro-1,3,4-thiadiazol-2-amine (267 mg, 1.973 mmol) and a stir bar. NMP (20 mL) was added and the solution was stirred at 100° C. for 1 hour. The mixture was partitioned between EtOAc (20 mL) and water (20 mL). The aqueous phase was extracted with EtOAc (2×20 mL) and the combined organics were washed with brine, dried over sodium sulfate, filtered, and concentrated under reduced pressure. Purification by flash chromatography (0-100% EtOAc/heptanes, then 0-10% MeOH/EtOAc w/0.1% TEA) provided tert-butyl (1-(5-amino-1,3,4-thiadiazol-2-yl)-4-methylpiperidin-4-yl)carbamate (300 mg, 0.9571 mmol) as an off-white solid.

Step 2: A disposable tube was charged with tert-butyl (1-(5-amino-1,3,4-thiadiazol-2-yl)-4-methylpiperidin-4-yl)carbamate (300 mg, 0.9571 mmol), 2,3-dichlorobenzaldehyde (334 mg, 1.91 mmol), and a stirbar. EtOH (5 mL) was added, followed by a few drops of acetic acid, and the solution was stirred at 100° C. After 18 hours the reaction mixture was cooled and the resulting precipitate collected, washed with EtOH, and dried to produce tert-butyl (E)-(1-(5-((2,3-dichlorobenzylidene)amino)-1,3,4-thiadiazol-2-yl)-4-methylpiperidin-4-yl)carbamate, which was used as is in subsequent reactions

Step 3: A disposable tube was charged with tert-butyl (E)-(1-(5-((2,3-dichlorobenzylidene)amino)-1,3,4-thiadiazol-2-yl)-4-methylpiperidin-4-yl)carbamate (277 mg, 0.5888 mmol) and a stirbar. MeCN (5 mL) was added, followed by the addition of chlorotrimethylsilane (89.6 μL, 0.7065 mmol). The reaction mixture was stirred at RT for 30 min followed by the addition of N-tert-butylformonitrile (80.8 μL, 0.7065 mmol). The reaction mixture was stirred at 70° C. for 18 hours. The volatiles were removed under reduced pressure and the residue was purified by flash chromatography to yield tert-butyl (1-(5-(tert-butylamino)-6-(2,3-dichlorophenyl)imidazo[2,1-b][1,3,4]thiadiazol-2-yl)-4-methylpiperidin-4-yl)carbamate as an off-white amorphous solid: ESMS (M+H)⁺=553/555.

Step 4: A sealed tube was charged with tert-butyl (1-(5-(tert-butylamino)-6-(2,3-dichlorophenyl)imidazo[2,1-b][1,3,4]thiadiazol-2-yl)-4-methylpiperidin-4-yl)carbamate (120 mg, 0.2167 mmol) and a stirbar. Trifluoroacetic acid (3 mL) was added and the solution was stirred at 75° C. for 3 hours. The volatiles were removed under reduced pressure. The residue was taken up in toluene, which was also removed under reduced pressure (3×). Purification by flash chromatography (Hept/EtOAc/MeOH) yielded N-(2-(4-amino-4-methylpiperidin-1-yl)-6-(2,3-dichlorophenyl)imidazo[2,1-b][1,3,4]thiadiazol-5-yl)-2,2,2-trifluoroacetamide.

Step 5: A disposable tube was charged with N-(2-(4-amino-4-methylpiperidin-1-yl)-6-(2,3-dichlorophenyl)imidazo[2,1-b][1,3,4]thiadiazol-5-yl)-2,2,2-trifluoroacetamide (236 mg, 0.4783 mmol), potassium carbonate (197 mg, 1.43 mmol), and a stirbar. 1:1 MeOH:H₂O (10 mL) was added and the reaction mixture stirred at RT for 18 hours. The mixture was diluted with 10 mL of dioxane and the final product purified by reversed-phase HPLC to produce Compound 4 as off-white amorphous solid: ESMS (M+H)⁺=397/399; ¹H-NMR (400 MHz, DMSO-d₆) δ 7.52 (dd, J=1.46, 7.81 Hz, 1H), 7.44 (dd, J=1.71, 7.81 Hz, 1H), 7.34 (t, J=7.81 Hz, 1H), 4.87 (br s, 2H), 3.63-3.74 (m, 2H), 1.71-1.91 (m, 4H), 1.38 (s, 3H).

### Preparation of 2-(3-(aminomethyl)-3-methylpyrrolidin-1-yl)-6-(2,3-dichlorophenyl) imidazo[2,1-b][1,3,4]thiadiazol-5-amine (Compound A5)

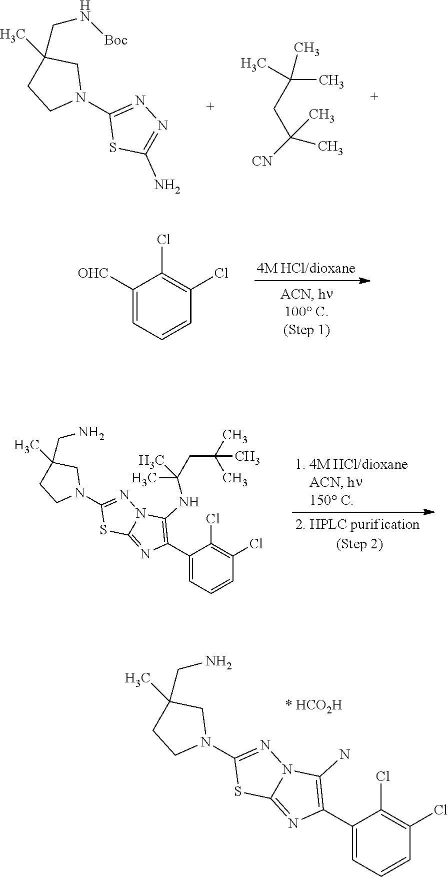

Step 1: A microwavable vial was charged with tert-butyl ((1-(5-amino-1,3,4-thiadiazol-2-yl)-3-methylpyrrolidin-3-yl)methyl)carbamate (100 mg, 0.3190 mmol), a stirbar, and MECN (2 mL). 2,3-Dichlorobenzaldehyde (50.2 mg, 0.2870 mmol) was added, followed by the addition of 2-isocyano-2,4,4-trimethylpentane (44.4 mg, 0.319 mmol) and 4M hydrogen chloride in dioxane (7.97 μL, 0.03190 mmol). The mixture was stirred at 100° C. for 30 min under microwave irradiation. Water (2 mL) was added to the orange solution and the mixture stirred at RT for 1 h. EtOAc (5 mL) was added to the resulting oily biphasic mixture. The organics were washed with sat'd NaCl, dried over Na₂SO₄, filtered, and concentrated under reduced pressure to produce intermediate 2-(3-(aminomethyl)-3-methylpyrrolidin-1-yl)-6-(2,3-dichlorophenyl)-N-(2,4,4-trimethylpentan-2-yl)imidazo[2,1-b][1,3,4]thiadiazol-5-amine along with some final product.

Step 2: The product from Step 1 was placed in a microwavable vial and treated with 4M HCl/dioxane (797 μL, 3.19 mmol). The mixture was heated at 150° C. with stirring under microwave irradiation for 1 h. After cooling, the mixture was quenched with 15% NH₄₀H, extracted with EtOAc (5 mL). The organics were washed with sat'd NaCl, dried over Na₂SO₄, filtered, and concentrated under reduced pressure. The crude product was dissolved in a minimum of DMSO and purified by reversed-phase HPLC (10-40% MECN/0.1% formic acid). Fractions containing product were combined and lyophilized to yield Compound 5, 2-(3-(aminomethyl)-3-methylpyrrolidin-1-yl)-6-(2,3-dichlorophenyl)imidazo[2,1-b][1,3,4]thiadiazol-5-amine: ESMS (M+H)⁺=397; ¹H NMR (400 MHz, DMSO-d6) δ ppm 1.02-1.10 (m, 3H) 1.67-1.76 (m, 1H) 1.92-2.04 (m, 2H) 2.59 (s, 1H) 3.12 (br d, J=9.77 Hz, 1H) 3.40 (br d, J=9.77 Hz, 2H) 3.51 (br t, J=7.20 Hz, 2H) 4.74 (s, 1H) 6.28 (br s, 1H) 7.27-7.35 (m, 1H) 7.44 (dd, J=7.81, 1.71 Hz, 1H) 7.49 (dd, J=7.81, 1.46 Hz, 1H) 8.30 (s, 1H).

### Preparation of 2-(3-(aminomethyl)-3-methylpyrrolidin-1-yl)-6-(2-chlorophenyl)imidazo[2,1-b][1,3,4]thiadiazol-5-amine (Compound A6)

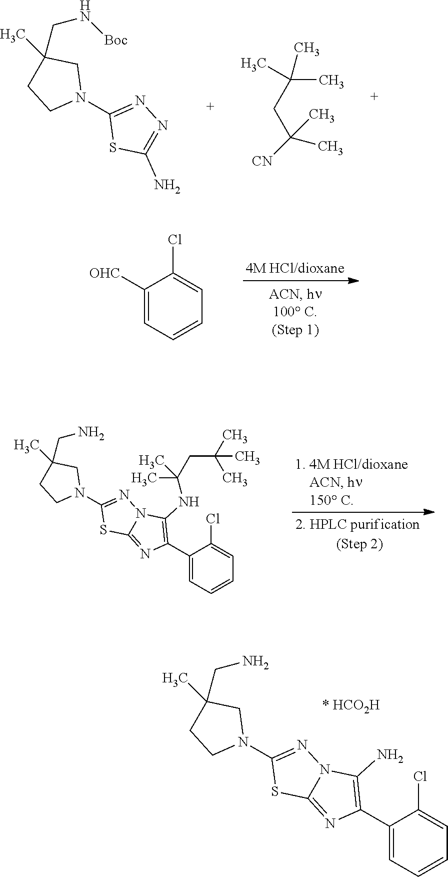

Step 1: A microwavable vial was charged with tert-butyl ((1-(5-amino-1,3,4-thiadiazol-2-yl)-3-methylpyrrolidin-3-yl)methyl)carbamate (85 mg, 0.2712 mmol), a stirbar, and MECN (2 mL). 2-Chlorobenzaldehyde (34.2 mg, 0.2440 mmol) was added, followed by the addition of 2-isocyano-2,4,4-trimethylpentane (37.7 mg, 0.2712 mmol) and 4M hydrogen chloride in dioxane (6.77 μL, 0.02712 mmol). The mixture was stirred at 100° C. for 30 min under microwave irradiation. Water (2 mL) was added to the orange solution and the mixture stirred at RT for 1 h. EtOAc (5 mL) was added to the resulting oily biphasic mixture. The organics were washed with sat'd NaCl, dried over Na₂SO₄, filtered, and concentrated under reduced pressure to yield 2-(3-(aminomethyl)-3-methylpyrrolidin-1-yl)-6-(2-chlorophenyl)-N-(2,4,4-trimethylpentan-2-yl)imidazo[2,1-b][1,3,4]thiadiazol-5-amine, which was used in subsequent reactions as is.

Step 2: 2-(3-(Aminomethyl)-3-methylpyrrolidin-1-yl)-6-(2-chlorophenyl)-N-(2,4,4-trimethylpentan-2-yl)imidazo[2,1-b][1,3,4]thiadiazol-5-amine was placed in a microwavable vial and treated with 4M HCl/dioxane (677 μL, 2.71 mmol). The mixture was heated at 100° C. with stirring overnight. After cooling, the mixture was quenched with 15% NH₄OH, extracted with EtOAc (5 mL). The organics were washed with sat'd NaCl, dried over Na₂SO₄, filtered, and concentrated under reduced pressure. The crude product was dissolved in 5 mL MeOH and 10 drops of conc. HCl was added. The reaction mixture was stirred at 50° C. for 1 h. After removal of the volatiles under reduced pressure, the crude product was dissolved in a minimum of DMSO and purified by reversed-phase HPLC (5-30% MECN/0.1% formic acid). Fractions containing product were combined and lyophilized to yield Compound 6, 2-(3-(aminomethyl)-3-methylpyrrolidin-1-yl)-6-(2-chlorophenyl)imidazo[2,1-b][1,3,4]thiadiazol-5-amine (2.0 mg, 0.0049 mmol): ESMS (M+H)⁺=363.3; ¹H NMR (400 MHz, DMSO-d6) δ ppm 1.04-1.11 (m, 2H) 1.67-1.76 (m, 1H) 1.93-2.03 (m, 1H) 2.58 (s, 1H) 3.12 (br d, J=9.77 Hz, 1H) 3.40 (br d, J=9.77 Hz, 3H) 3.51 (br t, J=7.08 Hz, 3H) 4.58 (br s, 2H) 7.21-7.27 (m, 1H) 7.31 (td, J=7.45, 1.22 Hz, 1H) 7.42-7.50 (m, 2H) 8.34 (s, 1H).

### Preparation of (1-(6-(2-chlorophenyl)-5-methylimidazo[2,1-b][1,3,4]thiadiazol-2-yl)-4-methylpiperidin-4-yl)methanamine formate (Compound A7)

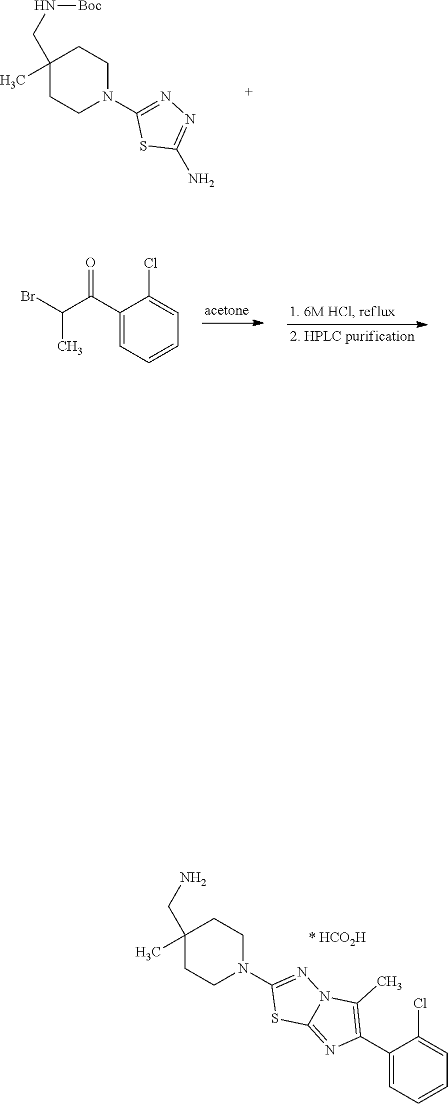 [SMILES: CC1(CNC(=O)OC(C)(C)C)CCN(c2nnc(N)s2)CC1 ; CC(Br)C(=O)c1ccccc1Cl ; Cc1c(-c2ccccc2Cl)nc2sc(N3CCC(C)(CN)CC3)nn12]

tert-Butyl ((1-(5-amino-1,3,4-thiadiazol-2-yl)-4-methylpiperidin-4-yl)methyl)carbamate (85 mg, 0.2595 mmol) was dissolved in 2 mL acetone. 2-Bromo-1-(2-chlorophenyl)propan-1-one (64.2 mg, 0.2595 mmol) was added and the reaction mixture was refluxed for 24 hours. The reaction mixture was cooled and the volatiles removed under reduced pressure. 6M HCl (2 mL) was added to the residue and the mixture was refluxed for 1 hour. After cooling to RT, the reaction was quenched with 15% NH₄OH to a neutral pH. The mixture was extracted with EtOAc (2×20 mL) and the combined organics dried over Na₂SO₄, filtered, and concentrated under reduced pressure. The resulting crude product was taken up in a minimum of DMSO and purified by reversed phase HPLC (10-40% MECN/0.1% formic acid). Fractions containing product were combined and lyophilized to yield Compound 7, (1-(6-(2-chlorophenyl)-5-methylimidazo[2,1-b][1,3,4]thiadiazol-2-yl)-4-methylpiperidin-4-yl)methanamine formate (31.5 mg, 0.07465 mmol): ¹H NMR (400 MHz, DMSO-d6) δ ppm 0.97 (s, 2H) 1.39 (dt, J=13.43, 4.15 Hz, 2H) 1.56 (ddd, J=13.55, 9.52, 4.27 Hz, 2H) 2.26 (s, 3H) 2.53-2.56 (m, 2H) 3.38 (ddd, J=13.18, 9.64, 3.54 Hz, 3H) 3.55 (dt, J=13.37, 4.79 Hz, 2H) 7.33-7.40 (m, 2H) 7.43-7.48 (m, 1H) 7.49-7.55 (m, 1H) 8.35 (s, 1H).

### Preparation of (2-(3-(aminomethyl)-3-methylpyrrolidin-1-yl)imidazo[2,1-b][1,3,4]thiadiazol-6-yl)(3,4-dihydro-1,5-naphthyridin-1(2H)-yl)methanone formate (Compound A8)

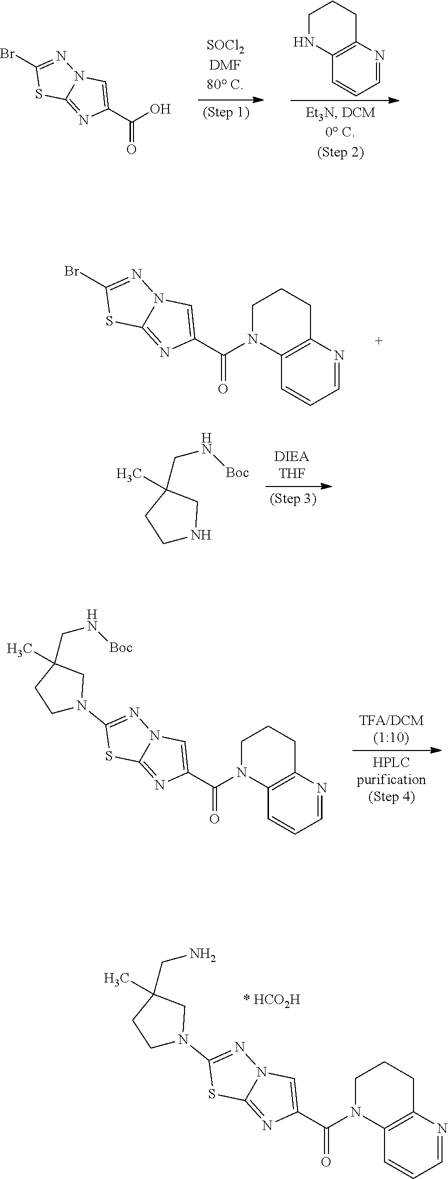 [SMILES: O=C(O)c1cn2nc(Br)sc2n1 ; c1cnc2c(c1)NCCC2 ; O=C(c1cn2nc(Br)sc2n1)N1CCCc2ncccc21 ; CC1(CNC(=O)OC(C)(C)C)CCNC1 ; CC1(CNC(=O)OC(C)(C)C)CCN(c2nn3cc(C(=O)N4CCCc5ncccc54)nc3s2)C1 ; CC1(CN)CCN(c2nn3cc(C(=O)N4CCCc5ncccc54)nc3s2)C1]

Steps 1 & 2: A reaction vial was charged with crude 2-bromoimidazo[2,1-b][1,3,4]thiadiazole-6-carboxylic acid (135 mg, 0.5442 mmol), sulfurous dichloride (987 μL, 13.6 mmol), and 5 drops of DMF. The resulting suspension was stirred at 80° C. for 2 hours. The volatiles were removed under reduced pressure and the residue was taken up in toluene, which was also removed under reduced pressure (3×). The resulting 2-bromoimidazo[2,1-b][1,3,4]thiadiazole-6-carbonyl chloride was suspended in DCM (2 mL), cooled to 0-5° C. with an ice bath and treated with triethylamine (701 μL, 5.04 mmol) and a solution of 1,2,3,4-tetrahydro-1,5-naphthyridine (169 mg, 1.26 mmol) in DCM (1 mL). The reaction mixture was allowed to warm to RT overnight. The mixture was treated with 1 mL of sat'd NaHCO₃ and the aqueous layer extracted with DCM. The combined organics were dried over Na₂SO₄, filtered, and concentrated under reduced pressure to yield (2-bromoimidazo[2,1-b][1,3,4]thiadiazol-6-yl)(3,4-dihydro-1,5-naphthyridin-1(2H)-yl)methanone as a brown oil. This material was used directly in subsequent reactions as is.

Step 3: (2-Bromoimidazo[2,1-b][1,3,4]thiadiazol-6-yl)(3,4-dihydro-1,5-naphthyridin-1(2H)-yl)methanone from Step 2 was dissolved in THE (2 mL) and tert-butyl ((3-methylpyrrolidin-3-yl)methyl)carbamate (88.2 mg, 0.4118 mmol) was added followed by the addition of diisopropylethylamine (142 μL, 0.8236 mmol). The reaction mixture was stirred at RT overnight then partitioned between EtOAc (10 mL) and water (2 mL). The organics were washed with brine, and dried over Na₂SO₄, filtered and the volatiles removed under reduced pressure to provide tert-butyl ((3-methyl-1-(6-(1,2,3,4-tetrahydro-1,5-naphthyridine-1-carbonyl)imidazo[2,1-b][1,3,4]thiadiazol-2-yl)pyrrolidin-3-yl)methyl)carbamate, which was used in subsequent reactions as is.

Step 4: tert-Butyl ((3-methyl-1-(6-(1,2,3,4-tetrahydro-1,5-naphthyridine-1-carbonyl)imidazo[2,1-b][1,3,4]thiadiazol-2-yl)pyrrolidin-3-yl)methyl)carbamate from Step 3 was dissolved in 4 mL DCM and 0.4 mL of TFA was added. After stirring for 30 minutes at RT, the reaction mixture was concentrated under reduced pressure. The residue was dissolved in about 5 mL toluene and the volatiles were again removed under reduced pressure (2×) to remove any residual TFA. The crude product was dissolved in a minimum of DMSO and purified by reversed-phase HPLC (5-10% MECN/0.1% formic acid). The fractions containing product were combined and lyophilized after removal of the bulk of the MECN to yield Compound 8, (2-(3-(aminomethyl)-3-methylpyrrolidin-1-yl)imidazo[2,1-b][1,3,4]thiadiazol-6-yl)(3,4-dihydro-1,5-naphthyridin-1(2H)-yl)methanone formate (5.0 mg, 0.011 mmol): ESMS (M+H)⁺=398.2; ¹H NMR (400 MHz, DMSO-d6) δ ppm 1.16 (s, 3H) 1.87 (dt, J=12.82, 6.53 Hz, 1H) 1.94-2.08 (m, 3H) 2.90-3.03 (m, 4H) 3.26 (d, J=10.25 Hz, 1H) 3.46 (d, J=10.25 Hz, 1H) 3.56 (br t, J=7.08 Hz, 2H) 4.11-4.18 (m, 2H) 7.27 (br dd, J=8.18, 5.00 Hz, 1H) 7.82-7.93 (m, 4H) 8.30 (br d, J=4.39 Hz, 1H).

### Preparation of 2-(4-(aminomethyl)-4-methylpiperidin-1-yl)-6-(2,3-dichlorophenyl) imidazo[2,1-b][1,3,4]thiadiazol-5-amine formate (Compound A9)

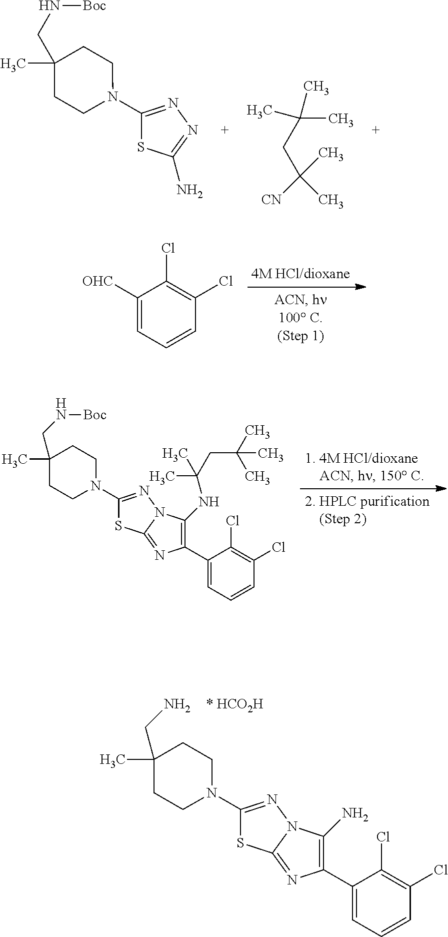

Step 1: A microwavable vial was charged with tert-butyl ((1-(5-amino-1,3,4-thiadiazol-2-yl)-4-methylpiperidin-4-yl)methyl)carbamate (141 mg, 0.4306 mmol), a stirbar, and MECN (2 mL). 2,3-Dichlorobenzaldehyde (82.8 mg, 0.4736 mmol) was added, followed by the addition of 2-isocyano-2,4,4-trimethylpentane (82.9 μL, 0.4736 mmol) and 4M hydrogen chloride in dioxane (10.7 μL, 0.04306 mmol). The mixture was stirred at 100° C. for 30 min under microwave irradiation. The volatiles were removed under reduced pressure and water (2 mL) was added to the orange solution and the mixture sonicated for 1 min. The resulting biphasic mixture was extracted with EtOAc (3×5 mL) and the combined organics were washed with brine, dried over Na₂SO₄, filtered, and concentrated under reduced pressure. The resulting crude material was purified by silica gel chromatography (10 g column; 0-100% EtOAc) to give intermediate product tert-butyl ((1-(6-(2,3-dichlorophenyl)-5-((2,4,4-trimethylpentan-2-yl)amino)imidazo[2,1-b][1,3,4]thiadiazol-2-yl)-4-methylpiperidin-4-yl)methyl)carbamate (98.0 mg, 0.1571 mmol) as a yellow oil.

Step 2: The tert-butyl ((1-(6-(2,3-dichlorophenyl)-5-((2,4,4-trimethylpentan-2-yl)amino)imidazo[2,1-b][1,3,4]thiadiazol-2-yl)-4-methylpiperidin-4-yl)methyl)carbamate from Step 1 was placed in a microwavable vial and treated with 4M HCl/dioxane (1.5 mL, 6.00 mmol). The mixture was heated at 150° C. with stirring under microwave irradiation for 1 h. Water (5 mL) was added and the homogenous crude product was purified by reversed-phase HPLC (0-40% MECN/0.1% formic acid). Fractions containing product were combined and lyophilized to yield Compound 9, 2-(4-(aminomethyl)-4-methylpiperidin-1-yl)-6-(2,3-dichlorophenyl)imidazo[2,1-b][1,3,4]thiadiazol-5-amine formate (11.9 mg, 0.02601 mmol): ESMS (M+H)⁺=411.3; ¹H NMR (400 MHz, DMSO-d6) δ ppm 0.06 (s, 1H) 0.96 (s, 2H) 1.33-1.41 (m, 2H) 1.55 (ddd, J=13.55, 9.52, 4.27 Hz, 2H) 2.47-2.53 (m, 13H) 3.37 (ddd, J=13.00, 9.58, 3.30 Hz, 11H) 3.54 (dt, J=13.18, 4.88 Hz, 3H) 4.82 (s, 2H) 7.28-7.35 (m, 1H) 7.43 (dd, J=7.81, 1.46 Hz, 1H) 7.49 (dd, J=7.93, 1.59 Hz, 1H) 8.36 (s, 1H).

### Preparation of (1-(6-(2-chlorophenyl)-5-methylimidazo[2,1-b][1,3,4]thiadiazol-2-yl)-3-methylpyrrolidin-3-yl)methanamine formate (Compound A10)

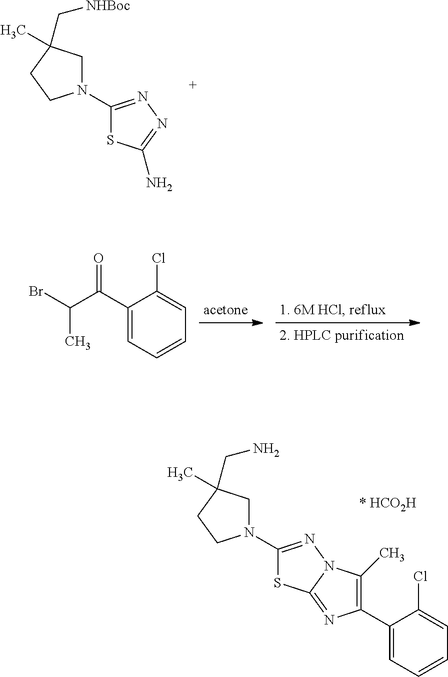 [SMILES: CC1(CNC(=O)OC(C)(C)C)CCN(c2nnc(N)s2)C1 ; CC(Br)C(=O)c1ccccc1Cl ; Cc1c(-c2ccccc2Cl)nc2sc(N3CCC(C)(CN)C3)nn12]

tert-Butyl ((1-(5-amino-1,3,4-thiadiazol-2-yl)-3-methylpyrrolidin-3-yl)methyl)carbamate (100 mg, 0.3190 mmol) was dissolved in 5 mL acetone. 2-Bromo-1-(2-chlorophenyl)propan-1-one (118 mg, 0.4785 mmol) was added and the reaction mixture was refluxed for 24 hours. The reaction mixture was cooled and the volatiles removed under reduced pressure. 6M HCl (4 mL) was added to the residue and the mixture was refluxed for 1 hour and then stirred at RT overnight. The reaction was quenched with 15% NH₄OH to a neutral pH. The mixture was extracted with EtOAc (2×20 mL) and the combined organics dried over Na₂SO₄, filtered, and concentrated under reduced pressure. The resulting crude product was taken up in a minimum amount of DMSO and purified by reversed phase HPLC (10-40% MeCN/0.1% formic acid). Fractions containing product were combined and lyophilized to yield Compound 10, (1-(6-(2-chlorophenyl)-5-methylimidazo[2,1-b][1,3,4]thiadiazol-2-yl)-3-methylpyrrolidin-3-yl)methanamine formate (30.0 mg, 0.07354 mmol): ESMS (M+H)⁺=362.1; ¹H NMR (400 MHz, DMSO-d6) δ ppm 1.09 (s, 3H) 1.75 (dt, J=12.63, 6.50 Hz, 1H) 2.00 (dt, J=12.57, 7.51 Hz, 1H) 2.26 (s, 3H) 2.66 (s, 2H) 3.17 (d, J=10.01 Hz, 1H) 3.43 (d, J=10.01 Hz, 1H) 3.53 (t, J=7.08 Hz, 2H) 7.33-7.40 (m, 2H) 7.44-7.48 (m, 1H) 7.49-7.53 (m, 1H) 8.29 (s, 1H).

### Preparation of 1-(6-(2-chlorophenyl)-5-methylimidazo[2,1-b][1,3,4]thiadiazol-2-yl)-3-methylpyrrolidin-3-amine formate (Compound A11)

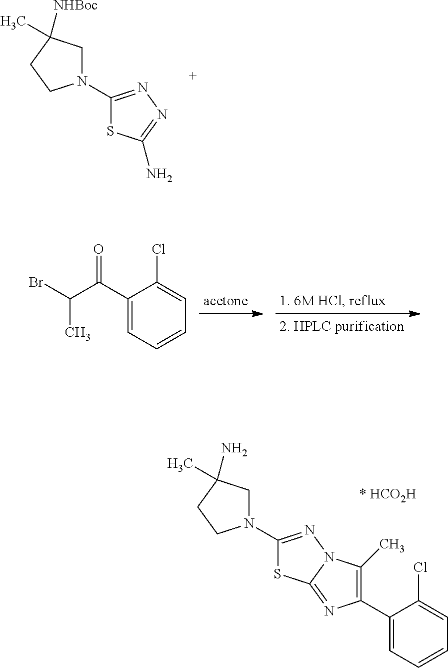 [SMILES: CC1(NC(=O)OC(C)(C)C)CCN(c2nnc(N)s2)C1 ; CC(Br)C(=O)c1ccccc1Cl ; Cc1c(-c2ccccc2Cl)nc2sc(N3CCC(C)(N)C3)nn12]

tert-Butyl (1-(5-amino-1,3,4-thiadiazol-2-yl)-3-methylpyrrolidin-3-yl)carbamate (35 mg, 0.1169 mmol) was dissolved in 2 mL acetone. 2-Bromo-1-(2-chlorophenyl)propan-1-one (37.7 μL, 0.2338 mmol) was added and the reaction mixture was refluxed for 24 hours. The reaction mixture was cooled and the volatiles removed under reduced pressure. 6M HCl (2 mL) was added to the residue and the mixture was refluxed for 2 hours and then cooled to RT. The reaction was quenched with 15% NH₄OH to a neutral pH. The mixture was extracted with EtOAc (2×20 mL) and the combined organics dried over Na₂SO₄, filtered, and concentrated under reduced pressure. The resulting crude product was taken up in a minimum amount of DMSO and purified by reversed phase HPLC (5-30% MECN/0.1% formic acid). Fractions containing product were combined and lyophilized to yield Compound 11, 1-(6-(2-chlorophenyl)-5-methylimidazo[2,1-b][1,3,4]thiadiazol-2-yl)-3-methylpyrrolidin-3-amine formate (13.6 mg, 0.03452 mmol): ESMS (M+H)⁺=348.1; ¹H NMR (400 MHz, DMSO-d6) δ ppm 1.49 (s, 3H) 2.16-2.27 (m, 1H) 2.27-2.35 (m, 4H) 3.55 (d, J=11.23 Hz, 1H) 3.60 (td, J=9.28, 3.91 Hz, 1H) 3.68-3.76 (m, 2H) 7.37-7.43 (m, 2H) 7.45-7.51 (m, 1H) 7.52-7.57 (m, 1H) 8.47 (brs, 3H).

### Preparation of 6-(2,3-dichlorophenyl)-2-(piperazin-1-yl)imidazo[2,1-b][1,3,4]thiadiazol-5-amine (Compound A12)

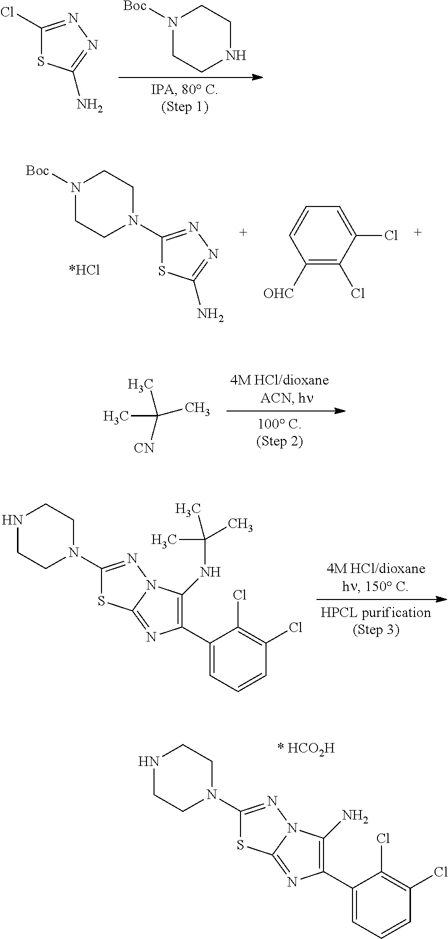

Step 1: A round bottomed was charged with 5-chloro-1,3,4-thiadiazol-2-amine (3 g, 22.1 mmol), tert-butyl piperazine-1-carboxylate (4.93 g, 26.5 mmol), and a stirbar. Isopropyl alcohol (30 mL) was added, and the solution was stirred at 80° C. for 2.5 days. The reaction mixture was cooled and the resulting precipitate was collected to yield tert-butyl 4-(5-amino-1,3,4-thiadiazol-2-yl)piperazine-1-carboxylate. This material was used in subsequent reactions as is.

Step 2: A microwave vial was charged with tert-butyl 4-(5-amino-1,3,4-thiadiazol-2-yl)piperazine-1-carboxylate (300 mg, 1.05 mmol), a stirbar, and MeCN (5 mL). 2,3-Dichlorobenzaldehyde (201 mg, 1.15 mmol) was added, followed by the addition of 2-isocyano-2-methylpropane (130 μL, 1.15 mmol) and 4M hydrogen chloride in dioxane (26.2 μL, 0.105 mmol). The mixture was stirred at 100° C. under microwave irradiation for 30 minutes and then at RT for 16 hours. The resulting precipitate was collected to yield N-(tert-butyl)-6-(2,3-dichlorophenyl)-2-(piperazin-1-yl)imidazo[2,1-b][1,3,4]thiadiazol-5-amine as an off-white amorphous solid. This material was used as is in subsequent reactions.

Step 3: A microwave vial was charged with N-(tert-butyl)-6-(2,3-dichlorophenyl)-2-(piperazin-1-yl)imidazo[2,1-b][1,3,4]thiadiazol-5-amine, 4M HCl in dioxane, and a stirbar. The reaction mixture was stirred at 150° C. under microwave irradiation for 1 hour. The resulting precipitate was collected and purified by reversed-phase HPLC to yield 6-(2,3-dichlorophenyl)-2-(piperazin-1-yl)imidazo[2,1-b][1,3,4]thiadiazol-5-amine (Compound 12) as an off-white amorphous solid: ESMS (M+H)⁺=369.

Using the general procedures described above, with reagents, starting materials, and conditions familiar to those skilled in the art, the following compounds representative of the disclosure are prepared:

| Compound | Structure |
|---|---|
| A13 | 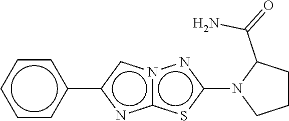 |
| A14 | 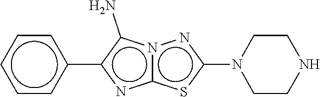 |
| A15 | 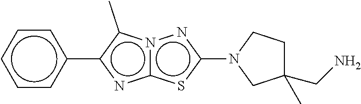 |

### Preparation of 2-(4-amino-4-methyl-1-piperidyl)-5-(2,3-dichlorophenyl)thiazol-4-amine, dihydrochloride (Compound B1)

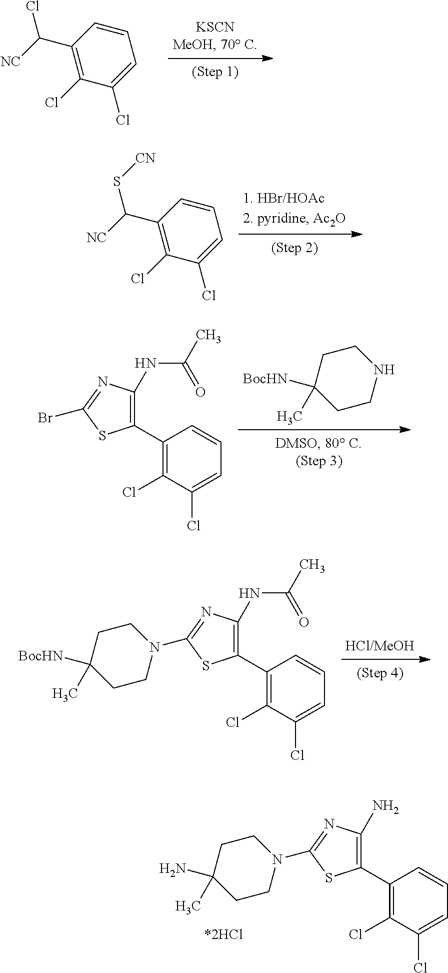

Step 1: Sodium thiocyanate (735.4 mg, 9.1 mmol) was added to a solution of 2-chloro-2-(2,3-dichlorophenyl)acetonitrile (2.0 g, 9.1 mmol) in MeOH (30.0 mL). The reaction mixture was stirred at 70° C. for 22 hours and the resulting precipitate was filtered off. The filtrate was concentrated under reduced pressure to give a residue, which was triturated with DCM (30 mL). The resulting solid was filtered off and the filtrate concentrated under reduced pressure to afford 1,2-dichloro-3-(isocyano(thiocyanato)methyl)benzene (2.1 g, 8.8 mmol, 96.6% yield) as a yellow solid. This material used in the next step without further purification.

Step 2: To a solution of 1,2-dichloro-3-(isocyano(thiocyanato)methyl)benzene (2.1 g, 8.7 mmol) in AcOH (25.0 mL) was added HBr/AcOH (10.0 mL, 33%). The mixture was stirred at 10° C. for 2 hours. Acetic anhydride (4.5 g, 43.8 mmol, 4.1 mL) was added at 10° C. and the reaction mixture stirred for another 2 hours. The mixture was poured into 20% NaOAc (100 mL) and stirred at 20° C. for 10 min. The mixture was extracted with DCM (2×100 m) and the combined organics were washed with water (3×100 mL), brine 100 mL, dried over anhydrous Na₂SO₄, filtered, and concentrated under reduced pressure. The resulting residue was purified by silica gel column chromatography (10-20% EtOAc/petroleum ether) to afford N-[2-bromo-5-(2,3-dichlorophenyl)thiazol-4-yl]acetamide (2.2 g, 5.9 mmol, 67.8% yield) as a white solid.

Step 3: To a solution of N-[2-bromo-5-(2,3-dichlorophenyl)thiazol-4-yl]acetamide (500.0 mg, 1.4 mmol) in DMSO (20.0 mL) was added tert-butyl N-(4-methyl-4-piperidyl)carbamate (323.0 mg, 1.5 mmol) and DIEA (1.1 g, 8.6 mmol, 1.5 mL). The reaction mixture was stirred at 80° C. for 20 hours. The mixture was cooled to room temperature and ethyl acetate (150 mL) was added. The organics were washed with H₂O (3×80 mL), brine (80 mL), dried over anhydrous Na₂SO₄, filtered, and concentrated under reduced pressure to afford tert-butyl (1-(4-acetamido-5-(2,3-dichlorophenyl)thiazol-2-yl)-4-methylpiperidin-4-yl)carbamate (747.0 mg, 84.3% yield) as a brown solid. This material was used as is without further purification.

Step 4: 4N HCl/MeOH (2.0 mL) was added to a solution of tert-butyl N-[1-[4-acetamido-5-(2,3-dichlorophenyl)thiazol-2-yl]-4-methyl-4-piperidyl]carbamate (200.0 mg, 0.4 mmol) in MeOH (10.0 mL). The reaction mixture was stirred at 25° C. for 20 h. The mixture was concentrated under reduced pressure to give a residue, which was purified by reversed phase HPLC to afford Compound 1, 2-(4-amino-4-methyl-1-piperidyl)-5-(2,3-dichlorophenyl)thiazol-4-amine, dihydrochloride (61.0 mg, 35.4% yield) as a off-white solid: ESMS [M+H]⁺=357.0; ¹HNMR (400 MHz, DMSO-d₆): δ 7.71-7.69 (d, 1H), 7.36-7.32 (m, 7.50-7.44, 1H), 6.75-6.74 (br, 1H), 4.74-4.66 (m, 1H), 3.92-3.84 (m, 3H), 2.10-2.06 (t, 4H), 1.58-1.60 (d, 3H).

### Preparation of (2-(3-(aminomethyl)-3-methylpyrrolidin-1-yl)thiazol-5-yl)(3,4-dihydro-1,5-naphthyridin-1(2H)-yl)methanone (Compound B2)

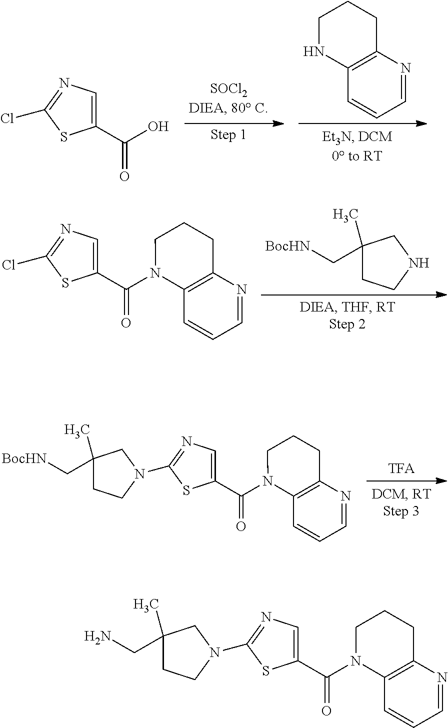 [SMILES: O=C(O)c1cnc(Cl)s1 ; c1cnc2c(c1)NCCC2 ; O=C(c1cnc(Cl)s1)N1CCCc2ncccc21 ; CC1(CNC(=O)OC(C)(C)C)CCNC1 ; CC1(CNC(=O)OC(C)(C)C)CCN(c2ncc(C(=O)N3CCCc4ncccc43)s2)C1 ; CC1(CN)CCN(c2ncc(C(=O)N3CCCc4ncccc43)s2)C1]

Step 1: A resealable reaction vial was charged with 5-chloropyrazine-2-carboxylic acid (200 mg, 1.26 mmol) and thionyl chloride (2.29 mL, 31.5 mmol). The resulting mixture was stirred at 80° C. for 3 hours. After cooling to RT, the volatiles were removed under reduced pressure and the residue taken up in 10 mL of toluene, which was also removed under reduced pressure (3×) to remove any trace thionyl chloride. The resulting solid was suspended in DCM (2 mL), cooled to 0-5° C. and treated with triethylamine (701 μL, 5.04 mmol) (become a light brown solution) and a solution of 1,2,3,4-tetrahydro-1,5-naphthyridine (169 mg, 1.26 mmol) in DCM (1 mL). The reaction mixture was allowed to come to RT slowly and stirred overnight at RT. To the mixture was added 2 mL of sat'd NaHCO₃ and the aqueous layer extracted with DCM (2×5 mL), dried over Na₂SO₄, filtered, and concentrated under reduced pressure to give (2-chlorothiazol-5-yl)(3,4-dihydro-1,5-naphthyridin-1(2H)-yl)methanone as a brown oil. This material was used as is in subsequent reactions.

Step 2: (2-Chlorothiazol-5-yl)(3,4-dihydro-1,5-naphthyridin-1(2)-yl)methanone (198 mg, 0.71 mmol) was dissolved in THF (2 mL) and tert-butyl ((3-methylpyrrolidin-3-yl)methyl)carbamate (153 mg, 0.71 mmol) and N-ethyl-N-isopropylpropan-2-amine (246 μL, 1.42 mmol) was added. The reaction mixture was stirred at RT overnight then partitioned between EtOAc (20 mL) and satd. NaCl (10 mL). The organics were dried over Na₂SO₄, filtered, and concentrated under reduced pressure. The residue was purified by reversed-phase HPLC (10-40% ACN/0.1% formic acid). Fractions containing tert-butyl ((3-methyl-1-(5-(1,2,3,4-tetrahydro-1,5-naphthyridine-1-carbonyl)thiazol-2-yl)pyrrolidin-3-yl)methyl)carbamate were combined and the ACN removed under reduced pressure followed by lyophilization.

Step 3: The tert-butyl ((3-methyl-1-(5-(1,2,3,4-tetrahydro-1,5-naphthyridine-1-carbonyl)thiazol-2-yl)pyrrolidin-3-yl)methyl)carbamate from Step 3 was treated with DCM (5 mL) and 0.5 mL of TFA. The reaction mixture was stirred at RT for 4 hours and the volatiles removed under reduced pressure. The residue was dissolved in 5 mL toluene and the volatiles were removed under reduced pressure (2×) to remove any residual TFA. The crude product was taken up in a minimum of EtOAc and purified by flash chromatography (0-100% EtOAc in heptanes then 0-10% MeOH in EtOAc w/1% NH₄OH). Fractions containing pure product were combined, dissolved in 1:1 acetonitrile/water and lyophilized to yield Compound 2, (2-(3-(aminomethyl)-3-methylpyrrolidin-1-yl)thiazol-5-yl)(3,4-dihydro-1,5-naphthyridin-1(2H)-yl)methanone (165 mg, 0.4615 mmol): ESMS (M+H)⁺358.2; ¹H NMR (400 MHz, DMSO-d6) δ ppm 1.03 (s, 3H) 1.62-1.71 (m, 1H) 1.90-2.01 (m, 3H) 2.89 (t, J=6.71 Hz, 2H) 3.04 (br d, J=10.25 Hz, 1H) 3.28-3.36 (m, 2H) 3.45 (br t, J=6.84 Hz, 2H) 3.85-3.91 (m, 2H) 7.11 (dd, J=8.30, 4.64 Hz, 1H) 7.43 (s, 1H) 7.66 (dd, J=8.30, 1.22 Hz, 1H) 8.21 (dd, J=4.64, 1.46 Hz, 1H).

### Preparation of (1-(5-(2-chlorophenyl)thiazol-2-yl)-3-methylpyrrolidin-3-yl) methanamine formate (Compound B3)

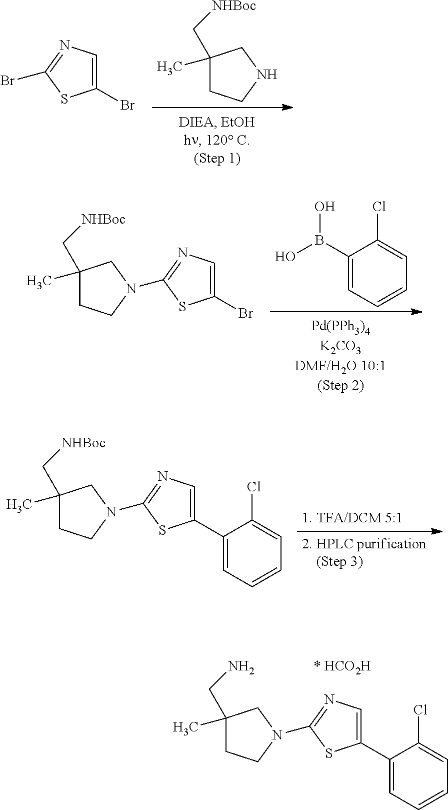 [SMILES: Brc1cnc(Br)s1 ; CC1(CNC(=O)OC(C)(C)C)CCNC1 ; CC1(CNC(=O)OC(C)(C)C)CCN(c2ncc(Br)s2)C1 ; OB(O)c1ccccc1Cl ; CC1(CNC(=O)OC(C)(C)C)CCN(c2ncc(-c3ccccc3Cl)s2)C1 ; CC1(CN)CCN(c2ncc(-c3ccccc3Cl)s2)C1]

Step 1: A mixture of 2,4-dibromothiazole (200 mg, 0.8233 mmol), tert-butyl ((3-methylpyrrolidin-3-yl)methyl)carbamate (176 mg, 0.8233 mmol), and N-ethyl-N-isopropylpropan-2-amine (282 μL, 1.64 mmol) was taken up in 5 mL ethanol in a sealed tube and heated under microwave irradiation at 120° C. for 2 hours. The reaction mixture was cooled to RT, diluted with water (5 mL), and extracted with EtOAc (2×15 mL). The combined organics were dried over Na₂SO₄, filtered, and concentrated under reduced pressure. The residue was purified by flash chromatography (0-100% EtOAc/hexanes) to give tert-butyl ((1-(4-bromothiazol-2-yl)-3-methylpyrrolidin-3-yl)methyl)carbamate (48.0 mg, 0.1275 mmol) as an oil.

Step 2: tert-Butyl ((1-(5-bromothiazol-2-yl)-3-methylpyrrolidin-3-yl)methyl)carbamate (48 mg, 0.1275 mmol), (2-chlorophenyl)boronic acid (21.9 mg, 0.1402 mmol), potassium carbonate (35.2 mg, 0.255 mmol), and tetrakis(triphenylphosphine)palladium(0) (14.7 mg, 0.01275 mmol) were dissolved in 2.5 mL DMF/water (10:1). The reaction mixture was heated under microwave irradiation at 100° C. for 30 min. The mixture was then partitioned between 5 mL water and 10 mL EtOAc and the aqueous layer washed 2× with EtOAc (10 mL). The combined organics were washed with brine (5 mL), dried over Na₂SO₄, filtered, and concentrated under reduced pressure. The intermediate tert-butyl ((1-(5-(2-chlorophenyl)thiazol-2-yl)-3-methylpyrrolidin-3-yl)methyl)carbamate was purified by reversed-phase HPLC (30-60% ACN/0.1% formic acid). Fractions containing product were combined and lyophilized to produce tert-butyl ((1-(5-(2-chlorophenyl)thiazol-2-yl)-3-methylpyrrolidin-3-yl)methyl)carbamate.

Step 3: tert-Butyl ((1-(5-(2-chlorophenyl)thiazol-2-yl)-3-methylpyrrolidin-3-yl)methyl)carbamate was taken up in 5 mL of DCM and 1 mL of TFA was added. The reaction mixture was stirred for 30 min., concentrated under reduced pressure, and the residue taken up in a minimum of DMSO then purified by reversed-phase HPLC (10-40% ACN/0.1% formic acid). Fractions containing product were combined and lyophilized to yield Compound 3, (1-(5-(2-chlorophenyl)thiazol-2-yl)-3-methylpyrrolidin-3-yl)methanamine formate (11.1 mg, 0.03136 mmol): ESMS (M+H)⁺308.0; ¹H NMR (400 MHz, DMSO-d6) δ ppm 1.15 (s, 3H) 1.84 (dt, J=12.63, 6.50 Hz, 1H) 2.01 (dt, J=12.63, 7.60 Hz, 1H) 2.86-2.97 (m, 2H) 3.23 (d, J=10.25 Hz, 1H) 3.43 (d, J=10.50 Hz, 1H) 3.49-3.55 (m, 2H) 7.25-7.31 (m, 1H) 7.35 (td, J=7.57, 1.22 Hz, 1H) 7.51 (dd, J=8.06, 1.22 Hz, 1H) 7.54 (s, 1H) 7.58 (dd, J=7.81, 1.46 Hz, 1H).

### Preparation of ((1-(5-(2-chlorophenyl)-4-methylthiazol-2-yl)-3-methylpyrrolidin-3-yl)methanamine formate (Compound B4)

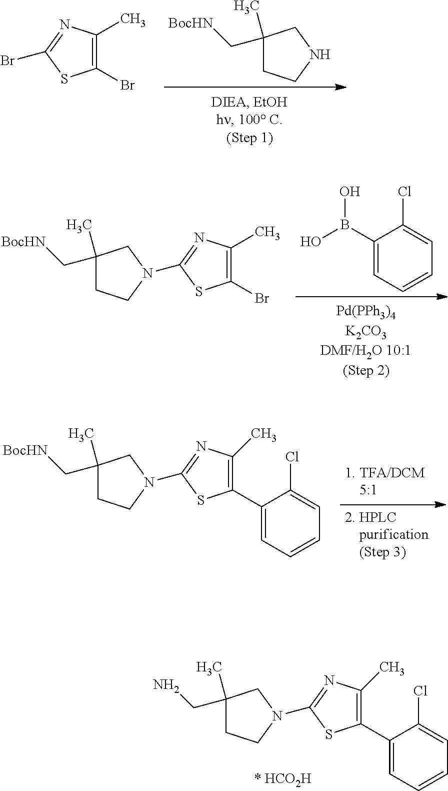

Step 1: A mixture of 2,5-dibromo-4-methylthiazole (200 mg, 0.7783 mmol), tert-butyl ((3-methylpyrrolidin-3-yl)methyl)carbamate (166 mg, 0.7783 mmol), and N-ethyl-N-isopropylpropan-2-amine (268 μL, 1.55 mmol) was taken up in 5 mL ethanol in a sealed tube and heated under microwave irradiation at 100° C. for 2 hours. The reaction mixture was cooled to RT, diluted with water (5 mL), and extracted with EtOAc (2×15 mL). The combined organics were dried over Na₂SO₄, filtered, and concentrated under reduced pressure. The residue was purified by flash chromatography (0-50% EtOAc/hexanes) to give tert-butyl ((1-(5-bromo-4-methylthiazol-2-yl)-3-methylpyrrolidin-3-yl)methyl)carbamate (96.0 mg, 0.2459 mmol) as an oil.

Step 2: tert-Butyl ((1-(5-bromo-4-methylthiazol-2-yl)-3-methylpyrrolidin-3-yl)methyl)carbamate (96 mg, 0.2459 mmol), (2-chlorophenyl)boronic acid (42.2 mg, 0.2704 mmol), potassium carbonate (67.9 mg, 0.4918 mmol), and tetrakis(triphenylphosphine)palladium(0) (28.4 mg, 0.02459 mmol) were dissolved in 5 mL DMF/water (10:1). The reaction mixture was heated under microwave irradiation at 100° C. for 1 hour. The mixture was then partitioned between 10 mL water and 20 mL EtOAc and the aqueous layer washed 2× with EtOAc (20 mL). The combined organics were washed with brine (10 mL), dried over MgSO₄, filtered, and concentrated under reduced pressure. The intermediate tert-butyl ((1-(5-(2-chlorophenyl)-4-methylthiazol-2-yl)-3-methylpyrrolidin-3-yl)methyl)carbamate was purified by flash chromatography (first 0-50% EtOAc/heptanes, then EtOAc, then 10% MeOH and 0.1% NH₄OH). Fractions containing product were combined the volatiles removed under reduced pressure to provide tert-butyl ((1-(5-(2-chlorophenyl)-4-methylthiazol-2-yl)-3-methylpyrrolidin-3-yl)methyl)carbamate.

Step 3: tert-Butyl ((1-(5-(2-chlorophenyl)-4-methylthiazol-2-yl)-3-methylpyrrolidin-3-yl)methyl)carbamate was taken up in 5 mL of DCM and 1 mL of TFA was added. The reaction mixture was stirred for 30 min., concentrated under reduced pressure, and the residue taken up in a minimum of DMSO then purified by reversed-phase HPLC (5-30% ACN/0.1% formic acid). Fractions containing product were combined and lyophilized to yield Compound 4, ((1-(5-(2-chlorophenyl)-4-methylthiazol-2-yl)-3-methylpyrrolidin-3-yl)methanamine formate (8.90 mg, 0.02419 mmol) as a white solid: ESMS (M+H)⁺322.1; ¹H NMR (400 MHz, DMSO-d6) δ ppm 1.15 (s, 3H) 1.83 (dt, J=12.70, 6.35 Hz, 1H) 1.95-2.04 (m, 4H) 2.93 (br d, J=2.20 Hz, 2H) 3.20 (d, J=10.25 Hz, 1H) 3.39 (d, J=10.25 Hz, 1H) 3.47 (br t, J=7.20 Hz, 2H) 6.51 (br s, 1H) 7.35-7.43 (m, 3H) 7.52-7.58 (m, 1H) 7.82 (br s, 3H) 8.12 (br s, 1H).

Using the general procedures described above, with reagents, starting materials, and conditions familiar to those skilled in the art, the following compounds representative of the disclosure are prepared:

| Compound | Structure |
|---|---|
| B5 | 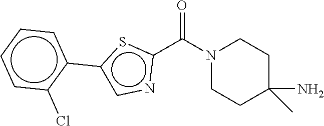 |

### Preparation of (4-amino-4-methylpiperidin-1-yl)(2-(2-chlorophenyl)thiazol-4-yl) methanone formate (Compound C1)

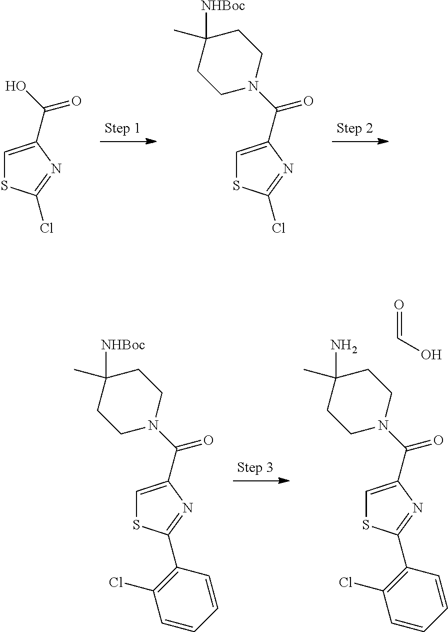

Step 1: A round bottomed flask was charged with 2-chlorothiazole-4-carboxylic acid (75 mg, 0.4584 mmol), N-ethyl-N-isopropylpropan-2-amine (118 mg, 0.9168 mmol), and a stirbar. Dimethylformamide (2 mL) was added, followed by 1-((dimethylamino)(dimethyliminio)methyl)-3-oxo-1,3-dihydro-[1,2,3]triazolo[4,5-b]pyridin-3-ium-2-ide hexafluorophosphate (V) (209 mg, 0.5500 mmol) and tert-butyl (4-methylpiperidin-4-yl)carbamate (117 mg, 0.5500 mmol). The solution was stirred at 20° C. for 5 h. DMF was removed in a Genevac. The resulting crude material was purified by silica gel chromatography (10 g column; eluting with heptanes/ethyl acetate). Concentration in vacuo resulted in tert-butyl (1-(2-chlorothiazole-4-carbonyl)-4-methylpiperidin-4-yl)carbamate (118 mg, 70%) as a white amorphous solid. LCMS M/Z: 382 (M+Na).

Step 2: A microwave vial was charged with tert-butyl (1-(2-chlorothiazole-4-carbonyl)-4-methylpiperidin-4-yl)carbamate (118 mg, 0.3278 mmol), (2-chlorophenyl)boronic acid (61.5 mg, 0.3933 mmol), K₂CO₃ (54.3 mg, 0.3933 mmol), and a stirbar. Dioxane (2 mL) and water (0.2 mL) were added, and the solution was degassed for 5 min. X-Phos G4 (2.82 mg, 0.003278 mmol) was added, the vial was sealed, and the mixture was stirred in the microwave at 120° C. for 30 min. The solvent was removed in vacuo and the resulting crude material was purified by silica gel chromatography (10 g column; eluting with heptanes/ethyl acetate; ratio). Concentration in vacuo resulted in tert-butyl (1-(2-(2-chlorophenyl)thiazole-4-carbonyl)-4-methylpiperidin-4-yl)carbamate (69 mg, 48%) as a green glass. LCMS M/Z: 458 (M+Na).

Step 3: A round bottomed flask was charged with tert-butyl (1-(2-(2-chlorophenyl)thiazole-4-carbonyl)-4-methylpiperidin-4-yl)carbamate (69 mg, 0.1582 mmol) in dioxane (1 mL) and a stirbar. 4 N HCl in dioxane (600 uL) was added, and the solution was stirred at 20° C. The solvent was removed in vacuo, and the resulting crude material was purified by HPLC (acetonitrile/water/0.1% formic acid). Lyophilization yielded (4-amino-4-methylpiperidin-1-yl)(2-(2-chlorophenyl)thiazol-4-yl)methanone formate (30 mg, 50%) as a white amorphous solid. LCMS M/Z: 358/360 (M+Na). ¹H NMR (400 MHz, METHANOL-d₄) δ ppm 1.48-1.54 (m, 4H) 1.84-2.01 (m, 5H) 3.36-3.48 (m, 1H) 3.57-3.70 (m, 1H) 4.29-4.40 (m, 3H), 7.43-7.54 (m, 2H) 7.57-7.65 (m, 1H) 8.10-8.14 (m, 1H) 8.19-8.25 (m, 1H) 8.50-8.56 (m, 1H).

Additional compounds of the present disclosure are shown below.

| Compound | Structure |
|---|---|
| C2 | 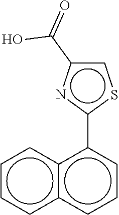 |
| C3 | 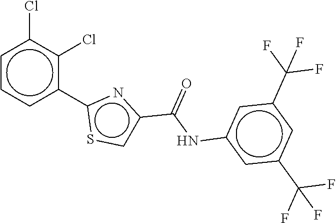 |
| C4 | 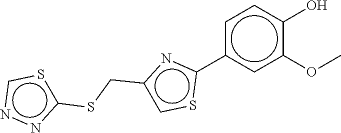 |
| C5 | 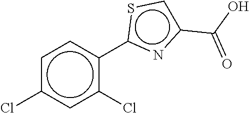 |
| C6 | 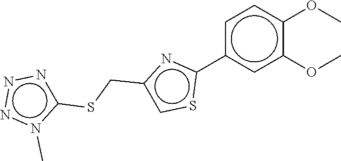 |
| C7 | 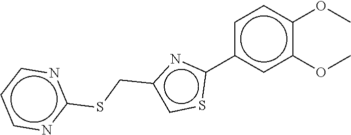 |
| C8 | 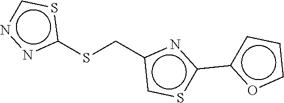 |
| C9 | 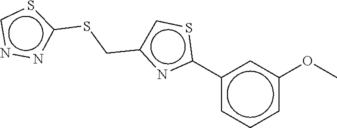 |
| C10 | 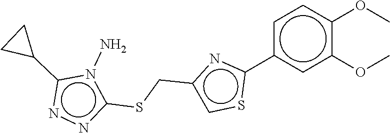 |
| C11 | 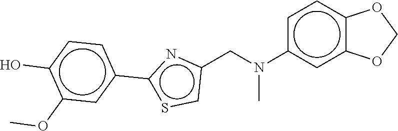 |
| C12 | 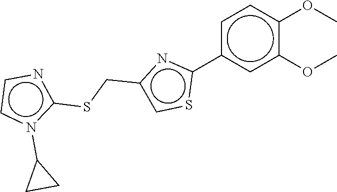 |
| C13 | 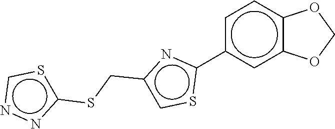 |
| C14 | 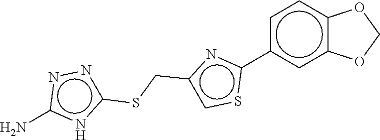 |
| C15 | 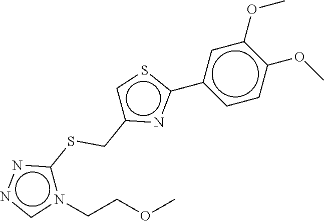 |
| C16 | 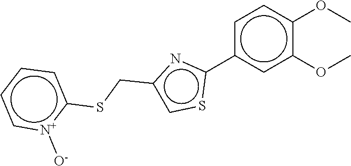 |
| C17 | 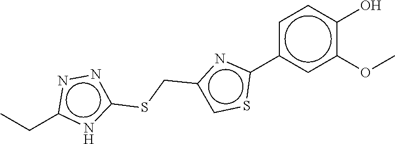 |
| C18 | 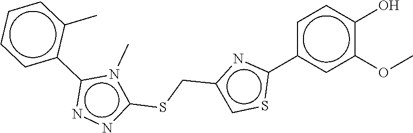 |
| C19 | 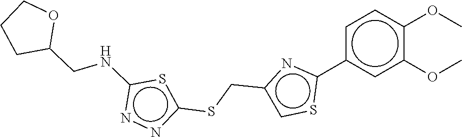 |
| C20 | 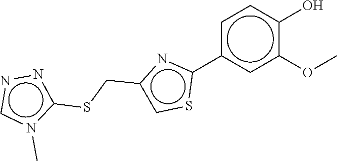 |
| C21 | 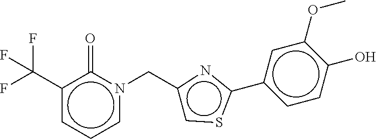 |
| C22 | 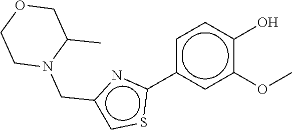 |
| C23 | 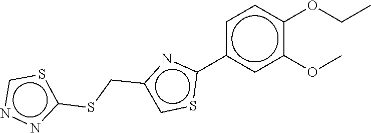 |
| C24 | 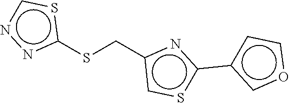 |
| C25 | 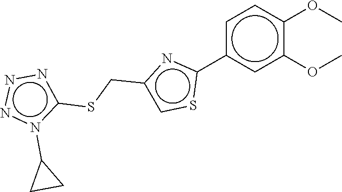 |
| C26 | 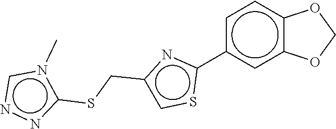 |
| C27 | 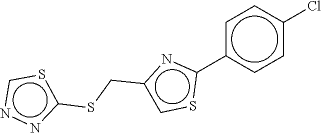 |
| C28 | 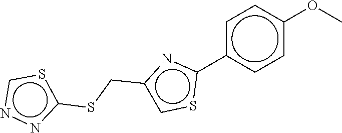 |
| C29 | 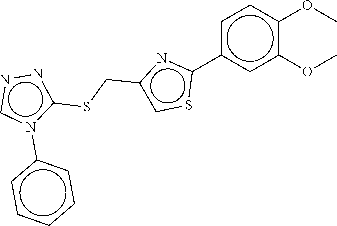 |
| C30 | 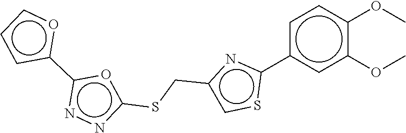 |
| C31 | 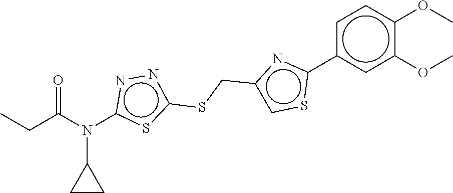 |
| C32 | 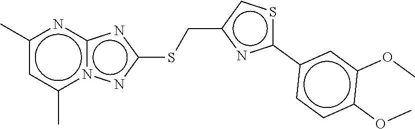 |
| C33 | 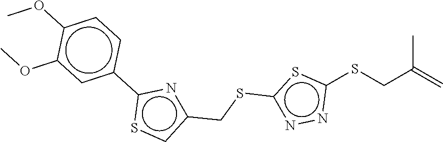 |
| C34 | 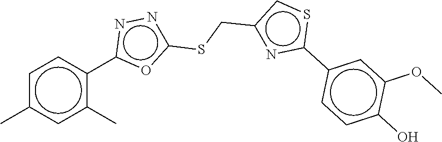 |
| C35 | 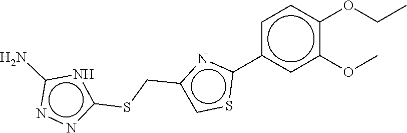 |
| C36 | 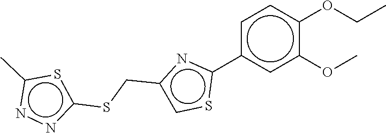 |
| C37 | 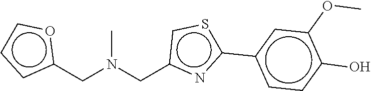 |
| C38 | 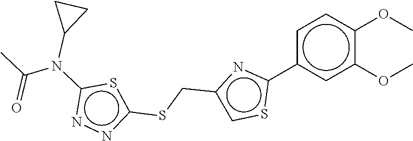 |
| C39 | 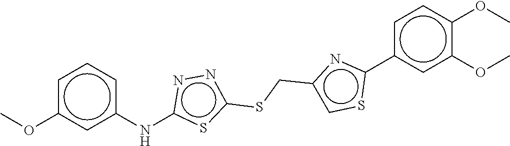 |
| C40 | 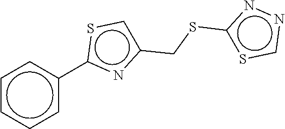 |
| C41 | 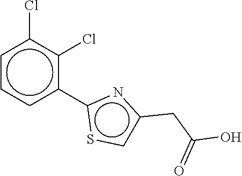 |
| C42 | 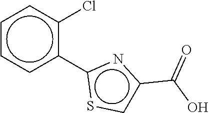 |
| C43 | 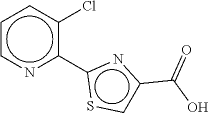 |
| C44 | 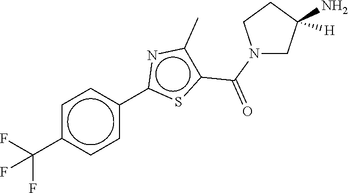 |
| C45 | 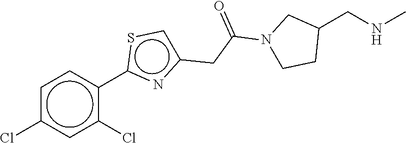 |
| C46 | 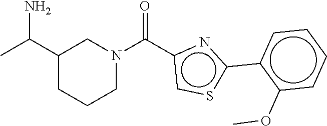 |
| C47 | 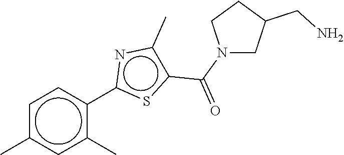 |
| C48 | 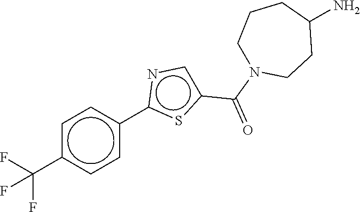 |
| C49 | 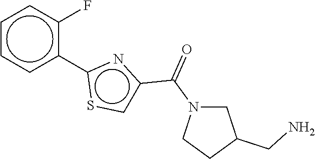 |

### Preparation of 1-(7-(2,3-dichlorophenyl)thieno[3,2-d]pyrimidin-4-yl)-4-methylpiperidin-4-amine formate (Compound D1)

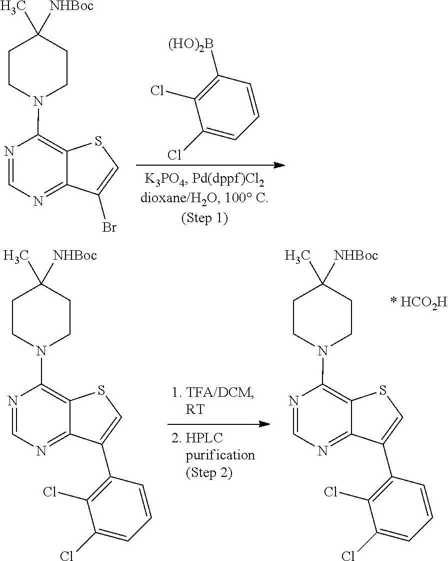 [SMILES: CC1(NC(=O)OC(C)(C)C)CCN(c2ncnc3c(Br)csc23)CC1 ; OB(O)c1cccc(Cl)c1Cl ; CC1(NC(=O)OC(C)(C)C)CCN(c2ncnc3c(-c4cccc(Cl)c4Cl)csc23)CC1 ; CC1(NC(=O)OC(C)(C)C)CCN(c2ncnc3c(-c4cccc(Cl)c4Cl)csc23)CC1]

Step 1: tert-Butyl (1-(7-bromothieno[3,2-d]pyrimidin-4-yl)-4-methylpiperidin-4-yl)carbamate (45.3 mg, 0.106 mmol), (2,3-dichlorophenyl)boronic acid (30.3 mg, 0.158 mmol), and Methanesulfonato(2-dicyclohexylphosphino-2′,4′,6′-tri-i-propyl-1,1′-biphenyl)(2′-methylamino-1,1′-biphenyl-2-yl)palladium(II) (4.6 mg, 0.0053 mmol) were placed in a reaction vessel and the reaction mixture was evacuated and refilled for 3 times with nitrogen gas. A degassed solution of K₃PO₄ (106 mg, 0.318 mmol in 0.1 mL H₂O) and tetrahydrofuran (0.1 mL) were added and the reaction was stirred at 80° C. for 12 hours. The reaction mixture was partitioned between diethyl ether and brine and the organic layer was extracted with diethyl ether (3×). Combined organics were concentrated under reduced pressure and purified by flash chromatography (8-66% EtOAc/hexanes) to provide tert-butyl (1-(7-(2,3-dichlorophenyl)thieno[3,2-d]pyrimidin-4-yl)-4-methylpiperidin-4-yl)carbamate (22.8 mg, 0.046 mmol) as a yellow oil.

Step 2: tert-Butyl (1-(7-(2,3-dichlorophenyl)thieno[3,2-d]pyrimidin-4-yl)-4-methylpiperidin-4-yl)carbamate (22.8 mg, 0.046 mmol) was dissolved in 0.5 mL DCM and 35 uL of TFA was added. After stirring for 2 h at RT, the reaction mixture was concentrated under reduced pressure. The residue was dissolved in about 5 mL toluene and the volatiles were again removed under reduced pressure (2×) to remove any residual TFA. The crude product was dissolved in a minimum of DMSO and purified by reversed-phase HPLC (10-40% ACN/0.1% formic acid). The fractions containing product were combined and lyophilized after removal of the bulk of the ACN to yield Compound 1, 1-(7-(2,3-dichlorophenyl)thieno[3,2-d]pyrimidin-4-yl)-4-methylpiperidin-4-amine formate (6.1 mg, 0.0155 mmol): ESMS (M+H)⁺=393.199; ¹H NMR (400 MHz, DMSO-d6) δ ppm 1.56 (s, 3H) 1.89-2.01 (m, 4H) 3.71-3.78 (ddd, 2H) 4.60-4.65 (dt, 2H) 7.35-7.40 (m, 2H) 7.63-7.64 (dd, 1H) 8.10 (s, 1H) 8.45 (s, 1H).

### Preparation of (1-(7-(2,3-dichlorophenyl)thieno[3,2-d]pyrimidin-4-yl)-4-methylpiperidin-4-yl)methanamine (Compound D2)

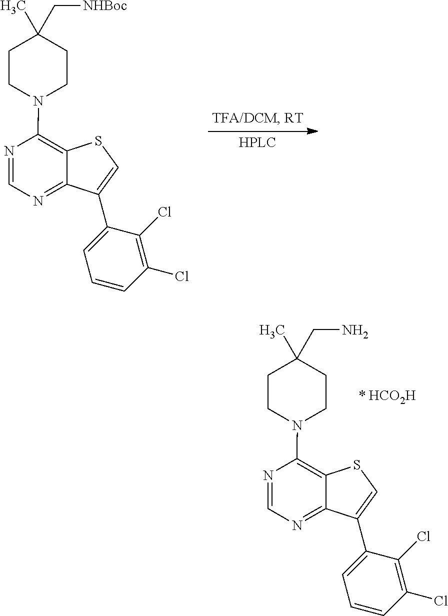 [SMILES: CC1(CNC(=O)OC(C)(C)C)CCN(c2ncnc3c(-c4cccc(Cl)c4Cl)csc23)CC1 ; CC1(CN)CCN(c2ncnc3c(-c4cccc(Cl)c4Cl)csc23)CC1]

tert-butyl((1-(7-(2,3-dichlorophenyl)thieno[3,2-d]pyrimidin-4-yl)-4-methylpiperidin-4-yl)methyl)carbamate (39 mg, 0.077 mmol) was dissolved in 0.75 mL DCM and 30 uL of TFA was added. After stirring for 1 h at RT, the reaction mixture was concentrated under reduced pressure. The residue was dissolved in about 5 mL toluene and the volatiles were again removed under reduced pressure (2×) to remove any residual TFA. The crude product was dissolved in a minimum of DMSO and purified by flash silica gel chromatography (1-10% MeOH in Dichloromethane with 1% NH₄OH). The fractions containing product were combined to yield Compound 2, (1-(7-(2,3-dichlorophenyl) thieno[3,2-d]pyrimidin-4-yl)-4-methylpiperidin-4-yl)methanamine (31 mg, 0.076 mmol): ESMS (M+H)⁺=406.951/408.802; ¹H NMR (400 MHz, DMSO-d₆) δ ppm 8.43 (s, 1H) 8.32 (s, 1H) 7.60-7.77 (m, 1H) 7.33-7.54 (m, 2H) 4.24 (dt, J=9.16, 4.46 Hz, 2H) 3.72 (ddd, J=13.31, 9.89, 2.93 Hz, 2H) 3.34-3.46 (m, 2H) 1.53-1.67 (m, 2H) 1.38-1.52 (m, 2H) 1.00-1.15 (m, 3H).

Using the general procedures described above, with reagents, starting materials, and conditions familiar to those skilled in the art, the following compounds representative of the disclosure are prepared:

| Compound | Structure |
|---|---|
| D3 | 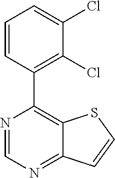 [SMILES: Clc1cccc(-c2ncnc3ccsc23)c1Cl] |
| D4 | 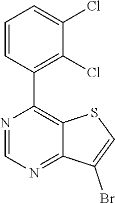 [SMILES: Clc1cccc(-c2ncnc3c(Br)csc23)c1Cl] |
| D5 | 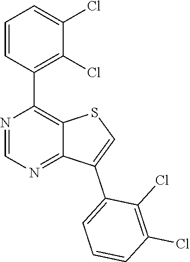 [SMILES: Clc1cccc(-c2csc3c(-c4cccc(Cl)c4Cl)ncnc23)c1Cl] |

### Preparation of 1-(3-(2-chlorophenyl)pyrazolo[5,1-b]oxazol-7-yl)-4-methylpiperidin-4-amine formate (Compound E1)

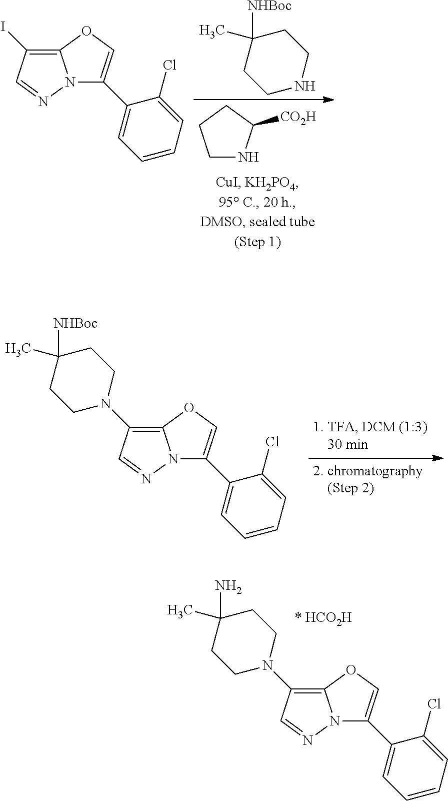

Step 1: To a resealable reaction vial was added 3-(2-chlorophenyl)-7-iodopyrazolo[5,1-b]oxazole (100 mg, 0.29 mmol), tert-butyl (4-methylpiperidin-4-yl)carbamate (124 mg, 0.58 mmol), L-proline (13.3 mg, 0.116 mmol), potassium dihydrogenphosphate (78.9 mg, 0.584 mmol) and copper(I) iodide (11.0 mg, 0.058 mmol). The tube was sealed, evacuated, backfilled with nitrogen gas, followed by the addition of DMSO (2 mL). The reaction mixture was stirred at 95° C. for 20 hours. After cooling to room temperature, the reaction mixture was diluted with sat. NaHCO₃ (5 mL) and extracted twice with EtOAc (10 mL). The organics were combined, washed with brine, dried over Na₂SO₄, filtered, concentrated under reduced pressure, and purified by flash chromatography on silica gel (0-30% EtOAc/heptanes) to produce tert-butyl (1-(3-(2-chlorophenyl)pyrazolo[5,1-b]oxazol-7-yl)-4-methylpiperidin-4-yl)carbamate.

Step 2: tert-Butyl (1-(3-(2-chlorophenyl)pyrazolo[5,1-b]oxazol-7-yl)-4-methylpiperidin-4-yl)carbamate was dissolved in 3 mL DCM and 1 mL of TFA was added. After stirring for 30 minutes at RT, the reaction mixture was concentrated under reduced pressure. The residue was dissolved in 5 mL toluene and the volatiles removed under reduced pressure (2×) to remove any residual TFA. The crude product was dissolved in a minimum of DMSO and purified by reversed-phase HPLC (10-40% ACN/0.1% formic acid). The fractions containing product were combined and lyophilized to yield Compound 1, 1-(3-(2-chlorophenyl)pyrazolo[5,1-b]oxazol-7-yl)-4-methylpiperidin-4-amine formate (9.20 mg, 0.02441 mmol): ESMS (M+H)⁺=331.09; ¹H NMR (400 MHz, DMSO-d6) δ ppm 1.24 (s, 3H) 1.64-1.78 (m, 5H) 2.95 (ddd, J=11.96, 8.42, 3.54 Hz, 2H) 3.15-3.22 (m, 3H) 7.53 (dtd, J=18.98, 7.54, 7.54, 1.59 Hz, 3H) 7.60 (d, J=0.98 Hz, 1H) 7.68 (dd, J=7.81, 1.46 Hz, 1H) 8.31 (s, 1H) 8.48 (dd, J=7.69, 1.83 Hz, 1H) 8.58 (d, J=0.98 Hz, 1H).

### Preparation of (1-(3-(2-chlorophenyl)pyrazolo[5,1-b]oxazol-7-yl)-3-methylpyrrolidin-3-yl)methanamine (Compound E2)

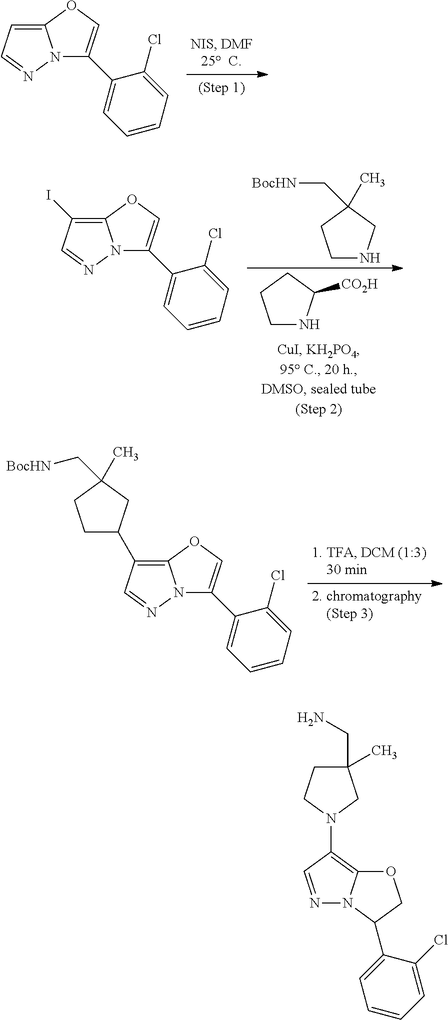

Step 1: To a solution of 3-(2-chlorophenyl)pyrazolo[5,1-b]oxazole (0.200 g, 0.9147 mmol) in DMF (5 mL) was added 1-iodopyrrolidine-2,5-dione (245 mg, 1.09 mmol). The reaction mixture was stirred at RT for 4 hours and diluted with 20 mL EtOAc. The organics were washed with 10% sodium thiosulfate (5 mL), brine, and dried over Na₂SO₄, filtered, and concentrated under reduced pressure. Purification by flash chromatography (0-30% EtOAc/heptanes) provided 3-(2-chlorophenyl)-7-iodopyrazolo[5,1-b]oxazole (334 mg, 0.9694 mmol) as a white solid.

Step 2: To a resealable reaction vial was added 3-(2-chlorophenyl)-7-iodopyrazolo[5,1-b]oxazole (50 mg, 0.145 mmol), tert-butyl ((3-methylpyrrolidin-3-yl)methyl)carbamate (62 mg, 0.29 mmol), L-proline (6.7 mg, 0.058 mmol), potassium dihydrogenphosphate (39.4 mg, 0.29 mmol) and copper(I) iodide (5.5 mg, 0.029 mmol). The tube was sealed, evacuated, backfilled with nitrogen gas, and DMSO (2 mL) was added. The reaction mixture was stirred at 95° C. for 18 hours. After cooling to room temperature, the mixture was diluted with sat. NaHCO₃ (5 mL) and extracted with EtOAc (2×10 mL). The organics were combined, washed with brine, dried over Na₂SO₄, filtered, concentrated under reduced pressure, and purified by flash chromatography (0-30% EtOAc/heptanes) to produce tert-butyl ((1-(3-(2-chlorophenyl)pyrazolo[5,1-b]oxazol-7-yl)-3-methylpyrrolidin-3-yl)methyl)carbamate.

Step 3: tert-Butyl ((1-(3-(2-chlorophenyl)pyrazolo[5,1-b]oxazol-7-yl)-3-methylpyrrolidin-3-yl)methyl)carbamate was dissolved in 3 mL DCM and 1 mL of TFA was added. After stirring for 30 minutes at RT, the reaction mixture was concentrated under reduced pressure. The residue was dissolved in 5 mL toluene and the volatiles were removed under reduced pressure (2×) to remove any residual TFA. The crude product was dissolved in a minimum of DMSO and purified by reversed-phase HPLC (5-40% ACN/0.1% formic acid). The fractions containing product were combined and lyophilized to yield Compound 2, (1-(3-(2-chlorophenyl)pyrazolo[5,1-b]oxazol-7-yl)-3-methylpyrrolidin-3-yl)methanamine (2 mg, 0.002 mmol): ESMS (M+H)⁺=331.09.

### Preparation of 3-(2-chlorophenyl-6-methylpyrazolo[5,1-b]oxazole (Compound E3)

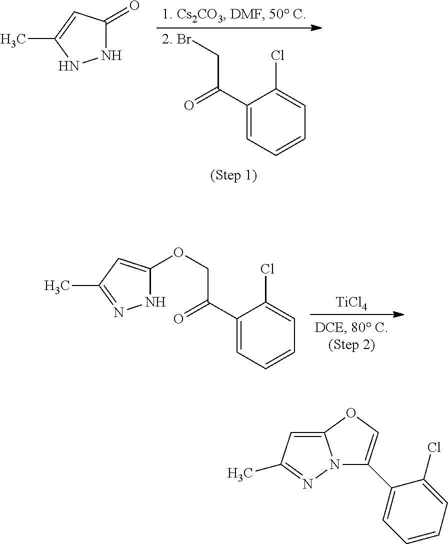

Step 1: 5-Methyl-1H-pyrazol-3(2H)-one (1 g, 10.1 mmol) was dissolved in DMF (10 mL) and cesium carbonate (3.61 g, 11.1 mmol) was added. The reaction mixture was heated to 50° C. After 5 min. 2-bromo-1-(2-chlorophenyl)ethanone (2.35 g, 10.1 mmol) in DMF (12 mL) was slowly added over 10 min. The reaction mixture was stirred for 1 hour at 50° C. After cooling to RT, the mixture was partitioned between EtOAc (50 mL) and saturated NaHCO₃ (20 mL). The aqueous layer was extracted with EtOAc and the combined organics were washed with water, washed with brine, dried over Na₂SO₄, filtered, and concentrated under reduced pressure. The residue was purified by flash chromatography (0-40% EtOAc) to give 1-(2-chlorophenyl)-2-((3-methyl-1H-pyrazol-5-yl)oxy)ethanone (1.24 g, 4.97 mmol) as a yellow oil.

Step 2: 1-(2-Chlorophenyl)-2-((3-methyl-1H-pyrazol-5-yl)oxy)ethan-1-one (250 mg, 1.00 mmol) was dissolved in dichloroethane (5 mL) and titanium(IV) chloride (130 μL, 1.19 mmol) was added under an atmosphere of nitrogen. The reaction mixture was stirred at 80° C. under N₂ for 4 hours. After cooling, satd. NaHCO₃ (about 5 mL) was added followed by the addition of EtOAc (10 mL). An emulsion was formed which was broken up by passing the mixture through a pad of diatomaceous earth. The aqueous layer was extracted with EtOAc (10 mL) and the combined organics dried over Na₂SO₄, filtered, and concentrated under reduced pressure. The residue was purified by flash chromatography on silica gel (0-30% EtOAc/heptanes) to yield Compound 3, 3-(2-chlorophenyl)-6-methylpyrazolo[5,1-b]oxazole (154 mg, 0.66 mmol), as a white solid: ESMS (M+H)⁺=233.

### Preparation of 3-(2-chlorophenyl)pyrazolo[5,1-b]oxazole (Compound E4)

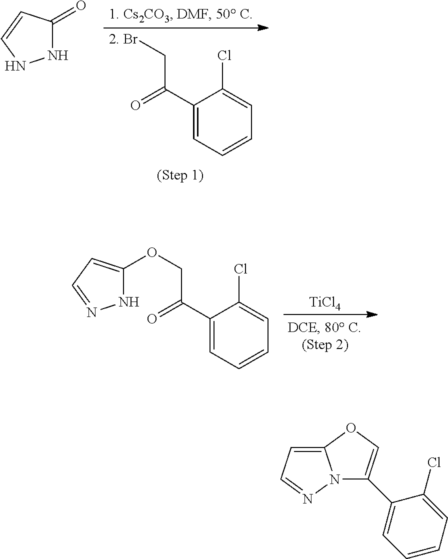

Step 1: 1H-pyrazol-3(2H)-one (1 g, 11.8 mmol) was dissolved in DMF (10 mL) and cesium carbonate (4.20 g, 12.9 mmol) was added. The reaction mixture was heated to 50° C. After 5 minutes, 2-bromo-1-(2-chlorophenyl)ethanone (2.35 g, 10.1 mmol) in DMF (12 mL) was slowly added over 10 min. The reaction mixture was stirred for 1 hour at 50° C. After cooling to RT, the mixture was partitioned between EtOAc (50 mL) and saturated NaHCO₃ (20 mL). The aqueous layer was washed with EtOAc and the combined organics were washed with water, washed with brine, dried over Na₂SO₄, filtered, and concentrated under reduced pressure. The residue was purified by flash chromatography (0-40% EtOAc) to give 2-((1H-pyrazol-5-yl)oxy)-1-(2-chlorophenyl)ethanone (1.08 g, 4.57 mmol) as a yellow oil: ¹H NMR (400 MHz, DMSO-d6) δ ppm 5.25 (s, 2H) 5.66 (t, J=2.20 Hz, 1H) 7.43-7.48 (m, 1H) 7.49 (t, J=1.95 Hz, 1H) 7.51-7.57 (m, 2H) 7.72 (d, J=8.06 Hz, 1H) 11.88 (br s, 1H).

Step 2: 2-((1H-Pyrazol-5-yl)oxy)-1-(2-chlorophenyl)ethan-1-one (1.00 g, 4.22 mmol) was dissolved in dichloroethane (21 mL) and titanium(IV) chloride (554 μL, 5.06 mmol) was added under an atmosphere of nitrogen. The reaction mixture was stirred at 80° C. under N₂ for 2 hours. After cooling, satd. NaHCO₃ (about 20 mL) was added followed by the addition of EtOAc (40 mL). An emulsion formed, which was broken up by passing the mixture through a pad of diatomaceous earth. The aqueous layer was extracted with EtOAc (40 mL) and the combined organics dried over Na₂SO₄, filtered, and concentrated under reduced pressure. The residue was purified by flash chromatography on silica gel (0-30% EtOAc/heptanes) to yield Compound 4, 3-(2-chlorophenyl)pyrazolo[5,1-b]oxazole (429 mg, 1.96 mmol), as a white solid: ESMS (M+H)⁺=219; ¹H NMR (400 MHz, DMSO-d6) δ ppm 6.13 (d, J=2.20 Hz, 1H) 7.49-7.61 (m, 2H) 7.69 (dd, J=7.81, 1.22 Hz, 1H) 7.81 (dd, J=1.95, 1.22 Hz, 1H) 8.50 (dd, J=7.57, 1.71 Hz, 1H) 8.64 (d, J=0.98 Hz, 1H).

### Preparation of 6-(4-(aminomethyl)-4-methylpiperidin-1-yl)-3-(3,4-dihydro-1,5-naphthyridin-1(2H)-yl)-5-methyl-1H-pyrazolo[3,4-d]pyrimidin-4(5H)-one, hydrochloride (Example F1)

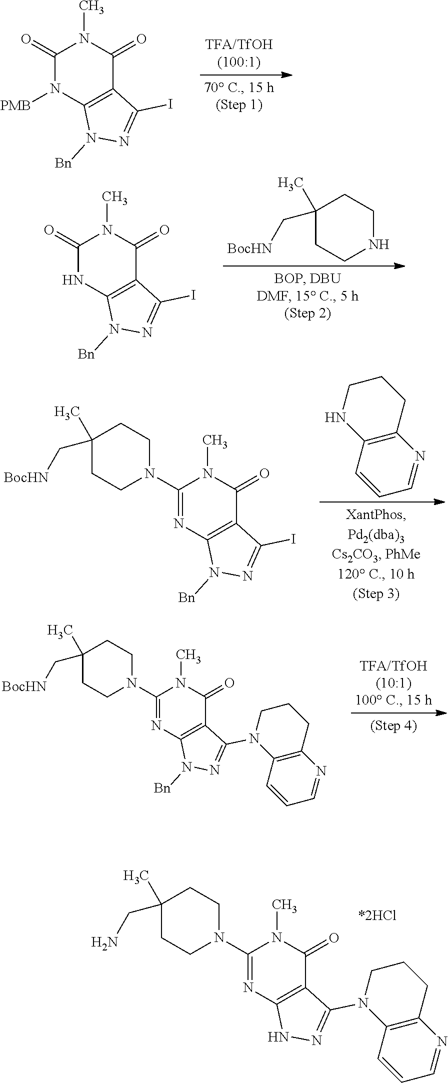

Step 1: A mixture of 1-benzyl-3-iodo-7-(4-methoxybenzyl)-5-methyl-1H-pyrazolo[3,4-d]pyrimidine-4,6(5H, 7H)-dione (1.0 g, 2.0 mmol) in TFA (10 mL) and TfOH (0.1 mL) was stirred at 70° C. for 15 hours. The mixture was concentrated to give a residue, which was triturated with saturated aqueous NH₄Cl (100 mL) and extracted with EtOAc (100 mL×2). The combined organics were concentrated under reduced pressure to give a residue, which was triturated with EtOAc (3 mL). The resulting precipitate was collected by filtration to afford 1-benzyl-3-iodo-5-methyl-1H-pyrazolo[3,4-d]pyrimidine-4,6(5H,7H)-dione (600 mg, 79% yield) as a green solid: ESMS (M+H)⁺=383.

Step 2: A mixture of 1-benzyl-3-iodo-5-methyl-1H-pyrazolo[3,4-d]pyrimidine-4,6(5H,7H)-dione (200 mg, 0.5 mmol, 1.0 eq), tert-butyl ((4-methylpiperidin-4-yl)methyl)carbamate (119 mg, 0.5 mmol, 1.0 eq), BOP (658 mg, 1.6 mmol, 3.0 eq) and DBU (476 mg, 3.1 mmol, 6.0 eq) in DMF (5 mL) was stirred at 15° C. for 5 hours. The mixture was diluted with H₂O (20 mL) and extracted with EtOAc (50 mL). The organics were washed with brine (2×20 mL), dried over anhydrous Na₂SO₄, filtered, and concentrated under reduced pressure to give a residue which was purified by flash chromatography (0-50% EtOAc/petroleum ether) to afford tert-butyl ((1-(1-benzyl-3-iodo-5-methyl-4-oxo-4,5-dihydro-1H-pyrazolo[3,4-d]pyrimidin-6-yl)-4-methylpiperidin-4-yl)methyl)carbamate (220 mg, 71% yield) as a yellow solid.

Step 3: A mixture of tert-butyl ((1-(1-benzyl-3-iodo-5-methyl-4-oxo-4,5-dihydro-1H-pyrazolo[3,4-d]pyrimidin-6-yl)-4-methylpiperidin-4-yl)methyl)carbamate (220 mg, 0.4 mmol), 1,2,3,4-tetrahydro-1,5-naphthyridine (55 mg, 0.4 mmol), XantPhos (64 mg, 0.1 mmol, 0.3 eq), Pd₂(dba)₃ (101 mg, 0.1 mmol, 0.3 eq) and Cs₂CO₃ (361 mg, 1.1 mmol, 3.0 eq) in toluene (5 mL) was evacuated and refilled 3 times with nitrogen gas. The mixture was stirred at 120° C. for 10 hours under N₂ and concentrated under reduced pressure to give a residue which was purified by flash chromatography (10-100% EtOAc/petroleum ether) to afford tert-butyl ((1-(1-benzyl-3-(3,4-dihydro-1,5-naphthyridin-1(2H)-yl)-5-methyl-4-oxo-4,5,6,7-tetrahydro-1H-pyrazolo[3,4-d]pyrimidin-6-yl)-4-methylpiperidin-4-yl)methyl)carbamate (50 mg, 22% yield) as a yellow oil.

Step 4: A mixture of tert-butyl ((1-(1-benzyl-3-(3,4-dihydro-1,5-naphthyridin-1(2H)-yl)-5-methyl-4-oxo-4,5-dihydro-1H-pyrazolo[3,4-d]pyrimidin-6-yl)-4-methylpiperidin-4-yl)methyl)carbamate (35 mg, 0.06 mmol) in TFA (2 mL) and TfOH (0.2 mL) was stirred at 100° C. for 15 hours. The mixture was concentrated under reduced pressure to give a residue which was purified by reversed-phase HPLC to afford 6-(4-(aminomethyl)-4-methylpiperidin-1-yl)-3-(3,4-dihydro-1,5-naphthyridin-1(2H)-yl)-5-methyl-1H-pyrazolo[3,4-d]pyrimidin-4(5H)-one, hydrochloride (28.9 mg) as a yellow solid: ESMS (M+H)⁺=409.1; ¹HNMR (400 MHz, methanol-d₄) δ 7.86-7.84 (d, J=5.2 Hz, 1H), 7.60-7.57 (d, J=8.8 Hz, 1H), 7.37-7.34 (m, 1H), 3.75-3.72 (m, 2H), 3.12 (s, 3H), 3.10-3.01 (m, 4H), 2.76 (s, 2H), 2.10-2.06 (m, 2H), 1.58-1.55 (m, 2H), 1.47-1.43 (m, 2H), 1.14-1.10 (m, 2H), 1.00 (s, 3H).

### Preparation of 5-(4-amino-4-methylpiperidin-1-yl)-3-(3-chloro-2-fluorophenyl)-[1,2,4]triazolo[4,3-a]pyrimidin-7(1H)-one hydrochloride (Example 2) and 7-(4-amino-4-methylpiperidin-1-yl)-3-(3-chloro-2-fluorophenyl)-[1,2,4]triazolo[4,3-a]pyrimidin-5(1H)-one hydrochloride (Example F3)

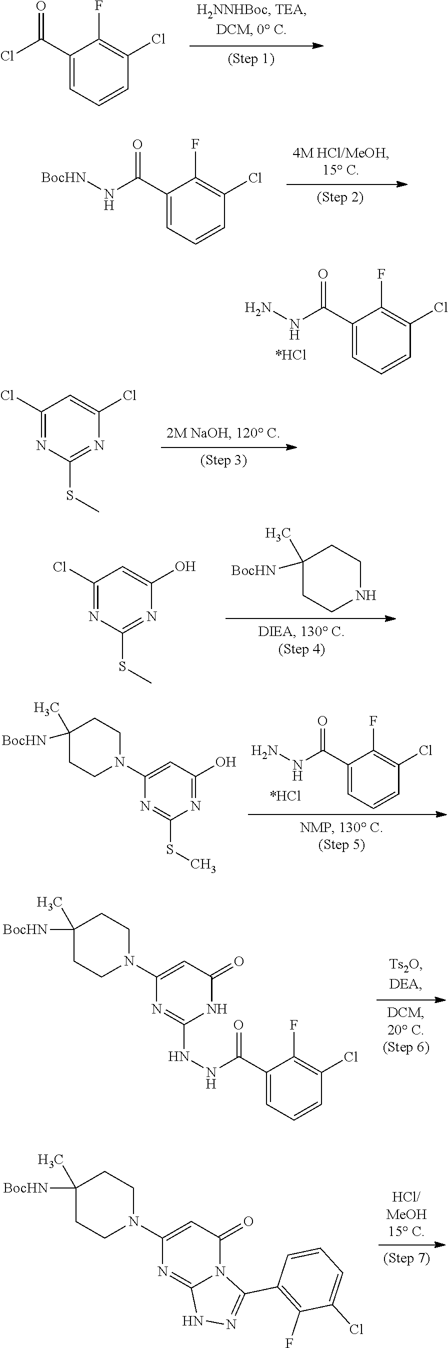

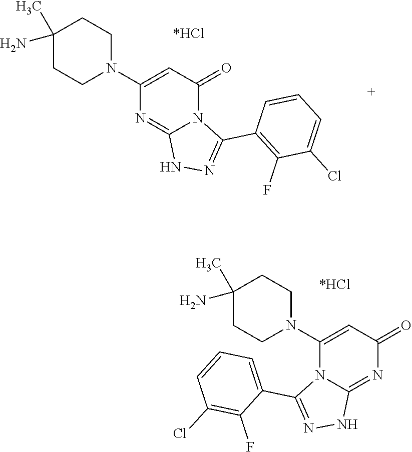 [SMILES: CC1(N)CCN(c2cc(=O)n3c(-c4cccc(Cl)c4F)n[nH]c3n2)CC1 ; CC1(N)CCN(c2cc(=O)nc3[nH]nc(-c4cccc(Cl)c4F)n23)CC1]

Step 1: To a mixture of tert-butyl hydrazinecarboxylate (820 mg, 6.2 mmol) and TEA (1.0 g, 10.3 mmol) in DCM (25 mL) at 0° C. was slowly added 3-chloro-2-fluorobenzoyl chloride (1.0 g, 5.2 mmol). After addition, the reaction mixture was stirred at 0° C. for 0.5 hour. The mixture was concentrated under reduced pressure to give a residue, which was purified by silica gel column chromatography (20-33% EtOAc/petroleum ether) to afford tert-butyl 2-(3-chloro-2-fluorobenzoyl)hydrazinecarboxylate (1.4 g, 92% yield) as a white solid.

Step 2: A mixture of tert-butyl 2-(3-chloro-2-fluorobenzoyl)hydrazinecarboxylate (1.4 g, 4.7 mmol) in HCl/MeOH (4 M, 10 mL) was stirred at 15° C. for 0.5 hour. The reaction mixture was concentrated under reduced pressure to afford 3-chloro-2-fluorobenzohydrazide hydrochloride (1.1 g, 100% yield) as a white solid: ¹HNMR (400 MHz, methanol-d₄) δ 7.75-7.80 (m, 2H), 7.35-7.39 (m, 1H).

Step 3: A mixture of 4,6-dichloro-2-(methylthio)pyrimidine (5.0 g, 25.6 mmol) in aqueous NaOH (2N, 120 mL) was stirred at 120° C. for 5 hours. The reaction mixture was cooled to 20° C. and AcOH was added until pH=6. The resulting precipitate was filtered off and washed with water. The solid was triturated with petroleum ether (30 mL), filtered, and dried in vacuo to afford 6-chloro-2-(methylthio)pyrimidin-4-ol (4.2 g, 92.9% yield) as a white solid: ¹HNMR (400 MHz, methanol-d₄) δ 6.23 (s, 1H), 2.59 (s, 3H).

Step 4: A mixture of 6-chloro-2-(methylthio)pyrimidin-4-ol (4.2 g, 23.7 mmol) and tert-butyl (4-methylpiperidin-4-yl)carbamate (5.1 g, 23.7 mmol) in DIEA (40 mL) was stirred at 130° C. for 12 hours. The reaction mixture was concentrated under reduced pressure give a residue which was triturated with ethyl acetate (15 mL). The resulting precipitate was filtered and dried under vacuum to afford tert-butyl (1-(6-hydroxy-2-(methylthio)pyrimidin-4-yl)-4-methylpiperidin-4-yl)carbamate (3.5 g, 37.8% yield) as a white solid: ESMS (M+H)⁺=355.

Step 5: A mixture of tert-butyl (1-(6-hydroxy-2-(methylthio)pyrimidin-4-yl)-4-methylpiperidin-4-yl)carbamate (1.0 g, 2.8 mmol) and 3-chloro-2-fluorobenzohydrazide hydrochloride (760 mg, 3.38 mmol) in NMP (1 mL) was stirred at 130° C. for 5 hours. The reaction mixture was diluted with MeOH (4 mL) and purified by reversed-phase HPLC to afford tert-butyl (1-(2-(2-(3-chloro-2-fluorobenzoyl)hydrazinyl)-6-oxo-1,6-dihydropyrimidin-4-yl)-4-methylpiperidin-4-yl)carbamate (240 mg, 17.1% yield) as a brown solid: (M+H)⁺=495.1.

Step 6: To a mixture of tert-butyl (1-(2-(2-(3-chloro-2-fluorobenzoyl)hydrazinyl)-6-oxo-1,6-dihydropyrimidin-4-yl)-4-methylpiperidin-4-yl)carbamate (240 mg, 484 μmol) and TEA (146 mg, 1.5 mmol) in DCM (8 mL) was added Ts₂O (165 mg, 484 μmol). The reaction mixture was stirred at 20° C. for 12 hours. The reaction mixture was concentrated under reduced pressure to give a residue which was purified by reversed-phase HPLC to afford tert-butyl (1-(3-(3-chloro-2-fluorophenyl)-5-oxo-1,5-dihydro-[1,2,4]triazolo[4,3-a]pyrimidin-7-yl)-4-methylpiperidin-4-yl)carbamate (30 mg, 13.0% yield) as a white solid.

Step 7: A mixture of tert-butyl (1-(3-(3-chloro-2-fluorophenyl)-5-oxo-1,5-dihydro-[1,2,4]triazolo[4,3-a]pyrimidin-7-yl)-4-methylpiperidin-4-yl)carbamate (30 mg, 62.9 μmol) in HCl/MeOH (4 M, 1 mL) was stirred at 15° C. for 0.5 hour. The reaction mixture was concentrated under reduced pressure to give the crude product. The residue was purified by reversed-phase HPLC to afford 7-(4-amino-4-methylpiperidin-1-yl)-3-(3-chloro-2-fluorophenyl)-[1,2,4]triazolo[4,3-a]pyrimidin-5(1H)-one hydrochloride (6.4 mg, 24.6% yield), as a white solid: ESMS (M+H)⁺=377.0; ¹HNMR (400 MHz, DMSO-d₆): δ 7.83-7.79 (m, 1H), 7.70-7.66 (m, 1H), 7.44-7.40 (m, 1H), 5.58 (s, 1H), 3.09-2.63 (m, 4H), 1.44-1.12 (m, 4H), 0.87 (s, 3H) and 5-(4-amino-4-methylpiperidin-1-yl)-3-(3-chloro-2-fluorophenyl)-[1,2,4]triazolo[4,3-a]pyrimidin-7(1H)-one hydrochloride (4.7 mg, 18.1% yield), as a white solid: ESMS (M+H)⁺=377.0; ¹HNMR (400 MHz, DMSO-d₆): δ 8.03-7.99 (m, 1H), 7.73-7.70 (m, 1H), 7.39-7.35 (m, 1H), 5.33 (s, 1H), 3.61-3.54 (m, 4H), 1.64 (br, 4H), 1.19 (s, 3H).

### Preparation of 6-(4-(aminomethyl)-4-methylpiperidin-1-yl)-3-(indolin-1-yl)-5-methyl-1H-pyrazolo[3,4-d]pyrimidin-4(5H)-one (Example F4)

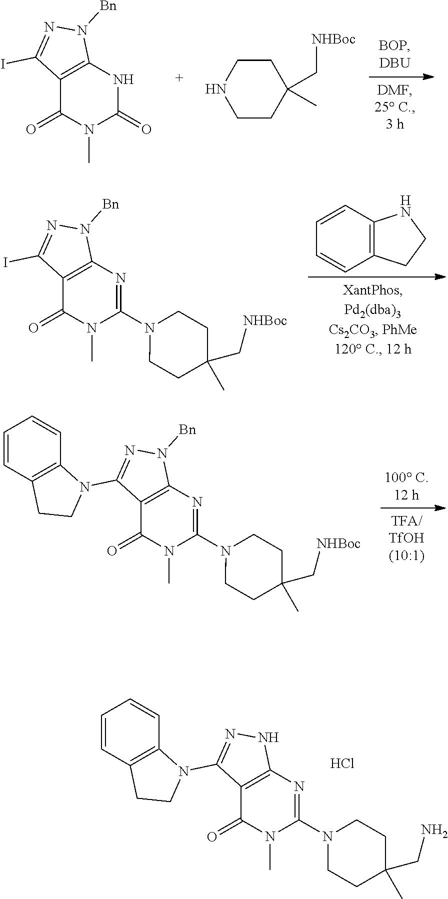

Step 1: A mixture of 1-benzyl-3-iodo-5-methyl-1H-pyrazolo[3,4-d]pyrimidine-4,6(5H,7H)-dione (400 mg, 1.0 mmol), tert-butyl ((4-methylpiperidin-4-yl)methyl)carbamate (237 mg, 1.0 mmol), BOP (1.3 g, 3.1 mmol) and DBU (949 mg, 6.2 mmol) in DMF (10 mL) was stirred at 25° C. for 3 hours. The mixture was diluted with H₂O (20 mL) and exacted with EtOAc (50 mL). The organics were washed with brine (2×20 mL), dried over anhydrous Na₂SO₄, filtered, and concentrated under reduced pressure. The residue was purified by silica gel column chromatography (0-50% EtOAc/petroleum ether) to afford tert-butyl ((1-(1-benzyl-3-iodo-5-methyl-4-oxo-4,5-dihydro-1H-pyrazolo[3,4-d]pyrimidin-6-yl)-4-methylpiperidin-4-yl)methyl)carbamate (420 mg, 68% yield) as a yellow solid.

Step 2: A mixture of tert-butyl ((1-(1-benzyl-3-iodo-5-methyl-4-oxo-4,5-dihydro-1H-pyrazolo[3,4-d]pyrimidin-6-yl)-4-methylpiperidin-4-yl)methyl)carbamate (300 mg, 506 μmol), indoline (78 mg, 657 μmol), XantPhos (58 mg, 101 μmol), Pd₂(dba)₃ (92 mg, 101 μmol) and Cs₂CO₃ (492 mg, 1.51 mmol) in toluene (10 mL) was evacuated and refilled for 3 times with nitrogen gas. The reaction mixture was then stirred at 120° C. for 12 hours. The mixture was concentrated under reduced pressure and the residue was purified by column chromatography (20-50% EtOAc/petroleum ether) to afford tert-butyl ((1-(1-benzyl-3-(indolin-1-yl)-5-methyl-4-oxo-4,5-dihydro-1H-pyrazolo[3,4-d]pyrimidin-6-yl)-4-methylpiperidin-4-yl)methyl)carbamate (230 mg, 78% yield) as a yellow solid.

Step 3. A mixture of tert-butyl ((1-(1-benzyl-3-(indolin-1-yl)-5-methyl-4-oxo-4,5-dihydro-1H-pyrazolo[3,4-d]pyrimidin-6-yl)-4-methylpiperidin-4-yl)methyl)carbamate (230 mg) in TFA (5 mL) and TfOH (0.5 mL) was stirred at 100° C. for 12 hours. The mixture was concentrated under reduced pressure and the residue was diluted with MeOH, purified by reversed-phase HPLC to afford 6-(4-(aminomethyl)-4-methylpiperidin-1-yl)-3-(indolin-1-yl)-5-methyl-1H-pyrazolo[3,4-d]pyrimidin-4(5H)-one (28.9 mg, HCl salt, 24% yield) as a green solid: ESMS (M+H)⁺=394.0; ¹HNMR (400 MHz, DMSO-d₆): δ 8.05 (br, 3H), 7.48-7.45 (d, J=8.0 Hz, 1H), 7.16-7.14 (d, J=6.8 Hz, 1H), 7.07-7.02 (m, 1H), 6.77-6.72 (m, 1H), 4.36-4.31 (m, 2H), 3.39 (s, 3H), 3.31-3.26 (m, 2H), 3.13-3.06 (m, 4H), 2.80-2.78 (m, 2H), 1.68-1.63 (m, 2H), 1.53-1.49 (m, 2H), 1.08 (s, 3H).

### Preparation of 3-(2,3-dichlorophenyl)-6-(piperidin-4-yl)-1H-pyrazolo[4,3-d]pyrimidin-7(6H)-one (Example F5)

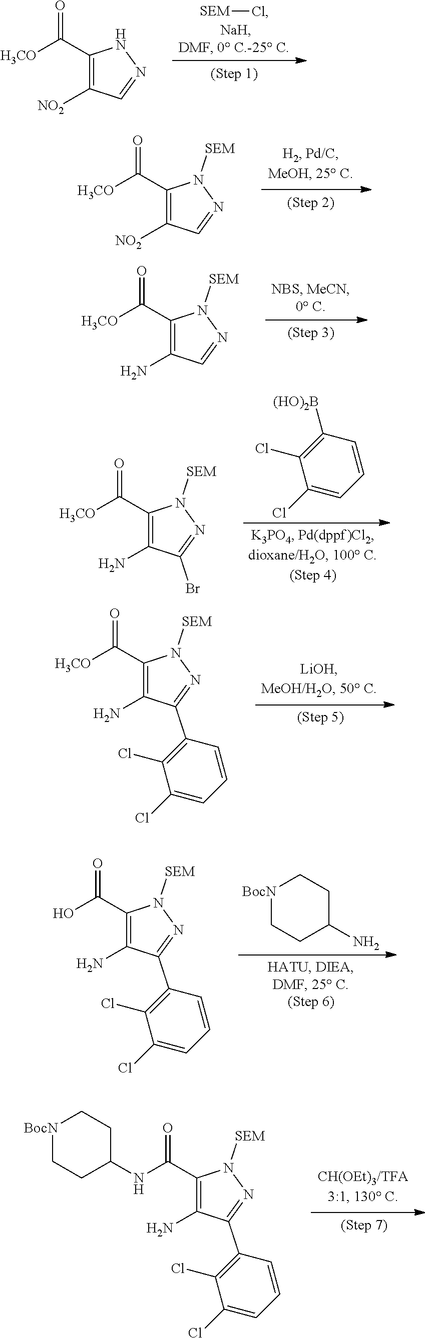

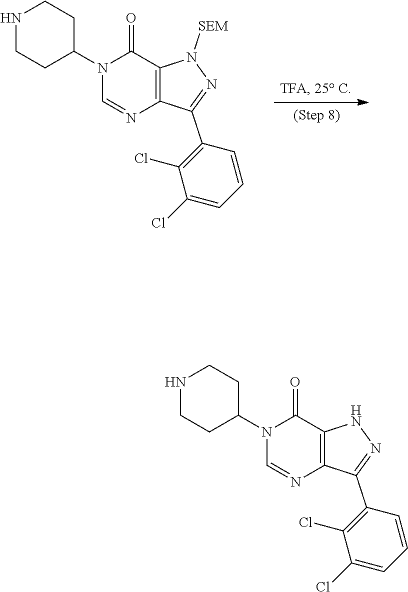 [SMILES: C[Si](C)(C)CCOCn1nc(-c2cccc(Cl)c2Cl)c2ncn(C3CCNCC3)c(=O)c21 ; O=c1c2[nH]nc(-c3cccc(Cl)c3Cl)c2ncn1C1CCNCC1]

Step 1: Methyl 4-nitro-1H-pyrazole-5-carboxylate (1.0 g, 5.8 mmol) was dissolved into DMF (15.0 mL) and NaH (233.6 mg, 5.8 mmol, 60% in mineral oil) was added at 0° C. The reaction mixture was stirred at 0° C. for 15 min, SEM-Cl (1.2 g, 7.0 mmol) was added under N₂. The reaction mixture was warmed to 25° C. and stirred for 12 hours. The reaction mixture was concentrated under reduced pressure and diluted with EtOAc (100 mL), washed with H₂O (50 mL), and brine (50 mL). The organics were dried over anhydrous Na₂SO₄, filtered, concentrated under reduced pressure, and purified by flash chromatography (0-10% EtOAc/petroleum ether) to afford methyl 4-nitro-2-(2-trimethylsilylethoxymethyl)pyrazole-3-carboxylate (500.0 mg, 1.66 mmol, 28.4% yield) as a white solid. Also isolated was methyl 4-nitro-1-(2-trimethylsilylethoxymethyl)pyrazole-3-carboxylate (700.0 mg, 2.3 mmol, 39.8% yield) as a white solid.

Step 2: Ethyl 4-nitro-2-(2-trimethylsilylethoxymethyl)pyrazole-3-carboxylate (1.0 g, 3.3 mmol) and 10% Pd/C (400.0 mg, wet) taken up in MeOH (50.0 mL) and the reaction mixture was evacuated and refilled for 3 times with hydrogen gas. The reaction was then stirred at 25° C. for 12 hours under an atmosphere of hydrogen (15 psi). The reaction mixture was filtered and concentrated under reduced pressure to give methyl 4-amino-2-(2-trimethylsilylethoxymethyl)pyrazole-3-carboxylate (780.0 mg, 2.9 mmol, 86.6% yield) was obtained as a pink oil.

Step 3: Methyl 4-amino-2-(2-trimethylsilylethoxymethyl)pyrazole-3-carboxylate (680.0 mg, 2.5 mmol) was dissolved into acetonitrile (20.0 mL) and NBS (491.4 mg, 2.8 mmol) was added in three portions. The reaction mixture was stirred at 0° C. for 12 hours then concentrated under reduced pressure to give a yellow solid. Purification by flash chromatography (0-20% EtOAc/petroleum ether) provided methyl4-amino-5-bromo-2-(2-trimethylsilylethoxymethyl)pyrazole-3-carboxylate (500.0 mg, 56.9% yield) as a yellow solid.

Step 4: Methyl 4-amino-5-bromo-2-(2-trimethylsilylethoxymethyl)pyrazole-3-carboxylate (500.0 mg, 1.4 mmol), (2,3-dichlorophenyl)boronic acid (409.3 mg, 2.1 mmol), Pd(dppf)Cl2 (104.6 mg, 143.0 umol) and K₃PO₄.3H₂O (1.1 g, 4.3 mmol) were taken up in dioxane (20.0 mL) and H₂O (2.0 mL). The reaction mixture was evacuated and refilled for 3 times with nitrogen gas, followed by stirring at 100° C. for 12 hours. The reaction mixture was concentrated under reduced pressure to give a yellow residue which was purified by flash chromatography (0-20% EtOAc/petroleum ether) to provide methyl 4-amino-5-(2,3-dichlorophenyl)-2-(2-trimethylsilylethoxymethyl)pyrazole-3-carboxylate (350.0 mg, 55.4% yield) as a yellow oil.

Step 5: Methyl 4-amino-5-(2,3-dichlorophenyl)-2-(2-trimethylsilylethoxymethyl)pyrazole-3-carboxylate (300.0 mg, 720.5 umol), LiOH.H₂O (51.8 mg, 2.2 mmol) were taken up in MeOH (20.0 mL) and H₂O (2.0 mL). The reaction mixture was stirred at 50° C. for 1 hour then concentrated under reduced pressure to give a white solid which was partitioned between H₂O (20.0 mL) and EtOAc (20.0 mL). The aqueous phase was adjusted to pH=4 with 1N HCl and extracted with EtOAc (20 mL×4). The combined organics were washed with H₂O (20 mL), brine (20 mL), dried over anhydrous Na₂SO₄, filtered, and concentrated under reduced pressure to provide 4-amino-5-(2,3-dichlorophenyl)-2-(2-trimethylsilylethoxymethyl)pyrazole-3-carboxylic acid (280.0 mg, 96.6% yield) as a yellow solid.

Step 6: 4-Amino-5-(2,3-dichlorophenyl)-2-(2-trimethylsilylethoxymethyl)pyrazole-3-carboxylic acid (270.0 mg, 671.1 umol), tert-butyl 4-aminopiperidine-1-carboxylate (161.3 mg, 805.3 umol), HATU (306.2 mg, 805.3 umol) and DIEA (173.5 mg, 1.3 mmol, 234.4 uL) were taken up in DMF (10.0 mL) the reaction was stirred at 25° C. for 12 hours. The reaction mixture was concentrated under reduced pressure and purified by preparative TLC (33% EtOAc/petroleum ether) to afford tert-butyl 4-[[4-amino-5-(2,3-dichlorophenyl)-2-(2-trimethylsilylethoxymethyl)pyrazole-3-carbonyl]amino]piperidine-1-carboxylate (300.0 mg, 74.2% yield) as a yellow solid.

Step 7: To a solution of tert-butyl 4-[[4-amino-5-(2,3-dichlorophenyl)-2-(2-trimethylsilylethoxymethyl)pyrazole-3-carbonyl]amino]piperidine-1-carboxylate (280.0 mg, 478.9 umol) in CH(OEt)₃ (18.0 mmol, 3.0 mL) was added TFA (1.5 g, 13.5 mmol, 1.0 mL). The reaction was stirred at 130° C. for 24 hours then concentrated under reduced pressure. Purification by flash chromatography (0-10% methanol/DCM) provided 3-(2,3-dichlorophenyl)-6-(4-piperidyl)-1-(2-trimethylsilylethoxymethyl)pyrazolo[4,3-d]pyrimidin-7-one (120.0 mg, 48.1% yield) as a yellow solid.

Step 8: tert-Butyl 4-(3-(2,3-dichlorophenyl)-7-oxo-1-((2-(trimethylsilyl)ethoxy)methyl)-1H-pyrazolo[4,3-d]pyrimidin-6(7H)-yl)piperidine-1-carboxylate (120 mg, 201 μmol) was taken up in TFA (3 mL) and the reaction stirred at 25° C. for 12 hours. The reaction mixture was concentrated under reduced pressure, diluted with MeOH, adjusted pH=9 by adding NH₃.H₂O and purified by reversed-phase HPLC to afford 3-(2,3-dichlorophenyl)-6-(piperidin-4-yl)-1H-pyrazolo[4,3-d]pyrimidin-7(6H)-one (5.8 mg, 15.9 μmol) as a white solid: ESMS [M+H]⁺=364.0; ¹HNMR (400 MHz, CDCl₃): δ 8.12 (s, 1H), 7.60-7.54 (m, 2H), 7.35-7.32 (m, 1H), 5.06-4.97 (m, 1H), 3.35-3.31 (m, 2H), 2.94-2.81 (m, 2H), 2.03-1.99 (m, 4H).

### Preparation of 6-(4-(aminomethyl)-4-methylpiperidin-1-yl)-3-(2,3-dichlorophenyl)-5-methyl-1H-pyrazolo[3,4-d]pyrimidin-4(5H)-one dihydrochloride (Example F6)

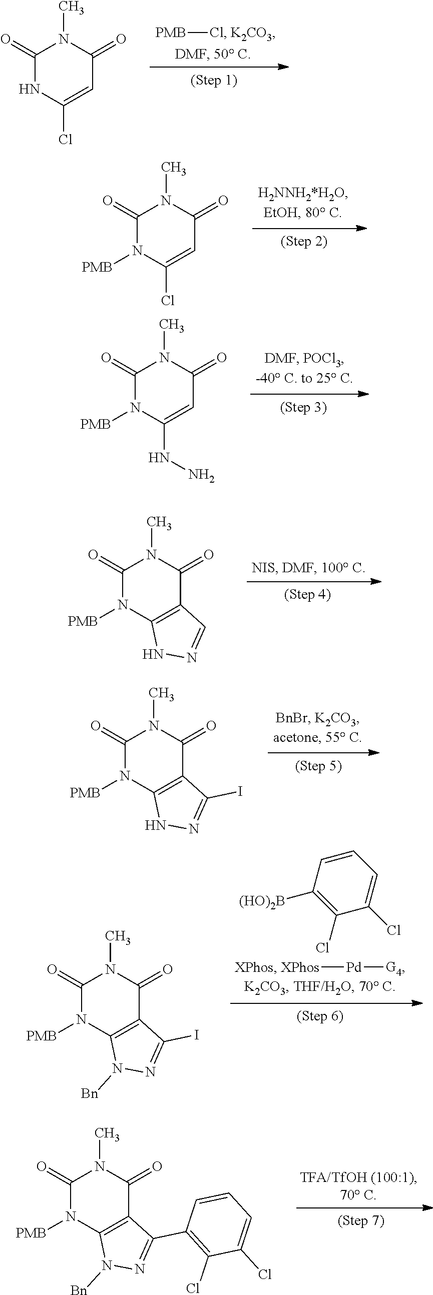

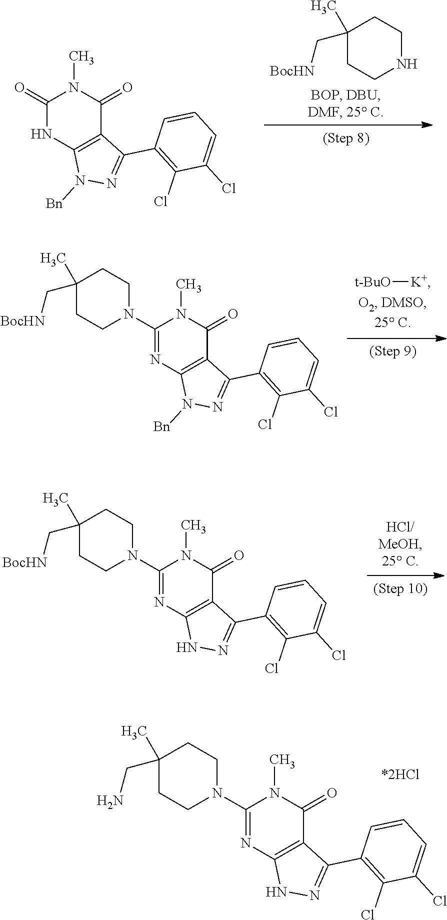

Step 1: 6-Chloro-3-methylpyrimidine-2,4 (1H,3H)-dione (43 g, 267 mmol), K₂CO₃ (92.1 g, 667 mmol) and PMB-Cl (1.4 g, 28.0 mmol) were taken up in DMF (500 mL). The reaction mixture was stirred at 50° C. for 16 hours. The mixture was partitioned between H₂O (1000 mL) and EtOAc (1500 mL) and the aqueous phase was extracted with EtOAc (800 mL×2). The combined organics were washed with brine (500 mL×2), dried over anhydrous Na₂SO₄, filtered, and concentrated under reduced pressure to give a residue. Trituration with petroleum ether/EtOAc (10:1) resulted in the formation of a precipitate which was collected by filtration, washed with petroleum ether/EtOAc (10:1, 100 mL), and dried to give 6-chloro-1-(4-methoxybenzyl)-3-methylpyrimidine-2,4(1H,3H)-dione (50.0 g, 66.7% yield) as a white solid.

Step 2: 6-Chloro-1-(4-methoxybenzyl)-3-methylpyrimidine-2,4(1H,3H)-dione (48 g, 170 mmol) was taken up in EtOH (400 mL) and NH₂NH₂—H₂O (29.1 mL, 510 mmol, 85% purity) was added. The reaction mixture was stirred at 80° C. for 2 hours. The mixture was concentrated by distilling off most of the EtOH (300 mL). After cooling to room temperature a precipitate formed which was collected by filtration. The filter cake was rinsed with water and dried to afford the product of 6-hydrazinyl-1-(4-methoxybenzyl)-3-methylpyrimidine-2,4(1H,3H)-dione (43.0 g, 91.6% yield) as a white solid.

Step 3: 6-Hydrazinyl-1-(4-methoxybenzyl)-3-methylpyrimidine-2,4(1H,3H)-dione (38 g, 137 mmol) was dissolved in DMF (500 mL). The reaction mixture was cooled to −40° C. and POCl₃ (127 mL, 1.37 mol) was added drop wise. The reaction mixture was stirred at −40° C. for 2 hours, then the mixture was warmed to 25° C. and stirred for 10 hours. The mixture was concentrated under reduced pressure to remove POCl₃. The mixture was cooled to 0° C. and added into ice-water (3000 mL). The resulting precipitate was collected by filtration, rinsed with water, and dried to afford 7-(4-methoxybenzyl)-5-methyl-1H-pyrazolo[3,4-d]pyrimidine-4,6(5H,7H)-dione (36.0 g, 91.8 yield) as a white solid.

Step 4: 7-(4-Methoxybenzyl)-5-methyl-1H-pyrazolo[3,4-d]pyrimidine-4,6(5H,7H)-dione (10 g, 34.9 mmol) and NIS (11.7 g, 52.3 mmol) were taken up in DMF (100 mL). The reaction mixture was stirred at 110° C. for 10 hours. The mixture was treated 100 mL of sat. Na₂SO₃/sat NaHCO₃ (1:1) and diluted with H₂O (400 mL). The resulting precipitate was collected by filtration, washed with H₂O, and dried to afford 3-iodo-7-(4-methoxybenzyl)-5-methyl-1H-pyrazolo[3,4-d]pyrimidine-4,6(5H,7H)-dione (14.0 g, 97.9% yield) as a white solid.

Step 5: 3-Iodo-7-(4-methoxybenzyl)-5-methyl-1H-pyrazolo[3,4-d]pyrimidine-4,6(5H,7H)-dione (10 g, 24.2 mmol), BnBr (3.2 mL, 26.6 mmol) and K₂CO₃ (16.7 g, 121 mmol) were taken up in acetone (200 mL). The mixture was stirred at 55° C. for 2 hours. The reaction mixture was filtered and concentrated under reduced pressure to give a residue. Trituration with petroleum ether/EtOAc (10:1) resulted in the formation of a precipitate which was collected by filtration, washed with petroleum ether/EtOAc (10:1), and dried to give 1-benzyl-3-iodo-7-(4-methoxybenzyl)-5-methyl-1H-pyrazolo[3,4-d]pyrimidine-4,6(5H,7H)-dione (10.0 g, 82.6% yield) as a white solid.

Step 6: 1-Benzyl-3-iodo-7-(4-methoxybenzyl)-5-methyl-1H-pyrazolo[3,4-d]pyrimidine-4,6(5H,7H)-dione (1 g, 2.0 mmol), (2,3-dichlorophenyl)boronic acid (948 mg, 5.0 mmol), XPhos (95 mg, 0.2 mmol), Xphos-Pd-G4 (171 mg, 0.2 mmol) and K₂CO₃ (1.1 g, 8.0 mmol) were taken up in THE (100.0 mL) and H₂O (20.0 mL). The reaction mixture was evacuated and refilled 3 times with nitrogen gas. The reaction mixture was stirred at 70° C. under an atmosphere of nitrogen for 10 hours. The reaction mixture was concentrated under reduced pressure, H₂O (50 mL) was added, and the mixture extracted with DCM (3×50 mL). The combined organics were washed with brine (50 mL), dried over anhydrous Na₂SO₄, filtered, and concentrated under reduced pressure to give a residue which was purified by flash chromatography (0-30% EtOAc/petroleum ether) to afford 1-benzyl-3-(2,3-dichlorophenyl)-7-(4-methoxybenzyl)-5-methyl-1H-pyrazolo[3,4-d]pyrimidine-4,6(5H,7H)-dione (800 mg, 77.6%) as a yellow solid.

Step 7: 1-Benzyl-3-(2,3-dichlorophenyl)-7-(4-methoxybenzyl)-5-methyl-1H-pyrazolo[3,4-d]pyrimidine-4,6(5H,7H)-dione (700 mg, 1.3 mmol) and TfOH (0.3 mL, 4.0 mmol) was taken up in TFA (30 mL). The mixture was stirred at 70° C. for 1 h. The reaction mixture was diluted with DCM (30 mL), concentrated under reduced pressure, and H₂O (10 mL) was added. The pH of the mixture was adjusted to pH=9-10 with aq. NaOH (1 N) and extracted with ethyl acetate (3×30 mL). The combined organic layers were washed with brine (50 mL), dried over anhydrous Na₂SO₄, filtered, and concentrated under reduced pressure to give a residue which was purified by flash chromatography (0-50% EtOAc/petroleum ether) to afford 1-benzyl-3-(2,3-dichlorophenyl)-5-methyl-1H-pyrazolo[3,4-d]pyrimidine-4,6(5H,7H)-dione (600 mg, crude) as a yellow solid.

Step 8: 1-Benzyl-3-(2,3-dichlorophenyl)-5-methyl-1H-pyrazolo[3,4-d]pyrimidine-4,6(5H,7H)-dione (550 mg, 1.4 mmol) was dissolved in DMF (5 mL) and BOP (1.8 g, 4.1 mmol) was added. The reaction mixture was stirred at 25° C. for 5 min and tert-butyl ((4-methylpiperidin-4-yl)methyl)carbamate (468 mg, 2.0 mmol) and DBU (0.1 mL, 0.7 mmol) were added. The reaction mixture was stirred at 25° C. for 10 hours. The mixture was partitioned between EtOAc (30 mL) and H₂O (30 mL) and the aqueous phase was extracted with ethyl acetate (2×10 mL). The combined organics were washed with H₂O (2×30 mL), brine (30 mL), dried over anhydrous Na₂SO₄, filtered, and concentrated under reduced pressure to give a residue which was purified by flash chromatography (0-50% EtOAc/petroleum ether) to afford tert-butyl ((1-(1-benzyl-3-(2,3-dichlorophenyl)-5-methyl-4-oxo-4,5-dihydro-1H-pyrazolo[3,4-d]pyrimidin-6-yl)-4-methylpiperidin-4-yl)methyl)carbamate (760 mg, 90.8% yield) as a yellow solid.

Steps 9 & 10: tert-Butyl ((1-(1-benzyl-3-(2,3-dichlorophenyl)-5-methyl-4-oxo-4,5-dihydro-1H-pyrazolo[3,4-d]pyrimidin-6-yl)-4-methylpiperidin-4-yl)methyl)carbamate (370 mg, 0.6 mmol) was dissolved in DMSO (10 mL) and t-BuOK (203 mg, 1.8 mmol) was added. The reaction mixture was evacuated and refilled 3 times with O₂. The reaction mixture was stirred at 25° C. for 12 h under and atmosphere of O₂ (15 psi). The reaction mixture was sparged with N₂ and HCl/MeOH (20 mL, 4N) was added. The mixture was stirred at 25° C. for 10 hours and concentrated under reduced pressure. The residue was purified by reversed-phase HPLC to afford 6-(4-(aminomethyl)-4-methylpiperidin-1-yl)-3-(2,3-dichlorophenyl)-5-methyl-1H-pyrazolo[3,4-d]pyrimidin-4(5H)-one dihydrochloride (101 mg, 55.4% yield) as a white solid: ESMS [M+H]⁺=421.1; ¹HNMR (400 MHz, DMSO_d₆): δ 8.01 (s, 3H), 7.75-7.71 (m, 1H), 7.47-7.41 (m, 2H), 3.37 (s, 3H), 3.32-3.28 (m, 2H), 3.14-3.08 (m, 2H), 2.81-2.78 (m, 2H), 1.69-1.63 (m, 2H), 1.54-1.49 (m, 2H), 1.08 (s, 3H).

### Preparation of 6-(4-(aminomethyl)-4-methylpiperidin-1-yl)-3-((2,3-dichlorophenyl)thio)-5-methyl-1H-pyrazolo[3,4-d]pyrimidin-4(5H)-one hydrochloride (Example F7)

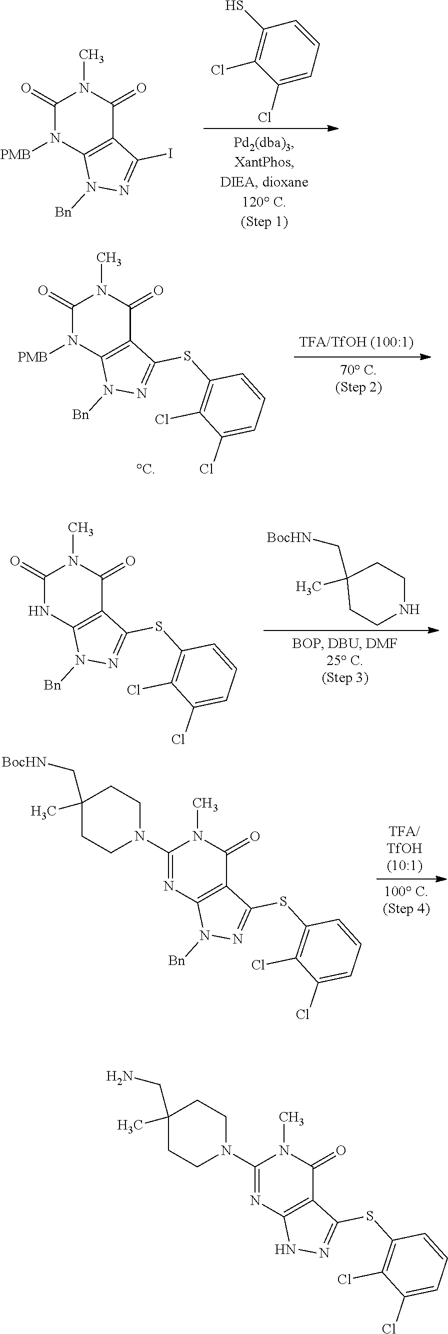

Step 1: 1-Benzyl-3-iodo-7-(4-methoxybenzyl)-5-methyl-1H-pyrazolo[3,4-d]pyrimidine-4,6(5H,7H)-dione (1.0 g, 2.0 mmol), 2,3-dichlorobenzenethiol (426 mg, 2.4 mmol), Xantphos (115 mg, 0.2 mmol), Pd₂(dba)₃ (91 mg, 0.1 mmol) and DIEA (1.0 mL, 6.0 mmol) were taken up in dioxane (50 mL). The mixture was evacuated and refilled for 3 times with nitrogen gas. The reaction mixture was stirred at 120° C. for 10 hours, concentrated under reduced pressure to give a residue which was purified by flash chromatography (0-30% EtOAc/petroleum ether) to afford 1-benzyl-3-((2,3-dichlorophenyl)thio)-7-(4-methoxybenzyl)-5-methyl-1H-pyrazolo[3,4-d]pyrimidine-4,6(5H,7H)-dione (440 mg, 40% yield) as a yellow solid.

Step 2: 1-Benzyl-3-((2,3-dichlorophenyl)thio)-7-(4-methoxybenzyl)-5-methyl-1H-pyrazolo[3,4-d]pyrimidine-4,6(5H,7H)-dione (390 mg, 0.7 mmol) and TfOH (0.2 mL, 2.1 mmol) were taken up in TFA (18 mL, 0.7 mmol). The mixture was stirred at 70° C. for 1 h. The reaction mixture was diluted with DCM (30 mL), concentrated under reduced pressure, and H₂O (10 mL) was added. The pH of the mixture was adjusted to 9-10 with aq. NaOH (1 N) and the mixture extracted with ethyl acetate (3×30 mL). The combined organics were washed with brine (50 mL), dried over anhydrous Na₂SO₄, filtered, and concentrated under reduced pressure to give a residue. The residue was triturated with petroleum ether/EtOAc (10:1) and the resulting solid was collected by filtration, washed with petroleum ether/EtOAc (10:1), and dried to give 1-benzyl-3-((2,3-dichlorophenyl)thio)-5-methyl-1H-pyrazolo[3,4-d]pyrimidine-4,6(5H,7H)-dione (300 mg, 98.3% yield) as a tan solid.

Step 3: 1-Benzyl-3-((2,3-dichlorophenyl)thio)-5-methyl-1H-pyrazolo[3,4-d]pyrimidine-4,6(5H,7H)-dione (250 mg, 0.6 mmol) was dissolved in DMF (2.5 mL) and BOP (761 mg, 1.7 mmol) was added. The mixture was stirred at 25° C. for 5 minutes followed by the addition of tert-butyl ((4-methylpiperidin-4-yl)methyl)carbamate (197 mg, 0.9 mmol) and DBU (0.5 mL, 3.5 mmol). The reaction mixture was stirred at 25° C. for 10 hours and partitioned between EtOAc (30 mL) and H₂O (20 mL). The organic layer was washed with H₂O (2×20 mL), brine (20 mL), dried over anhydrous Na₂SO₄, filtered, and concentrated under reduced pressure to give a residue. The residue was purified by flash chromatography (0-50% EtOAc/petroleum ether) to afford tert-butyl ((1-(1-benzyl-3-((2,3-dichlorophenyl)thio)-5-methyl-4-oxo-4,5-dihydro-1H-pyrazolo[3,4-d]pyrimidin-6-yl)-4-methylpiperidin-4-yl)methyl)carbamate (400 mg) as a white solid.

Step 4: tert-Butyl ((1-(1-benzyl-3-((2,3-dichlorophenyl)thio)-5-methyl-4-oxo-4,5-dihydro-1H-pyrazolo[3,4-d]pyrimidin-6-yl)-4-methylpiperidin-4-yl)methyl)carbamate (200 mg, 0.3 mmol) was taken up in TFA (10 mL) and TfOH (1 mL). The reaction mixture was stirred at 100° C. for 16 h. The solvent was diluted with DCM (20 mL) and concentrated to give a residue. The residue was triturated with NH₃/MeOH (1 mL, 7M), and DMSO (4.0 mL) was added. The mixture was filtered and purified by reversed phase HPLC to afford 6-(4-(aminomethyl)-4-methylpiperidin-1-yl)-3-((2,3-dichlorophenyl)thio)-5-methyl-1H-pyrazolo[3,4-d]pyrimidin-4(5H)-one hydrochloride, (103 mg, 68.2% yield) as a white solid: ESMS [M+H]⁺=453.0; ¹HNMR (400 MHz, DMSO_d₆): δ 8.10 (s, 3H), 7.50-7.47 (m, 1H), 7.24-7.19 (m, 1H), 6.96-6.93 (m, 1H), 3.34 (s, 3H), 3.31-3.27 (m, 2H), 3.13-3.07 (m, 2H), 2.79-2.77 (m, 2H), 1.68-1.62 (m, 2H), 1.53-1.48 (m, 2H), 1.07 (s, 3H).

### Preparation of 6-(4-(aminomethyl)-4-methylpiperidin-1-yl)-3-((3-chlorophenyl)thio)-5-methyl-1H-pyrazolo[3,4-d]pyrimidin-4(5H)-one (Example F8)

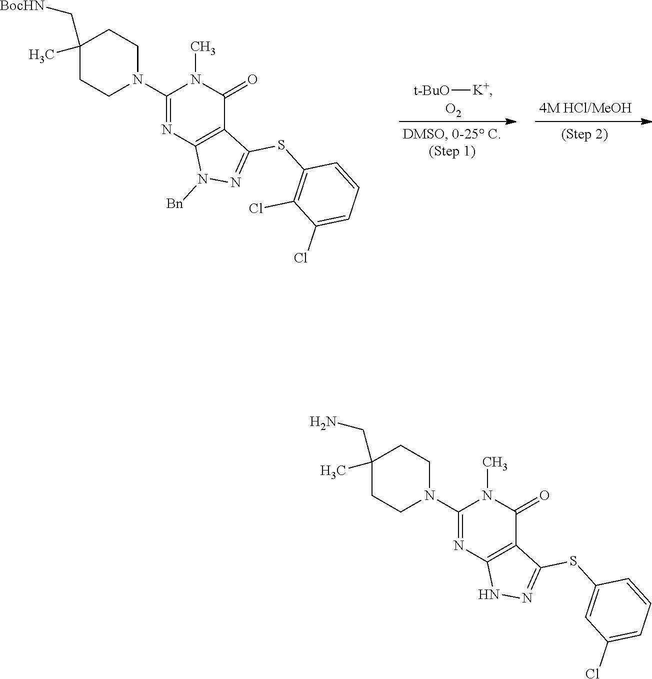

tert-Butyl ((1-(1-benzyl-3-((2,3-dichlorophenyl)thio)-5-methyl-4-oxo-4,5-dihydro-1H-pyrazolo[3,4-d]pyrimidin-6-yl)-4-methylpiperidin-4-yl)methyl)carbamate (20 mg, 0.03 mmol) was dissolved in DMSO (0.5 mL). The reaction mixture was evacuated and refilled for 3 times using O₂. t-BuOK (0.5 mL, 0.5 mmol, 1N in THF) was added at 0° C. The reaction mixture was stirred at 25° C. for 10 h under O₂ (15 psi). Then HCl/MeOH (1 mL, 4N) was added and the reaction mixture concentrated to give a residue. The mixture was purified by reversed-phase HPLC to afford 6-(4-(aminomethyl)-4-methylpiperidin-1-yl)-3-((3-chlorophenyl)thio)-5-methyl-1H-pyrazolo[3,4-d]pyrimidin-4(5H)-one, as a white solid: ESMS [M+H]⁺=419.0; ¹HNMR (400 MHz, D₂O): δ 7.10-7.04 (m, 4H), 3.30-3.10 (m, 5H), 3.09-2.90 (m, 2H), 2.90-2.75 (m, 2H), 1.54-1.47 (m, 4H), 0.99 (s, 3H).

### Preparation of 1-[3-(2,3-dichlorophenyl)-4-methoxy-1H-pyrazolo[3,4-d]pyrimidin-6-yl]-4-methyl-piperidin-4-amine (Example F9)

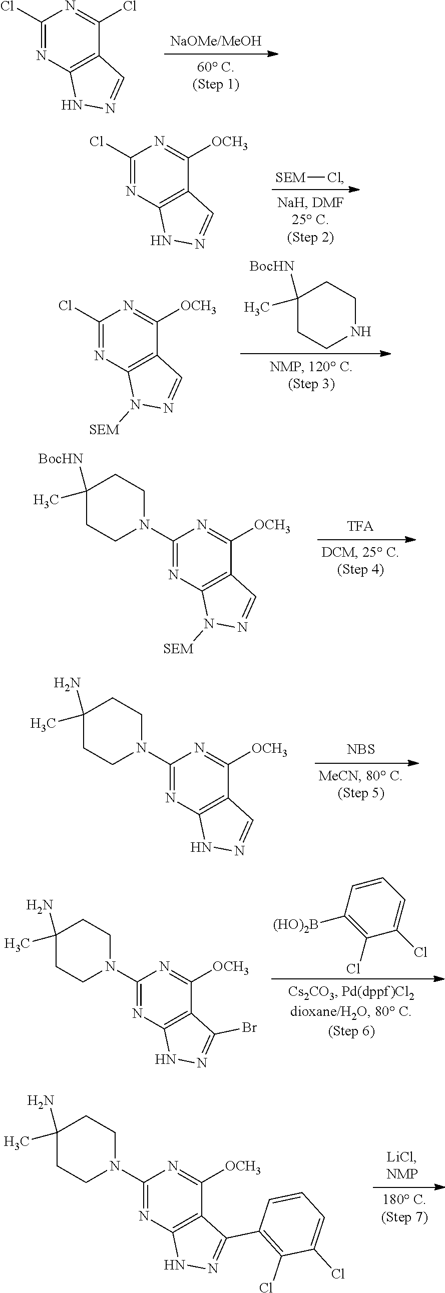

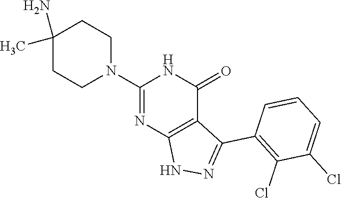 [SMILES: CC1(N)CCN(c2nc3[nH]nc(-c4cccc(Cl)c4Cl)c3c(=O)[nH]2)CC1]

Step 1: A mixture of 4,6-dichloro-1H-pyrazolo[3,4-d]pyrimidine (3.0 g, 15.9 mmol, 1.0 eq) and NaOMe (1.9 g, 47.6 mmol, 3.0 eq) in MeOH (20 mL) was stirred at 60° C. for 1 hour. The reaction mixture was quenched with water (30 mL) and the solution adjusted pH=6 with 1N HCl and extracted with EtOAc (3×50 mL). The combined organics were washed with water (2×50 mL), dried over anhydrous Na₂SO₄, filtered, and concentrated under reduced pressure. The resulting 6-chloro-4-methoxy-1H-pyrazolo[3,4-d]pyrimidine (1.8 g, combined product) was obtained as a white solid and used in next step as is.

Step 2: To a solution of 6-chloro-4-methoxy-H-pyrazolo[3,4-d]pyrimidine (1.7 g, 9.2 mmol) in DMF (20 mL) was added NaH (552.6 mg, 13.8 mmol, 60% in mineral oil). After 10 min, SEM-Cl (2.3 g, 13.8 mmol, 2.5 mL) was added. The reaction mixture was stirred at 25° C. for 1 hour under an atmosphere of nitrogen. The reaction mixture was concentrated under reduced pressure. The residue was diluted with water (30 mL) and extracted with EtOAc (2×30 mL). The combined organic layers were washed with water (2×30 mL), dried over anhydrous Na₂SO₄, filtered, and concentrated under reduced pressure to give a residue, which was purified by column chromatography (5-20% EtOAc/petroleum ether). The desired 6-chloro-4-methoxy-1-((2-(trimethylsilyl)ethoxy)methyl)-1H-pyrazolo[3,4-d]pyrimidine (1.3 g, 45.0% yield) was obtained as a light pink solid along with major impurity 6-chloro-4-methoxy-2-((2-(trimethylsilyl)ethoxy)methyl)-2H-pyrazolo[3,4-d]pyrimidine (520.0 mg, 17.9% yield), which was obtained as a light yellow solid.

Step 3: A mixture of 6-chloro-4-methoxy-1-((2-(trimethylsilyl)ethoxy)methyl)-1H-pyrazolo[3,4-d]pyrimidine (1.3 g, 4.2 mmol) and tert-butyl N-(4-methyl-4-piperidyl)carbamate (1.4 g, 6.3 mmol) in NMP (500 uL) was stirred at 120° C. for 12 h. The reaction mixture was partitioned between dichloromethane (50 mL) and water (50 mL). The organic phase was washed with water (2×50 mL), dried over anhydrous Na₂SO₄, filtered, and concentrated under reduced pressure. The residue was purified by column chromatography (5-50% EtOAc/petroleum ether). tert-Butyl N-[1-[4-methoxy-1-(2-trimethylsilylethoxymethyl)pyrazolo[3,4-d]pyrimidin-6-yl]-4-methyl-4-piperidyl]carbamate (1.3 g, 62.5% yield) was obtained as a yellow oil along with major impurity tert-butyl N-[1-[4-hydroxy-1-(2-trimethylsilylethoxymethyl)pyrazolo[3,4-d]pyrimidin-6-yl]-4-methyl-4-piperidyl]carbamate (466.0 mg, 23.1% yield) which was obtained as a yellow oil.

Step 4: tert-Butyl N-[1-[4-methoxy-1-(2-trimethylsilylethoxymethyl)pyrazolo[3,4-d]pyrimidin-6-yl]-4-methyl-4-piperidyl]carbamate (800.0 mg, 1.6 mmol) was taken up in TFA (8 mL) and DCM (15 mL) and the reaction mixture was stirred at 25° C. for 1 hour. The mixture was concentrated under reduced pressure, the residue diluted with DCM (15 mL) and the pH adjusted to pH=10 with NH₃H₂O. Purification by silica gel column chromatography (0-20% MeOH/EtOAc) produced 1-(4-methoxy-1H-pyrazolo[3,4-d]pyrimidin-6-yl)-4-methyl-piperidin-4-amine (2.5 g) as a white solid. This material was used in the next step as is.

Step 5: A mixture of 1-(4-methoxy-1H-pyrazolo[3,4-d]pyrimidin-6-yl)-4-methyl-piperidin-4-amine (2.5 g, 9.5 mmol) and NBS (2.0 g, 11.1 mmol) in DMF (30 mL) was stirred at 80° C. for 20 hours. The reaction mixture was concentrated under reduced pressure. the residue partitioned between water (30 mL) and EtOAc (30 mL). The aqueous layer was washed with EtOAc (30 mL) and the combined organics were washed with water (2×30 mL), dried over anhydrous Na₂SO₄, filtered, and concentrated under reduced pressure. The residue was purified by silica gel column chromatography (0-20% MeOH/EtOAc) to provide 1-(3-bromo-4-methoxy-1H-pyrazolo[3,4-d]pyrimidin-6-yl)-4-methyl-piperidin-4-amine (230.0 mg, 21.2% yield) as a light yellow solid.

Step 6: A mixture of 1-(3-bromo-4-methoxy-2H-pyrazolo[3,4-d]pyrimidin-6-yl)-4-methyl-piperidin-4-amine (200.0 mg, 586.2 umol), (2,3-dichlorophenyl)boronic acid (167.8 mg, 879.2 umol), K₃PO₄.3H₂O (312.2 mg, 1.2 mmol), Pd₂(dba)₃ (53.7 mg, 58.6 umol) and XPhos (27.9 mg, 58.6 umol) in DMSO (10 mL) was purged with nitrogen gas 3 times. The mixture was stirred at 100° C. for 20 h under an atmosphere of nitrogen. The reaction mixture was diluted with water (50 mL) and extracted with ethyl acetate (2×50 mL). The combined organics were washed with brine (3×50 mL), dried over anhydrous Na₂SO₄, filtered, and concentrated under reduced pressure. The residue was purified by reversed-phase HPLC to afford 1-[3-(2,3-dichlorophenyl)-4-methoxy-1H-pyrazolo[3,4-d]pyrimidin-6-yl]-4-methyl-piperidin-4-amine (13.0 mg, 5.5% yield, HCl salt) as a white solid: ESMS [M+H]⁺=407.1; ¹HNMR (400 MHz, methanol-d₄): δ 7.64-7.67 (m, 1H), 7.37-7.44 (m, 2H), 3.95 (s, 3H), 3.50-3.54 (m, 2H), 1.83-1.96 (m, 4H), 1.53 (s, 3H).

Step 7: A mixture of 1-(3-(2,3-dichlorophenyl)-4-methoxy-1H-pyrazolo[3,4-d]pyrimidin-6-yl)-4-methylpiperidin-4-amine (0.10 g, 27 μmol) and LiCl (114 mg) in NMP (0.5 mL) was stirred at 180° C. for 6 h. The residue was diluted with MeOH (5 mL) and adjusted pH=10 with NH₃H₂O. The residue was purified by reversed-phase HPLC to afford 6-(4-amino-4-methylpiperidin-1-yl)-3-(2,3-dichlorophenyl)-1H-pyrazolo[3,4-d]pyrimidin-4(5H)-one (7.0 mg, 17.7 μmol), as an off-white solid: ESMS [M+H]⁺=415.0; ¹H-NMR (400 MHz, methanol-d₄): δ 7.61-7.65 (m, 1H), 7.43-7.46 (m, 1H), 7.35-7.40 (m, 1H), 3.86-4.01 (m, 2H), 3.62-3.66 (m, 2H), 1.66-1.72 (m, 4H), 1.27-1.30 (d, 3H).

### Preparation of 6-(4-amino-4-methylpiperidin-1-yl)-3-(2,3-dichlorophenyl)-1H-pyrazolo[3,4-d]pyrimidin-4(5H)-one (Example F10)

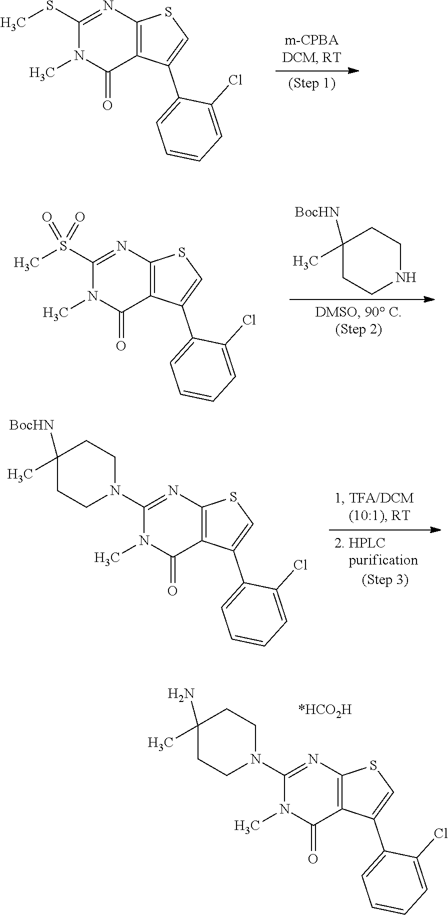 [SMILES: CSc1nc2scc(-c3ccccc3Cl)c2c(=O)n1C ; Cn1c(S(C)(=O)=O)nc2scc(-c3ccccc3Cl)c2c1=O ; CC1(NC(=O)OC(C)(C)C)CCNCC1 ; Cn1c(N2CCC(C)(NC(=O)OC(C)(C)C)CC2)nc2scc(-c3ccccc3Cl)c2c1=O ; Cn1c(N2CCC(C)(N)CC2)nc2scc(-c3ccccc3Cl)c2c1=O]

Step 1: To 5-(2-chlorophenyl)-3-methyl-2-(methylthio)thieno[2,3-d]pyrimidin-4(3H)-one (990 mg, 3.06 mmol) in 10 mL of DCM was added 3-chlorobenzoperoxoic acid (1.58 g, 9.18 mmol). The reaction mixture was stirred at room temperature for 2 hours, filtered through a pad of diatomaceous earth, and concentrated under reduced pressure to yield a residue which was purified by flash chromatography (10-50% EtOAc/hepanes to yield 5-(2-chlorophenyl)-3-methyl-2-(methylsulfonyl)thieno[2,3-d]pyrimidin-4(3H)-one.

Step 2: To 5-(2-chlorophenyl)-3-methyl-2-(methylsulfonyl)thieno[2,3-d]pyrimidin-4(3H)-one (150 mg, 0.42 mmol) and tert-butyl (4-methylpiperidin-4-yl)carbamate (135 mg, 0.6340 mmol) was added 3 mL of DMSO. The reaction mixture was stirred at 90° C. for 18 hours. After cooling the mixture was added to 10 mL of water and stirred for 15 minutes. The resulting precipitate was collected, taken up in 10 mL DCM, washed with water (3×5 mL), dried with sodium sulfate, filtered, and the volatiles removed under reduced pressure to produce tert-butyl (1-(5-(2-chlorophenyl)-3-methyl-4-oxo-3,4-dihydrothieno[2,3-d]pyrimidin-2-yl)-4-methylpiperidin-4-yl)carbamate, which was used in subsequent reactions as is.

Step 3: tert-Butyl (1-(5-(2-chlorophenyl)-3-methyl-4-oxo-3,4-dihydrothieno[2,3-d]pyrimidin-2-yl)-4-methylpiperidin-4-yl)carbamate taken up in DCM (10 mL) and TFA (1 mL) was added. The reaction mixture was stirred for 1 hour and the volatiles removed under reduced pressure. The residue was taken up in toluene (10 mL) and this also removed under reduced pressure (3×) to remove residual TFA. Purification by reversed phase HPLC provided 2-(4-amino-4-methylpiperidin-1-yl)-5-(2-chlorophenyl)-3-methylthieno[2,3-d]pyrimidin-4(3H)-one (41 mg): ESMS [M+H]⁺=389/391; ¹H-NMR (400 MHz, DMSO-d₆): δ 7.48 (d, J=7.57 Hz, 1H), 7.32-7.43 (m, 3H), 7.24 (s, 1H), 3.37-3.52 (m, 3H), 3.35 (s, 2H), 3.10-3.22 (m, 2H), 1.69-1.87 (m, 4H), 1.31 (s, 3H).

### Preparation of 6-[(3S,4S)-4-amino-3-methyl-2-oxa-8-azaspiro[4.5]decan-8-yl]-5-methyl-3-{1H,2H,3H-pyrrolo[3,2-b]pyridin-1-yl}-1H,4H,5H-pyrazolo[3,4-d]pyrimidin-4-one formate (Example F11)

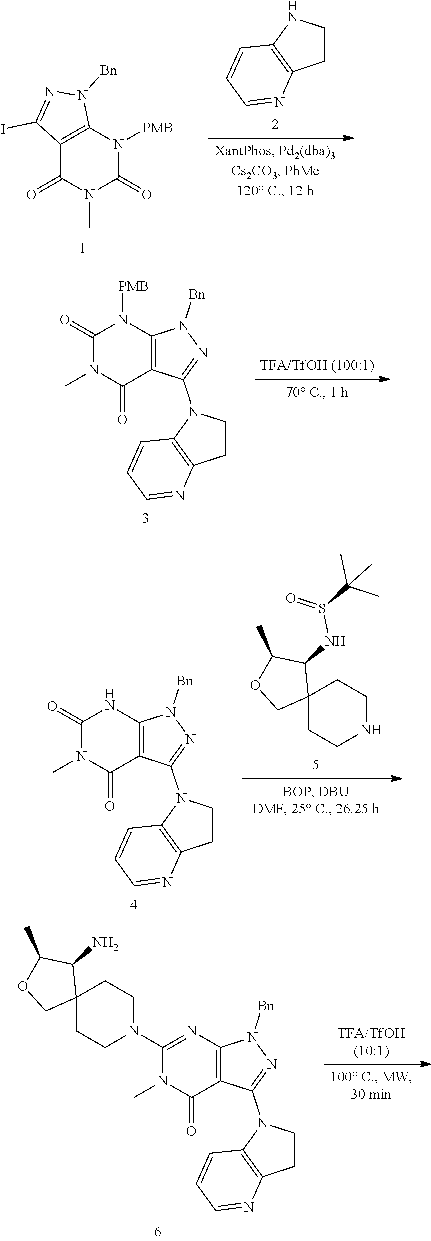

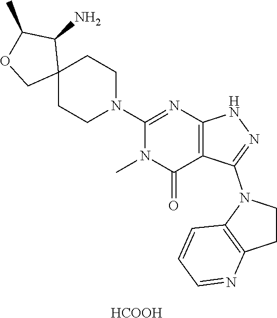 [SMILES: C[C@@H]1OCC2(CCN(c3nc4[nH]nc(N5CCc6ncccc65)c4c(=O)n3C)CC2)[C@@H]1N ; "HCOOH"]

1-Benzyl-3-iodo-7-[(4-methoxyphenyl)methyl]-5-methyl-1H,4H,5H,6H,7H-pyrazolo[3,4-d]pyrimidine-4,6-dione (1.0 g, 2.0 mmol), 1H,2H,3H-pyrrolo[3,2-b]pyridine (285.0 mg, 2.4 mmol), Pd₂(dba)₃ (728.0 mg, 796.0 μmol), XantPhos (460.0 mg, 796 μmol) and Cs₂CO₃ (2.6 g, 8.0 mmol) were added in PhMe (20.0 mL), the reaction mixture was evacuated and refilled for 3 times with N₂ and stirred at 120° C. for 12 hours. LCMS indicated 27% of desired product formed. The reaction mixture was concentrated under reduced pressure to give a residue and purified by flash silica gel chromatography (Petroleum ether: Ethyl acetate=100:0 to 100:70) to afford the product of 1-benzyl-7-[(4-methoxyphenyl)methyl]-5-methyl-3-{1H,2H,3H-pyrrolo[3,2-b]pyridin-1-yl}-1H,4H,5H,6H,7H-pyrazolo[3,4-d]pyrimidine-4,6-dione (360.0 mg, 36.5% yield) as a yellow solid.

1-Benzyl-7-[(4-methoxyphenyl)methyl]-5-methyl-3-{1H,2H,3H-pyrrolo[3,2-b]pyridin-1-yl}-1H,4H,5H,6H,7H-pyrazolo[3,4-d]pyrimidine-4,6-dione (180.0 mg, 363.0 μmol) and TfOH (95.2 μL, 1.1 mmol) were added in TFA (10.0 mL), the reaction mixture was stirred at 70° C. for 1 hour. LCMS indicated 58% of desired product formed. The combined reaction mixture was concentrated under reduced pressure, the residue was dissolved in Ethyl acetate (30.0 mL), washed with NaHCO₃ (30.0 mL) and brine (30.0 mL), the organic phase was concentrated under reduced pressure to give a residue and purified by prep-TLC (DCM:MeOH=10:1) to afford the product of 1-benzyl-5-methyl-3-{1H,2H,3H-pyrrolo[3,2-b]pyridin-1-yl}-1H,4H,5H,6H,7H-pyrazolo[3,4-d]pyrimidine-4,6-dione (200.0 mg, combined product) as a yellow solid.

1-Benzyl-5-methyl-3-{1H,2H,3H-pyrrolo[3,2-b]pyridin-1-yl}-1H,4H,5H,6H,7H-pyrazolo[3,4-d]pyrimidine-4,6-dione (200.0 mg, 534.0 μmol), BOP (675 mg, 1.6 mmol) were added in DMF (2.0 mL), the reaction mixture was stirred at 25° C. for 15 min, (S)-2-methyl-N-[(3S,4S)-3-methyl-2-oxa-8-azaspiro[4.5]decan-4-yl]propane-2-sulfinamide (146.0 mg, 534.0 μmol) and DBU (487.0 mg, 3.2 mmol) were added and stirred for 26 hours. LCMS indicated 38% of desired product formed. The reaction mixture was concentrated under reduced pressure to give a residue and purified by prep-TLC (Petroleum ether:Ethyl acetate=1:2) to afford the product of 6-[(3S,4S)-4-amino-3-methyl-2-oxa-8-azaspiro[4.5]decan-8-yl]-1-benzyl-5-methyl-3-{1H,2H,3H-pyrrolo[3,2-b]pyridin-1-yl}-1H,4H,5H-pyrazolo[3,4-d]pyrimidin-4-one (100.0 mg, 82% purity, 29.1% yield) as a yellow solid.

6-[(3S,4S)-4-amino-3-methyl-2-oxa-8-azaspiro[4.5]decan-8-yl]-1-benzyl-5-methyl-3-{1H,2H,3H-pyrrolo[3,2-b]pyridin-1-yl}-1H,4H,5H-pyrazolo[3,4-d]pyrimidin-4-one (90.0 mg, 82% purity, 140.0 μmol) was added in TFA (1.5 mL) and TfOH (0.15 mL), the reaction mixture was stirred under microwave irradiation at 100° C. for 30 min. LCMS indicated 66% of desired product formed. The reaction mixture concentrated under reduced pressure and the residue was dissolved in MeOH (10.0 mL), adjusted with K₂CO₃ (s) to pH=8 and purified by prep-HPLC (HCOOH) to afford the product of 6-[(3S,4S)-4-amino-3-methyl-2-oxa-8-azaspiro[4.5]decan-8-yl]-5-methyl-3-{1H,2H,3H-pyrrolo[3,2-b]pyridin-1-yl}-1H,4H,5H-pyrazolo[3,4-d]pyrimidin-4-one formate (2.5 mg, 3.7% yield) as a white solid. LCMS: calc. for C₂₂H₂₈N₈O₂: 436.2, found: [M+H]⁺ 437.1. HPLC: 99.0% purity at 254 nm. ¹HNMR (400 MHz, Methonal_d₄): δ 8.20 (s, 1H), 7.84-7.82 (m, 1H), 7.66-7.63 (m, 1H), 7.12-7.08 (m, 1H), 4.46-4.41 (m, 2H), 3.53-3.51 (m, 5H), 3.46 (s, 3H), 3.30-3.24 (m, 2H), 2.61-2.57 (m, 2H), 1.92-1.86 (m, 2H), 1.82-1.80 (m, 2H), 1.72-1.69 (m, 4H).

### Preparation of 6-[(3S,4S)-4-amino-3-methyl-2-oxa-8-azaspiro[4.5]decan-8-yl]-5-methyl-3-(1,2,3,4-tetrahydroisoquinolin-2-yl)-1H,4H,5H-pyrazolo[3,4-d]pyrimidin-4-one (Example F12)

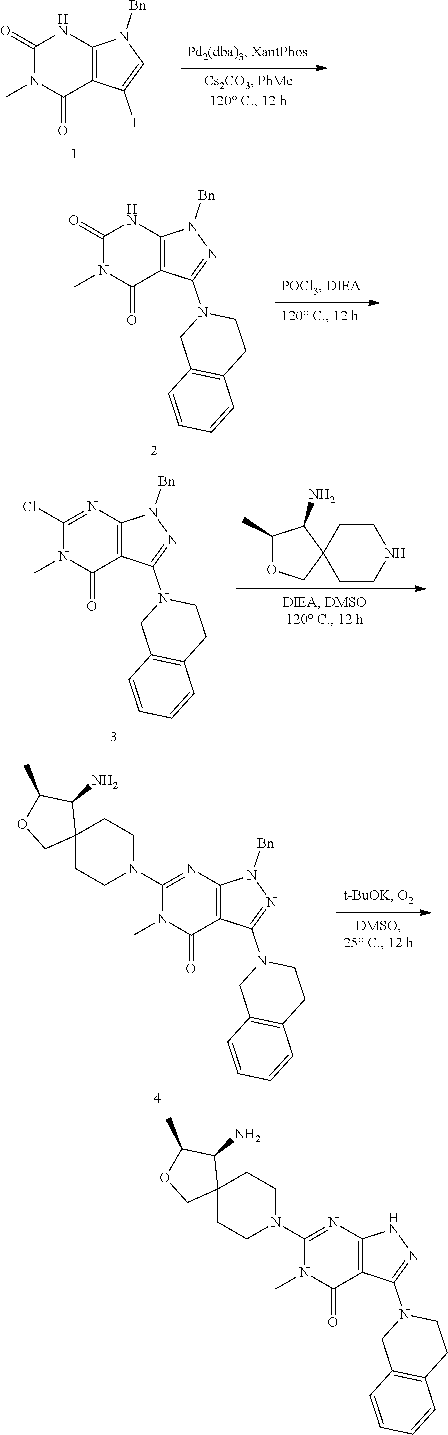 [SMILES: Cn1c(=O)[nH]c2c(c(I)cn2Cc2ccccc2)c1=O ; Cn1c(=O)[nH]c2c(c(N3CCc4ccccc4C3)nn2Cc2ccccc2)c1=O ; Cn1c(Cl)nc2c(c(N3CCc4ccccc4C3)nn2Cc2ccccc2)c1=O ; C[C@@H]1OCC2(CCNCC2)[C@@H]1N ; C[C@@H]1OCC2(CCN(c3nc4c(c(N5CCc6ccccc6C5)nn4Cc4ccccc4)c(=O)n3C)CC2)[C@@H]1N ; C[C@@H]1OCC2(CCN(c3nc4[nH]nc(N5CCc6ccccc6C5)c4c(=O)n3C)CC2)[C@@H]1N]

The mixture of 1-benzyl-3-iodo-5-methyl-1H,4H,5H,6H,7H-pyrazolo[3,4-d]pyrimidine-4,6-dione (1 g, 2.6 mmol), 1,2,3,4-tetrahydroisoquinoline (695 mg, 5.2 mmol), XantPhos (601 mg, 1.0 mmol), Pd₂(dba)₃ (952 mg, 1.0 mmol) and Cs₂CO₃ (2.5 g, 7.8 mmol) in PhMe (25 mL) was evacuated and refilled for 3 times using N₂ and stirred at 120° C. for 12 hours under N₂ atmosphere. The reaction mixture was diluted with H₂O (30 mL) and Ethyl acetate (100 mL). The partitioned layers were separated. The aqueous phase was extracted with ethyl acetate (100 mL×2). The combined organic layers were washed with brine (30 mL), dried over anhydrous Na₂SO₄, filtered and concentrated to give a residue, the residue was purified by column chromatography (Petroleum ether/Ethyl acetate=100:0 to 100:50) to afford the desired product of 11-benzyl-5-methyl-3-(1,2,3,4-tetrahydroisoquinolin-2-yl)-1H,4H,5H,6H,7H-pyrazolo[3,4-d]pyrimidine-4,6-dione (230 mg, 22.7% yield) as a brown solid.

The compound of 1-benzyl-5-methyl-3-(1,2,3,4-tetrahydroisoquinolin-2-yl)-1H,4H,5H,6H,7H-pyrazolo[3,4-d]pyrimidine-4,6-dione (230 mg, 0.6 mmol) and DIEA (0.3 mL, 1.8 mmol) were added in the POCl₃ (2 mL). The mixture was stirred at 120° C. for 12 h. TLC (Petroleum ether/Ethyl acetate=2:1) showed 2 was consumed completely and a new spot formed. The reaction mixture was concentrated to give a residue which was diluted with EtOAc (20 mL). The mixture was added slowly into ice cooled sat. NaHCO₃ (30 mL). The partitioned layers were separated. The aqueous phase was extracted with ethyl acetate (20 mL×3). The combined organic layers were washed with brine (50 mL), dried over anhydrous Na₂SO₄, filtered and concentrated to give a residue. The residue was purified by flash silica gel chromatography (Ethyl acetate/Petroleum ether=0/100 to 30/100) to give the product of 1-benzyl-6-chloro-5-methyl-3-(1,2,3,4-tetrahydroisoquinolin-2-yl)-1H,4H,5H-pyrazolo[3,4-d]pyrimidin-4-one (140 mg, 58% yield) as a yellow oil.

The compound of 1-benzyl-6-chloro-5-methyl-3-(1,2,3,4-tetrahydroisoquinolin-2-yl)-1H,4H,5H-pyrazolo[3,4-d]pyrimidin-4-one (120 mg, 0.3 mmol), (3S,4S)-3-methyl-2-oxa-8-azaspiro[4.5]decan-4-amine (100 mg, 0.6 mmol) and DIEA (0.15 mL, 0.9 mmol) were added in DMSO (2 mL). The reaction mixture was stirred at 120° C. for 12 hours. The mixture was diluted with EtOAc (100 mL) and H₂O (20 mL), the partitioned layers were separated. The organic phase was washed with H₂O (20 mL×5), brine (50 mL×2), dried over anhydrous Na₂SO₄, filtered and concentrated to give a residue. The residue was purified by prep-TLC (DCM:MeOH=10:1) to give the product of 6-[(3S,4S)-4-amino-3-methyl-2-oxa-8-azaspiro[4.5]decan-8-yl]-1-benzyl-5-methyl-3-(1,2,3,4-tetrahydroisoquinolin-2-yl)-1H,4H,5H-pyrazolo[3,4-d]pyrimidin-4-one (30.0 mg, 19% yield) as a yellow oil.

The compound of 6-[(3S,4S)-4-amino-3-methyl-2-oxa-8-azaspiro[4.5]decan-8-yl]-1-benzyl-5-methyl-3-(1,2,3,4-tetrahydroisoquinolin-2-yl)-1H,4H,5H-pyrazolo[3,4-d]pyrimidin-4-one (30 mg, 0.05 mmol) was dissolved in DMSO (1 mL). t-BuOK (1 mL, 1.0 mmol) was added dropwise at 25° C. O₂ was then bubbled into the solution for 10 min. The reaction mixture was quenched with saturated NH₄Cl. The mixture was concentrated and purified by prep-HPLC (HCl) to give the product of 6-[(3S,4S)-4-amino-3-methyl-2-oxa-8-azaspiro[4.5]decan-8-yl]-5-methyl-3-(1,2,3,4-tetrahydroisoquinolin-2-yl)-1H,4H,5H-pyrazolo[3,4-d]pyrimidin-4-one (6.3 mg, 1 HCl salt, 25% yield) as a yellow solid. LCMS: calc. for C₂₄H₃₁N₇O₂: 449.3, found: [M+H]⁺ 450.0. HPLC: 100% purity at 254 nm. ¹HNMR (400 MHz, CD₃OD): δ 7.09˜7.12 (m, 4H), 4.20˜4.22 (m, 1H), 3.97˜4.00 (m, 2H), 3.85˜3.88 (m, 1H), 3.73˜3.76 (m, 1H), 3.52˜3.65 (m, 2H), 3.38˜3.40 (m, 4H), 1.80˜1.92 (m, 3H), 1.64˜1.68 (m, 1H), 1.22˜1.24 (m, 3H). SFC: e.e. =100%, Column: Chiralcel OJ-3 100×4.6 mm I.D., 3 m Mobile phase: from 5% to 40% of ethanol (0.1% ethanolamine) in 4.5 min and hold 40% for 2.5 min, then 5% of ethanol (0.1% ethanolamine) for 1 min in CO₂, Flow rate: 2.8 mL/min, Column temperature: 40° C.

Using the general procedures described above, with reagents, starting materials, and conditions familiar to those skilled in the art, the following compounds representative of the disclosure are prepared:

| Compound | Structure |
|---|---|
| F2 | 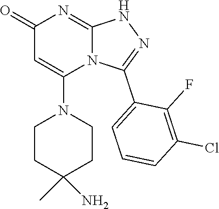 [SMILES: CC1(N)CCN(c2cc(=O)nc3[nH]nc(-c4cccc(Cl)c4F)n23)CC1] |

### SHP2 Allosteric Inhibition Assay.

SHP2 is allosterically activated through binding of bis-tyrosyl-phosphorylated peptides to its Src Homology 2 (SH2) domains. The latter activation step leads to the release of the auto-inhibitory interface of SHP2, which in turn renders the SHP2 protein tyrosine phosphatase (PTP) active and available for substrate recognition and reaction catalysis. The catalytic activity of SHP2 was monitored using the surrogate substrate DiFMUP in a prompt fluorescence assay format.

More specifically, the phosphatase reactions were performed at room temperature in 96-well black polystyrene plate, flat bottom, low flange, non-binding surface (Corning, Cat #3575) using a final reaction volume of 50 μl and the following assay buffer conditions: 60 mM HEPES, pH 7.2, 75 mM NaCl, 75 mM KCl, 1 mM EDTA 0.005% Brij-35, 5 mM DTT.

The inhibition of SHP2 by compounds of the disclosure (concentrations varying from 0.003-100 μM) was monitored using an assay in which 0.25 nM of SHP2 was incubated with of 0.5 UM of peptide IRS1_pY1172(dPEG8)pY1222 (sequence: H2N-LN(pY)IDLDLV(dPEG8)LST(pY)ASINFQK-amide). After 30-60 minutes incubation at 25° C., the surrogate substrate DiFMUP (Invitrogen, cat #D6567, 100 μM final) was added to the reaction and the conversion of DiFMUP to 6,8-difluoro-7-hydroxyl-4-methylcoumarin (DiFMU) was monitored continuously for 10 minutes with excitation at 355 nm and emission at 460 nm using a microplate reader (PolarStar, BMG). The inhibitor dose response curves were analyzed using normalized IC₅₀ regression curve fitting with control based normalization.

Biochemical assay results for compounds of the disclosure are shown in Table 1. In Table 1, A means an IC₅₀ of less than 5 μM; B means an IC₅₀ equal to 5 μM but less than 20 μM; and C means an IC₅₀ of 20 μM or more.

TABLE 1
SHP2 IC₅₀ Assay Results

| Compound | SHP2 IC₅₀ |
|---|---|
| A1 | A |
| A2 | B |
| A3 | A |
| A4 | C |
| A5 | A |
| A6 | B |
| A7 | A |
| A8 | C |
| A9 | B |
| A10 | A |
| A11 | A |
| A12 | C |

Biochemical assay results for compounds of the disclosure are shown in Table 2. In Table 2. A means an IC₅₀ of less than 100 μM; and B means an IC₅₀ of 100 μM or more.

TABLE 2
SHP2 IC₅₀ Assay Results

| Compound | SHP2 IC₅₀ |
|---|---|
| B1 | A |
| B2 | B |
| B3 | B |
| B4 | B |

Biochemical assay results for compounds of the disclosure are shown in examples and Table 3. In Table 3, A means an IC₅₀ of less than 500 M; B means an IC₅₀ equal to 500 μM but less than 1,000 μM and C means an IC₅₀ of 1,000 μM or more.

TABLE 3
SHP2 IC₅₀ Assay Results

| Compound | SHP2 IC₅₀ |
|---|---|
| C1 | A |
| C2 | A |
| C3 | B |
| C4 | A |
| C5 | B |
| C6 | B |
| C7 | B |
| C8 | B |
| C9 | B |
| C10 | B |
| C11 | A |
| C12 | A |
| C13 | A |
| C14 | B |
| C15 | A |
| C16 | A |
| C17 | B |
| C18 | B |
| C19 | B |
| C20 | A |
| C21 | A |
| C22 | C |
| C23 | C |
| C24 | C |
| C25 | C |
| C26 | C |
| C27 | C |
| C28 | C |
| C29 | C |
| C30 | C |
| C31 | C |
| C32 | C |
| C33 | C |
| C34 | C |
| C35 | C |
| C36 | C |
| C37 | C |
| C38 | C |
| C39 | C |
| C40 | C |
| C41 | C |
| C42 | C |
| C43 | C |
| C44 | C |
| C45 | C |
| C46 | C |
| C47 | C |
| C48 | C |
| C49 | C |

Biochemical assay results for compounds of the disclosure are shown in Table 4. In Table 4, A means an IC₅₀ of more than 1 μM but less than 10 μM.

TABLE 4
SHP2 IC₅₀ Assay Results

| Compound | SHP2 IC₅₀ |
|---|---|
| D1 | A |
| D2 | A |

Biochemical assay results for compounds of the disclosure are shown in Table 5. In Table 5, A means an IC₅₀ of less than 50 μM; B means an IC₅₀ equal to 50 μM but less than 100 μM; and C means an IC₅₀ of 100 μM or more.

TABLE 5
SHP2 IC₅₀ Assay Results

| Compound | SHP2 IC₅₀ |
|---|---|
| E1 | B |
| E2 | A |
| E3 | C |
| E4 | C |

Biochemical assay results for compounds of the disclosure are shown in Table 6. In Table 6, A means an IC₅₀ of less than 1 μM; B means an IC₅₀ equal to 1 μM but less than 20 μM; and C means an IC₅₀ of 20 μM or more.

TABLE 6
SHP2 IC₅₀ Assay Results

| Example | SHP2 IC₅₀ |
|---|---|
| F1 | A |
| F2 | C |
| F3 | C |
| F4 | A |
| F5 | B |
| F6 | A |
| F7 | A |
| F8 | A |
| F9 | A |
| F10 | C |
| F11 | B |
| F12 | A |

### INCORPORATION BY REFERENCE

All patents, patent applications and publications cited herein are hereby incorporated by reference in their entirety. The disclosures of these publications in their entireties are hereby incorporated by reference into this application in order to more fully describe the state of the art as known to those skilled therein as of the date of the disclosure described and claimed herein.

Although the disclosure has been described and illustrated in the foregoing illustrative embodiments, it is understood that the present disclosure has been made only by way of example, and that numerous changes in the details of implementation of the disclosure can be made without departing from the spirit and scope of the disclosure, which is limited only by the claims that follow. Features of the disclosed embodiments can be combined and rearranged in various ways within the scope and spirit of the disclosure.

What is claimed is:

1. A compound represented by Formula i, ii, or iii: 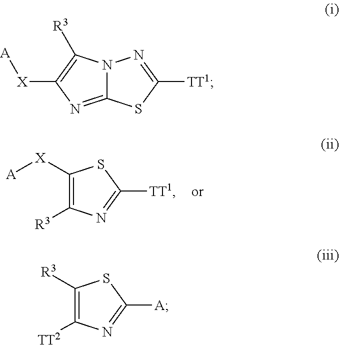
or a pharmaceutically acceptable salt or stereoisomer thereof, wherein:
TT¹ is selected from the group consisting of: 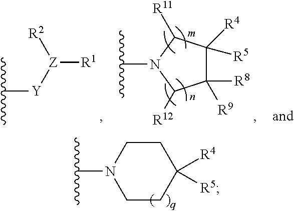
TT² is selected from the group consisting of: 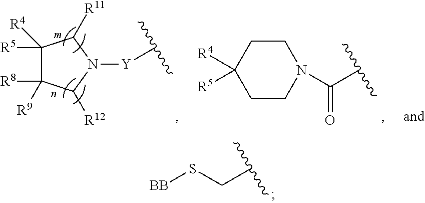
X is selected from the group consisting of a bond, —O—, —NR^{X1}—, —C(O)NR^{X11}—, —S(O)_{w}— (wherein w is 0, 1 or 2), —C(O)—, —C(R^{X2}R^{X3})—, —O—C(R^{X4}R^{X5})—, —C(R^{X4}R^{X5})—O—, and —C═C(R^{X6}R^{X7})—;
Y is selected from the group consisting of a bond, —C(O)—, —NR^{Y}—, —C₁₋₃alkylene-NR^{Y}—, and —NR^{Y}—C₁₋₃alkylene-;
Z is selected from the group consisting of N and C(R^{Z});
wherein if Y is a bond or —C(O)—, Z is N; and wherein if Y is —NR^{Y}—, —C₁₋₃alkylene-NR^{Y}—, or —NR^{Y}—C₁₋₃alkylene-, Z is C(R^{Z});
R^{Z} is selected from the group consisting of hydrogen and C₁₋₆alkyl; wherein C₁₋₆alkyl may optionally be substituted by halogen, hydroxyl, and —NR^{a}R^{b},
A is selected from the group consisting of a 6-10 membered monocyclic or bicyclic aryl, a 5-7 membered monocyclic heteroaryl having one or more heteroatoms each independently selected from O, S, or N, a 8-10 membered bicyclic heteroaryl having one or more heteroatoms each independently selected from O, S, or N; and a 4-7 membered heterocyclyl, wherein A may optionally be substituted by one, two, three or more substituents each independently selected from the group consisting of —R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹, —P(O)(R¹⁰)₂—C(O)R¹, —C(O)N(R¹⁰)₂, oxo, halogen and cyano;
R¹⁰ is independently selected from the group consisting of hydrogen, C₁₋₆alkyl, C₁₋₆ haloalkyl, C₃₋₆cycloalkyl, heterocycloalkyl, aryl, and heteroaryl; wherein R¹⁰ may optionally be substituted by one, two, three or more substituents each independently selected from the group consisting of halogen, —C(O)R²⁰, C₁₋₆alkyl and C₁₋₆haloalkyl;
R²⁰ is selected from the group consisting of hydroxyl, halogen, and C₁₋₆alkyl;
R¹ and R² are each selected from the group consisting of hydrogen and C₁₋₁₀alkyl; wherein C₁₋₁₀alkyl may optionally be substituted by one, two, three or more substituents each independently selected from R^{P};
or R¹ and R², together with carbon or nitrogen to which they are attached, form a 3-7 membered saturated carbocyclic or 4-7 membered saturated heterocyclic ring B having one or two heteroatoms each independently selected from the group consisting of 0, S(O)_{w} (wherein w is 0, 1, or 2), and NR^{h}; wherein carbocyclic or heterocyclic ring B may be monocyclic or bicyclic;
carbocyclic or heterocyclic ring B may optionally be substituted on one or more available carbons by one, two, three or more substituents each independently selected from the group consisting of halogen, hydroxyl, cyano, oxo, NR^{a}R^{b}, —CO₂H, —C(O)—NR^{a}R^{b}, —S(O)₂—NR^{a}R^{b}, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})—, C₁₋₆alkoxycarbonyl-N(R^{a})—, aryl and heteroaryl; wherein C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})— and C₁₋₆alkoxycarbonyl-N(R^{a})— may optionally be substituted by one or more substituents each independently selected from R^{p}; and wherein aryl and heteroaryl may optionally be substituted by one or more substituents each independently selected from R;
R^{X1} is selected from the group consisting of hydrogen, C₁₋₆alkyl and phenyl, or R^{X1} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹, —P(O)(R¹⁰)₂, —C(O)R¹⁰, —C(O)N(R¹⁰)₂, oxo, halogen and cyano;
R^{X11} is selected from the group consisting of hydrogen, C₁₋₆alkyl and phenyl, or R¹ and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹, —P(O)(R¹⁰)₂—C(O)R¹⁰, —C(O)N(R¹⁰)₂, oxo, halogen and cyano;
R^{X2} and R^{X3} are each independently selected from the group consisting of hydrogen, halogen, hydroxyl, —NR^{a}R^{b}, —C(O)—NR^{a}R^{b}, C₁₋₆alkyl and C₁₋₆alkoxy; wherein C₁₋₆alkyl and C₁₋₆alkoxy may optionally be substituted by one or more substituents each independently selected from R;
or R^{X2} and R^{X3} together with the carbon to which they are attached form a 3-6 membered carbocycle optionally substituted by one or more substituents each independently selected from the group consisting of hydrogen, halogen, NR^{a}R^{b}, —C(O)NR^{a}R^{b}, oxo, C₁₋₆alkyl and C₁₋₆alkoxy;
R^{X4} and R^{X5} are each independently selected from the group consisting of hydrogen and C₁₋₆alkyl; wherein C₁₋₆alkyl may optionally be substituted by one or more substituents each independently selected from R^{P};
R^{X6} and R^{X7} are each independently selected from the group consisting of hydrogen, halogen, —C(O)—NR^{a}R^{b}, cyano and C₁₋₆alkyl; wherein C₁₋₆alkyl may optionally be substituted by one or more substituents each independently selected from R^{P};
R^{Y} is selected from the group consisting of hydrogen, C₁₋₆alkyl and phenyl;
R³ is selected from the group consisting of hydrogen, halogen, hydroxyl, C₁₋₆alkyl and NR^{a}R^{b};
R⁴ and R⁵ are each independently selected from the group consisting of hydrogen, hydroxyl, C₁₋₆alkyl, C₁₋₆alkoxy, C₁₋₆alkyl-O—R⁶, —C(O)N(R⁶)₂, —N(R⁶)₂, halogen, C₁₋₆alkyl-N(R⁶)₂, and cyano; wherein C₁₋₆alkyl, C₁₋₆alkoxy, C₁₋₆alkyl-O—R⁶, and C₁₋₆alkyl-N(R⁶)₂ may optionally be substituted by one or more substituents each independently selected from the group consisting of hydroxyl, —NH₂, oxo, and halogen,
or R⁴ and R⁵, taken together with the atoms to which they are attached, form a 3-7 membered carbocyclic or heterocyclic saturated or partially unsaturated ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;
or R⁴ and R⁸, taken together with the atoms to which they are attached, form a 4-7 membered carbocyclic or heterocyclic ring, wherein the ring may optionally be substituted by one or more substituents each independently selected from the group consisting of hydroxyl, C₁₋₆alkyl, C₁₋₆-alkoxy, —N(R⁶)₂, halogen, oxo, and cyano;
or R⁴ is absent, and R⁴ and R⁸, taken together with the atoms to which they are attached, form a 3-membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;
R⁶ is independently for each occurrence selected from the group consisting of hydrogen, C₁₋₆alkyl and phenyl;
R¹¹ and R¹² are each independently selected from the group consisting of hydrogen, hydroxyl, C₁₋₆alkyl, C₁₋₆alkoxy, C₁₋₆alkyl-O—R⁶, aryl, arylalkylene, heteroaryl, heteroarylalkylene, —C(O)N(R⁶)₂, —N(R⁶)₂, halogen, C₁₋₆alkyl-N(R⁶)₂, —CO₂H, and cyano; wherein C₁₋₆alkyl, C₁₋₆alkoxy, C₁₋₆alkyl-O—R⁶, aryl, arylalkylene, heteroaryl, heteroarylalkylene, and C₁₋₆alkyl-N(R⁶)₂ may optionally be substituted by one or more substituents each independently selected from the group consisting of hydroxyl, —NH₂, and halogen;
or R¹¹ and R¹², taken together with the atoms to which they are attached, form a 5-7 membered heterocyclic ring;
or R⁴ and R¹², taken together with the atoms to which they are attached, form a 5-7 membered carbocyclic or heterocyclic ring;
or R⁸ and R¹¹, taken together with the atoms to which they are attached, form a 5-7 membered carbocyclic or heterocyclic ring;
R⁸ and R⁹ are each independently selected from the group consisting of hydrogen, C₁₋₆)alkyl, C₁₋₆alkyl-N(R⁶)₂, —OR⁶, C₁₋₆alkyl-O—R⁶, —C(O)NH₂, —N(R⁶)₂, halogen, and cyano;
each of m and n is, independently, 0, 1, 2, or 3, with m+n being at least 2 and no more than 4;
q is 0 or 1;
R^{f} is independently selected, for each occurrence, from the group consisting of R^{P}, C₁₋₆alkyl, —C₁₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, (wherein wherein w is 0, 1 or 2), C₁₋₆alkylcarbonyl-N(R^{a})— and C₁₋₆alkoxycarbonyl-N(R^{a})—; wherein C₁₋₆alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, C₁₋₆alkylcarbonyl-N(R^{a})—, and C₁₋₆alkoxycarbonyl-N(R^{a})— may be optionally substituted by one or more substituents selected from R^{p};
R^{h} is independently selected, for each occurrence, from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆ alkylcarbonyl-, C₁₋₆alkoxycarbonyl-, R^{a}R^{b}N-carbonyl- and R^{a}R^{b}N—SO₂—; wherein C₁₋₆alkyl, C₃₋₆alkenyl, C₃₋₆alkynyl, C₃₋₆cycloalkyl, C₁₋₆alkyl-S(O)₂—, C₁₋₆alkylcarbonyl- and C₁₋₆alkoxycarbonyl- may optionally be substituted by one or more substituents selected from R^{P};
R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl;
or R and R^{b}, together with the nitrogen to which they are attached, may form a 4-6 membered monocyclic heterocyclic ring, which may have an additional heteroatom selected from the group consisting of O, S, and N; wherein the 4-6 membered heterocyclic ring may optionally be substituted by one or more substituents selected from the group consisting of halogen, cyano, oxo or hydroxyl;
BB is optionally substituted monocyclic or bicyclic heteroaryl; and
R^{p} is independently selected, for each occurrence, from the group consisting of halogen, hydroxyl, cyano, C₁₋₆alkoxy, R^{a}R^{b}N—, R^{a}R^{b}N-carbonyl-, R^{a}R^{b}N—SO₂—, and R^{a}R^{b}N-carbonyl-N(R^{a}).

2. A compound of Formula I, or a pharmaceutically acceptable salt or stereoisomer thereof, wherein Formula I is represented by: 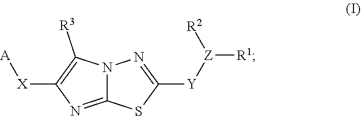
wherein
X is selected from the group consisting of a bond, —O—, —NR^{X1}— —C(O)NR^{X11}—, —S(O)_{w}— (wherein w is 0, 1 or 2), —C(O)—, —C(R^{X2}R^{X3})—, —O—C(R^{X3}R^{X4})—, —C(R^{X4}R^{X5})—O—, and —C(R^{X6}R^{X7})—;
Y is selected from the group consisting of a bond, —C(O)—, —NR^{Y}—, —C₁₋₃alkylene-NR^{Y}—, and —NR^{Y}—C₁₋₃alkylene-;
Z is selected from the group consisting of N and C(R^{Z});
wherein if Y is a bond or —C(O)—, Z is N; and wherein if Y is —NR^{Y}—, —C₁₋₃alkylene-NR^{Y}—, or —NR^{Y}—C₁₋₃alkylene-, Z is C(R^{Z});
R^{Z} is selected from the group consisting of hydrogen and C₁₋₆alkyl; wherein C₁₋₆alkyl may optionally be substituted by halogen, hydroxyl, and —NR^{a}R^{b},
A is selected from the group consisting of a 6-10 membered monocyclic or bicyclic aryl, a 5-7 membered monocyclic heteroaryl having one or more heteroatoms each independently selected from O, S, or N, a 8-10 membered bicyclic heteroaryl having one or more heteroatoms each independently selected from O, S, or N; and a 4-7 membered heterocyclyl, wherein A may optionally be substituted by one, two, three or more substituents each independently selected from the group consisting of —R¹⁰, —OR¹⁰, —S(O)_{w}R¹⁰ (wherein w is 0, 1 or 2), —N(R¹⁰)₂, —OS(O)—R¹⁰ (wherein w is 0, 1, or 2), —S(O)_{w}—N(R¹⁰)₂ (wherein w is 0, 1 or 2), —S(O)(NH)R¹, —P(O)(R¹⁰)₂, —C(O)R¹⁰, —C(O)N(R¹⁰)₂, oxo, halogen and cyano;
R¹⁰ is independently selected from the group consisting of hydrogen, C₁₋₆alkyl, C₁₋₆ haloalkyl, C₃₋₆cycloalkyl, heterocycloalkyl, aryl, and heteroaryl; wherein R¹⁰ may optionally be substituted by one, two, three or more substituents each independently selected from the group consisting of halogen, —C(O)R²⁰, C₁₋₆alkyl and C₁₋₆haloalkyl;
R²⁰ is selected from the group consisting of hydroxyl, halogen, and C₁₋₆alkyl;
R¹ and R² are each selected from the group consisting of hydrogen and C₁₋₁₀alkyl; wherein C₁₋₁₀alkyl may optionally be substituted by one, two, three or more substituents each independently selected from R^{P};
or R¹ and R², together with carbon or nitrogen to which they are attached, form a 3-7 membered saturated carbocyclic or 4-7 membered saturated heterocyclic ring B having one or two heteroatoms each independently selected from the group consisting of 0, S(O), (wherein w is 0, 1, or 2), and NR; wherein carbocyclic or heterocyclic ring B may be monocyclic or bicyclic;
carbocyclic or heterocyclic ring B may optionally be substituted on one or more available carbons by one, two, three or more substituents each independently selected from the group consisting of halogen, hydroxyl, cyano, oxo, NR^{a}R^{b}, —CO₂H, —C(O)—NR^{a}R^{b}, —S(O)₂—NR^{a}R^{b}, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})—, C₁₋₆alkoxycarbonyl-N(R^{a})—, aryl and heteroaryl; wherein C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})— and C₁₋₆alkoxycarbonyl-N(R^{a})— may optionally be substituted by one or more substituents each independently selected from R^{p}; and wherein aryl and heteroaryl may optionally be substituted by one or more substituents each independently selected from R;
R^{X1} is selected from the group consisting of hydrogen, C₁₋₆alkyl and phenyl, or R and ring A together with the nitrogen to which they are attached form an 8-10 membered saturated or partially unsaturated bicyclic heterocyclyl which may have one or more additional heteroatoms each independently selected from the group consisting of O, S, and N;
wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;
R^{X11} is selected from the group consisting of hydrogen, C₁₋₆alkyl and phenyl, or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;
R^{X2} and R^{X3} are each independently selected from the group consisting of hydrogen, halogen, hydroxyl, —NR^{a}R^{b}, —C(O)—NR^{a}R^{b}, C₁₋₆alkyl and C₁₋₆alkoxy; wherein C₁₋₆alkyl and C₁₋₆alkoxy may optionally be substituted by one or more substituents each independently selected from R^{p};
or R^{X4} and R^{X5} together with the carbon to which they are attached form a 3-6 membered carbocycle optionally substituted by one or more substituents each independently selected from the group consisting of hydrogen, halogen, NR^{a}R^{b}, —C(O)NR^{a}R^{b}, oxo, C₁₋₆alkyl and C₁₋₆alkoxy;
R^{X4} and R^{X5} are each independently selected from the group consisting of hydrogen and C₁₋₆alkyl; wherein C₁₋₆alkyl may optionally be substituted by one or more substituents each independently selected from R^{P};
R^{X6} and R^{X7} are each independently selected from the group consisting of hydrogen, halogen, —C(O)—NR^{a}R^{b}, cyano and C₁₋₆alkyl; wherein C₁₋₆alkyl may optionally be substituted by one or more substituents each independently selected from R^{P};
R^{Y} is selected from the group consisting of hydrogen, C₁₋₆alkyl and phenyl;
R³ is selected from the group consisting of hydrogen, halogen, hydroxyl, C₁₋₆alkyl and NR^{a}R^{b};
R^{f} is independently selected, for each occurrence, from the group consisting of R^{P}, C₁₋₆alkyl, —C₁₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, (wherein wherein w is 0, 1 or 2), C₁₋₆alkylcarbonyl-N(R^{a})— and C₁₋₆alkoxycarbonyl-N(R^{a})—; wherein C₁₋₆alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, C₁₋₆alkylcarbonyl-N(R^{a})—, and C₁₋₆alkoxycarbonyl-N(R^{a})— may be optionally substituted by one or more substituents selected from R^{p};
R^{h} is independently selected, for each occurrence, from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆alkylcarbonyl-, C₁₋₆alkoxycarbonyl-, R^{a}R^{b}N-carbonyl- and R^{a}R^{b}N—SO₂—; wherein C₁₋₆alkyl, C₃₋₆alkenyl, C₃₋₆alkynyl, C₃₋₆cycloalkyl, C₁₋₆alkyl-S(O)₂—, C₁₋₆alkylcarbonyl- and C₁₋₆alkoxycarbonyl- may optionally be substituted by one or more substituents selected from R^{P};
R and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl;
or R and R^{b}, together with the nitrogen to which they are attached, may form a 4-6 membered monocyclic heterocyclic ring, which may have an additional heteroatom selected from the group consisting of O, S, and N; wherein the 4-6 membered heterocyclic ring may optionally be substituted by one or more substituents selected from the group consisting of halogen, cyano, oxo or hydroxyl; and
R^{p} is independently selected, for each occurrence, from the group consisting of halogen, hydroxyl, cyano, C₁₋₆alkoxy, R^{a}N—, R^{a}R^{b}N-carbonyl-, R^{a}R^{b}N—SO₂—, and R^{a}R^{b}N-carbonyl-N(R^{a}).

3. The compound of claim 2, wherein X is selected from the group consisting of a bond, —O—, —NR^{X1}— and —NR^{X11}—.

4. The compound of claim 2 or 3, wherein A is selected from the group consisting of phenyl, pyridyl, quinolinyl, indolyl, and indolinyl, wherein phenyl, pyridyl, quinolinyl, indolyl and indolinyl may optionally be substituted by one, two or three substituents each independently selected from the group consisting of —R¹⁰, —OR¹⁰, —SR¹⁰, —N(R¹⁰)₂, —OSO₂R¹⁰, —SO₂R¹⁰, —C(O)N(R¹⁰)₂, halogen, and cyano.

5. The compound of any one of claims 2-4, wherein A is phenyl; wherein phenyl may optionally be substituted by one, two, or three substituents each independently selected from the group consisting of —OR¹⁰, halogen, and cyano.

6. The compound of any one of claims 2-4, wherein A is pyridyl; wherein pyridyl may optionally be substituted by one, two, or three substituents each independently selected from the group consisting of —OR¹⁰, halogen, and cyano.

7. The compound of claim 2 or 3, wherein R^{X1} and ring A together with the nitrogen to which they are attached form a 1,2,3,4-tetrahydro-1,5-naphthyridine moiety.

8. The compound of claim 2 or 3, wherein R^{X11} and ring A together with the nitrogen to which they are attached form a 1,2,3,4-tetrahydro-1,5-naphthyridine moiety.

9. The compound of claim 2 or 3, wherein R^{X1} and ring A together with the nitrogen to which they are attached form a 1,2,3,4-tetrahydroquinoline moiety.

10. The compound of claim 2 or 3, wherein R^{X11} and ring A together with the nitrogen to which they are attached form a 1,2,3,4-tetrahydroquinoline moiety.

11. The compound of any one of claims 2-10, wherein R³ is selected from the group consisting of hydrogen, CH₃ and —NH₂.

12. The compound of any one of claims 2-11, wherein Y is a bond and Z is N.

13. The compound of any one of claims 2-12, wherein R¹ and R² together with the nitrogen to which they are attached form heterocyclic ring B; wherein ring B is selected from the group consisting of pyrrolidinyl, piperidinyl, azepanyl and morpholinyl; wherein pyrrolidinyl, piperidinyl, azepanyl and morpholinyl may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃alkoxy; wherein C₁₋₃alkyl and C₁₋₃alkoxy may optionally be substituted by halogen, hydroxyl and NR^{a}R^{b}.

14. The compound of claim any one of claims 2-11, wherein Y is —NH—, —N(CH₃)—, —N(H)—CH₂—, or —N(CH₃)—CH₂— and Z is C(H).

15. The compound of claim 14, wherein R¹ and R² together with the carbon to which they are attached form carbocyclic ring B; wherein ring B is selected from the group consisting of cyclopentyl and cyclohexyl, and wherein cyclopentyl and cyclohexyl may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃alkoxy; wherein C₁₋₃alkyl and C₁₋₃alkoxy may optionally be substituted by halogen, hydroxyl and —NR^{a}R^{b}.

16. The compound of claim 14, wherein R¹ and R² together with the carbon to which they are attached form heterocyclic ring B; wherein ring B is selected from the group consisting of tetrahydrofuranyl and tetrahydropyranyl, wherein tetrahydrofuranyl and tetrahydropyranyl may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃alkoxy; wherein C₁₋₃alkyl and C₁₋₃alkoxy may optionally be substituted by halogen, hydroxyl and NR^{a}R^{b}.

17. The compound of any one of claims 2-16, wherein ring B is substituted on an available carbon by a substituent selected from the group consisting of —NH₂, —CH₃, and —CH₂NH₂.

18. The compound of any one of claims 2-12, wherein R¹ is hydrogen and R² is branched C₁₋₁₀alkyl; wherein C₁₋₁₀alkyl may optionally be substituted by one, two, three or more substituents each independently selected from the group consisting of halogen and —NH₂.

19. A compound of Formula II, or a pharmaceutically acceptable salt or stereoisomer thereof, wherein Formula II is represented by: 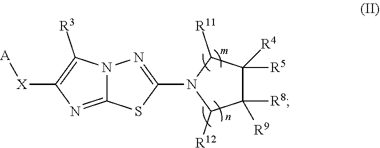
wherein
X is selected from the group consisting of a bond, —O—, NR, —C(O)NR^{X11}—, —S(O)_{w}— (wherein w is 0, 1 or 2), —C(O)— and —(R²R³)—;
A is selected from the group consisting of phenyl, a 5-7 membered monocyclic heteroaryl having one or more heteroatoms each independently selected from O, S, and N, a 8-10 membered bicyclic heteroaryl having one or more heteroatoms each independently selected from the group consisting of O, S, and N, and a 4-7 membered heterocyclyl; wherein A may optionally be substituted by one, two, three or more substituents each independently selected from the group consisting of —R¹⁰, —OR, —N(R¹⁰)₂, —C(O)N(R¹⁰)₂, oxo, halogen and cyano;
R¹⁰ is independently selected from the group consisting of hydrogen, C₁₋₆alkyl, C₁₋₆ haloalkyl, C₃₋₆cycloalkyl, heterocycloalkyl, aryl, and heteroaryl, where R¹⁰ may optionally be substituted by one, two, three or more substituents each independently selected from the group consisting of halogen, —C(O)R²⁰, C₁₋₆alkyl and C₁₋₆haloalkyl;
R²⁰ is selected from the group consisting of hydroxyl, halogen, and C₁₋₆alkyl;
R^{X1} is selected from the group consisting of hydrogen, C₁₋₆alkyl and phenyl; or R¹ and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —N(R¹⁰)₂, —C(O)N(R¹⁰)₂, oxo, halogen and cyano;
R^{X11} is selected from the group consisting of hydrogen, C₁₋₆alkyl and phenyl; or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —N(R¹⁰)₂, —C(O)N(R¹⁰)₂, oxo, halogen and cyano;
R^{X2} and R^{X3} are each independently selected from the group consisting of hydrogen, halogen, hydroxyl, —NR^{a}R^{b}, —(O)—NR^{a}R^{b}, C₁₋₆alkyl and C₁₋₆alkoxy; or R^{X2} and R^{X3} together with the carbon to which they are attached form a 3-6 membered carbocyclyl which may optionally be substituted by one or more substituents each independently selected from the group consisting of hydrogen, halogen, —NR^{a}R^{b}, —C(O)—NR^{a}R^{b}, oxo, C₁₋₆alkyl and C₁₋₆ alkoxy;
R³ is selected from the group consisting of hydrogen, halogen, hydroxyl, C₁₋₆alkyl and —NR^{a}R^{b};
R⁴ and R⁵ are each independently selected from the group consisting of hydrogen, hydroxyl, C₁₋₆alkyl, C₁₋₆alkoxy, C₁₋₆alkyl-O—R⁶, —C(O)N(R⁶)₂, —N(R⁶)₂, halogen, C₁₋₆alkyl-N(R⁶)₂, and cyano; wherein C₁₋₆alkyl, C₁₋₆alkoxy, C₁₋₆alkyl-O—R⁶, and C₁₋₆alkyl-N(R⁶)₂ may optionally be substituted by one or more substituents each independently selected from the group consisting of hydroxyl, —NH₂, oxo, and halogen,
or R⁴ and R⁵, taken together with the atoms to which they are attached, form a 3-7 membered carbocyclic or heterocyclic saturated or partially unsaturated ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;
or R⁴ and R⁸, taken together with the atoms to which they are attached, form a 4-7 membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;
or R⁴ is absent, and W and R, taken together with the atoms to which they are attached, form a 3-membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;
R⁶ is independently for each occurrence selected from the group consisting of hydrogen, C₁₋₆alkyl and phenyl;
R¹¹ and R¹² are each independently selected from the group consisting of hydrogen, hydroxyl, C₁₋₆alkyl, C₁₋₆alkoxy, C₁₋₆alkyl-O—R⁶, aryl, arylalkylene, heteroaryl, heteroarylalkylene, —C(O)N(R⁶)₂, —N(R⁶)₂, halogen, C₁₋₆alkyl-N(R⁶)₂, —CO₂H, and cyano; wherein C₁₋₆alkyl, C₁₋₆alkoxy, C₁₋₆alkyl-O—R⁶, aryl, arylalkylene, heteroaryl, heteroarylalkylene, and C₁₋₆alkyl-N(R⁶)₂ may optionally be substituted by one or more substituents each independently selected from the group consisting of hydroxyl, —NH₂, and halogen;
or R¹¹ and R¹², taken together with the atoms to which they are attached, form a 5-7 membered heterocyclic ring;
or R⁴ and R¹², taken together with the atoms to which they are attached, form a 5-7 membered carbocyclic or heterocyclic ring;
or R⁸ and R¹¹, taken together with the atoms to which they are attached, form a 5-7 membered carbocyclic or heterocyclic ring;
R⁸ and R⁹ are each independently selected from the group consisting of hydrogen, C₁₋₆)alkyl, C₁₋₆alkyl-N(R⁶)₂, —OR⁶, C₁₋₆alkyl-O—R⁶, —C(O)NH₂, —N(R⁶)₂, halogen, and cyano; and
each of m and n is, independently, 0, 1, 2, or 3, with m+n being at least 2 and no more than 4.

20. The compound of claim 19, wherein A is phenyl; wherein phenyl may optionally be substituted by one, two, or three substituents each independently selected from the group consisting of —OR¹⁰, halogen, and cyano.

21. The compound of claim 19, wherein A is pyridyl; wherein pyridyl may optionally be substituted by one, two, or three substituents each independently selected from the group consisting of —OR¹⁰, halogen, and cyano.

22. The compound of claim 19, wherein R^{X1} and ring A together with the nitrogen to which they are attached form a 1,2,3,4-tetrahydro-1,5-naphthyridine moiety.

23. The compound of claim 19, wherein R^{X11} and ring A together with the nitrogen to which they are attached form a 1,2,3,4-tetrahydro-1,5-naphthyridine moiety.

24. The compound of claim 19, wherein R^{X1} and ring A together with the nitrogen to which they are attached form a 1,2,3,4-tetrahydroquinoline moiety.

25. The compound of claim 19, wherein R^{X11} and ring A together with the nitrogen to which they are attached form a 1,2,3,4-tetrahydroquinoline moiety.

26. The compound of any one of claims 19-25, wherein R³ is selected from the group consisting of hydrogen, CH₃ and —NH₂.

27. The compound of any of claims 19-26, wherein m is an integer selected from 1 or 2; and n is 1.

28. The compound of any of claims 19-27, wherein R⁴ and R are each independently selected from the group consisting of hydrogen, hydroxyl, C₁₋₃alkyl, C₁₋₃alkoxy, C₁₋₃alkyl-O—R⁶, —C(O)NH₂, —N(R⁶)₂, halogen, C₁₋₃alkyl-N(R⁶)₂, and cyano; wherein C₁₋₃alkyl, C₁₋₃alkoxy, C₁₋₃alkyl-O—R⁶ and C₁₋₃alkyl-N(R⁶)₂ may optionally be substituted by one or more substituents each independently selected from the group consisting of hydroxyl, —N(R⁶)₂, and halogen;
or R⁴ and R⁵, taken together with the atoms to which they are attached, form a 3-7 membered carbocyclic or heterocyclic ring;
or R⁴ and R⁸, taken together with the atoms to which they are attached, form a 4-7 membered carbocyclic or heterocyclic ring;
or R⁴ is a bond, and R⁴ and R⁸, taken together with the atoms to which they are attached, form a 3-membered carbocyclic or heterocyclic ring; and
each R⁶ is independently hydrogen or C₁₋₃alkyl.

29. The compound of any one of claims 19-28, wherein R⁴ and R⁵ are each independently selected from the group consisting of C₁₋₃alkyl and —NR^{a}R^{b}; wherein C₁₋₃alkyl may optionally be substituted by one, two or three substituents each independently selected from the group consisting of halogen, hydroxyl and NR^{a}R^{b}.

30. The compound of any one of claims 19-29, wherein R⁴ and R⁵ are each independently selected from the group consisting of —NH₂, —CH₃, and —CH₂NH₂.

31. A compound of Formula III, or a pharmaceutically acceptable salt or stereoisomer thereof, wherein Formula III is represented by: 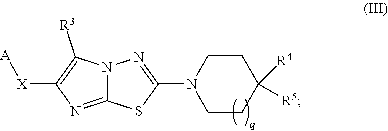
wherein
X is selected from the group consisting of a bond, —NR^{X1}—, and —C(O)NR^{X11};
A is selected from the group consisting of phenyl, a 5-7 membered monocyclic heteroaryl having one or more heteroatoms each independently selected from O, S, and N, a 8-10 membered bicyclic heteroaryl having one or more heteroatoms each independently selected from the group consisting of O, S, and N, and a 4-7 membered heterocyclyl; wherein A may optionally be substituted by one, two, three or more substituents each independently selected from the group consisting of —R, —OR, —N(R¹⁰)₂, —C(O)N(R¹⁰)₂, oxo, halogen and cyano;
R¹⁰ is independently selected from the group consisting of hydrogen, C₁₋₆alkyl, C₁₋₆ haloalkyl, C₃₋₆cycloalkyl, heterocycloalkyl, aryl, and heteroaryl, where R¹⁰ may optionally be substituted by one, two, three or more substituents each independently selected from the group consisting of halogen, —C(O)R²⁰, C₁₋₆alkyl and C₁₋₆haloalkyl;
R²⁰ is selected from the group consisting of hydroxyl, halogen, and C₁₋₆alkyl;
R^{X1} is selected from the group consisting of hydrogen, C₁₋₆alkyl and phenyl, or R^{X1} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —N(R¹⁰)₂, —C(O)N(R¹⁰)₂, oxo, halogen and cyano;
R^{X11} is selected from the group consisting of hydrogen, C₁₋₆alkyl and phenyl, or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —N(R¹⁰)₂, —C(O)N(R¹⁰)₂, oxo, halogen and cyano;
R³ is selected from the group consisting of hydrogen, halogen, hydroxyl, C₁₋₆alkyl and NR^{a}R^{b};
R⁴ and R⁵ are each independently selected from the group consisting of hydrogen, halogen, cyano, —NR^{a}R^{b}, —C(O)—NR^{a}R^{b} and C₁₋₆alkyl, wherein C₁₋₆alkyl may optionally be substituted by one, two or three or more substituents each independently selected from the group consisting of halogen, hydroxyl, —NR^{a}R^{b} and oxo;
R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl; and q is 0 or 1.

32. The compound of claim 31, wherein A is phenyl; wherein phenyl may optionally be substituted by one, two, or three substituents each independently selected from the group consisting of —OR¹⁰, halogen, and cyano.

33. The compound of claim 31, wherein A is pyridyl; wherein pyridyl may optionally be substituted by one, two, or three substituents each independently selected from the group consisting of —OR¹⁰, halogen, and cyano.

34. The compound of claim 31, wherein R^{X1} and ring A together with the nitrogen to which they are attached form a 1,2,3,4-tetrahydro-1,5-naphthyridine moiety.

35. The compound of claim 31, wherein R^{X1} and ring A together with the nitrogen to which they are attached form a 1,2,3,4-tetrahydro-1,5-naphthyridine moiety.

36. The compound of claim 31, wherein R^{X11} and ring A together with the nitrogen to which they are attached form a 1,2,3,4-tetrahydroquinoline moiety.

37. The compound of claim 31, wherein R^{X11} and ring A together with the nitrogen to which they are attached form a 1,2,3,4-tetrahydroquinoline moiety.

38. The compound of any one of claims 31-37, wherein R³ is selected from the group consisting of hydrogen, CH₃ and —NH₂.

39. The compound of any one of claims 31-38, wherein W and R⁵ are each independently selected from the group consisting of C₁₋₃alkyl and —NR^{a}R^{b}; wherein C₁₋₃alkyl may optionally be substituted by one, two or three substituents each independently selected from the group consisting of halogen, hydroxyl and NR^{a}R^{b}.

40. The compound of any one of claims 31-39, wherein W and R⁵ are each independently selected from the group consisting of —NH₂, —CH₃, and —CH₂NH₂.

41. A compound selected from the group consisting of: 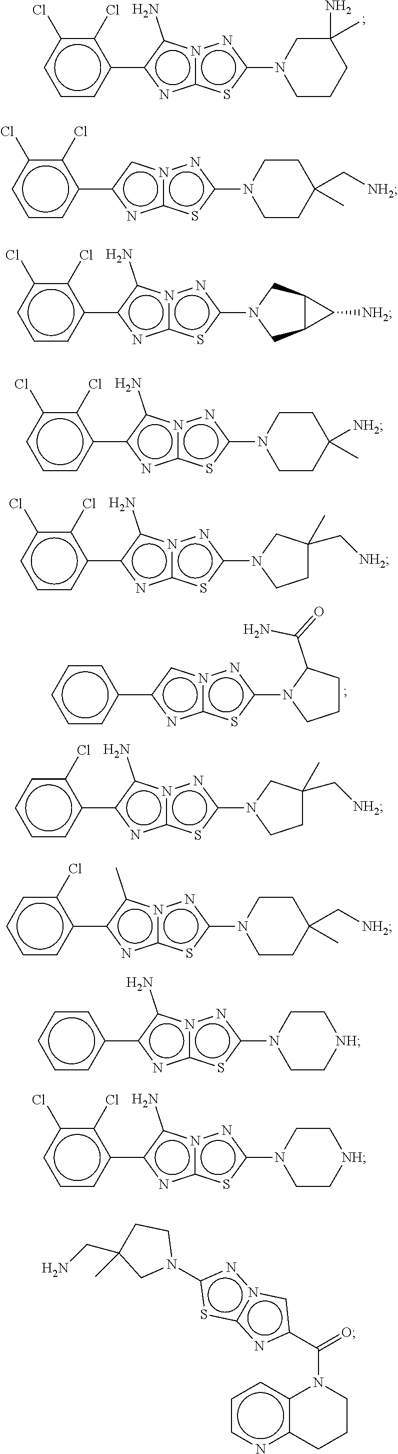 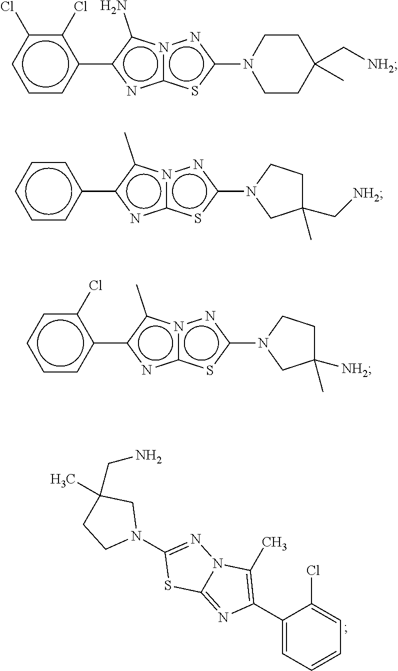
and pharmaceutically acceptable salts or stereoisomers thereof.

42. A compound of Formula IV, or a pharmaceutically acceptable salt or stereoisomer thereof, wherein Formula IV is represented by: 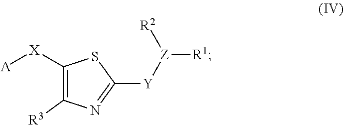
wherein
X is selected from the group consisting of a bond, —O—, —NR^{X1}—, —C(O)NR^{X11}—, —S(O)_{w}— (wherein w is 0, 1 or 2), —C(O)—, —C(R^{X2}R^{X3})—, —O—C(R^{X4}R^{X5})—, —C(R^{X4}R^{X5})—O—, and —C═C(R^{X6}R^{X7})—;
Y is selected from the group consisting of a bond, —C(O)—, —NR^{Y}—, —C₁₋₃alkylene-NR^{Y}—, and —NR^{Y}—C₁₋₃alkylene-;
Z is selected from the group consisting of N and C(R^{Z});
wherein if Y is a bond or —C(O)—, Z is N; and wherein if Y is —NR^{Y}—, —C₁₋₃alkylene-NR^{Y}—, or —NR^{Y}—C₁₋₃alkylene-, Z is C(R^{Z});
R^{Z} is selected from the group consisting of hydrogen and C₁₋₆alkyl; wherein C₁₋₆alkyl may optionally be substituted by halogen, hydroxyl, and —NR^{a}R^{b},
A is selected from the group consisting of a 6-10 membered monocyclic or bicyclic aryl, a 5-7 membered monocyclic heteroaryl having one or more heteroatoms each independently selected from O, S, or N, a 8-10 membered bicyclic heteroaryl having one or more heteroatoms each independently selected from O, S, or N; and a 4-7 membered heterocyclyl, wherein A may optionally be substituted by one, two, three or more substituents each independently selected from the group consisting of —R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂—C(O)R¹⁰, —C(O)N(R¹⁰)₂, oxo, halogen and cyano;
R¹⁰ is independently selected from the group consisting of hydrogen, C₁₋₆alkyl, C₁₋₆haloalkyl, C₃₋₆cycloalkyl, heterocycloalkyl, aryl, and heteroaryl; wherein R¹⁰ may optionally be substituted by one, two, three or more substituents each independently selected from the group consisting of halogen, —C(O)R²⁰, C₁₋₆alkyl and C₁₋₆haloalkyl;
R²⁰ is selected from the group consisting of hydroxyl, halogen, and C₁₋₆alkyl;
R¹ and R² are each selected from the group consisting of hydrogen and C₁₋₁₀alkyl; wherein C₁₋₁₀alkyl may optionally be substituted by one, two, three or more substituents each independently selected from R^{P};
or R¹ and R², together with carbon or nitrogen to which they are attached, form a 3-7 membered saturated carbocyclic or 4-7 membered saturated heterocyclic ring B having one or two heteroatoms each independently selected from the group consisting of O, S(O), (wherein w is 0, 1, or 2), and NR^{h}; wherein carbocyclic or heterocyclic ring B may be monocyclic or bicyclic;
carbocyclic or heterocyclic ring B may optionally be substituted on one or more available carbons by one, two, three or more substituents each independently selected from the group consisting of halogen, hydroxyl, cyano, oxo, —NR^{a}R^{b}, —CO₂H, —C(O)—NR^{a}R^{b}, —S(O)₂—NR^{a}R^{b}, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})—, C₁₋₆alkoxycarbonyl-N(R^{a})—, aryl and heteroaryl; wherein C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})— and C₁₋₆alkoxycarbonyl-N(R^{a})— may optionally be substituted by one or more substituents each independently selected from R^{p}; and wherein aryl and heteroaryl may optionally be substituted by one or more substituents each independently selected from R^{f};
R^{X1} is selected from the group consisting of hydrogen, C₁₋₆alkyl and phenyl, or R^{X1} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂—C(O)R¹⁰, —C(O)N(R¹⁰)₂, oxo, halogen and cyano;
R^{X11} is selected from the group consisting of hydrogen, C₁₋₆alkyl and phenyl, or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;
R^{X2} and R^{X3} are each independently selected from the group consisting of hydrogen, halogen, hydroxyl, —NR^{a}R^{b}, —C(O)—NR^{a}R^{b}, C₁₋₆alkyl and C₁₋₆alkoxy; wherein C₁₋₆alkyl and C₁₋₆alkoxy may optionally be substituted by one or more substituents each independently selected from R^{p};
or R^{X2} and R^{X3} together with the carbon to which they are attached form a 3-6 membered carbocycle optionally substituted by one or more substituents each independently selected from the group consisting of hydrogen, halogen, —NR^{a}R^{b}, —C(O)—NR^{a}R^{b}, oxo, C₁₋₆alkyl and C₁₋₆alkoxy;
R^{X4} and R^{X5} are each independently selected from the group consisting of hydrogen and C₁₋₆alkyl; wherein C₁₋₆alkyl may optionally be substituted by one or more substituents each independently selected from R^{P};
R^{X6} and R^{X7} are each independently selected from the group consisting of hydrogen, halogen, —C(O)—NR^{a}R^{b}, cyano and C₁₋₆alkyl; wherein C₁₋₆alkyl may optionally be substituted by one or more substituents each independently selected from R^{P};
R^{Y} is selected from the group consisting of hydrogen, C₁₋₆alkyl and phenyl;
R³ is selected from the group consisting of hydrogen, halogen, hydroxyl, C₁₋₆alkyl and —NR^{a}R^{b};
R^{f} is independently selected, for each occurrence, from the group consisting of R^{P}, C₁₋₆alkyl, —C₁₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, (wherein wherein w is 0, 1 or 2), C₁₋₆alkylcarbonyl-N(R^{a}) and C₁₋₆alkoxycarbonyl-N(R^{a})—; wherein C₁₋₆alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, C₁₋₆alkylcarbonyl-N(R^{a})—, and C₁₋₆alkoxycarbonyl-N(R^{a})— may be optionally substituted by one or more substituents selected from R^{p};
R^{h} is independently selected, for each occurrence, from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆alkylcarbonyl-, C₁₋₆alkoxycarbonyl-, R^{a}R^{b}N-carbonyl- and R^{a}R^{b}N—SO₂—; wherein C₁₋₆alkyl, C₃₋₆alkenyl, C₃₋₆alkynyl, C₃₋₆cycloalkyl, C₁₋₆alkyl-S(O)₂—, C₁₋₆alkylcarbonyl- and C₁₋₆alkoxycarbonyl- may optionally be substituted by one or more substituents selected from R^{P};
R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl;
or R^{a} and R^{b}, together with the nitrogen to which they are attached, may form a 4-6 membered monocyclic heterocyclic ring, which may have an additional heteroatom selected from the group consisting of O, S, and N; wherein the 4-6 membered heterocyclic ring may optionally be substituted by one or more substituents selected from the group consisting of halogen, cyano, oxo or hydroxyl; and
R^{P} is independently selected, for each occurrence, from the group consisting of halogen, hydroxyl, cyano, C₁₋₆alkoxy, R^{a}R^{b}N—, R^{a}R^{b}N-carbonyl-, R^{a}R^{b}N—SO₂—, and R^{a}R^{b}N-carbonyl-N(R^{a})—.

43. The compound of claim 42, wherein X is selected from the group consisting of a bond, —O—, —NR^{X1}— and —NR^{X11}—.

44. The compound of claim 42 or 43, wherein A is selected from the group consisting of phenyl, pyridyl, quinolinyl, indolyl, and indolinyl, wherein phenyl, pyridyl, quinolinyl, indolyl and indolinyl may optionally be substituted by one, two or three substituents each independently selected from the group consisting of —R¹, —OR, —SR, —N(R¹⁰)₂, —OSO₂R¹⁰, —SO₂R¹⁰, —C(O)N(R¹⁰)₂, halogen, and cyano.

45. The compound of any one of claims 42-44, wherein A is phenyl; wherein phenyl may optionally be substituted by one, two, or three substituents each independently selected from the group consisting of —OR¹⁰, halogen, and cyano.

46. The compound of any one of claims 42-44, wherein A is pyridyl; wherein pyridyl may optionally be substituted by one, two, or three substituents each independently selected from the group consisting of —OR¹⁰, halogen, and cyano.

47. The compound of claim 42 or 43, wherein R^{X1} and ring A together with the nitrogen to which they are attached form a 1,2,3,4-tetrahydro-1,5-naphthyridine moiety.

48. The compound of claim 42 or 43, wherein R^{X11} and ring A together with the nitrogen to which they are attached form a 1,2,3,4-tetrahydro-1,5-naphthyridine moiety.

49. The compound of claim 42 or 43, wherein R^{X1} and ring A together with the nitrogen to which they are attached form a 1,2,3,4-tetrahydroquinoline moiety.

50. The compound of claim 42 or 43, wherein R^{X1} and ring A together with the nitrogen to which they are attached form a 1,2,3,4-tetrahydroquinoline moiety.

51. The compound of any one of claims 42-50, wherein R³ is selected from the group consisting of hydrogen, CH₃ and —NH₂.

52. The compound of any one of claims 42-51, wherein Y is a bond and Z is N.

53. The compound of any one of claims 42-52, wherein R¹ and R² together with the nitrogen to which they are attached form heterocyclic ring B; wherein ring B is selected from the group consisting of pyrrolidinyl, piperidinyl, azepanyl and morpholinyl; wherein pyrrolidinyl, piperidinyl, azepanyl and morpholinyl may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃alkoxy; wherein C₁₋₃alkyl and C₁₋₃alkoxy may optionally be substituted by halogen, hydroxyl and NR^{a}R^{b}.

54. The compound of claim any one of claims 42-51, wherein Y is —NH—, —N(CH₃)—, —N(H)—CH₂-, or —N(CH₃)—CH₂— and Z is C(H).

55. The compound of claim 54, wherein R¹ and R² together with the carbon to which they are attached form carbocyclic ring B; wherein ring B is selected from the group consisting of cyclopentyl and cyclohexyl, and wherein cyclopentyl and cyclohexyl may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃alkoxy; wherein C₁₋₃alkyl and C₁₋₃alkoxy may optionally be substituted by halogen, hydroxyl and —NR^{a}R^{b}.

56. The compound of claim 54, wherein R¹ and R² together with the carbon to which they are attached form heterocyclic ring B; wherein ring B is selected from the group consisting of tetrahydrofuranyl and tetrahydropyranyl, wherein tetrahydrofuranyl and tetrahydropyranyl may optionally be substituted by one, two, three or more substituents each independently selected from the group consisting of halogen, hydroxyl, cyano, —NR^{a}R^{b}, —C(O)—NR^{a}R^{b}, C₁₋₃ alkyl, and C₁₋₃alkoxy; wherein C₁₋₃alkyl and C₁₋₃alkoxy may optionally be substituted by halogen, hydroxyl and NR^{a}R^{b}.

57. The compound of any one of claims 42-56, wherein ring B is substituted on an available carbon by a substituent selected from the group consisting of —NH₂, —CH₃, and —CH₂NH₂.

58. The compound of any one of claims 42-52, wherein R¹ is hydrogen and R² is branched C₁₋₁₀alkyl; wherein C₁₋₁₀alkyl may optionally be substituted by one, two, three or more substituents each independently selected from the group consisting of halogen and —NH₂.

59. A compound of Formula V, or a pharmaceutically acceptable salt or stereoisomer thereof, wherein Formula V is represented by: 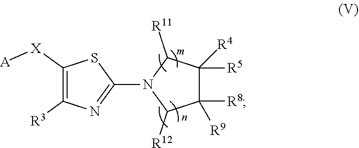
wherein
X is selected from the group consisting of a bond, —O—, —NR^{X1}—, —C(O)NR^{X11}—, —S(O)_{w}— (wherein w is 0, 1 or 2), —C(O)— and —(R^{X2}R^{X3})—;
A is selected from the group consisting of phenyl, a 5-7 membered monocyclic heteroaryl having one or more heteroatoms each independently selected from O, S, and N, a 8-10 membered bicyclic heteroaryl having one or more heteroatoms each independently selected from the group consisting of O, S, and N, and a 4-7 membered heterocyclyl; wherein A may optionally be substituted by one, two, three or more substituents each independently selected from the group consisting of —R, —OR¹⁰, —N(R¹⁰)₂, —C(O)N(R¹⁰)₂, oxo, halogen and cyano;
R¹⁰ is independently selected from the group consisting of hydrogen, C₁₋₆alkyl, C₁₋₆haloalkyl, C₃₋₆cycloalkyl, heterocycloalkyl, aryl, and heteroaryl, where R¹⁰ may optionally be substituted by one, two, three or more substituents each independently selected from the group consisting of halogen, —C(O)R²⁰, C₁₋₆alkyl and C₁₋₆haloalkyl;
R²⁰ is selected from the group consisting of hydroxyl, halogen, and C₁₋₆alkyl;
R^{X1} is selected from the group consisting of hydrogen, C₁₋₆alkyl and phenyl; or R^{X1} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —N(R′)₂, —C(O)N(R¹⁰)₂, oxo, halogen and cyano;
R^{X11} is selected from the group consisting of hydrogen, C₁₋₆alkyl and phenyl; or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —N(R′)₂, —C(O)N(R¹⁰)₂, oxo, halogen and cyano;
R^{X2} and R^{X3} are each independently selected from the group consisting of hydrogen, halogen, hydroxyl, —NR^{a}R^{b}, —(O)—NR^{a}R^{b}, C₁₋₆alkyl and C₁₋₆alkoxy; or R^{X2} and R^{X3} together with the carbon to which they are attached form a 3-6 membered carbocyclyl which may optionally be substituted by one or more substituents each independently selected from the group consisting of hydrogen, halogen, —NR^{a}R^{b}, —C(O)—NR^{a}R^{b}, oxo, C₁₋₆alkyl and C₁₋₆ alkoxy;
R³ is selected from the group consisting of hydrogen, halogen, hydroxyl, C₁₋₆alkyl and —NR^{a}R^{b};
R⁴ and R⁵ are each independently selected from the group consisting of hydrogen, hydroxyl, C₁₋₆alkyl, C₁₋₆alkoxy, C₁₋₆alkyl-O—R⁶, —C(O)N(R⁶)₂, —N(R⁶)₂, halogen, C₁₋₆alkyl-N(R⁶)₂, and cyano; wherein C₁₋₆alkyl, C₁₋₆alkoxy, C₁₋₆alkyl-O—R⁶, and C₁₋₆alkyl-N(R⁶)₂ may optionally be substituted by one or more substituents each independently selected from the group consisting of hydroxyl, —NH₂, oxo, and halogen,
or R⁴ and R⁵, taken together with the atoms to which they are attached, form a 3-7 membered carbocyclic or heterocyclic saturated or partially unsaturated ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;
or R⁴ and R⁸, taken together with the atoms to which they are attached, form a 4-7 membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;
or R⁴ is absent, and R and R⁸, taken together with the atoms to which they are attached, form a 3-membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;
R⁶ is independently for each occurrence selected from the group consisting of hydrogen, C₁₋₆alkyl and phenyl;
R¹¹ and R¹² are each independently selected from the group consisting of hydrogen, hydroxyl, C₁₋₆alkyl, C₁₋₆alkoxy, C₁₋₆alkyl-O—R⁶, aryl, arylalkylene, heteroaryl, heteroarylalkylene, —C(O)N(R⁶)₂, —N(R %)₂, halogen, C₁₋₆alkyl-N(R⁶)₂, —CO₂H, and cyano; wherein C₁₋₆alkyl, C₁₋₆alkoxy, C₁₋₆alkyl-O—R⁶, aryl, arylalkylene, heteroaryl, heteroarylalkylene, and C₁₋₆alkyl-N(R⁶)₂ may optionally be substituted by one or more substituents each independently selected from the group consisting of hydroxyl, —NH₂, and halogen;
or R¹¹ and R¹², taken together with the atoms to which they are attached, form a 5-7 membered heterocyclic ring;
or R⁴ and R¹², taken together with the atoms to which they are attached, form a 5-7 membered carbocyclic or heterocyclic ring;
or R⁸ and R¹¹, taken together with the atoms to which they are attached, form a 5-7 membered carbocyclic or heterocyclic ring;
R⁸ and R⁹ are each independently selected from the group consisting of hydrogen, C₁₋₆)alkyl, C₁₋₆alkyl-N(R⁶)₂, —OR⁶, C₁₋₆alkyl-O—R⁶, —C(O)NH₂, —N(R⁶)₂, halogen, and cyano; and
each of m and n is, independently, 0, 1, 2, or 3, with m+n being at least 2 and no more than 4.

60. The compound of claim 59, wherein A is phenyl; wherein phenyl may optionally be substituted by one, two, or three substituents each independently selected from the group consisting of —OR¹⁰, halogen, and cyano.

61. The compound of claim 59, wherein A is pyridyl; wherein pyridyl may optionally be substituted by one, two, or three substituents each independently selected from the group consisting of —OR¹⁰, halogen, and cyano.

62. The compound of claim 59, wherein R^{X1} and ring A together with the nitrogen to which they are attached form a 1,2,3,4-tetrahydro-1,5-naphthyridine moiety.

63. The compound of claim 59, wherein R^{X11} and ring A together with the nitrogen to which they are attached form a 1,2,3,4-tetrahydro-1,5-naphthyridine moiety.

64. The compound of claim 59, wherein R^{X1} and ring A together with the nitrogen to which they are attached form a 1,2,3,4-tetrahydroquinoline moiety.

65. The compound of claim 59, wherein R^{X11} and ring A together with the nitrogen to which they are attached form a 1,2,3,4-tetrahydroquinoline moiety.

66. The compound of any one of claims 59-65, wherein R³ is selected from the group consisting of hydrogen, CH₃ and —NH₂.

67. The compound of any of claims 59-66, wherein m is an integer selected from 1 or 2; and n is 1.

68. The compound of any of claims 59-67, wherein R⁴ and R are each independently selected from the group consisting of hydrogen, hydroxyl, C₁₋₃alkyl, C₁₋₃alkoxy, C₁₋₃alkyl-O—R⁶, —C(O)NH₂, —N(R⁶)₂, halogen, C₁₋₃alkyl-N(R⁶)₂, and cyano; wherein C₁₋₃alkyl, C₁₋₃alkoxy, C₁₋₃alkyl-O—R⁶ and C₁₋₃alkyl-N(R⁶)₂ may optionally be substituted by one or more substituents each independently selected from the group consisting of hydroxyl, —N(R⁶)₂, and halogen;
or R⁴ and R⁵, taken together with the atoms to which they are attached, form a 3-7 membered carbocyclic or heterocyclic ring;
or R⁴ and R⁸, taken together with the atoms to which they are attached, form a 4-7 membered carbocyclic or heterocyclic ring;
or R⁴ is a bond, and R and R⁸, taken together with the atoms to which they are attached, form a 3-membered carbocyclic or heterocyclic ring; and
each R⁶ is independently hydrogen or C₁₋₃alkyl.

69. The compound of any one of claims 59-68, wherein W and R⁵ are each independently selected from the group consisting of C₁₋₃alkyl and —NR^{a}R^{b}; wherein C₁₋₃alkyl may optionally be substituted by one, two or three substituents each independently selected from the group consisting of halogen, hydroxyl and NR^{a}R^{b}.

70. The compound of any one of claims 59-69, wherein W and R⁵ are each independently selected from the group consisting of —NH₂, —CH₃, and —CH₂NH₂.

71. A compound of Formula VI, or a pharmaceutically acceptable salt or stereoisomer thereof, wherein Formula VI is represented by: 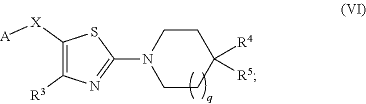
wherein
X is selected from the group consisting of a bond, —NR^{X1}—, and —C(O)NR^{X11}—;
A is selected from the group consisting of phenyl, a 5-7 membered monocyclic heteroaryl having one or more heteroatoms each independently selected from O, S, and N, a 8-10 membered bicyclic heteroaryl having one or more heteroatoms each independently selected from the group consisting of O, S, and N, and a 4-7 membered heterocyclyl; wherein A may optionally be substituted by one, two, three or more substituents each independently selected from the group consisting of —R¹, —OR, —N(R¹⁰)₂, —C(O)N(R¹⁰)₂, oxo, halogen and cyano;
R¹⁰ is independently selected from the group consisting of hydrogen, C₁₋₆alkyl, C₁₋₆ haloalkyl, C₃₋₆cycloalkyl, heterocycloalkyl, aryl, and heteroaryl, where R¹⁰ may optionally be substituted by one, two, three or more substituents each independently selected from the group consisting of halogen, —C(O)R²⁰, C₁₋₆alkyl and C₁₋₆haloalkyl;
R²⁰ is selected from the group consisting of hydroxyl, halogen, and C₁₋₆alkyl;
R^{X1} is selected from the group consisting of hydrogen, C₁₋₆alkyl and phenyl, or R^{X1} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —N(R¹⁰)₂, —C(O)N(R¹⁰)₂, oxo, halogen and cyano;
R^{X11} is selected from the group consisting of hydrogen, C₁₋₆alkyl and phenyl, or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —N(R¹⁰)₂, —C(O)N(R¹⁰)₂, oxo, halogen and cyano;
R³ is selected from the group consisting of hydrogen, halogen, hydroxyl, C₁₋₆alkyl and NR^{a}R^{b};
R⁴ and R⁵ are each independently selected from the group consisting of hydrogen, halogen, cyano, —NR^{a}R^{b}, —C(O)—NR^{a}R^{b} and C₁₋₆alkyl, wherein C₁₋₆alkyl may optionally be substituted by one, two or three or more substituents each independently selected from the group consisting of halogen, hydroxyl, —NR^{a}R^{b} and oxo;
R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl; and q is 0 or 1.

72. The compound of claim 71, wherein A is phenyl; wherein phenyl may optionally be substituted by one, two, or three substituents each independently selected from the group consisting of —OR¹⁰, halogen, and cyano.

73. The compound of claim 71, wherein A is pyridyl; wherein pyridyl may optionally be substituted by one, two, or three substituents each independently selected from the group consisting of —OR¹⁰, halogen, and cyano.

74. The compound of claim 71, wherein R^{X1} and ring A together with the nitrogen to which they are attached form a 1,2,3,4-tetrahydro-1,5-naphthyridine moiety.

75. The compound of claim 71, wherein R^{X11} and ring A together with the nitrogen to which they are attached form a 1,2,3,4-tetrahydro-1,5-naphthyridine moiety.

76. The compound of claim 71, wherein R^{X1} and ring A together with the nitrogen to which they are attached form a 1,2,3,4-tetrahydroquinoline moiety.

77. The compound of claim 71, wherein R^{X11} and ring A together with the nitrogen to which they are attached form a 1,2,3,4-tetrahydroquinoline moiety.

78. The compound of any one of claims 71-77, wherein R³ is selected from the group consisting of hydrogen, CH₃ and —NH₂.

79. The compound of any one of claims 71-78, wherein W and R⁵ are each independently selected from the group consisting of C₁₋₃alkyl and —NR^{a}R^{b}; wherein C₁₋₃alkyl may optionally be substituted by one, two or three substituents each independently selected from the group consisting of halogen, hydroxyl and NR^{a}R^{b}.

80. The compound of any one of claims 71-79, wherein W and R⁵ are each independently selected from the group consisting of —NH₂, —CH₃, and —CH₂NH₂.

81. A compound selected from the group consisting of: 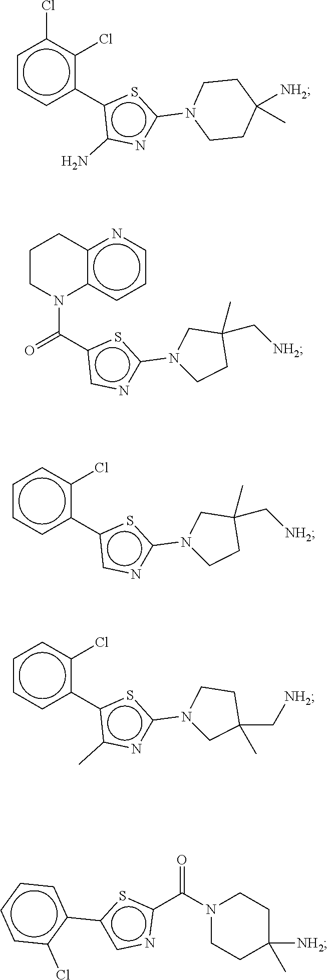
and a pharmaceutically acceptable salt or stereoisomer thereof.

82. A compound of Formula VII, or a pharmaceutically acceptable salt, stereoisomer or N-oxide thereof, wherein Formula VII is represented by: 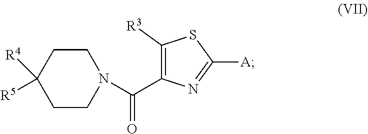
wherein
A is phenyl, wherein A may optionally be substituted by one, two, three or more substituents each independently selected from the group consisting of —R¹⁰, —OR¹⁰, methylenedioxy, —N(R¹⁰)₂, —C(O)N(R¹⁰)₂, halogen and cyano;
R¹⁰ is independently selected from the group consisting of hydrogen, C₁₋₆alkyl, C₁₋₆haloalkyl, C₃₋₆cycloalkyl, heterocycloalkyl, aryl, and heteroaryl, where R¹⁰ may optionally be substituted one, two, three or more substituents each independently selected from the group consisting of halogen, —C(O)R²⁰, C₁₋₆alkyl and C₁₋₆haloalkyl;
R²⁰ is selected from the group consisting of hydroxyl, halogen, and C₁₋₆alkyl;
R³ is selected from the group consisting of hydrogen, halogen, hydroxyl, C₁₋₆alkyl and —NR^{a}R^{b};
R⁴ and R⁵ are each independently selected from the group consisting of hydrogen, halogen, cyano, —NR^{a}R^{b}, —C(O)—NR^{a}R^{b} and C₁₋₆alkyl, wherein C₁₋₆alkyl may optionally be substituted by one, two or three or more substituents each independently selected from the group consisting of halogen, hydroxyl, —NR^{a}R^{b} and oxo; and
R^{a} and R are independently selected, for each occurrence, from the group consisting of hydrogen and C₁₋₆alkyl; wherein C₁₋₆alkyl may optionally be substituted by one or more substituents selected from halogen, cyano, oxo and hydroxyl.

83. The compound of claim 82, wherein R⁴ and R⁵ are each independently selected from the group consisting of C₁₋₃alkyl and —NR^{a}R^{b}; wherein C₁₋₃alkyl may optionally be substituted by one, two or three substituents each independently selected from the group consisting of halogen, hydroxyl and NR^{a}R^{b}.

84. The compound of claim 82 or 83, wherein R⁴ and R⁵ are each independently selected from the group consisting of —NH₂, —CH₃, and —CH₂NH₂, —C(H)NH₂CH₃ and —CH₂NHCH₃.

85. A compound of Formula VIII, or a pharmaceutically acceptable salt or stereoisomer thereof, wherein Formula VIII is represented by: 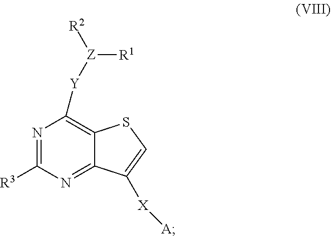
wherein
X is selected from the group consisting of a bond, —O—, —NR^{X1}—, —C(O)NR^{X11}—, —S(O)_{w}— (wherein w is 0, 1 or 2), —C(O)—, —C(R^{X2}R^{X3})—, —O—C(R^{X4}R^{X5})—, —C(R^{X4}R^{X5})—O—, and —C═C(R^{X6}R^{X7})—;
Y is selected from the group consisting of a bond, —NR^{Y}—, —C₁₋₃alkylene-NR^{Y}—, and —NR^{Y}—C₁₋₃alkylene-;
Z is selected from the group consisting of N, C(R^{Z}) and a bond;
wherein if Y is a bond, Z is N or a bond; and wherein if Y is —NR^{Y}—, —C₁₋₃alkylene-NR^{Y}—, or —NR^{Y}—C₁₋₃alkylene-, Z is C(R^{Z});
R^{Z} is selected from the group consisting of hydrogen and C₁₋₆alkyl; wherein C₁₋₆alkyl may optionally be substituted by halogen, hydroxyl, and —NR^{a}R^{b},
A is selected from the group consisting of a 6-10 membered monocyclic or bicyclic aryl, a 5-7 membered monocyclic heteroaryl having one or more heteroatoms each independently selected from O, S, or N, a 8-10 membered bicyclic heteroaryl having one or more heteroatoms each independently selected from O, S, or N; and a 4-7 membered heterocyclyl, wherein A may optionally be substituted by one, two, three or more substituents each independently selected from the group consisting of —R¹⁰, —OR¹⁰, —S(O)_{w}R¹⁰ (wherein w is 0, 1 or 2), —N(R¹⁰)₂, —OS(O)—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano, or when X is a bond, A may also be selected from the group consisting of H and halogen;
R¹⁰ is independently selected from the group consisting of hydrogen, C₁₋₆alkyl, C₁₋₆haloalkyl, C₃₋₆cycloalkyl, heterocycloalkyl, aryl, and heteroaryl; wherein R¹⁰ may optionally be substituted by one, two, three or more substituents each independently selected from the group consisting of halogen, —C(O)R²⁰, C₁₋₆alkyl and C₁₋₆haloalkyl;
R²⁰ is selected from the group consisting of hydroxyl, halogen, and C₁₋₆alkyl;
R¹ and R² are each selected from the group consisting of hydrogen, C₁₋₁₀alkyl and phenyl; wherein C₁₋₁₀alkyl and phenyl may optionally be substituted by one, two, three or more substituents each independently selected from R^{P}, and wherein when Z and Y are each a bond R² is not present;
or R¹ and R², together with carbon or nitrogen to which they are attached, form a 3-7 membered saturated carbocyclic or 4-7 membered saturated heterocyclic ring B having one or two heteroatoms each independently selected from the group consisting of 0, S(O), (wherein w is 0, 1, or 2), and NR^{h}; wherein carbocyclic or heterocyclic ring B may be monocyclic or bicyclic;
carbocyclic or heterocyclic ring B may optionally be substituted on one or more available carbons by one, two, three or more substituents each independently selected from the group consisting of halogen, hydroxyl, cyano, oxo, NR^{a}R^{b}, —CO₂H, —C(O)—NR^{a}R^{b}, —S(O)₂—NR^{a}R^{b}, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})—, C₁₋₆alkoxycarbonyl-N(R^{a})—, aryl and heteroaryl; wherein C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})— and C₁₋₆alkoxycarbonyl-N(R^{a})— may optionally be substituted by one or more substituents each independently selected from R^{p}; and wherein aryl and heteroaryl may optionally be substituted by one or more substituents each independently selected from R;
R^{X1} is selected from the group consisting of hydrogen, C₁₋₆alkyl and phenyl, or R^{X1} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂—C(O)R¹⁰, —C(O)N(R¹⁰)₂, oxo, halogen and cyano;
R^{X11} is selected from the group consisting of hydrogen, C₁₋₆alkyl and phenyl, or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹, —P(O)(R¹⁰)₂—C(O)R¹⁰, —C(O)N(R¹⁰)₂, oxo, halogen and cyano;
R^{X2} and R^{X3} are each independently selected from the group consisting of hydrogen, halogen, hydroxyl, —NR^{a}R^{b}, —C(O)—NR^{a}R^{b}, C₁₋₆alkyl and C₁₋₆alkoxy; wherein C₁₋₆alkyl and C₁₋₆alkoxy may optionally be substituted by one or more substituents each independently selected from R^{p};
or R^{X2} and R^{X3} together with the carbon to which they are attached form a 3-6 membered carbocycle optionally substituted by one or more substituents each independently selected from the group consisting of hydrogen, halogen, NR^{a}R^{b}, —C(O)—NR^{a}R^{b}, oxo, C₁₋₆ alkyl and C₁₋₆alkoxy;
R^{X4} and R^{X5} are each independently selected from the group consisting of hydrogen and C₁₋₆alkyl; wherein C₁₋₆alkyl may optionally be substituted by one or more substituents each independently selected from R^{P};
R^{X6} and R^{X7} are each independently selected from the group consisting of hydrogen, halogen, —C(O)—NR^{a}R^{b}, cyano and C₁₋₆alkyl; wherein C₁₋₆alkyl may optionally be substituted by one or more substituents each independently selected from R^{P};
R^{Y} is selected from the group consisting of hydrogen, C₁₋₆alkyl and phenyl;
R³ is selected from the group consisting of hydrogen, halogen, hydroxyl, C₁₋₆alkyl and NR^{a}R^{b};
R^{f} is independently selected, for each occurrence, from the group consisting of R^{P}, C₁₋₆alkyl, —C₁₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, (wherein wherein w is 0, 1 or 2), C₁₋₆alkylcarbonyl-N(R^{a}) and C₁₋₆alkoxycarbonyl-N(R^{a})—; wherein C₁₋₆alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, C₁₋₆alkylcarbonyl-N(R^{a})—, and C₁₋₆alkoxycarbonyl-N(R^{a})— may be optionally substituted by one or more substituents selected from R^{p};
R^{h} is independently selected, for each occurrence, from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆ alkylcarbonyl-, C₁₋₆alkoxycarbonyl-, R^{a}R^{b}N-carbonyl- and R^{a}R^{b}N—SO₂—; wherein C₁₋₆alkyl, C₃₋₆alkenyl, C₃₋₆alkynyl, C₃₋₆cycloalkyl, C₁₋₆alkyl-S(O)₂—, C₁₋₆alkylcarbonyl- and C₁₋₆ alkoxycarbonyl- may optionally be substituted by one or more substituents selected from R^{P};
R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl;
or R and R^{b}, together with the nitrogen to which they are attached, may form a 4-6 membered monocyclic heterocyclic ring, which may have an additional heteroatom selected from the group consisting of O, S, and N; wherein the 4-6 membered heterocyclic ring may optionally be substituted by one or more substituents selected from the group consisting of halogen, cyano, oxo or hydroxyl; and
R^{P} is independently selected, for each occurrence, from the group consisting of halogen, hydroxyl, cyano, C₁₋₆alkoxy, R^{a}R^{b}N—, R^{a}R^{b}N-carbonyl-, R^{a}R^{b}N—SO₂—, and R^{a}R^{b}N-carbonyl-N(R^{a})—.

86. The compound of claim 85, wherein X is selected from the group consisting of a bond, —O—, —NR^{X1}— and NR^{X11}—.

87. The compound of claim 85 or 86, wherein A is selected from the group consisting of phenyl, pyridyl, quinolinyl, indolyl, and indolinyl, wherein phenyl, pyridyl, quinolinyl, indolyl and indolinyl may optionally be substituted by one, two or three substituents each independently selected from the group consisting of —R¹, —OR, —SR¹⁰, —N(R¹⁰)₂, —OSO₂R¹⁰, —SO₂R¹⁰, —C(O)N(R¹⁰)₂, halogen, and cyano.

88. The compound of any one of claims 85-87, wherein A is phenyl; wherein phenyl may optionally be substituted by one, two, or three substituents each independently selected from the group consisting of —OR¹⁰, halogen, and cyano.

89. The compound of any one of claims 85-87, wherein A is pyridyl; wherein pyridyl may optionally be substituted by one, two, or three substituents each independently selected from the group consisting of —OR¹⁰, halogen, and cyano.

90. The compound of claim 85 or 86, wherein R^{X1} and ring A together with the nitrogen to which they are attached form a 1,2,3,4-tetrahydro-1,5-naphthyridine moiety.

91. The compound of claim 85 or 86, wherein R^{X1} and ring A together with the nitrogen to which they are attached form a 1,2,3,4-tetrahydro-1,5-naphthyridine moiety.

92. The compound of claim 85 or 86, wherein R^{X1} and ring A together with the nitrogen to which they are attached form a 1,2,3,4-tetrahydroquinoline moiety.

93. The compound of claim 85 or 86, wherein R^{X1} and ring A together with the nitrogen to which they are attached form a 1,2,3,4-tetrahydroquinoline moiety.

94. The compound of any one of claims 85-93, wherein R³ is selected from the group consisting of hydrogen, CH₃ and —NH₂.

95. The compound of any one of claims 85-94, wherein Y is a bond and Z is N.

96. The compound of any one of claims 85-95, wherein R¹ and R² together with the nitrogen to which they are attached form heterocyclic ring B; wherein ring B is selected from the group consisting of pyrrolidinyl, piperidinyl, azepanyl and morpholinyl; wherein pyrrolidinyl, piperidinyl, azepanyl and morpholinyl may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃alkoxy; wherein C₁₋₃alkyl and C₁₋₃alkoxy may optionally be substituted by halogen, hydroxyl and NR^{a}R^{b}.

97. The compound of claim any one of claims 85-94, wherein Y is —NH—, —N(CH₃)—, —N(H)—CH₂—, or —N(CH₃)—CH₂— and Z is C(H).

98. The compound of claim 97, wherein R¹ and R² together with the carbon to which they are attached form carbocyclic ring B; wherein ring B is selected from the group consisting of cyclopentyl and cyclohexyl, and wherein cyclopentyl and cyclohexyl may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃ alkoxy; wherein C₁₋₃alkyl and C₁₋₃alkoxy may optionally be substituted by halogen, hydroxyl and —NR^{a}R^{b}.

99. The compound of claim 97, wherein R¹ and R² together with the carbon to which they are attached form heterocyclic ring B; wherein ring B is selected from the group consisting of tetrahydrofuranyl and tetrahydropyranyl, wherein tetrahydrofuranyl and tetrahydropyranyl may optionally be substituted by one, two, three or more substituents each independently selected from the group consisting of halogen, hydroxyl, cyano, —NR^{a}R^{b}, —C(O)—NR^{a}R^{b}, C₁₋₃ alkyl, and C₁₋₃alkoxy; wherein C₁₋₃alkyl and C₁₋₃alkoxy may optionally be substituted by halogen, hydroxyl and NR^{a}R^{b}.

100. The compound of any one of claims 85-99, wherein ring B is substituted on an available carbon by a substituent selected from the group consisting of —NH₂, —CH₃, and —CH₂NH₂.

101. The compound of any one of claims 85-95, wherein R¹ is hydrogen and R² is branched C₁₋₁₀alkyl; wherein C₁₋₁₀alkyl may optionally be substituted by one, two, three or more substituents each independently selected from the group consisting of halogen and —NH₂.

102. The compound of any one of claims 85-101, wherein —Y—Z—(R¹)(R²) is: 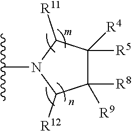
wherein
R⁴ and R⁵ are each independently selected from the group consisting of hydrogen, hydroxyl, C₁₋₆alkyl, C₁₋₆alkoxy, C₁₋₆alkyl-O—R⁶, —C(O)N(R⁶)₂, —N(R⁶)₂, halogen, C₁₋₆alkyl-N(R⁶)₂, and cyano; wherein C₁₋₆alkyl, C₁₋₆alkoxy, C₁₋₆alkyl-O—R⁶, and C₁₋₆alkyl-N(R⁶)₂ may optionally be substituted by one or more substituents each independently selected from the group consisting of hydroxyl, —NH₂, oxo, and halogen,
or R⁴ and R⁵, taken together with the atoms to which they are attached, form a 3-7 membered carbocyclic or heterocyclic saturated or partially unsaturated ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;
or R⁴ and R⁸, taken together with the atoms to which they are attached, form a 4-7 membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;
or R⁴ is absent, and W and R, taken together with the atoms to which they are attached, form a 3-membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;
R⁶ is independently for each occurrence selected from the group consisting of hydrogen, C₁₋₆alkyl and phenyl;
R¹¹ and R¹² are each independently selected from the group consisting of hydrogen, hydroxyl, C₁₋₆alkyl, C₁₋₆alkoxy, C₁₋₆alkyl-O—R⁶, aryl, arylalkylene, heteroaryl, heteroarylalkylene, —C(O)N(R⁶)₂, —N(R %)₂, halogen, C₁₋₆alkyl-N(R⁶)₂, —CO₂H, and cyano; wherein C₁₋₆alkyl, C₁₋₆alkoxy, C₁₋₆alkyl-O—R⁶, aryl, arylalkylene, heteroaryl, heteroarylalkylene, and C₁₋₆alkyl-N(R⁶)₂ may optionally be substituted by one or more substituents each independently selected from the group consisting of hydroxyl, —NH₂, and halogen;
or R¹¹ and R¹², taken together with the atoms to which they are attached, form a 5-7 membered heterocyclic ring;
or R⁴ and R¹², taken together with the atoms to which they are attached, form a 5-7 membered carbocyclic or heterocyclic ring;
or R⁸ and R¹¹, taken together with the atoms to which they are attached, form a 5-7 membered carbocyclic or heterocyclic ring;
R⁸ and R⁹ are each independently selected from the group consisting of hydrogen, C₁₋₆)alkyl, C₁₋₆alkyl-N(R⁶)₂, —OR⁶, C₁₋₆alkyl-O—R⁶, —C(O)NH₂, —N(R⁶)₂, halogen, and cyano; and
each of m and n is, independently, 0, 1, 2, or 3, with m+n being at least 2 and no more than 4.

103. The compound of claim 102, wherein m is an integer selected from 1 or 2; and n is 1.

104. The compound of claim 102 or 103, wherein R⁴ and R⁵ are each independently selected from the group consisting of hydrogen, hydroxyl, C₁₋₃alkyl, C₁₋₃alkoxy, C₁₋₃alkyl-O—R⁶, —C(O)NH₂, —N(R⁶)₂, halogen, C₁₋₃alkyl-N(R⁶)₂, and cyano; wherein C₁₋₃alkyl, C₁₋₃alkoxy, C₁₋₃alkyl-O—R⁶ and C₁₋₃alkyl-N(R⁶)₂ may optionally be substituted by one or more substituents each independently selected from the group consisting of hydroxyl, —N(R⁶)₂, and halogen;
or R⁴ and R⁵, taken together with the atoms to which they are attached, form a 3-7 membered carbocyclic or heterocyclic ring;
or R⁴ and R⁸, taken together with the atoms to which they are attached, form a 4-7 membered carbocyclic or heterocyclic ring;
or R⁴ is a bond, and R and R⁸, taken together with the atoms to which they are attached, form a 3-membered carbocyclic or heterocyclic ring; and
each R⁶ is independently hydrogen or C₁₋₃alkyl.

105. The compound of any one of claims 102-104, wherein W and R⁵ are each independently selected from the group consisting of C₁₋₃alkyl and NR^{a}R^{b}; wherein C₁₋₃alkyl may optionally be substituted by one, two or three substituents each independently selected from the group consisting of halogen, hydroxyl and NR^{a}R^{b}.

106. The compound of any one of claims 102-105, wherein R⁴ and R⁵ are each independently selected from the group consisting of —NH₂, —CH₃, and —CH₂NH₂.

107. The compound of any one of claims 85-106, wherein —Y—Z—(R¹)(R²) is represented by: 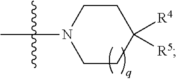
wherein
R⁴ and R⁵ are each independently selected from the group consisting of hydrogen, halogen, cyano, —NR^{a}R^{b}, —C(O)—NR^{a}R^{b} and C₁₋₆alkyl, wherein C₁₋₆alkyl may optionally be substituted by one, two or three or more substituents each independently selected from the group consisting of halogen, hydroxyl, —NR^{a}R^{b} and oxo;
R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl; and q is 0 or 1.

108. A compound selected from the group consisting of: 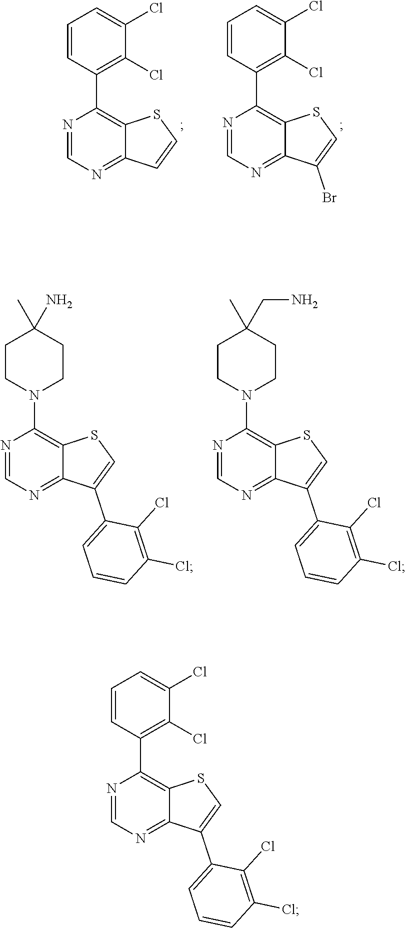 [SMILES: Clc1cccc(-c2ncnc3ccsc23)c1Cl ; Clc1cccc(-c2ncnc3c(Br)csc23)c1Cl ; CC1(N)CCN(c2ncnc3c(-c4cccc(Cl)c4Cl)csc23)CC1 ; CC1(CN)CCN(c2ncnc3c(-c4cccc(Cl)c4Cl)csc23)CC1 ; Clc1cccc(-c2csc3c(-c4cccc(Cl)c4Cl)ncnc23)c1Cl]
and a pharmaceutically acceptable salt or stereoisomer thereof.

109. A compound of Formula IX, or a pharmaceutically acceptable salt or stereoisomer thereof, wherein Formula IX is represented by: 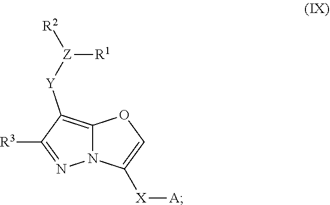
wherein
X is selected from the group consisting of a bond, —O—, —NR^{X1}—, —C(O)NR^{X11}—, —S(O)_{w}— (wherein w is 0, 1 or 2), —C(O)—, —C(R^{X2}R^{X3})—, —O—C(R^{X4}R^{X5})—, —C(R^{X4}R^{X5})—O—, and —C═C(R^{X6}R^{X7})—;
Y is selected from the group consisting of a bond, —NR^{Y}—, —C₁₋₃alkylene-NR^{Y}—, and —NR^{Y}—C₁₋₃alkylene-;
Z is selected from the group consisting of N and C(R^{Z}), or Z and R¹ are absent;
wherein if Y is a bond, Z is N or absent; and wherein if Y is —NR^{Y}—, —C₁₋₃alkylene-NR^{Y}—, or —NR^{Y}—C₁₋₃alkylene-, Z is C(R^{Z});
R^{Z} is selected from the group consisting of hydrogen and C₁₋₆alkyl; wherein C₁₋₆alkyl may optionally be substituted by halogen, hydroxyl, and —NR^{a}R^{b},
A is selected from the group consisting of a 6-10 membered monocyclic or bicyclic aryl, a 5-7 membered monocyclic heteroaryl having one or more heteroatoms each independently selected from O, S, or N, a 8-10 membered bicyclic heteroaryl having one or more heteroatoms each independently selected from O, S, or N; and a 4-7 membered heterocyclyl, wherein A may optionally be substituted by one, two, three or more substituents each independently selected from the group consisting of —R¹⁰, —OR¹⁰, —S(O)_{w}R¹⁰ (wherein w is 0, 1 or 2), —N(R¹⁰)₂, —OS(O)—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;
R¹⁰ is independently selected from the group consisting of hydrogen, C₁₋₆alkyl, C₁₋₆haloalkyl, C₃₋₆cycloalkyl, heterocycloalkyl, aryl, and heteroaryl; wherein R¹⁰ may optionally be substituted by one, two, three or more substituents each independently selected from the group consisting of halogen, —C(O)R²⁰, C₁₋₆alkyl and C₁₋₆haloalkyl;
R²⁰ is selected from the group consisting of hydroxyl, halogen, and C₁₋₆alkyl;
R¹ and R² are each selected from the group consisting of hydrogen and C₁₋₁₀alkyl; wherein C₁₋₁₀alkyl may optionally be substituted by one, two, three or more substituents each independently selected from R^{P};
or R¹ and R², together with carbon or nitrogen to which they are attached, form a 3-7 membered saturated carbocyclic or 4-7 membered saturated heterocyclic ring B having one or two heteroatoms each independently selected from the group consisting of O, S(O), (wherein w is 0, 1, or 2), and NR^{h}; wherein carbocyclic or heterocyclic ring B may be monocyclic or bicyclic;
carbocyclic or heterocyclic ring B may optionally be substituted on one or more available carbons by one, two, three or more substituents each independently selected from the group consisting of halogen, hydroxyl, cyano, oxo, —NR^{a}R^{b}, —CO₂H, —C(O)—NR^{a}R^{b}, —S(O)₂—NR^{a}R^{b}, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})—, C₁₋₆alkoxycarbonyl-N(R^{a})—, aryl and heteroaryl; wherein C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})— and C₁₋₆alkoxycarbonyl-N(R^{a})— may optionally be substituted by one or more substituents each independently selected from R^{p}; and wherein aryl and heteroaryl may optionally be substituted by one or more substituents each independently selected from R;
R^{X1} is selected from the group consisting of hydrogen, C₁₋₆alkyl and phenyl, or R^{X1} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R, —P(O)(R¹⁰)₂, —C(O)R¹⁰, —C(O)N(R¹⁰)₂, oxo, halogen and cyano;
R^{X11} is selected from the group consisting of hydrogen, C₁₋₆alkyl and phenyl, or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;
R^{X2} and R^{X3} are each independently selected from the group consisting of hydrogen, halogen, hydroxyl, —NR^{a}R^{b}, —C(O)—NR^{a}R^{b}, C₁₋₆alkyl and C₁₋₆alkoxy; wherein C₁₋₆alkyl and C₁₋₆alkoxy may optionally be substituted by one or more substituents each independently selected from R^{P};
or R^{X2} and R^{X3} together with the carbon to which they are attached form a 3-6 membered carbocycle optionally substituted by one or more substituents each independently selected from the group consisting of hydrogen, halogen, —NR^{a}R^{b}, —C(O)—NR^{a}R^{b}, oxo, C₁₋₆alkyl and C₁₋₆alkoxy;
R^{X4} and R^{X5} are each independently selected from the group consisting of hydrogen and C₁₋₆alkyl; wherein C₁₋₆alkyl may optionally be substituted by one or more substituents each independently selected from R^{P};
R^{X6} and R^{X7} are each independently selected from the group consisting of hydrogen, halogen, —C(O)—NR^{a}R^{b}, cyano and C₁₋₆alkyl; wherein C₁₋₆alkyl may optionally be substituted by one or more substituents each independently selected from R^{P};
R^{Y} is selected from the group consisting of hydrogen, C₁₋₆alkyl and phenyl;
R³ is selected from the group consisting of hydrogen, halogen, hydroxyl, C₁₋₆alkyl and NR^{a}R^{b};
R^{f} is independently selected, for each occurrence, from the group consisting of R^{P}, C₁₋₆alkyl, —C₁₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, (wherein wherein w is 0, 1 or 2), C₁₋₆alkylcarbonyl-N(R^{a}) and C₁₋₆alkoxycarbonyl-N(R^{a})—; wherein C₁₋₆alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, C₁₋₆alkylcarbonyl-N(R^{a})—, and C₁₋₆alkoxycarbonyl-N(R^{a})— may be optionally substituted by one or more substituents selected from R^{p};
R^{h} is independently selected, for each occurrence, from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆alkylcarbonyl-, C₁₋₆alkoxycarbonyl-, R^{a}R^{b}N-carbonyl- and R^{a}R^{b}N—SO₂—; wherein C₁₋₆alkyl, C₃₋₆alkenyl, C₃₋₆alkynyl, C₃₋₆cycloalkyl, C₁₋₆alkyl-S(O)₂—, C₁₋₆alkylcarbonyl- and C₁₋₆alkoxycarbonyl- may optionally be substituted by one or more substituents selected from R^{P};
R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl;
or R^{a} and R^{b}, together with the nitrogen to which they are attached, may form a 4-6 membered monocyclic heterocyclic ring, which may have an additional heteroatom selected from the group consisting of O, S, and N; wherein the 4-6 membered heterocyclic ring may optionally be substituted by one or more substituents selected from the group consisting of halogen, cyano, oxo or hydroxyl; and
R^{P} is independently selected, for each occurrence, from the group consisting of halogen, hydroxyl, cyano, C₁₋₆alkoxy, R^{a}R^{b}N—, R^{a}R^{b}N-carbonyl-, R^{a}R^{b}N—SO₂—, and R^{a}R^{b}N-carbonyl-N(R^{a})—.

110. The compound of claim 109, wherein X is selected from the group consisting of a bond, —O—, —NR^{X1}— and —NR^{X11}—.

111. The compound of claim 109 or 110, wherein A is selected from the group consisting of phenyl, pyridyl, quinolinyl, indolyl, and indolinyl, wherein phenyl, pyridyl, quinolinyl, indolyl and indolinyl may optionally be substituted by one, two or three substituents each independently selected from the group consisting of —R¹, —OR, —SR¹⁰, —N(R¹⁰)₂, —OSO₂R¹⁰, —SO₂R¹⁰, —C(O)N(R¹⁰)₂, halogen, and cyano.

112. The compound of any one of claims 109-111, wherein A is phenyl; wherein phenyl may optionally be substituted by one, two, or three substituents each independently selected from the group consisting of —OR¹⁰, halogen, and cyano.

113. The compound of any one of claims 109-111, wherein A is pyridyl; wherein pyridyl may optionally be substituted by one, two, or three substituents each independently selected from the group consisting of —OR¹⁰, halogen, and cyano.

114. The compound of claim 109 or 110, wherein R^{X1} and ring A together with the nitrogen to which they are attached form a 1,2,3,4-tetrahydro-1,5-naphthyridine moiety.

115. The compound of claim 109 or 110, wherein R^{X1} and ring A together with the nitrogen to which they are attached form a 1,2,3,4-tetrahydro-1,5-naphthyridine moiety.

116. The compound of claim 109 or 110, wherein R^{X1} and ring A together with the nitrogen to which they are attached form a 1,2,3,4-tetrahydroquinoline moiety.

117. The compound of claim 109 or 110, wherein R^{X1} and ring A together with the nitrogen to which they are attached form a 1,2,3,4-tetrahydroquinoline moiety.

118. The compound of any one of claims 109-117, wherein R³ is selected from the group consisting of hydrogen, CH₃ and —NH₂.

119. The compound of any one of claims 109-118, wherein Y is a bond and Z is N.

120. The compound of any one of claims 109-119, wherein R¹ and R² together with the nitrogen to which they are attached form heterocyclic ring B; wherein ring B is selected from the group consisting of pyrrolidinyl, piperidinyl, azepanyl and morpholinyl; wherein pyrrolidinyl, piperidinyl, azepanyl and morpholinyl may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃alkoxy; wherein C₁₋₃alkyl and C₁₋₃alkoxy may optionally be substituted by halogen, hydroxyl and NR^{a}R^{b}.

121. The compound of claim any one of claims 100-118, wherein Y is —NH—, —N(CH₃)—, —N(H)—CH₂—, or —N(CH₃)—CH₂— and Z is C(H).

122. The compound of claim 121, wherein R¹ and R² together with the carbon to which they are attached form carbocyclic ring B; wherein ring B is selected from the group consisting of cyclopentyl and cyclohexyl, and wherein cyclopentyl and cyclohexyl may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃alkoxy; wherein C₁₋₃alkyl and C₁₋₃alkoxy may optionally be substituted by halogen, hydroxyl and —NR^{a}R^{b}.

123. The compound of claim 121, wherein R¹ and R² together with the carbon to which they are attached form heterocyclic ring B; wherein ring B is selected from the group consisting of tetrahydrofuranyl and tetrahydropyranyl, wherein tetrahydrofuranyl and tetrahydropyranyl may optionally be substituted by one, two, three or more substituents each independently selected from the group consisting of halogen, hydroxyl, cyano, —NR^{a}R^{b}, —C(O)—NR^{a}R^{b}, C₁₋₃ alkyl, and C₁₋₃alkoxy; wherein C₁₋₃alkyl and C₁₋₃alkoxy may optionally be substituted by halogen, hydroxyl and NR^{a}R^{b}.

124. The compound of any one of claims 109-123, wherein ring B is substituted on an available carbon by a substituent selected from the group consisting of —NH₂, —CH₃, and —CH₂NH₂.

125. The compound of any one of claims 109-119, wherein R¹ is hydrogen and R² is branched C₁₋₁₀alkyl; wherein C₁₋₁₀alkyl may optionally be substituted by one, two, three or more substituents each independently selected from the group consisting of halogen and —NH₂.

126. The compound of any one of claims 109-125, wherein —Y—Z—(R¹)(R²) is: 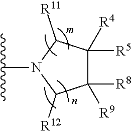
wherein
R⁴ and R⁵ are each independently selected from the group consisting of hydrogen, hydroxyl, C₁₋₆alkyl, C₁₋₆alkoxy, C₁₋₆alkyl-O—R⁶, —C(O)N(R⁶)₂, —N(R⁶)₂, halogen, C₁₋₆alkyl-N(R⁶)₂, and cyano; wherein C₁₋₆alkyl, C₁₋₆alkoxy, C₁₋₆alkyl-O—R⁶, and C₁₋₆alkyl-N(R⁶)₂ may optionally be substituted by one or more substituents each independently selected from the group consisting of hydroxyl, —NH₂, oxo, and halogen,
or R⁴ and R⁵, taken together with the atoms to which they are attached, form a 3-7 membered carbocyclic or heterocyclic saturated or partially unsaturated ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;
or R⁴ and R⁸, taken together with the atoms to which they are attached, form a 4-7 membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;
or R⁴ is absent, and R and R, taken together with the atoms to which they are attached, form a 3-membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;
R⁶ is independently for each occurrence selected from the group consisting of hydrogen, C₁₋₆alkyl and phenyl;
R¹¹ and R¹² are each independently selected from the group consisting of hydrogen, hydroxyl, C₁₋₆alkyl, C₁₋₆alkoxy, C₁₋₆alkyl-O—R⁶, aryl, arylalkylene, heteroaryl, heteroarylalkylene, —C(O)N(R⁶)₂, —N(R⁶)₂, halogen, C₁₋₆alkyl-N(R⁶)₂, —CO₂H, and cyano; wherein C₁₋₆alkyl, C₁₋₆alkoxy, C₁₋₆alkyl-O—R⁶, aryl, arylalkylene, heteroaryl, heteroarylalkylene, and C₁₋₆alkyl-N(R⁶)₂ may optionally be substituted by one or more substituents each independently selected from the group consisting of hydroxyl, —NH₂, and halogen;
or R¹¹ and R¹², taken together with the atoms to which they are attached, form a 5-7 membered heterocyclic ring;
or R⁴ and R¹², taken together with the atoms to which they are attached, form a 5-7 membered carbocyclic or heterocyclic ring;
or R⁸ and R¹¹, taken together with the atoms to which they are attached, form a 5-7 membered carbocyclic or heterocyclic ring;
R⁸ and R⁹ are each independently selected from the group consisting of hydrogen, C₁₋₆)alkyl, C₁₋₆alkyl-N(R⁶)₂, —OR⁶, C₁₋₆alkyl-O—R⁶, —C(O)NH₂, —N(R⁶)₂, halogen, and cyano; and
each of m and n is, independently, 0, 1, 2, or 3, with m+n being at least 2 and no more than 4.

127. The compound of claim 126, wherein m is an integer selected from 1 or 2; and n is 1.

128. The compound of claim 126 or 127, wherein R⁴ and R⁵ are each independently selected from the group consisting of hydrogen, hydroxyl, C₁₋₃alkyl, C₁₋₃alkoxy, C₁₋₃alkyl-O—R⁶, —C(O)NH₂, —N(R⁶)₂, halogen, C₁₋₃alkyl-N(R⁶)₂, and cyano; wherein C₁₋₃alkyl, C₁₋₃alkoxy, C₁₋₃alkyl-O—R⁶ and C₁₋₃alkyl-N(R⁶)₂ may optionally be substituted by one or more substituents each independently selected from the group consisting of hydroxyl, —N(R⁶)₂, and halogen;
or R⁴ and R⁵, taken together with the atoms to which they are attached, form a 3-7 membered carbocyclic or heterocyclic ring;
or R⁴ and R⁸, taken together with the atoms to which they are attached, form a 4-7 membered carbocyclic or heterocyclic ring;
or R⁴ is a bond, and R and R⁸, taken together with the atoms to which they are attached, form a 3-membered carbocyclic or heterocyclic ring; and
each R⁶ is independently hydrogen or C₁₋₃alkyl.

129. The compound of any one of claims 126-128, wherein W and R⁵ are each independently selected from the group consisting of C₁₋₃alkyl and NR^{a}R^{b}; wherein C₁₋₃alkyl may optionally be substituted by one, two or three substituents each independently selected from the group consisting of halogen, hydroxyl and NR^{a}R^{b}.

130. The compound of any one of claims 126-129, wherein W and R⁵ are each independently selected from the group consisting of —NH₂, —CH₃, and —CH₂NH₂.

131. The compound of any one of claims 109-130, wherein —Y—Z—(R¹)(R²) is represented by: 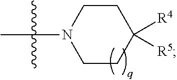
wherein
R⁴ and R⁵ are each independently selected from the group consisting of hydrogen, halogen, cyano, —NR^{a}R^{b}, —C(O)—NR^{a}R^{b} and C₁₋₆alkyl, wherein C₁₋₆alkyl may optionally be substituted by one, two or three or more substituents each independently selected from the group consisting of halogen, hydroxyl, —NR^{a}R^{b} and oxo;
R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl; and q is 0 or 1.

132. A compound selected from the group consisting of: 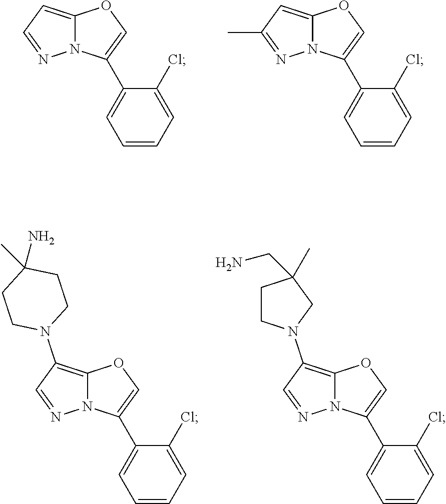 [SMILES: Clc1ccccc1-c1coc2ccnn12 ; Cc1cc2occ(-c3ccccc3Cl)n2n1 ; CC1(N)CCN(c2cnn3c(-c4ccccc4Cl)coc23)CC1 ; CC1(CN)CCN(c2cnn3c(-c4ccccc4Cl)coc23)C1]
and a pharmaceutically acceptable salt or stereoisomer thereof.

133. A compound of Formula X, or a pharmaceutically acceptable salt or stereoisomer thereof, wherein Formula X is represented by: 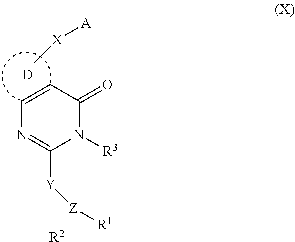
wherein
Ring D is absent or together with carbons to which it is attached, is a 5 membered heteroaryl having one or two heteroatoms each selected from S, O, N, or NR^{a};
X is bound to a free carbon on the pyrimidinone when D is absent, or on a free carbon of ring D, and selected from the group consisting of a bond, —O—, —NR^{X1}— —C(O)NR^{X11}—, —S(O)_{w}— (wherein w is 0, 1 or 2), —C(O)—, —C(R^{X2}R^{X3})—, —O—C(R^{X4}R^{X5})—, —C(R^{X4}R^{X5})—O—, and —C═C(R^{X6}R^{X7})—;
Y is selected from the group consisting of a bond, —NR^{Y}—, —C₁₋₃alkylene-NR^{Y}—, and —NR^{Y}—C₁₋₃alkylene-;
Z is selected from the group consisting of N and C(R^{Z}), or Z and R are absent;
wherein if Y is a bond, Z is N or is absent, and wherein if Y is —N^{Y}—, —C₁₋₃alkylene-NR^{Y}—, or —NR^{Y}—C₁₋₃alkylene-, Z is C(R^{Z});
R^{Z} is selected from the group consisting of hydrogen and C₁₋₆alkyl; wherein C₁₋₆alkyl may optionally be substituted by halogen, hydroxyl, and —NR^{a}R^{b},
A is selected from the group consisting of a 6-10 membered monocyclic or bicyclic aryl, a 5-7 membered monocyclic heteroaryl having one or more heteroatoms each independently selected from O, S, or N, a 8-10 membered bicyclic heteroaryl having one or more heteroatoms each independently selected from O, S, or N; and a 4-7 membered heterocyclyl, wherein A may optionally be substituted by one, two, three or more substituents each independently selected from the group consisting of —R¹⁰, —OR¹⁰, —S(O)_{w}R¹⁰ (wherein w is 0, 1 or 2), —N(R¹⁰)₂, —OS(O)—R¹⁰ (wherein w is 0, 1, or 2), —S(O)_{w}—N(R¹⁰)₂ (wherein w is 0, 1 or 2), —S(O)(NH)R¹, —P(O)(R¹⁰)₂, —C(O)R¹⁰, —C(O)N(R¹⁰)₂, oxo, halogen and cyano;
R¹⁰ is independently selected from the group consisting of hydrogen, C₁₋₆alkyl, C₁₋₆ haloalkyl, C₃₋₆cycloalkyl, heterocycloalkyl, aryl, and heteroaryl; wherein R¹⁰ may optionally be substituted by one, two, three or more substituents each independently selected from the group consisting of halogen, —C(O)R²⁰, C₁₋₆alkyl and C₁₋₆haloalkyl;
R²⁰ is selected from the group consisting of hydroxyl, halogen, and C₁₋₆alkyl;
R¹ and R² are each selected from the group consisting of hydrogen and C₁₋₁₀alkyl; wherein C₁₋₁₀alkyl may optionally be substituted by one, two, three or more substituents each independently selected from R^{P};
or R¹ and R², together with carbon or nitrogen to which they are attached, form a 3-7 membered saturated carbocyclic or 4-7 membered saturated heterocyclic ring B having one or two heteroatoms each independently selected from the group consisting of 0, S(O), (wherein w is 0, 1, or 2), and NR^{h}; wherein carbocyclic or heterocyclic ring B may be monocyclic or bicyclic;
carbocyclic or heterocyclic ring B may optionally be substituted on one or more available carbons by one, two, three or more substituents each independently selected from the group consisting of halogen, hydroxyl, cyano, oxo, NR^{a}R^{b}, —CO₂H, —C(O)—NR^{a}R^{b}, —S(O)₂—NR^{a}R^{b}, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})—, C₁₋₆alkoxycarbonyl-N(R^{a})—, aryl and heteroaryl; wherein C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})— and C₁₋₆alkoxycarbonyl-N(R^{a})— may optionally be substituted by one or more substituents each independently selected from R; and wherein aryl and heteroaryl may optionally be substituted by one or more substituents each independently selected from R;
R^{X1} is selected from the group consisting of hydrogen, C₁₋₆alkyl and phenyl, or R and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;
R^{X11} is selected from the group consisting of hydrogen, C₁₋₆alkyl and phenyl, or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)^{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;
R^{X2} and R^{X3} are each independently selected from the group consisting of hydrogen, halogen, hydroxyl, —NR^{a}R^{b}, —C(O)—NR^{a}R^{b}, C₁₋₆alkyl and C₁₋₆alkoxy; wherein C₁₋₆alkyl and C₁₋₆alkoxy may optionally be substituted by one or more substituents each independently selected from R^{p};
or R^{X2} and R^{X3} together with the carbon to which they are attached form a 3-6 membered carbocycle optionally substituted by one or more substituents each independently selected from the group consisting of hydrogen, halogen, NR^{a}R^{b}, —C(O)—NR^{a}R^{b}, oxo, C₁₋₆alkyl and C₁₋₆alkoxy;
R^{X4} and R^{X5} are each independently selected from the group consisting of hydrogen and C₁₋₆alkyl; wherein C₁₋₆alkyl may optionally be substituted by one or more substituents each independently selected from R^{P};
R^{X6} and R^{X7} are each independently selected from the group consisting of hydrogen, halogen, —C(O)—NR^{a}R^{b}, cyano and C₁₋₆alkyl; wherein C₁₋₆alkyl may optionally be substituted by one or more substituents each independently selected from R^{P};
R^{Y} is selected from the group consisting of hydrogen, C₁₋₆alkyl and phenyl;
R³ is selected from the group consisting of hydrogen, C₁₋₆alkyl, C₃₋₆alkyl, phenyl, and heterocycle;
R^{f} is independently selected, for each occurrence, from the group consisting of R^{P}, C₁₋₆alkyl, —C₁₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, (wherein wherein w is 0, 1 or 2), C₁₋₆alkylcarbonyl-N(R^{a}) and C₁₋₆alkoxycarbonyl-N(R^{a})—; wherein C₁₋₆alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, C₁₋₆alkylcarbonyl-N(R^{a})—, and C₁₋₆alkoxycarbonyl-N(R^{a})— may be optionally substituted by one or more substituents selected from R^{p};
R^{h} is independently selected, for each occurrence, from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆ alkylcarbonyl-, C₁₋₆alkoxycarbonyl-, R^{a}R^{b}N-carbonyl- and R^{a}R^{b}N—SO₂—; wherein C₁₋₆alkyl, C₃₋₆alkenyl, C₃₋₆alkynyl, C₃₋₆cycloalkyl, C₁₋₆alkyl-S(O)₂—, C₁₋₆alkylcarbonyl- and C₁₋₆alkoxycarbonyl- may optionally be substituted by one or more substituents selected from R^{p};
R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl;
or R^{a} and R^{b}, together with the nitrogen to which they are attached, may form a 4-6 membered monocyclic heterocyclic ring, which may have an additional heteroatom selected from the group consisting of O, S, and N; wherein the 4-6 membered heterocyclic ring may optionally be substituted by one or more substituents selected from the group consisting of halogen, cyano, oxo or hydroxyl; and
R^{p} is independently selected, for each occurrence, from the group consisting of halogen, hydroxyl, cyano, C₁₋₆alkoxy, R^{a}R^{b}N—, R^{a}R^{b}N-carbonyl-, R^{a}R^{b}N—SO₂—, and R^{a}R^{b}N-carbonyl-N(R^{a}).

134. The compound of claim 133, wherein X is selected from the group consisting of a bond, —O—, —NR^{X1}— and —NR^{X11}—.

135. The compound of claim 133 or 134, wherein A is selected from the group consisting of phenyl, pyridyl, quinolinyl, indolyl, and indolinyl, wherein phenyl, pyridyl, quinolinyl, indolyl and indolinyl may optionally be substituted by one, two or three substituents each independently selected from the group consisting of —R¹⁰, —OR¹⁰, —SR¹⁰, —N(R¹⁰)₂, —OSO₂R¹⁰, —SO₂R¹⁰, —C(O)N(R¹⁰)₂, halogen, and cyano.

136. The compound of any one of claims 133-135, wherein A is phenyl; wherein phenyl may optionally be substituted by one, two, or three substituents each independently selected from the group consisting of —OR¹⁰, halogen, and cyano.

137. The compound of any one of claims 133-135, wherein A is pyridyl; wherein pyridyl may optionally be substituted by one, two, or three substituents each independently selected from the group consisting of —OR¹⁰, halogen, and cyano.

138. The compound of claim 133 or 134, wherein R^{X1} and ring A together with the nitrogen to which they are attached form a 1,2,3,4-tetrahydro-1,5-naphthyridine moiety.

139. The compound of claim 133 or 134, wherein R^{X1} and ring A together with the nitrogen to which they are attached form a 1,2,3,4-tetrahydro-1,5-naphthyridine moiety.

140. The compound of claim 133 or 134, wherein R^{X1} and ring A together with the nitrogen to which they are attached form a 1,2,3,4-tetrahydroquinoline moiety.

141. The compound of claim 133 or 134, wherein R^{X1} and ring A together with the nitrogen to which they are attached form a 1,2,3,4-tetrahydroquinoline moiety.

142. The compound of any one of claims 133-141, wherein R³ is selected from the group consisting of hydrogen, CH₃ and —NH₂.

143. The compound of any one of claims 133-142, wherein Y is a bond and Z is N.

144. The compound of any one of claims 133-143, wherein R¹ and R² together with the nitrogen to which they are attached form heterocyclic ring B; wherein ring B is selected from the group consisting of pyrrolidinyl, piperidinyl, azepanyl and morpholinyl; wherein pyrrolidinyl, piperidinyl, azepanyl and morpholinyl may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃alkoxy; wherein C₁₋₃ alkyl and C₁₋₃alkoxy may optionally be substituted by halogen, hydroxyl and NR^{a}R^{b}.

145. The compound of claim any one of claims 133-142, wherein Y is —NH—, —N(CH₃)—, —N(H)—CH₂—, or —N(CH₃)—CH₂— and Z is C(H).

146. The compound of claim 145, wherein R¹ and R² together with the carbon to which they are attached form carbocyclic ring B; wherein ring B is selected from the group consisting of cyclopentyl and cyclohexyl, and wherein cyclopentyl and cyclohexyl may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃ alkoxy; wherein C₁₋₃alkyl and C₁₋₃alkoxy may optionally be substituted by halogen, hydroxyl and —NR^{a}R^{b}.

147. The compound of claim 145, wherein R¹ and R² together with the carbon to which they are attached form heterocyclic ring B; wherein ring B is selected from the group consisting of tetrahydrofuranyl and tetrahydropyranyl, wherein tetrahydrofuranyl and tetrahydropyranyl may optionally be substituted by one, two, three or more substituents each independently selected from the group consisting of halogen, hydroxyl, cyano, —NR^{a}R^{b}, —C(O)—NR^{a}R^{b}, C₁₋₃ alkyl, and C₁₋₃alkoxy; wherein C₁₋₃alkyl and C₁₋₃alkoxy may optionally be substituted by halogen, hydroxyl and NR^{a}R^{b}.

148. The compound of any one of claims 133-147, wherein ring B is substituted on an available carbon by a substituent selected from the group consisting of —NH₂, —CH₃, and —CH₂NH₂.

149. The compound of any one of claims 133-143, wherein R¹ is hydrogen and R² is branched C₁₋₁₀alkyl; wherein C₁₋₁₀alkyl may optionally be substituted by one, two, three or more substituents each independently selected from the group consisting of halogen and —NH₂.

150. The compound of any one of claims 133-149, wherein ring D is absent, and formula X is represented by formula Xa:

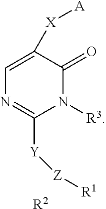

151. The compound of any one of claims 133-149, wherein ring D is present, and formula X is represented by Xb or Xc:

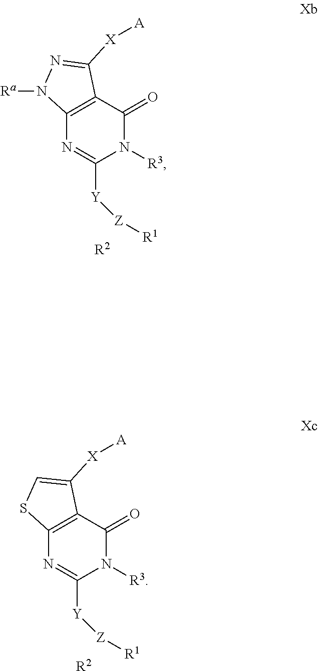

152. A compound of Formula XI or a pharmaceutically acceptable salt or stereoisomer thereof, wherein Formula XI is represented by: 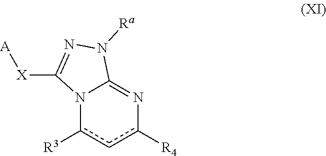
wherein
X is selected from the group consisting of a bond, —O—, —NR^{X1}—, —C(O)NR^{X11}—, —S(O)_{w}— (wherein w is 0, 1 or 2), —C(O)—, —(R^{X2}R^{X3})—, —O—C(R^{X4}R^{X5})—, —C(R^{X4}R^{X5})—O—, and —C═C(R^{X6}R^{X7})—;
R³ is —Y—Z(R¹)(R²) and R⁴ is ═O; or R³ is ═O and R⁴ is —Y—Z(R¹)(R²);
Y is selected from the group consisting of a bond, —NR^{Y}—, —C₁₋₃alkylene-NR^{Y}—, and —NR^{Y}—C₁₋₃alkylene-;
Z is selected from the group consisting of N and C(R^{Z});
wherein if Y is a bond, Z is N, and wherein if Y is —NR^{Y}—, —C₁₋₃alkylene-NR^{Y}—, or —NR^{Y}—C₁₋₃alkylene-, Z is C(R^{Z});
R^{Z} is selected from the group consisting of hydrogen and C₁₋₆alkyl; wherein C₁₋₆alkyl may optionally be substituted by halogen, hydroxyl, and —NR^{a}R^{b},
A is selected from the group consisting of a 6-10 membered monocyclic or bicyclic aryl, a 5-7 membered monocyclic heteroaryl having one or more heteroatoms each independently selected from O, S, or N, a 8-10 membered bicyclic heteroaryl having one or more heteroatoms each independently selected from O, S, or N; and a 4-7 membered heterocyclyl, wherein A may optionally be substituted by one, two, three or more substituents each independently selected from the group consisting of —R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;
R¹⁰ is independently selected from the group consisting of hydrogen, C₁₋₆alkyl, C₁₋₆haloalkyl, C₃₋₆cycloalkyl, heterocycloalkyl, aryl, and heteroaryl; wherein R¹⁰ may optionally be substituted by one, two, three or more substituents each independently selected from the group consisting of halogen, —C(O)R²⁰, C₁₋₆alkyl and C₁₋₆haloalkyl;
R²⁰ is selected from the group consisting of hydroxyl, halogen, and C₁₋₆alkyl;
R¹ and R² are each selected from the group consisting of hydrogen and C₁₋₁₀alkyl; wherein C₁₋₁₀alkyl may optionally be substituted by one, two, three or more substituents each independently selected from R^{P};
or R¹ and R², together with carbon or nitrogen to which they are attached, form a 3-7 membered saturated carbocyclic or 4-7 membered saturated heterocyclic ring B having one or two heteroatoms each independently selected from the group consisting of O, S(O), (wherein w is 0, 1, or 2), and NR^{h}; wherein carbocyclic or heterocyclic ring B may be monocyclic or bicyclic;
carbocyclic or heterocyclic ring B may optionally be substituted on one or more available carbons by one, two, three or more substituents each independently selected from the group consisting of halogen, hydroxyl, cyano, oxo, —NR^{a}R^{b}, —CO₂H, —C(O)—NR^{a}R^{b}, —S(O)₂—NR^{a}R^{b}, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})—, C₁₋₆alkoxycarbonyl-N(R^{a})—, aryl and heteroaryl; wherein C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})— and C₁₋₆alkoxycarbonyl-N(R^{a})— may optionally be substituted by one or more substituents each independently selected from R^{p}; and wherein aryl and heteroaryl may optionally be substituted by one or more substituents each independently selected from R;
R^{X1} is selected from the group consisting of hydrogen, C₁₋₆alkyl and phenyl, or R and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;
R^{X11} is selected from the group consisting of hydrogen, C₁₋₆alkyl and phenyl, or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;
R^{X2} and R^{X3} are each independently selected from the group consisting of hydrogen, halogen, hydroxyl, —NR^{a}R^{b}, —C(O)—NR^{a}R^{b}, C₁₋₆alkyl and C₁₋₆alkoxy; wherein C₁₋₆alkyl and C₁₋₆alkoxy may optionally be substituted by one or more substituents each independently selected from R^{p};
or R^{X2} and R^{X3} together with the carbon to which they are attached form a 3-6 membered carbocycle optionally substituted by one or more substituents each independently selected from the group consisting of hydrogen, halogen, —NR^{a}R^{b}, —C(O)—NR^{a}R^{b}, oxo, C₁₋₆ alkyl and C₁₋₆alkoxy;
R^{X4} and R^{X3} are each independently selected from the group consisting of hydrogen and C₁₋₆alkyl; wherein C₁₋₆alkyl may optionally be substituted by one or more substituents each independently selected from R^{P};
R^{X6} and R^{X7} are each independently selected from the group consisting of hydrogen, halogen, —C(O)—NR^{a}R^{b}, cyano and C₁₋₆alkyl; wherein C₁₋₆alkyl may optionally be substituted by one or more substituents each independently selected from R^{P};
R^{Y} is selected from the group consisting of hydrogen, C₁₋₆alkyl and phenyl;
R³ is selected from the group consisting of hydrogen, C₁₋₆alkyl, C₃₋₆alkyl, phenyl, and heterocycle;
R^{f} is independently selected, for each occurrence, from the group consisting of R^{P}, C₁₋₆alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, (wherein wherein w is 0, 1 or 2), C₁₋₆alkylcarbonyl-N(R^{a}) and C₁₋₆alkoxycarbonyl-N(R^{a})—; wherein C₁₋₆alkyl, C₃₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, C₁₋₆alkylcarbonyl-N(R^{a})—, and C₁₋₆alkoxycarbonyl-N(R^{a})— may be optionally substituted by one or more substituents selected from R^{p};
R^{h} is independently selected, for each occurrence, from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆alkylcarbonyl-, C₁₋₆alkoxycarbonyl-, R^{a}R^{b}N-carbonyl- and R^{a}R^{b}N—SO₂—; wherein C₁₋₆alkyl, C₃₋₆alkenyl, C₃₋₆alkynyl, C₃₋₆cycloalkyl, C₁₋₆alkyl-S(O)₂—, C₁₋₆alkylcarbonyl- and C₁₋₆alkoxycarbonyl- may optionally be substituted by one or more substituents selected from R^{P};
R^{a} and R^{b} are independently selected, for each occurrence, from the group consisting of hydrogen and C₁₋₆alkyl; wherein C₁₋₆alkyl may optionally be substituted by one or more substituents selected from halogen, cyano, oxo and hydroxyl;
or R^{a} and R^{b}, together with the nitrogen to which they are attached, may form a 4-6 membered monocyclic heterocyclic ring, which may have an additional heteroatom selected from the group consisting of O, S, and N; wherein the 4-6 membered heterocyclic ring may optionally be substituted by one or more substituents selected from the group consisting of halogen, cyano, oxo or hydroxyl; and
R is independently selected, for each occurrence, from the group consisting of halogen, hydroxyl, cyano, C₁₋₆alkoxy, R^{a}R^{b}N—, R^{a}R^{b}N-carbonyl-, R^{a}R^{b}N—SO₂—, and R^{a}R^{b}N-carbonyl-N(R^{a})—.

153. The compound of claim 152, wherein the compound is represented by XIb or XIc:

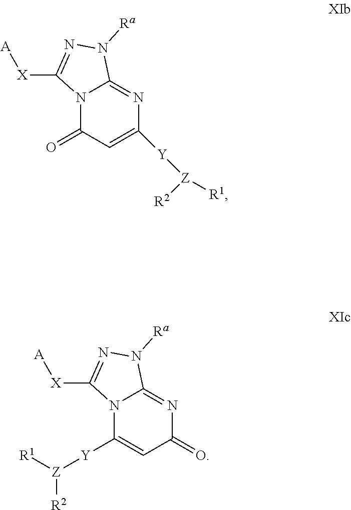

154. The compound of any one of claims 133-153, wherein —Y—Z—(R¹)(R²) is: 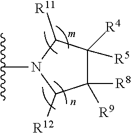
wherein
R⁴ and R⁵ are each independently selected from the group consisting of hydrogen, hydroxyl, C₁₋₆alkyl, C₁₋₆alkoxy, C₁₋₆alkyl-O—R⁶, —C(O)N(R⁶)₂, —N(R⁶)₂, halogen, C₁₋₆alkyl-N(R⁶)₂, and cyano; wherein C₁₋₆alkyl, C₁₋₆alkoxy, C₁₋₆alkyl-O—R⁶, and C₁₋₆alkyl-N(R⁶)₂ may optionally be substituted by one or more substituents each independently selected from the group consisting of hydroxyl, —NH₂, oxo, and halogen,
or R⁴ and R⁵, taken together with the atoms to which they are attached, form a 3-7 membered carbocyclic or heterocyclic saturated or partially unsaturated ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;
or R⁴ and R⁸, taken together with the atoms to which they are attached, form a 4-7 membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;
or R⁴ is absent, and R and R⁸, taken together with the atoms to which they are attached, form a 3-membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;
R⁶ is independently for each occurrence selected from the group consisting of hydrogen, C₁₋₆alkyl and phenyl;
R¹¹ and R¹² are each independently selected from the group consisting of hydrogen, hydroxyl, C₁₋₆alkyl, C₁₋₆alkoxy, C₁₋₆alkyl-O—R⁶, aryl, arylalkylene, heteroaryl, heteroarylalkylene, —C(O)N(R⁶)₂, —N(R⁶)₂, halogen, C₁₋₆alkyl-N(R⁶)₂, —CO₂H, and cyano; wherein C₁₋₆alkyl, C₁₋₆alkoxy, C₁₋₆alkyl-O—R⁶, aryl, arylalkylene, heteroaryl, heteroarylalkylene, and C₁₋₆alkyl-N(R⁶)₂ may optionally be substituted by one or more substituents each independently selected from the group consisting of hydroxyl, —NH₂, and halogen;
or R¹¹ and R¹², taken together with the atoms to which they are attached, form a 5-7 membered heterocyclic ring;
or R⁴ and R¹², taken together with the atoms to which they are attached, form a 5-7 membered carbocyclic or heterocyclic ring;
or R⁸ and R¹¹, taken together with the atoms to which they are attached, form a 5-7 membered carbocyclic or heterocyclic ring;
R⁸ and R⁹ are each independently selected from the group consisting of hydrogen, C₁₋₆)alkyl, C₁₋₆alkyl-N(R⁶)₂, —OR⁶, C₁₋₆alkyl-O—R⁶, —C(O)NH₂, —N(R⁶)₂, halogen, and cyano; and
each of m and n is, independently, 0, 1, 2, or 3, with m+n being at least 2 and no more than 4.

155. The compound of claim 154, wherein m is an integer selected from 1 or 2; and n is 1.

156. The compound of claim 154 or 155, wherein R⁴ and R⁵ are each independently selected from the group consisting of hydrogen, hydroxyl, C₁₋₃alkyl, C₁₋₃alkoxy, C₁₋₃alkyl-O—R⁶, —C(O)NH₂, —N(R⁶)₂, halogen, C₁₋₃alkyl-N(R⁶)₂, and cyano; wherein C₁₋₃alkyl, C₁₋₃alkoxy, C₁₋₃alkyl-O—R⁶ and C₁₋₃alkyl-N(R⁶)₂ may optionally be substituted by one or more substituents each independently selected from the group consisting of hydroxyl, —N(R⁶)₂, and halogen;
or R⁴ and R⁵, taken together with the atoms to which they are attached, form a 3-7 membered carbocyclic or heterocyclic ring;
or R⁴ and R⁸, taken together with the atoms to which they are attached, form a 4-7 membered carbocyclic or heterocyclic ring;
or R⁴ is a bond, and R and R⁸, taken together with the atoms to which they are attached, form a 3-membered carbocyclic or heterocyclic ring; and
each R⁶ is independently hydrogen or C₁₋₃alkyl.

157. The compound of any one of claims 154-156, wherein W and R⁵ are each independently selected from the group consisting of C₁₋₃alkyl and NR^{a}R^{b}; wherein C₁₋₃alkyl may optionally be substituted by one, two or three substituents each independently selected from the group consisting of halogen, hydroxyl and NR^{a}R^{b}.

158. The compound of any one of claims 154-157, wherein W and R⁵ are each independently selected from the group consisting of —NH₂, —CH₃, and —CH₂NH₂.

159. The compound of any one of claims 133-158, wherein —Y—Z—(R¹)(R²) is represented by: 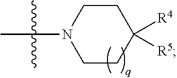
wherein
R⁴ and R⁵ are each independently selected from the group consisting of hydrogen, halogen, cyano, —NR^{a}R^{b}, —C(O)—NR^{a}R^{b} and C₁₋₆alkyl, wherein C₁₋₆alkyl may optionally be substituted by one, two or three or more substituents each independently selected from the group consisting of halogen, hydroxyl, —NR^{a}R^{b} and oxo;
R and R are independently selected, for each occurrence, from the group consisting of hydrogen and C₁₋₆alkyl; wherein C₁₋₆alkyl may optionally be substituted by one or more substituents selected from halogen, cyano, oxo and hydroxyl; and q is 0 or 1.

160. A compound selected from the group consisting of: 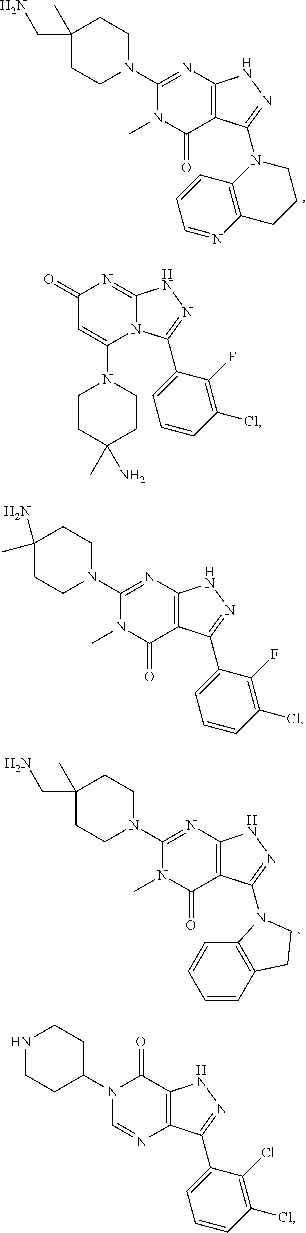 [SMILES: Cn1c(N2CCC(C)(CN)CC2)nc2[nH]nc(N3CCCc4ncccc43)c2c1=O ; CC1(N)CCN(c2cc(=O)nc3[nH]nc(-c4cccc(Cl)c4F)n23)CC1 ; Cn1c(N2CCC(C)(N)CC2)nc2[nH]nc(-c3cccc(Cl)c3F)c2c1=O ; Cn1c(N2CCC(C)(CN)CC2)nc2[nH]nc(N3CCc4ccccc43)c2c1=O ; O=c1c2[nH]nc(-c3cccc(Cl)c3Cl)c2ncn1C1CCNCC1] 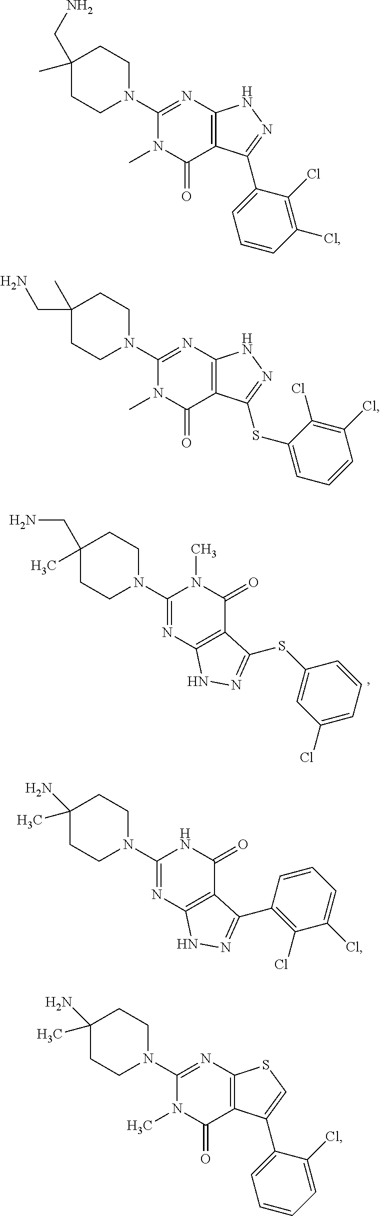 [SMILES: Cn1c(N2CCC(C)(CN)CC2)nc2[nH]nc(-c3cccc(Cl)c3Cl)c2c1=O ; Cn1c(N2CCC(C)(CN)CC2)nc2[nH]nc(Sc3cccc(Cl)c3Cl)c2c1=O ; Cn1c(N2CCC(C)(CN)CC2)nc2[nH]nc(Sc3cccc(Cl)c3)c2c1=O ; CC1(N)CCN(c2nc3[nH]nc(-c4cccc(Cl)c4Cl)c3c(=O)[nH]2)CC1 ; Cn1c(N2CCC(C)(N)CC2)nc2scc(-c3ccccc3Cl)c2c1=O] 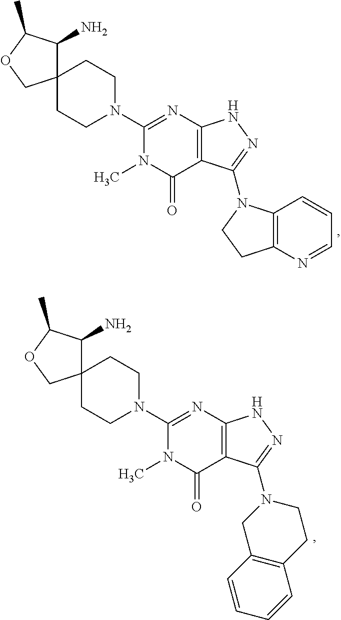 [SMILES: C[C@@H]1OCC2(CCN(c3nc4[nH]nc(N5CCc6ncccc65)c4c(=O)n3C)CC2)[C@@H]1N ; C[C@@H]1OCC2(CCN(c3nc4[nH]nc(N5CCc6ccccc6C5)c4c(=O)n3C)CC2)[C@@H]1N]
and a pharmaceutically acceptable salt or stereoisomer thereof.

161. A pharmaceutical composition comprising a compound of any of claims 1-160 and a pharmaceutically acceptable carrier.

162. A method of inhibiting SHP2 phosphatase activity in a subject comprising administration of a therapeutically effective amount of a compound of any of claims 1-160, or a composition of claim 161, to a subject in need thereof.

163. The method of claim 162, further comprising administration of a therapeutically effective amount of an antibody, an antibody-drug conjugate, an immunomodulator, or a histone deacetylase inhibitor.

164. The method of claim 162 or 163, wherein the subject is a human.

165. A method of treating a disorder in a subject comprising administration of a therapeutically effective amount of a compound of any of claims 1-160, or a composition of claim 161, to a subject in need thereof.

166. The method of claim 165, further comprising administration of a therapeutically effective amount of an antibody, an antibody-drug conjugate, an immunomodulator, or a histone deacetylase inhibitor.

167. The method of claim 165 or 166, wherein the disorder is Noonan syndrome.

168. The method of claim 165 or 166, wherein the disorder is neutropenia.

169. The method of claim 165 or 166, wherein the disorder is diabetes.

170. The method of claim 165 or 166, wherein the disorder is neuroblastoma.

171. The method of claim 165 or 166, wherein the disorder is melanoma.

172. The method of claim 165 or 166, wherein the disorder is acute myeloid leukemia.

173. The method of claim 165 or 166, wherein the disorder is juvenile leukemia.

174. The method of claim 165 or 166, wherein the disorder is juvenile myelomonocytic leukemia.

175. The method of claim 165 or 166, wherein the disorder is breast cancer.

176. The method of claim 165 or 166, wherein the disorder is lung cancer.

177. The method of claim 165 or 166, wherein the disorder is colorectal cancer.

178. A method of treating a disorder in a patient in need thereof, comprising administering to the patient an effective amount of a compound of Formula XII, or a pharmaceutically acceptable salt, stereoisomer or N-oxide thereof, wherein Formula XII is represented by: 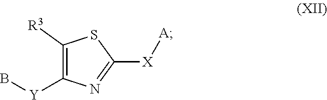
wherein
X is selected from the group consisting of a bond, —O—, —NR^{X1}—, —C(O)NR^{X11}—, —S(O)_{w}— (wherein w is 0, 1 or 2), —C(O)—, —C(R^{X2}R^{X3})—, —O—C(R^{X4}R^{X5})—, —C(R^{X4}R^{X5})—O—, and —C═C(R^{X6}R^{X7})—;
A is selected from the group consisting of a 6-10 membered monocyclic or bicyclic aryl, a 5-7 membered monocyclic heteroaryl having one or more heteroatoms each independently selected from O, S, or N, a 8-10 membered bicyclic heteroaryl having one or more heteroatoms each independently selected from O, S, or N; and a 4-7 membered heterocyclyl, wherein A may optionally be substituted by one, two, three or more substituents each independently selected from the group consisting of —R¹⁰, —OR¹⁰, methylenedioxy, —S(O)_{w}R¹⁰ (wherein w is 0, 1 or 2), —N(R¹)₂, —OS(O)_{w}—R¹⁰ (wherein w is 0, 1, or 2), —S(O)—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;
R¹⁰ is independently selected from the group consisting of hydrogen, C₁₋₆alkyl, C₁₋₆ haloalkyl, C₃₋₆cycloalkyl, heterocycloalkyl, aryl, and heteroaryl; wherein R¹⁰ may optionally be substituted by one, two, three or more substituents each independently selected from the group consisting of halogen, —C(O)R²⁰, C₁₋₆alkyl and C₁₋₆haloalkyl;
R²⁰ is selected from the group consisting of hydroxyl, halogen, and C₁₋₆alkyl;
Y is selected from the group consisting of C₁₋₆alkylene and C₁₋₆alkenylene; wherein C₁₋₆alkylene and C₁₋₆alkenylene may optionally be substituted by one or two substituents each independently selected from the group consisting of halogen and hydroxyl; and wherein one or two methylene units of L may optionally and independently be replaced by a moiety selected from the group consisting of a bond, —O—, —C(O)—, —O—C(O)—, —C(O)—, —NR^{a}—, —C(O)—NR^{a}—, —NR^{a}—C(O)—, —O—C(O)—NR^{a}—, —NR^{a}—C(O)—O—, —S(O)_{w}— (wherein w is 0, 1, or 2), —S(O)—NR^{a}—, and —NR^{a}—S(O)_{w}—;
B is selected from the group consisting of aryl, heteroaryl, heterocyclyl, and hydrogen; wherein aryl and heteroaryl may optionally be substituted by one, two, three or more substituents each independently selected from R^{e}; and wherein heterocyclyl is bound to L through a ring carbon or ring nitrogen and may optionally be substituted by one, two, three or more substituents each independently selected from R^{g}; and wherein if said heterocyclyl contains a —NH moiety that nitrogen may optionally be substituted by R^{h};
R^{X1} is selected from the group consisting of hydrogen, C₁₋₆alkyl and phenyl; or R^{X1} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹⁰, —P(O)(R¹⁰)₂, —C(O)R¹⁰, —C(O)N(R¹⁰)₂, oxo, halogen and cyano;
R^{X11} is selected from the group consisting of hydrogen, C₁₋₆alkyl and phenyl; or R^{X11} and ring A together with the nitrogen to which they are attached form an 8-10 membered saturated or partially unsaturated bicyclic heterocyclyl which may have one or more additional heteroatoms each independently selected from the group consisting of O, S, and N; wherein the heterocyclyl may optionally be substituted by one, two, three or more substituents each independently selected from the group consisting of R¹⁰, —OR¹⁰, —S(O)_{w}R¹⁰ (wherein w is 0, 1 or 2), —N(R¹⁰)₂, —OS(O)_{w}—R¹⁰ (wherein w is 0, 1, or 2), —S(O)_{w}—N(R¹⁰)₂ (wherein w is 0, 1 or 2), —S(O)(NH)R¹, —P(O)(R¹⁰)₂—C(O)R¹⁰, —C(O)N(R¹⁰)₂, oxo, halogen and cyano;
R^{X2} and R^{X3} are each independently selected from the group consisting of hydrogen, halogen, hydroxyl, —NR^{a}R^{b}, —C(O)—NR^{a}R^{b}, C₁₋₆alkyl and C₁₋₆alkoxy; wherein C₁₋₆alkyl and C₁₋₆alkoxy may optionally be substituted by one or more substituents each independently selected from R^{p};
or R^{X2} and R^{X3} together with the carbon to which they are attached form a 3-6 membered carbocycle optionally substituted by one or more substituents each independently selected from the group consisting of hydrogen, halogen, —NR^{a}R^{b}, —C(O)—NR^{a}R^{b}, oxo, C₁₋₆alkyl and C₁₋₆alkoxy;
R^{X4} and R^{X5} are each independently selected from the group consisting of hydrogen and C₁₋₆alkyl; wherein C₁₋₆alkyl may optionally be substituted by one or more substituents each independently selected from R^{P};
R^{X6} and R^{X7} are each independently selected from the group consisting of hydrogen, halogen, —C(O)—NR^{a}R^{b}, cyano and C₁₋₆alkyl; wherein C₁₋₆alkyl may optionally be substituted by one or more substituents each independently selected from R^{P};
R³ is selected from the group consisting of hydrogen, halogen, hydroxyl, C₁₋₆alkyl and NR^{a}R^{b};
R is independently selected, for each occurrence, from the group consisting of halogen, hydroxyl, cyano, oxo, thio, —NR^{a}R^{b}, —CO₂H, —C(O)—NR^{a}R^{b}, —S(O)₂—NR^{a}R^{b}, C₁₋₆alkyl, C₂₋₆alkenyl, C₂₋₆alkynyl, C₃₋₆cycloalkyl, C₁₋₆alkoxy, —S—C₁₋₆alkyl, —S—C₂₋₆alkenyl, C₁₋₆alkyl-O—C₁₋₆alkyl, C₃₋₆alkenyloxy, C₃₋₆alkynyloxy, C₃₋₆cycloalkoxy, C₁₋₆alkylcarbonyl-, C₁₋₆ alkoxycarbonyl, C₁₋₆alkyl-S(O)_{w}— (wherein w is 0, 1 or 2), C₁₋₆alkylcarbonyl-N(R^{a})—, C₁₋₆alkoxycarbonyl-N(R^{a})—, aryl and heteroaryl; wherein C₁₋₆alkyl, C₂₋₆alkenyl, C₂₋₆alkynyl, C₃₋₆cycloalkyl, C₁₋₆alkoxy, —S—C₁₋₆alkyl, —S—C₂₋₆alkenyl, C₁₋₆alkyl-O—C₁₋₆alkyl, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})— and C₁₋₆alkoxycarbonyl-N(R^{a})— may optionally be substituted by one or more substituents each independently selected from R^{P}; and wherein aryl and heteroaryl may optionally be substituted by one or more substituents each independently selected from R^{f};
R^{f} is independently selected, for each occurrence, from the group consisting of R^{P}, C₁₋₆alkyl, —C₁₋₆cycloalkyl, C₂₋₆alkenyl, C₂₋₆alkynyl, C₁₋₆alkoxy, C₁₋₆alkylcarbonyl-, C₁₋₆alkoxycarbonyl, C₁₋₆alkyl-S(O)_{w}—, (wherein w is 0, 1 or 2), C₁₋₆alkylcarbonyl-N(R^{a})— and C₁₋₆ alkoxycarbonyl-N(R^{a})—; wherein C₁₋₆alkyl, C₃₋₆cycloalkyl, C₂₋₆alkenyl, C₂₋₆alkynyl, C₁₋₆ alkoxy, C₁₋₆alkylcarbonyl-, C₁₋₆alkoxycarbonyl, C₁₋₆alkyl-S(O)_{w}—, C₁₋₆alkylcarbonyl-N(R^{a})—, and C₁₋₆alkoxycarbonyl-N(R^{a})— may be optionally substituted by one or more substituents selected from R^{p};
R⁹ is independently selected, for each occurrence, from the group consisting of halogen, hydroxyl, cyano, oxo, thio, —NR^{a}R^{b}, —CO₂H, —C(O)—NR^{a}R^{b}, —S(O)₂—NR^{a}R^{b}, C₁₋₆alkyl, C₂₋₆alkenyl, C₂₋₆alkynyl, C₃₋₆cycloakyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})—, C₁₋₆alkoxycarbonyl-N(R^{a})—, aryl and heteroaryl; wherein C₁₋₆ alkyl, C₂₋₆alkenyl, C₂₋₆alkynyl, C₃₋₆cycloakyl, C₁₋₆alkoxy, C₃₋₆alkenyloxy, C₃₋₆alkynyloxy, C₃₋₆cycloalkoxy, C₁₋₆alkylcarbonyl-, C₁₋₆alkoxycarbonyl, C₁₋₆alkyl-S(O)_{w}— (wherein w is 0, 1 or 2), C₁₋₆alkylcarbonyl-N(R^{a})— and C₁₋₆alkoxycarbonyl-N(R^{a})— may optionally be substituted by one or more substituents each independently selected from R^{P}; and wherein aryl and heteroaryl may optionally be substituted by one or more substituents each independently selected from R^{f};
R^{h} is independently selected, for each occurrence, from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆alkylcarbonyl-, C₁₋₆alkoxycarbonyl-, R^{a}R^{b}N-carbonyl-, R^{a}R^{b}N—SO₂— and aryl; wherein C₁₋₆alkyl, —C₁₋₆alkenyl, C₃₋₆alkynyl, C₃₋₆cycloakyl, C₁₋₆alkyl-S(O)₂—, C₁₋₆alkylcarbonyl- and C₁₋₆alkoxycarbonyl- may optionally be substituted by one or more substituents selected from R^{P}; and wherein aryl may optionally be substituted by one or more substituents each independently selected from R^{f};
R^{a} and R^{b} are independently selected, for each occurrence, from the group consisting of hydrogen, C₁₋₆alkyl, C₃₋₆cycloalkyl, and phenyl; wherein C₁₋₆alkyl may optionally be substituted by one or more substituents selected from halogen, cyano, oxo, heterocyclyl and hydroxyl; and wherein phenyl may optionally be substituted by C₁₋₃alkyl, C₁₋₃alkoxy, halogen, hydroxyl;
or R^{a} and R^{b}, together with the nitrogen to which they are attached, may form a 4-6 membered monocyclic heterocyclic ring, which may have an additional heteroatom selected from the group consisting of O, S, and N; wherein the 4-6 membered heterocyclic ring may optionally be substituted by one or more substituents selected from the group consisting of halogen, cyano, oxo, hydroxyl and C₁₋₃alkyl (optionally substituted with —NH₂); and
R^{P} is independently selected, for each occurrence, from the group consisting of halogen, hydroxyl, cyano, C₁₋₆alkoxy, R^{a}R^{b}N—, R^{a}R^{b}N-carbonyl-, R^{a}R^{b}N—SO₂—, and R^{a}R^{b}N-carbonyl-N(R^{a})—.

179. The method of claim 178, wherein X is selected from the group consisting of a bond, —O—, —NR^{X1}— and —NR^{X11}—.

180. The method of claim 178 or 179, wherein A is selected from the group consisting of phenyl, pyridyl, quinolinyl, indolyl, and indolinyl, wherein phenyl, pyridyl, quinolinyl, indolyl and indolinyl may optionally be substituted by one, two or three substituents each independently selected from the group consisting of —R¹, —OR¹⁰, methylenedioxy, —SR¹, —N(R¹⁰)₂, —OSO₂R¹⁰, —SO₂R¹⁰, —C(O)N(R¹⁰)₂, halogen, and cyano.

181. The method of any one of claims 178-180, wherein A is phenyl; wherein phenyl may optionally be substituted by one, two, or three substituents each independently selected from the group consisting of —R¹⁰, —OR¹⁰, methylenedioxy, halogen, and cyano.

182. The method of any one of claims 177-180, wherein A is pyridyl; wherein pyridyl may optionally be substituted by one, two, or three substituents each independently selected from the group consisting of —R¹⁰, —OR¹⁰, halogen, and cyano.

183. The method of claim 178 or 179, wherein R^{X1} and ring A together with the nitrogen to which they are attached form a 1,2,3,4-tetrahydro-1,5-naphthyridine moiety.

184. The method of claim 178 or 179, wherein R^{X1} and ring A together with the nitrogen to which they are attached form a 1,2,3,4-tetrahydro-1,5-naphthyridine moiety.

185. The method of claim 178 or 179, wherein R^{X1} and ring A together with the nitrogen to which they are attached form a 1,2,3,4-tetrahydroquinoline moiety.

186. The method of claim 178 or 179, wherein R^{X11} and ring A together with the nitrogen to which they are attached form a 1,2,3,4-tetrahydroquinoline moiety.

187. The method of any one of claims 178-186, wherein R³ is selected from the group consisting of hydrogen, CH₃ and —NH₂.

188. The method of any one of claims 178-187, wherein Y is selected from the group consisting of —C(O)—N(R^{a})—, —C(O)—O—, —CH₂—C(O)—O—, —C(O)—O—CH₂—CH₂—, —C(O)—, —CH₂—, —CH₂C(O)—, —CH₂—S(O)_{w}—, —CH₂—N(R^{a})—CH₂—, and —CH₂—N(R^{a})—.

189. The method of any one of claims 178-188, wherein Y is selected from the group consisting of —CH₂—S—, —CH₂—N(Me)-CH₂— and —CH₂—NH—CH₂—.

190. The method of any one of claims 178-189, wherein B is selected from the group consisting of 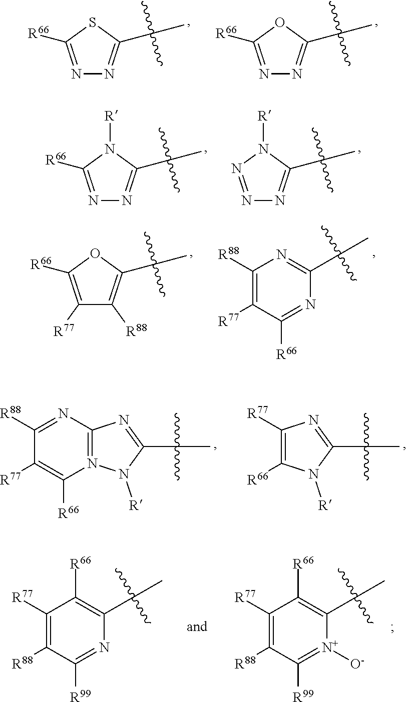
wherein
R 6, R⁷⁷, R⁸⁸ and R⁹⁹ are each independently selected from the group consisting of is selected from the group of hydrogen, halogen, —NR^{a}R^{b}, C₁₋₆alkyl, C₃₋₆cycloalkyl, C₁₋₆alkoxy, —S—C₂₋₆alkenyl, C₁₋₆alkylcarbonyl-N(R^{a})—, heteroaryl and phenyl (optionally substituted by one, two or three hydroxyl, halogen, C₁₋₃alkyl or C₁₋₃alkoxy groups); and
R′ is selected from the group consisting of hydrogen, —NR^{a}R^{b}, C₁₋₆alkyl, C₃₋₆cycloalkyl, C₁₋₃alkyl-O—C₁₋₃alkyl, and phenyl (optionally substituted by hydroxyl or C₁₋₃alkoxy).

191. The method of any one of claims 178-190, wherein B is selected from the group consisting of

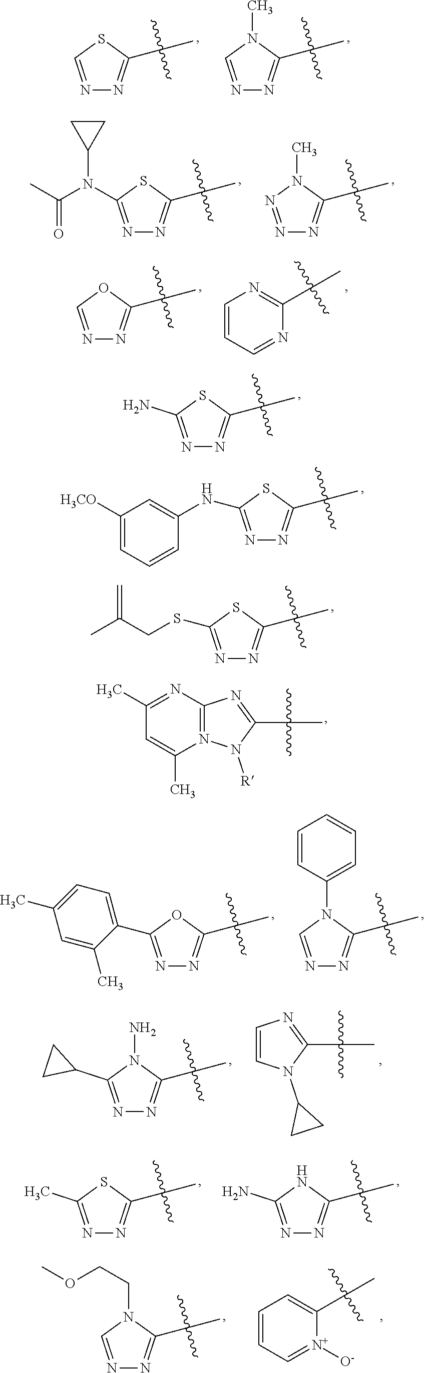

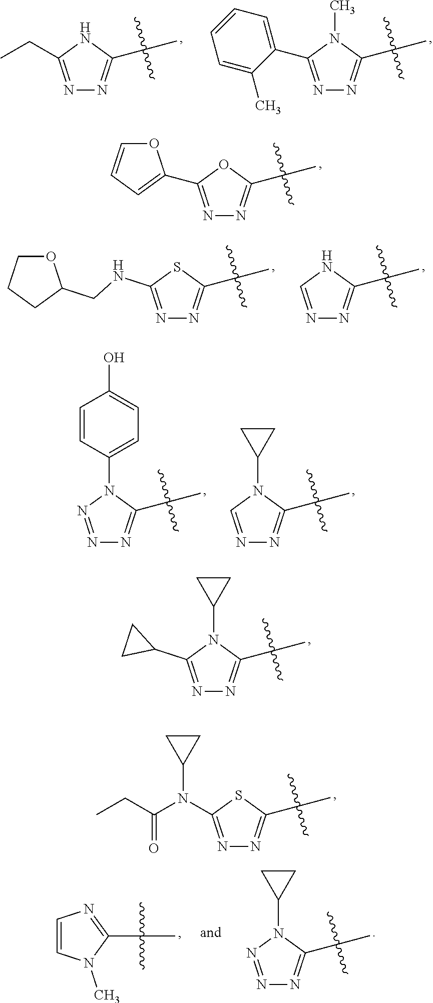

192. The method of any one of claims 178-189, wherein B is selected from the group consisting of 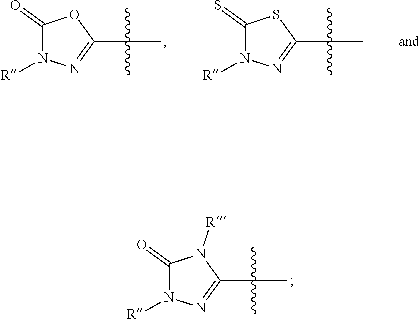
wherein
R″ and R″′ are each independently selected from the group consisting of hydrogen, C₁₋₆alkyl, C₃₋₆cycloalkyl, and phenyl; wherein phenyl may optionally be substituted by one or two substituents each independently selected from the group consisting of halogen, hydroxyl, C₁₋₁₀alkyl and C₁₋₃alkoxy.

193. The method of claim 192, wherein B is selected from the group consisting of

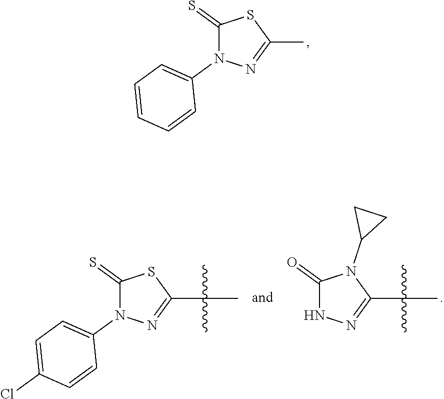

194. The method of any one of claims 178-187, wherein Y is selected from the group consisting of a bond, —C(O)—, —CH₂—, and —CH₂C(O)—.

195. The method of claim 194, wherein B is represented by: 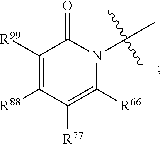
wherein
R⁶⁶, R⁷⁷, R⁸⁸ and R⁹⁹ are each independently selected from the group consisting of is selected from the group of hydrogen, halogen, C₁₋₃alkyl, C₃₋₆cycloalkyl, and C₁₋₃alkoxy; wherein C₁₋₃alkyl, C₃₋₆cycloalkyl, and C₁₋₃alkoxy may optionally be substituted by one, two or three halogens.

196. The method of claim 194 or 195, wherein B is represented by:

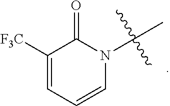 [SMILES: *n1cccc(C(F)(F)F)c1=O]

197. The method of claim 194, wherein B is selected from the group consisting of pyrrolidinyl, piperidinyl, azepanyl and morpholinyl; wherein pyrrolidinyl, piperidinyl, azepanyl and morpholinyl are bound to L through a ring nitrogen and may optionally be substituted by one, two, three or more substituents each independently selected from the group consisting of halogen, hydroxyl, cyano, —NR^{a}R^{b}, —C(O)—NR^{a}R^{b}, C₁₋₃alkyl, and C₁₋₃ alkoxy; wherein C₁₋₃alkyl and C₁₋₃alkoxy may optionally be substituted by halogen, hydroxyl and —NR^{a}R^{b}.

198. The method of claim 197, wherein ring B is substituted by one, two, three or more substituents each independently selected from the group consisting of —NH₂, —CH₃, and —CH₂NH₂, —C(H)NH₂CH₃ and —CH₂NHCH₃.

199. The method of any one of claims 178-187, wherein Y is selected from the group consisting of —C(O)—NH—, —C(O)—NMe-, —CH₂—NH—, and, —CH₂—NMe-.

200. The method of claim 199, wherein B is phenyl; wherein phenyl may optionally be substituted by one, two or three substituents each independently selected from the group consisting of halogen, hydroxyl, methylenedioxy, C₁₋₃alkyl, and C₁₋₃alkoxy; and wherein C₁₋₃ alkyl, and C₁₋₃alkoxy may optionally be substituted by one or more fluorine atoms.

201. The method of any one of claims 178-187, wherein Y is selected from the group consisting of —C(O)—O—, —CH₂—C(O)—O— and —C(O)—O—CH₂—CH₂—CH₂—.

202. The method of claim 201, wherein B is hydrogen.

203. A method of treating a disorder in a patient in need thereof, comprising administering to the patient an effective amount of a compound of Formula XIII, or a pharmaceutically acceptable salt or stereoisomer thereof, wherein Formula XIII is represented by: 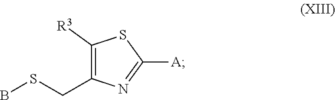
wherein
A is selected from the group consisting of phenyl, a 5-7 membered monocyclic heteroaryl having one or more heteroatoms each independently selected from O, S, and N, a 8-10 membered bicyclic heteroaryl having one or more heteroatoms each independently selected from the group consisting of O, S, and N, and a 4-7 membered heterocyclyl; wherein A may optionally be substituted by one, two, three or more substituents each independently selected from the group consisting of —R¹⁰, —OR¹⁰, methylenedioxy, —N(R¹⁰)₂, —C(O)N(R¹⁰)₂, oxo, halogen and cyano;
R¹⁰ is independently selected from the group consisting of hydrogen, C₁₋₆alkyl, C₁₋₆haloalkyl, C₃₋₆cycloalkyl, heterocycloalkyl, aryl, and heteroaryl, where R¹⁰ may optionally be substituted by one, two, three or more substituents each independently selected from the group consisting of halogen, —C(O)R²⁰, C₁₋₆alkyl and C₁₋₆haloalkyl;
R²⁰ is selected from the group consisting of hydroxyl, halogen, and C₁₋₆alkyl;
B is selected from the group consisting of: 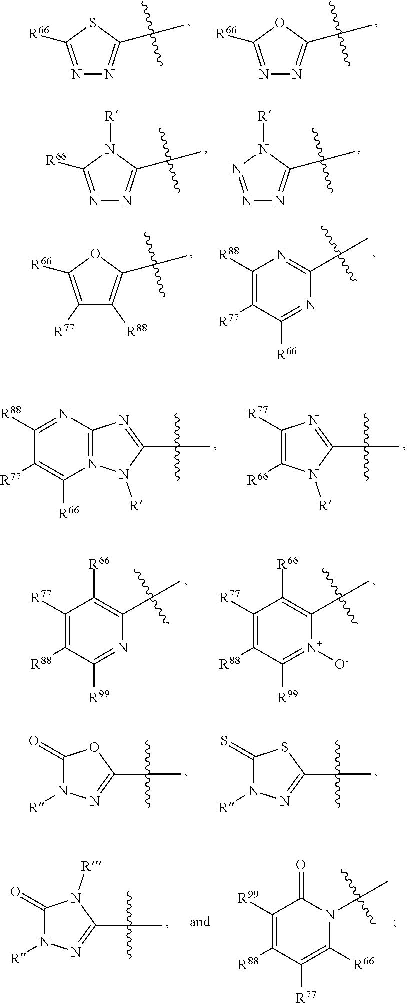
R³ is selected from the group consisting of hydrogen, halogen, hydroxyl, C₁₋₆alkyl and —NR^{a}R^{b};
R⁶⁶, R⁷⁷, R⁸⁸ and R⁹⁹ are each independently selected from the group consisting of hydrogen, halogen, —NR^{a}R^{b}, C₁₋₆alkyl, C₃₋₆cycloalkyl, C₁₋₆alkoxy, —S—C₂₋₆alkenyl, C₁₋₆alkylcarbonyl-N(R^{a})—, heteroaryl and phenyl; wherein heteroaryl and phenyl may optionally be substituted by one, two or three substituents each independently selected from the group consisting of hydroxyl, halogen, C₁₋₃alkyl and C₁₋₃alkoxy; and wherein C₁₋₆alkyl, C₃₋₆cycloalkyl, and C₁₋₆alkoxy may optionally be substituted by one, two or three halogens; and
R^{a} and R^{b} are independently selected, for each occurrence, from the group consisting of hydrogen, C₁₋₆alkyl, C₃₋₆cycloalkyl, and phenyl; wherein C₁₋₆alkyl may optionally be substituted by one or more substituents selected from halogen, cyano, oxo, heterocyclyl and hydroxyl; and wherein phenyl may optionally be substituted by C₁₋₃alkyl, C₁₋₃alkoxy, halogen, hydroxyl;
or R^{a} and R^{b}, together with the nitrogen to which they are attached, may form a 4-6 membered monocyclic heterocyclic ring, which may have an additional heteroatom selected from the group consisting of O, S, and N; wherein the 4-6 membered heterocyclic ring may optionally be substituted by one or more substituents selected from the group consisting of halogen, cyano, oxo, hydroxyl and C₁₋₃alkyl (optionally substituted with —NH₂).

204. A method of treating a disorder in a patient in need thereof, comprising administering to the patient an effective amount of a compound of Formula XIV, or a pharmaceutically acceptable salt or stereoisomer thereof, wherein Formula XIV is represented by: 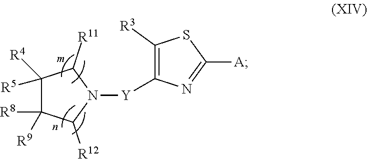
wherein
A is selected from the group consisting of phenyl, a 5-7 membered monocyclic heteroaryl having one or more heteroatoms each independently selected from O, S, and N, a 8-10 membered bicyclic heteroaryl having one or more heteroatoms each independently selected from the group consisting of O, S, and N, and a 4-7 membered heterocyclyl; wherein A may optionally be substituted by one, two, three or more substituents each independently selected from the group consisting of —R¹⁰, —OR¹⁰, methylenedioxy, —N(R¹⁰)₂, —C(O)N(R¹⁰)₂, oxo, halogen and cyano;
R¹⁰ is independently selected from the group consisting of hydrogen, C₁₋₆alkyl, C₁₋₆ haloalkyl, C₃₋₆cycloalkyl, heterocycloalkyl, aryl, and heteroaryl, where R¹⁰ may optionally be substituted by one, two, three or more substituents each independently selected from the group consisting of halogen, —C(O)R²⁰, C₁₋₆alkyl and C₁₋₆haloalkyl;
R²⁰ is selected from the group consisting of hydroxyl, halogen, and C₁₋₆alkyl;
R³ is selected from the group consisting of hydrogen, halogen, hydroxyl, C₁₋₆alkyl and —NR^{a}R^{b};
Y is selected from the group consisting of a bond, —C(O)—, —CH₂—, and —CH₂C(O)—;
R⁴ and R⁵ are each independently selected from the group consisting of hydrogen, hydroxyl, C₁₋₆alkyl, C₁₋₆alkoxy, C₁₋₆alkyl-O—R⁶, —C(O)N(R⁶)₂, —N(R⁶)₂, halogen, C₁₋₆alkyl-N(R⁶)₂, and cyano; wherein C₁₋₆alkyl, C₁₋₆alkoxy, C₁₋₆alkyl-O—R⁶, and C₁₋₆alkyl-N(R⁶)₂ may optionally be substituted by one or more substituents each independently selected from the group consisting of hydroxyl, —NH₂, oxo, and halogen,
or R⁴ and R⁵, taken together with the atoms to which they are attached, form a 3-7 membered carbocyclic or heterocyclic saturated or partially unsaturated ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;
or R⁴ and R⁸, taken together with the atoms to which they are attached, form a 4-7 membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;
or R⁴ is absent, and W and R, taken together with the atoms to which they are attached, form a 3-membered carbocyclic or heterocyclic ring, wherein the ring may optionally be substituted by one or more substituents each independently selected from the group consisting of hydroxyl, C₁₋₆alkyl, C₁₋₆alkoxy, —N(R⁶)₂, halogen, oxo, and cyano;
R⁶ is independently for each occurrence selected from the group consisting of hydrogen, C₁₋₆alkyl and phenyl;
R¹¹ and R¹² are each independently selected from the group consisting of hydrogen, hydroxyl, C₁₋₆alkyl, C₁₋₆alkoxy, C₁₋₆alkyl-O—R⁶, aryl, arylalkylene, heteroaryl, heteroarylalkylene, —C(O)N(R⁶)₂, —N(R⁶)₂, halogen, C₁₋₆alkyl-N(R⁶)₂, —CO₂H, and cyano; wherein C₁₋₆alkyl, C₁₋₆alkoxy, C₁₋₆alkyl-O—R⁶, aryl, arylalkylene, heteroaryl, heteroarylalkylene, and C₁₋₆alkyl-N(R⁶)₂ may optionally be substituted by one or more substituents each independently selected from the group consisting of hydroxyl, —NH₂, and halogen;
or R¹¹ and R¹², taken together with the atoms to which they are attached, form a 5-7 membered heterocyclic ring;
or R⁴ and R¹², taken together with the atoms to which they are attached, form a 5-7 membered carbocyclic or heterocyclic ring;
or R⁸ and R¹¹, taken together with the atoms to which they are attached, form a 5-7 membered carbocyclic or heterocyclic ring;
R⁸ and R⁹ are each independently selected from the group consisting of hydrogen, C₁₋₆)alkyl, C₁₋₆alkyl-N(R⁶)₂, —OR⁶, C₁₋₆alkyl-O—R⁶, —C(O)NH₂, —N(R⁶)₂, halogen, and cyano; and
each of m and n is, independently, 0, 1, 2, or 3, with m+n being at least 2 and no more than 4.

205. The method of claim 204, wherein A is phenyl; wherein phenyl may optionally be substituted by one, two, or three substituents each independently selected from the group consisting of —OR¹⁰, methylenedioxy, halogen, and cyano.

206. The method of claim 204 or 205, wherein R³ is selected from the group consisting of hydrogen, CH₃ and —NH₂.

207. The method of any of claims 204-206, wherein m is an integer selected from 1 or 2; and n is 1.

208. The method of any of claims 204-207, wherein R⁴ and R are each independently selected from the group consisting of hydrogen, hydroxyl, C₁₋₃alkyl, C₁₋₃alkoxy, C₁₋₃alkyl-O—R⁶, —C(O)NH₂, —N(R⁶)₂, halogen, C₁₋₃alkyl-N(R⁶)₂, and cyano; wherein C₁₋₃alkyl, C₁₋₃alkoxy, C₁₋₃alkyl-O—R⁶ and C₁₋₃alkyl-N(R⁶)₂ may optionally be substituted by one or more substituents each independently selected from the group consisting of hydroxyl, —N(R⁶)₂, and halogen;
or R⁴ and R⁵, taken together with the atoms to which they are attached, form a 3-7 membered carbocyclic or heterocyclic ring;
or R⁴ and R⁸, taken together with the atoms to which they are attached, form a 4-7 membered carbocyclic or heterocyclic ring;
or R⁴ is a bond, and R⁴ and R, taken together with the atoms to which they are attached, form a 3-membered carbocyclic or heterocyclic ring; and
each R⁶ is independently hydrogen or C₁₋₃alkyl.

209. The method of any one of claims 204-208, wherein R⁴ and R⁵ are each independently selected from the group consisting of C₁₋₃alkyl and —NR^{a}R^{b}; wherein C₁₋₃alkyl may optionally be substituted by one, two or three substituents each independently selected from the group consisting of halogen, hydroxyl and —NR^{a}R^{b}.

210. The method of any one of claims 204-209, wherein R⁴ and R⁵ are each independently selected from the group consisting of —NH₂, —CH₃, and —CH₂NH₂, —C(H)NH₂CH₃ and —CH₂NHCH₃.

211. The method of any one of claims 178-210, wherein the compound is selected from the group consisting of: 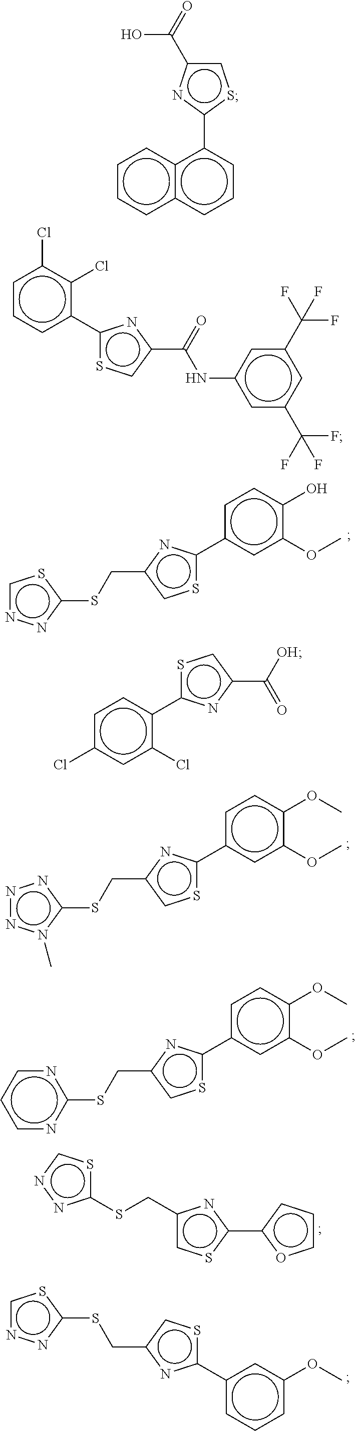 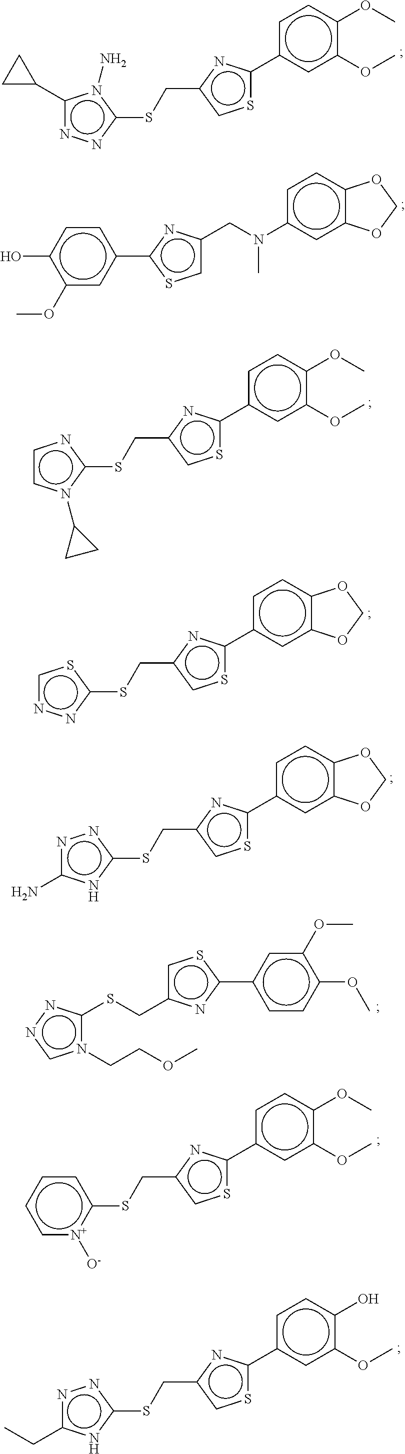 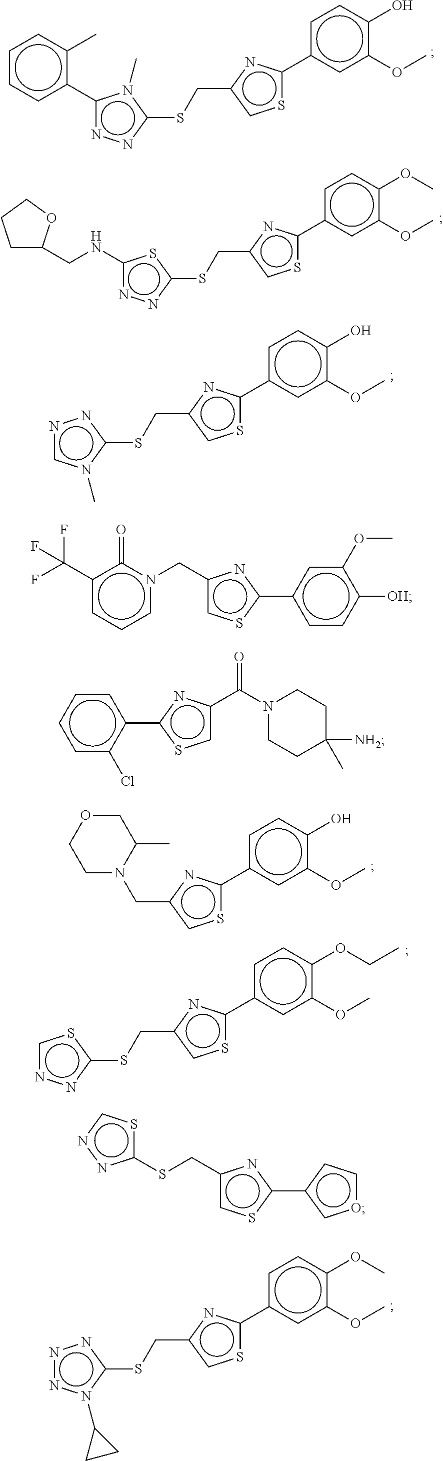 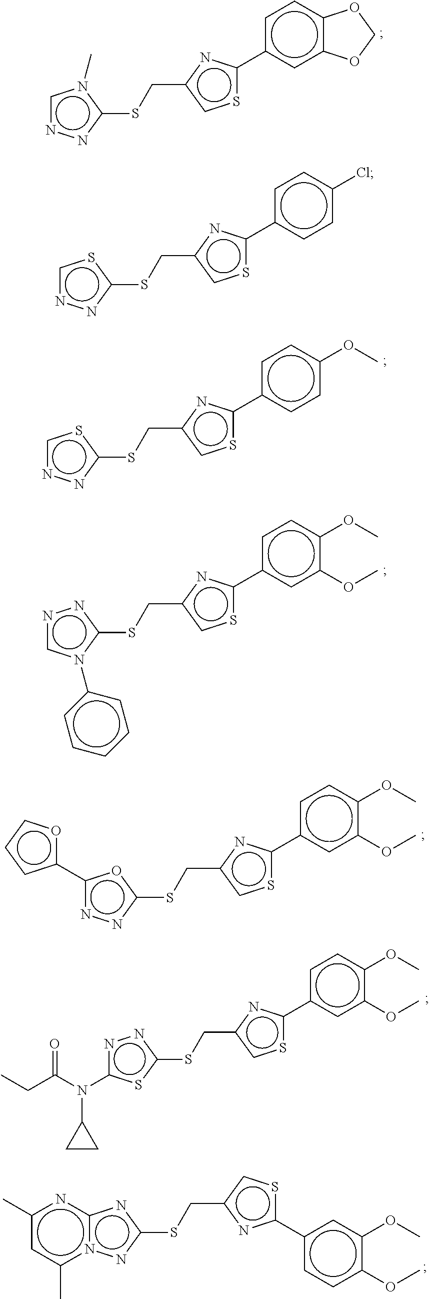 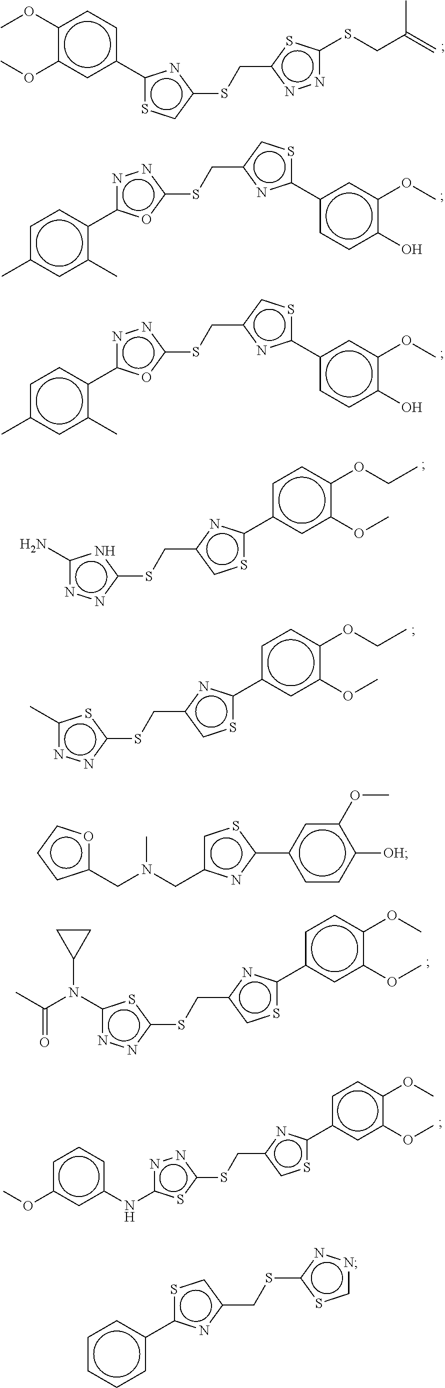 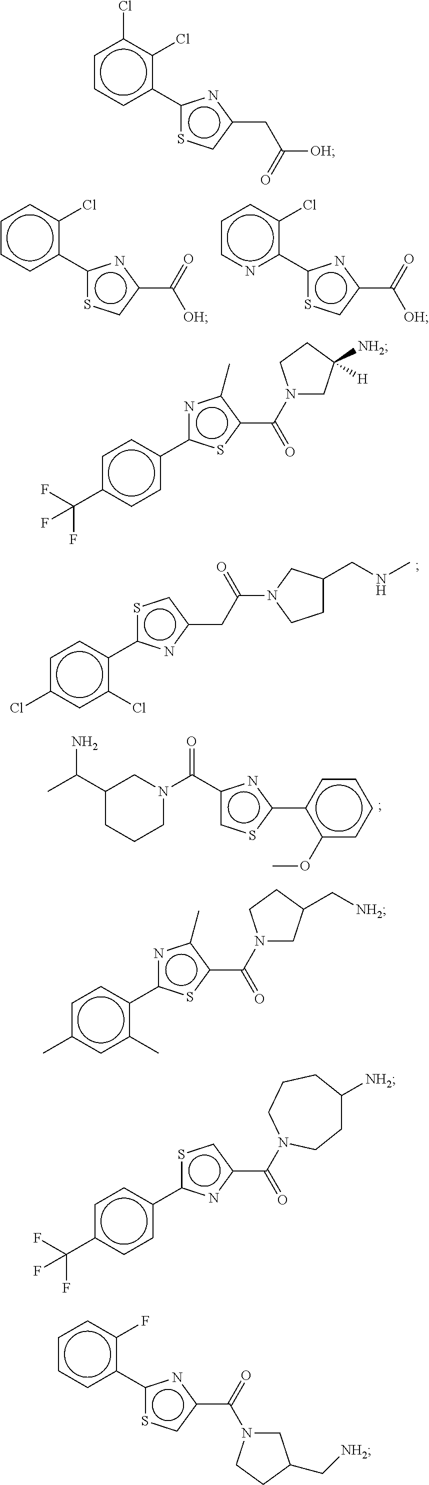
and pharmaceutically acceptable salts or stereoisomers thereof.

212. The method of any one of claims 178-211, further comprising administration of a therapeutically effective amount of an antibody, an antibody-drug conjugate, an immunomodulator, or a histone deacetylase inhibitor.

213. The method of any one of claims 178-212, wherein the disorder is Noonan syndrome.

214. The method of any one of claims 178-212, wherein the disorder is neutropenia.

215. The method of any one of claims 178-212, wherein the disorder is diabetes.

216. The method of any one of claims 178-212, wherein the disorder is neuroblastoma.

217. The method of any one of claims 178-212, wherein the disorder is melanoma.

218. The method of any one of claims 178-212, wherein the disorder is acute myeloid leukemia.

219. The method of any one of claims 178-212, wherein the disorder is juvenile leukemia.

220. The method of any one of claims 178-212, wherein the disorder is juvenile myelomonocytic leukemia.

221. The method of any one of claims 178-212, wherein the disorder is breast cancer.

222. The method of any one of claims 178-212, wherein the disorder is lung cancer.

223. The method of any one of claims 178-212, wherein the disorder is colorectal cancer.
